(19) United States
(12) Patent Application Publication
(10) Pub. No.: US 2021/0386788 A1
(43) Pub. Date: Dec. 16, 2021

(54) ER TUNABLE PROTEIN REGULATION

(71) Applicant: Obsidian Therapeutics, Inc., Cambridge, MA (US)

(72) Inventors: Vipin SURI, Belmont, MA (US); Dexue SUN, Cambridge, MA (US); Michelle Lynn OLS, Northborough, MA (US); Scott Francis HELLER, Stoughton, MA (US); Dan Jun LI, Cambridge, MA (US); Celeste RICHARDSON, Brookline, MA (US); Mara Christine INNISS, Beverly, MA (US); Jennifer Leah GORI, Jamaica Plain, MA (US); Benjamin J. PRIMACK, Brookline, MA (US); Abhishek KULKARNI, Brookline, MA (US); Brian DOLINSKI, Cambridge, MA (US); Tucker Read EZELL, Cambridge, MA (US); Michael SCHEBESTA, Cambridge, MA (US); James A. STORER, Medford, MA (US); Kutlu Goksu ELPEK, Arlington, MA (US)

(73) Assignee: Obsidian Therapeutics, Inc., Cambridge, MA (US)

(21) Appl. No.: 17/288,373

(22) PCT Filed: Oct. 23, 2019
(86) PCT No.: PCT/US2019/057698

§ 371 Date: Apr. 23, 2021

Related U.S. Application Data
(60) Provisional application No. 62/860,368, filed on Jun. 12, 2019.
(60) Provisional application No. 62/749,763, filed on Oct. 24, 2018.

(51) Int. Cl. A61K 35/28; A61K 31/4535; A61K 31/55; C07K 14/705; C07K 16/28; C07K 14/725; C07K 14/54; C07K 14/715; A61P 35/00

(52) U.S. Cl. CPC A61K 35/28; A61K 31/4535; A61K 31/55; C07K 14/70517; C07K 14/70578; A61P 35/00; C07K 14/7051; C07K 14/5434; C07K 14/5443; C07K 14/7155; C07K 16/2803

(57) ABSTRACT

The present disclosure is related to compositions and methods for the regulated and controlled expression of proteins.

### CROSS REFERENCE TO RELATED APPLICATIONS

This application claims priority to the U.S. Provisional Patent Application No. 62/749,763 filed on Oct. 24, 2018, entitled ER TUNABLE PROTEIN REGULATION, and the U.S. Provisional Patent Application No. 62/860,368 filed on Jun. 12, 2019, entitled ER TUNABLE PROTEIN REGULATION, the contents of each of which are herein incorporated by reference in their entirety.

### REFERENCE TO THE SEQUENCE LISTING

The present application is being filed along with a Sequence Listing in electronic format. The Sequence Listing is provided as a file entitled 2095_1006PCT2_SL_ST25.txt created on Oct. 23, 2019, which is 1,185,388 bytes in size. The information in the electronic format of the sequence listing is incorporated herein by reference in its entirety.

### FIELD

The present disclosure relates to regulatable tunable biocircuit systems for the development of controlled and/or regulated therapeutic systems. In particular, the present disclosure provides destabilizing domains derived from human estrogen receptor (ER) which can tune protein stability of at least one payload, compositions and methods of use thereof. Regulatable biocircuits, effector modules and stimulus response elements containing destabilizing domains (DD) derived from human estrogen receptor protein (ER) are also disclosed.

### BACKGROUND

Safe and effective gene therapy requires tightly regulated expression of a therapeutic transgenic product (e.g., the protein product). Similarly, the analysis of gene function in development, cell differentiation and other physiological activities requires the controllable expression of a protein under investigation. However, current technologies do not allow titration of the timing or levels of target protein induction. Inadequate exogenous and/or endogenous gene control is a critical issue in numerous gene therapy settings. This lack of tunability also makes it difficult to safely express proteins with narrow or uncertain therapeutic windows or those requiring more titrated or transient expression.

However, when a specific small molecule ligand binds its intended DD as a ligand binding partner, the instability is reversed, and protein function is restored. Such a system is herein referred to as a biocircuit, with the canonical DD-containing biocircuit described above being the prototypical model biocircuit

It is believed that improvements of biocircuits, including those containing DDs can form the basis of a new class of cell and gene therapies that employ tunable and temporal control of gene expression and function. Such novel moieties are described by the present inventors as stimulus response elements (SREs) which act in the context of an effector module to complete a biocircuit arising from a stimulus and ultimately producing a signal or outcome. When properly formatted with a polypeptide payload, and when activated by a particular stimulus, e.g., a small molecule, biocircuit systems can be used to regulate transgene and/or protein levels either up or down by perpetuating a stabilizing signal or destabilizing signal. This approach has many advantages over existing methods of regulating protein function and/or expression, which are currently focused on top level transcriptional regulation via inducible promoters.

The present disclosure provides novel protein domains, in particular destabilizing domains (DDs) derived from human estrogen receptor (ER) that display small molecule dependent stability, and the biocircuit systems and effector modules comprising such DDs. Methods for tuning transgene functions using the same are also provided.

### SUMMARY

The present disclosure provides novel protein domains displaying small molecule dependent stability. Such protein domains are called destabilizing domains (DDs). In the absence of its binding ligand, the DD is destabilizing and causes degradation of a payload fused to the DD (e.g., a protein of interest (POI), while in the presence of its binding ligand, the fused DD and payload can be stabilized, and its stability is dose dependent. These systems are further taught in co-owned U.S. Provisional Patent Application No. 62/320,864 filed Apr. 11, 2016, 62/466,596 filed Mar. 3, 2017 and the International Publication WO2017/180587 (the contents of each of which are herein incorporated by reference in their entirety).

In some embodiments, the present disclosure describes biocircuit systems. Such systems may include at least one effector module. The effector module may include (a) a stimulus response element (SRE) and (b) at least one payload. The payload may include a protein of interest which is attached, appended or associated with the SRE. In one aspect, the SRE includes a destabilizing domain (DD). The DD may include in whole or in part, the human estrogen receptor (ER, having the amino acid sequence of:

SEQ ID NO. 1)
Met Thr Met Thr Leu His Thr Lys Ala Ser Gly Met
Ala Leu Leu His Gln Ile Gln Gly Asn Glu Leu Glu
Pro Leu Asn Arg Pro Gln Leu Lys Ile Pro Leu Glu
Arg Pro Leu Gly Glu Val Tyr Leu Asp Ser Ser Lys
Pro Ala Val Tyr Asn Tyr Pro Glu Gly Ala Ala Tyr
Glu Phe Asn Ala Ala Ala Ala Ala Asn Ala Gln Val
Tyr Gly Gln Thr Gly Leu Pro Tyr Gly Pro Gly Ser
Glu Ala Ala Ala Phe Gly Ser Asn Gly Leu Gly Gly
Phe Pro Pro Leu Asn Ser Val Ser Pro Ser Pro Leu
Met Leu Leu His Pro Pro Pro Gln Leu Ser Pro Phe
Leu Gln Pro His Gly Gln Gln Val Pro Tyr Tyr Leu
Glu Asn Glu Pro Ser Gly Tyr Thr Val Arg Glu Ala
Gly Pro Pro Ala Phe Tyr Arg Pro Asn Ser Asp Asn
Arg Arg Gln Gly Gly Arg Glu Arg Leu Ala Ser Thr
Asn Asp Lys Gly Ser Met Ala Met Glu Ser Ala Lys
Glu Thr Arg Tyr Cys Ala Val Cys Asn Asp Tyr Ala
Ser Gly Tyr His Tyr Gly Val Trp Ser Cys Glu Gly
Cys Lys Ala Phe Phe Lys Arg Ser Ile Gln Gly His
Asn Asp Tyr Met Cys Pro Ala Thr Asn Gln Cys Thr
Ile Asp Lys Asn Arg Arg Lys Ser Cys Gln Ala Cys
Arg Leu Arg Lys Cys Tyr Glu Val Gly Met Met Lys
Gly Gly Ile Arg Lys Asp Arg Arg Gly Gly Arg Met
Leu Lys His Lys Arg Gln Arg Asp Asp Gly Glu Gly
Arg Gly Glu Val Gly Ser Ala Gly Asp Met Arg Ala
Ala Asn Leu Trp Pro Ser Pro Leu Met Ile Lys Arg
Ser Lys Lys Asn Ser Leu Ala Leu Ser Leu Thr Ala
Asp Gln Met Val Ser Ala Leu Leu Asp Ala Glu Pro
Pro Ile Leu Tyr Ser Glu Tyr Asp Pro Thr Arg Pro
Phe Ser Glu Ala Ser Met Met Gly Leu Leu Thr Asn
Leu Ala Asp Arg Glu Leu Val His Met Ile Asn Trp
Ala Lys Arg Val Pro Gly Phe Val Asp Leu Thr Leu
His Asp Gln Val His Leu Leu Glu Cys Ala Trp Leu
Glu Ile Leu Met Ile Gly Leu Val Trp Arg Ser Met
Glu His Pro Gly Lys Leu Leu Phe Ala Pro Asn Leu
Leu Leu Asp Arg Asn Gln Gly Lys Cys Val Glu Gly
Met Val Glu Ile Phe Asp Met Leu Leu Ala Thr Ser
Ser Arg Phe Arg Met Met Asn Leu Gln Gly Glu Glu
Phe Val Cys Leu Lys Ser Ile Ile Leu Leu Asn Ser
Gly Val Tyr Thr Phe Leu Ser Ser Thr Leu Lys Ser
Leu Glu Glu Lys Asp His Ile His Arg Val Leu Asp
Lys Ile Thr Asp Thr Leu Ile His Leu Met Ala Lys
Ala Gly Leu Thr Leu Gln Gln Gln His Gln Arg Leu
Ala Gln Leu Leu Leu Ile Leu Ser His Ile Arg His
Met Ser Asn Lys Gly Met Glu His Leu Tyr Ser Met
Lys Cys Lys Asn Val Val Pro Leu Tyr Asp Leu Leu
Leu Glu Met Leu Asp Ala His Arg Leu His Ala Pro
Thr Ser Arg Gly Gly Ala Ser Val Glu Glu Thr Asp
Gln Ser His Leu Ala Thr Ala Gly Ser Thr Ser Ser
His Ser Leu Gln Lys Tyr Tyr Ile Thr Gly Glu Ala
Glu Gly Phe Pro Ala Thr Val.

In one embodiment, the DD includes a ligand binding domain of ER (SEQ ID NO. 1). The ligand, binding domain may include amino acids 305 to 509 of ER (SEQ ID NO. 2). In some aspects, the DD may include least one mutation, occurring at position 413 (N413) or at position 502 (Q502), relative to SEQ ID NO. 1. In one embodiment, the mutation may be at position 413 (N413) relative to SEQ ID NO. 1. The mutation at N413 may be N413D, N413T, N413H, N413A, N413Q, N413V, N413C, N413K, N413M, N413R, N413S, N413W, N413I, N413E, N413L, N413P, N413F, N413Y, and N413G. In another aspect, the mutation may be at position 502 (Q502) relative to SEQ ID NO. 1. The mutation at Q502 may be Q502H, Q502D, Q502E, Q502V, Q502A, Q502T, Q502N, Q502K, Q5025, Q502L, Q502Y, Q502W, Q502F, Q502I, Q502G, Q502P, Q502M, and Q502C. In one aspect, at least one mutation may be N413D. In one aspect at least one mutation may be N413T. In one aspect, at least one mutation may be Q502H. In one aspect, the DD includes at least two mutations such as, but not limited to N413T and Q502H or N413D and Q502H.

The DD may further comprise one or more mutations independently selected from L384M, M421G, G521R or Y537S.

In some embodiments, the DD may be selected from but not limited to ER (aa 305-549 of WT, L384M, N413D, M421G, G521R, Y537S) (SEQ ID NO. 19), ER (aa 305-549 of WT, L384M, N413F, M421G, G521R, Y537S) (SEQ ID NO. 27), ER (aa 305-549 of WT, L384M, N413L, M421G, G521R, Y537S) (SEQ ID NO. 29), ER (aa 305-549 of WT, L384M, N413Y, M421G, G521R, Y537S) (SEQ ID NO. 31), ER (aa 305-549 of WT, L384M, N413H, M421G, G521R, Y537S) (SEQ ID NO. 33), ER (aa 305-549 of WT, L384M, N413Q, M421G, G521R, Y537S) (SEQ ID NO. 35), ER (aa 305-549 of WT, L384M, N413I, M421G, G521R, Y537S) (SEQ ID NO. 37), ER (aa 305-549 of WT, L384M, N413M, M421G, G521R, Y537S) (SEQ ID NO. 39), ER (aa 305-549 of WT, L384M, N413K, M421G, G521R, Y537S) (SEQ ID NO. 41), ER (aa 305-549 of WT, L384M, N413V, M421G, G521R, Y537S) (SEQ ID NO. 43), ER (aa 305-549 of WT, L384M, N413S, M421G, G521R, Y537S) (SEQ ID NO. 45), ER (aa 305-549 of WT, L384M, N413C, M421G, G521R, Y537S) (SEQ ID NO. 47), ER (aa 305-549 of WT, L384M, N413W, M421G, G521R, Y537S) (SEQ ID NO. 49), ER (aa 305-549 of WT, L384M, N413P, M421G, G521R, Y537S) (SEQ ID NO. 51), ER (aa 305-549 of WT, L384M, N413R, M421G, G521R, Y537S) (SEQ ID NO. 53), ER (aa 305-549 of WT, L384M, N413T, M421G, G521R, Y537S) (SEQ ID NO. 55), ER (aa 305-549 of WT, L384M, N413A, M421G, G521R, Y537S) (SEQ ID NO. 57), ER (aa 305-549 of WT, L384M, N413E, M421G, G521R, Y537S) (SEQ ID NO. 59), ER (aa 305-549 of WT, L384M, N413G, M421G, G521R, Y537S) (SEQ ID NO. 61), ER (aa 305-549 of WT, L384M, M421G, Q502F, G521R, Y537S) (SEQ ID NO. 63), ER (aa 305-549 of WT, L384M, M421G, Q502L, G521R, Y537S) (SEQ ID NO. 65), ER (aa 305-549 of WT, L384M, M421G, Q502Y, G521R, Y537S) (SEQ ID NO. 67), ER (aa 305-549 of WT, L384M, M421G, Q502H, G521R, Y537S) (SEQ ID NO. 69), ER (aa 305-549 of WT, L384M, M421G, Q502I, G521R, Y537S) (SEQ ID NO. 71), ER (aa 305-549 of WT, L384M, M421G, Q502M, G521R, Y537S) (SEQ ID NO. 73), ER (aa 305-549 of WT, L384M, M421G, Q502N, G521R, Y537S) (SEQ ID NO. 75), ER (aa 305-549 of WT, L384M, M421G, Q502K, G521R, Y537S) (SEQ ID NO. 77), ER (aa 305-549 of WT, L384M, M421G, Q502V, G521R, Y537S) (SEQ ID NO. 79), ER (aa 305-549 of WT, L384M, M421G, Q5025, G521R, Y537S) (SEQ ID NO. 81), ER (aa 305-549 of WT, L384M, M421G, Q502C, G521R, Y537S) (SEQ ID NO. 83), ER (aa 305-549 of WT, L384M, M421G, Q502W, G521R, Y537S) (SEQ ID NO. 85), ER (aa 305-549 of WT, L384M, M421G, Q502P, G521R, Y537S) (SEQ ID NO. 87), ER (aa 305-549 of WT, L384M, M421G, Q502T, G521R, Y537S) (SEQ ID NO. 89), ER (aa 305-549 of WT, L384M, M421G, Q502A, G521R, Y537S) (SEQ ID NO. 91), ER (aa 305-549 of WT, L384M, M421G, Q502D, G521R, Y537S) (SEQ ID NO. 93), ER (aa 305-549 of WT, L384M, M421G, Q502E, G521R, Y537S) (SEQ ID NO. 95), and ER (aa 305-549 of WT, L384M, M421G, Q502G, G521R, Y537S) (SEQ ID NO. 97).

In one embodiment, the DD is ER (aa 305-549 of WT, L384M, M421G, Q502D, G521R, Y537S) (SEQ ID NO. 93).

In one embodiment, the DD is ER (aa 305-549 of WT, L384M, M421G, Q502H, G521R, Y537S) (SEQ ID NO. 69).

In one embodiment, the DD is ER (aa 305-549 of WT, L384M, N413T, M421G, G521R, Y537S) (SEQ ID NO. 55)

In one embodiment, the DD is ER (aa 305-549 of WT, L384M, N413H, M421G, G521R, Y537S) (SEQ ID NO. 33).

In one embodiment, the DD is ER (aa 305-549 of WT, L384M, N413D, M421G, G521R, Y537S) (SEQ ID NO. 19).

The DDs described herein may further include one or more mutations such as but not limited to K303R, N3045, 5305N, R335G, T371A, T431I, N5195, E523G, A546T, or G442V.

The SREs described herein may include one or more payloads. The payload may be a natural protein or a variant thereof or a fusion polypeptide, or an antibody or a fragment thereof or a therapeutic agent or a gene therapy.

In one embodiment, the payload may be a therapeutic agent. The therapeutic agent may be chimeric antigen receptor, a cytokine or a cytokine-cytokine receptor fusion protein. In some aspects, the payload may be a cytokine. In some embodiments, the cytokine may be IL12. The IL12 may include a p40 subunit (SEQ ID NO. 111) appended to a p35 subunit (SEQ ID NO. 113). The effector modules that include IL12 may have an amino acid sequence such as but not limited to SEQ ID NO. 232; 208; 158-217; 219-231; and/or SEQ ID NO. 233-242. In one embodiment, the effector module may include the amino acid sequence of SEQ ID NO. 232. In one embodiment, the effector module may include the amino acid sequence of SEQ ID NO. 208.

The SRE of the biocircuit system may be SRE is responsive to one or more stimuli. The stimulus may be a small molecule. The small molecule may include but is not limited to Bazedoxifene, raloxifene 4-hydroxytamoxifen (4-OHT), fulvestrant, oremifene, lasofoxifene, clomifene, femarelle and ormeloxifene. In one embodiment, the small molecule is Bazedoxifene.

In one embodiment, the small molecule is raloxifene.

Provided herein are compositions comprising an effector module that includes a stimulus response element as well as at least one payload such as but not limited to chimeric antigen receptor, a cytokine or a cytokine-cytokine receptor fusion protein. The SRE may be a destabilizing domain derived from a region of human estrogen receptor (SEQ ID NO. 1) which may include one or more mutations relative to SEQ ID NO.1 such as but not limited to T371A, N5195, K303R, N3045, 5305N, R335G, T431I, E523G, A546T, and G442V. The DD may further include one or mutations such as but not limited to L384M, M421G, G521R, and/or Y537S. Non-limiting examples of DDs may include ER (aa 305-549 of WT, T371A, L384M, M421G, N5195, G521R, Y537S) (SEQ ID NO. 8), ER (aa 303-549 of WT, K303R, N3045, T371A, L384M, M421G, N5195, G521R, Y537S) (SEQ ID NO. 12), ER (aa 305-549 of WT, R335G, L384M, M421G, N5195, G521R, Y537S) (SEQ ID NO. 13), ER (aa 305-549 of WT, R335G, L384M, M421G, G521R, E523G, Y537S, A546T) (SEQ ID NO. 15), ER (aa 305-549 of WT, L384M, M421G, T431I, G521R, Y537S) (SEQ ID NO. 17), ER (aa 305-549 of WT, L384M, N413D, M421G, G521R, Y537S) (SEQ ID NO. 19), ER (aa 305-549 of WT, L384M, M421G, N5195, G521R, Y537S) (SEQ ID NO. 21), ER (aa 305-549 of WT, L384M, M421G, Q502R, G521R, Y537S) (SEQ ID NO. 23), ER (aa 305-549 of WT, 5305N, L384M, M421G, G442V, G521R, Y537S) (SEQ ID NO. 25), and/or ER (aa 305-549 of WT, L384M, M421G, G521R, Y537S) (SEQ ID NO. 4).

In one embodiment, the composition may include a DD such as is ER (aa 305-549 of WT, T371A, L384M, M421G, N5195, G521R, Y537S) (SEQ ID NO. 8).

In some aspects, the payload may be a chimeric antigen receptor which may include (a) an extracellular target moiety; (b) a hinge and transmembrane domain; (c) an intracellular signaling domain; and (d) optionally, one or more co-stimulatory domains. The extracellular target moiety of the CAR may have an affinity or bind to a target molecule on the surface of a cancer cell. In some embodiments, the extracellular moiety of the CAR may be an scFv. In some aspects, the target molecule may be CD19. In some aspects, the extracellular target moiety of the CAR may be a CD19 scFv which may include SEQ ID NO. 103; or SEQ ID NO. 368.

The intracellular signaling domain of the CAR described herein may be the signaling domain derived from CD3zeta or a cell surface molecule, which may be derived from FcR gamma, FcR beta, CD3 gamma, CD3 delta, CD3 epsilon, CD5, CD22, CD79a, CD79b, and CD66d. In some aspects, the co-stimulatory domain of the CAR may be present and may be derived from 4-1BB (CD137) 2B4, HVEM, ICOS, LAG3, DAP10, DAP12, CD27, CD28, OX40 (CD134), CD30, CD40, ICOS (CD278), glucocorticoid-induced tumor necrosis factor receptor (GITR), lymphocyte function-associated antigen-1 (LFA-1), CD2, CD7, LIGHT, NKG2C, and/or B7-H3.

In one aspect, the intracellular signaling domain of the CAR may be derived from CD3 zeta, and such CD3 zeta domains may have the amino acid sequence of SEQ ID NO. 109. In some aspects, the co-stimulatory domain of the CAR may be present and be derived from 4-1BB. In one aspect, the costimulatory domain may include the amino acid sequence of SEQ ID NO. 107. In some aspects, the CAR may further include a signal sequence. The signal sequence may be derived from CD8a. In one embodiment, the signal sequence may include the amino acid sequence of SEQ ID NO. 99. Non-limiting examples of effector module that include CARs may be SEQ ID NO. 412, 410, 414, 416, 418, 420, 422, 394, 398, 388, 390, 392, 396, 400, 402, 404, and 406. In one embodiment, the effector module may be SEQ ID NO. 412. In one embodiment, the effector module may be SEQ ID NO. 410. In one embodiment, the effector module may be SEQ ID NO. 414. In one embodiment, the effector module may be SEQ ID NO. 418.

In some embodiments, the effector module may be a cytokine such as but not limited to IL12. Non-limiting examples of IL12 effector modules include SEQ ID NO. 240, 160, 242, 130, 133, 136, 139, 142, 145, 148, 151, 154, and 382. In one embodiment, the effector module may be SEQ ID NO. 240. In one embodiment, the effector module may be SEQ ID NO. 160. In one embodiment, the effector module may be SEQ ID NO. 242. In some aspects, the compositions that include IL12 effector modules may further include a chimeric antigen receptor such as but not limited to CD19 CAR. The CAR in some aspects may be SEQ ID NO. 128, SEQ ID NO. 380, and/or SEQ ID NO. 386. In some aspects, compositions that include IL12 effector modules and a CAR may have an amino acid sequence of SEQ ID NO. 156, 373, 375, 377, 384, and 426. In one embodiment, the composition may be SEQ ID NO. 156. In one embodiment, the composition may be SEQ ID NO. 373. In one embodiment, the composition may be SEQ ID NO. 426.

In some embodiments, the payload may be a cytokine-cytokine receptor fusion protein such as but not limited to the whole or a portion of IL15, fused to the whole or a portion of IL15Ra to produce an IL15-IL15Ra fusion protein. In one aspect, the cytokine-cytokine receptor fusion protein may be SEQ ID NO. 246 fused to SEQ ID NO. 248 to produce a fusion polypeptide. Non limiting examples of IL15-IL15Ra fusion protein may be SEQ ID NO. 252, 254, 256, 258, 260, 262, 264, 266, and 268. In one embodiment, the IL15-IL15Ra fusion protein may be SEQ ID NO. 252. In one embodiment, the IL15-IL15Ra fusion protein may be SEQ ID NO. 256. In one embodiment, the IL15-IL15Ra fusion protein may be SEQ ID NO. 268.

Also provided herein are polynucleotides encoding the compositions described herein, a vector that includes the polynucleotides, a pharmaceutical composition that includes the compositions described herein and a pharmaceutically acceptable excipient, as well as immune cells for adoptive cell transfer (ACT) which may express the compositions, the pharmaceutical composition, the polynucleotide or vectors described herein.

Also provided herein are polynucleotides and vectors encoding the biocircuit systems as well as pharmaceutical compositions which include the biocircuit systems described herein and a pharmaceutically acceptable excipient.

In some embodiments, the present disclosure describes methods of reducing tumor burden in a subject. Such methods may include contacting an immune cell with a composition, which may include an effector module. In some aspects, the effector module may further include a stimulus response element (SRE) operably linked to an immunotherapeutic agent. In some aspects, the immunotherapeutic agent may be a chimeric antigen receptor. The SRE described herein may be a DD which may be derived from human estrogen receptor (ER; SEQ ID NO.1). The SRE may be responsive to or interact with a stimulus. The methods of reducing tumor burden may further include administering a therapeutically effective amount of the immune cell to the subject; and administering to the subject, a therapeutically effective amount of the stimulus. In some aspects, the stimulus may be able to modulate the expression of the immunotherapeutic agent. In one embodiment, the immunotherapeutic agent may be able to reduce the tumor burden by inducing an immune response. In some aspects, the stimulus may be a ligand. Non-limiting examples of ligands include bazedoxifene, raloxifene 4-hydroxytamoxifen (4-OHT), fulvestrant, oremifene, lasofoxifene, clomifene, femarelle and ormeloxifene. In some aspects, the ligand may be Bazedoxifene. In some embodiments, the method of reducing tumor burden may involve administering Bazedoxifene at a dose ranging from about 10 mg/kg to about 1000 mg/kg body weight of the subject. In one embodiment, the Bazedoxifene may be administered to the subject at a dose of 200 mg/kg body weight of the subject. In one aspect, the Bazedoxifene may be administered to the subject at a dose of 100 mg/kg body weight of the subject. In one embodiment, the Bazedoxifene may be administered to the subject at a dose of 50 mg/kg body weight of the subject. In some aspects, the Bazedoxifene may be administered to the subject once a day.

Also provided herein are methods of inducing an immune response in a subject. Such methods may involve contacting an immune cell with the compositions described herein. In some embodiments, the compositions described herein may include an immunotherapeutic agent, which may be able to induce an immune response. In some aspects, the immune cell may be treated with the stimulus prior to being administered to the subject. In one embodiment, the immune response in the subject may be measured by an induction of at least one cell marker, such as but not limited to cell markers such as interferon gamma or granzyme B.

In one embodiment, the immunotherapeutic agent may be a CAR such as a CD19 CAR. In some embodiments, the effector modules useful in the present disclosure may be SEQ ID NO. 412, 410, 414, 416, 418, 420, 422, 394, 398, 388, 390, 392, 396, 400, 402, 404, and 406. In one aspect, the effector module may be SEQ ID NO. 412. In one aspect, the effector module may be SEQ ID NO. 410. In one aspect, the effector module may be SEQ ID NO. 418.

### DETAILED DESCRIPTION

The details of one or more embodiments of the disclosure are set forth in the accompanying description below. Although any materials and methods similar or equivalent to those described herein can be used in the practice or testing of the present disclosure, the preferred materials and methods are now described. Other features, objects and advantages of the disclosure will be apparent from the description. In the description, the singular forms also include the plural unless the context clearly dictates otherwise. Unless defined otherwise, all technical and scientific terms used herein have the same meaning as commonly understood by one of ordinary skill in the art to which this disclosure belongs. In the case of conflict, the present description will control.

### I. Introduction

### Protein Regulation

The ability to conditionally control protein levels is a powerful tool in gene and cell therapy. Techniques to control protein expression on a genetic level have been widely studied. The Cre-Lox technology provides a useful approach to activate or inactivate genes. Tissue or cell specific promoters can be used to control spatial and temporal expression of genes of interest. However, this system is limited in application due to the irreversible nature of the perturbation. The transcription of the gene of interest can be conditionally regulated using tools such as Doxycycline (Dox)-inducible system. Alternatively, the stability of mRNA can be regulated using RNA interference techniques. However, methods targeting DNA or RNA are slow acting, irreversible and have low efficiency.

One approach to regulated protein expression or function is the use of Destabilizing Domains (DDs). Destabilizing domains are small protein domains that can be appended to a target protein of interest. DDs render the attached protein of interest unstable in the absence of a DD-binding ligand such that the protein is rapidly degraded by the ubiquitin-proteasome system of the cell

Direct manipulation of activities at the protein level provides significant advantages in flexibility, reversibility and speed. Strategies which directly trigger a cell's natural degradation system have been developed. Szostak and colleagues showed that a small peptide sequence could be fused to the N-terminus of a protein of interest to modulate protein stability. Varshaysky and coworkers isolated a temperature-sensitive peptide sequence that greatly reduced the half-life of dihydrofolate reductase (DHFR) at the non-permissive temperatures. These mutants have been widely used to study protein functions in yeast.

Subsequently, reversible systems employing a rapamycin derivative for the regulation of GSK-3β kinase fused to an unstable triple-mutant of the FRB domain (FRB*) were developed. The rapamycin derivative induces dimerization of the FRB*-GSK-3β and endogenous FKBP12 and stabilizes the FRB* fusion thus restoring the function of the fused kinase.

Building on the FRB* domain system, Banaszynski, et al., developed a cell-permeable ligand system using mutants of FKBP12 protein which were engineered to be unstable in the absence of a high-affinity ligand, Shield-1. (Banaszynski et al., Cell. 2006; 126:995-1004). They termed these unstable domains, destabilizing domains (DDs).

The FKBP/shield-1 tuning system has been successfully used in several studies to control target proteins. For example, Dettwier et al., fused FKBP to tune the express of NADPH P450 oxidoreductase (POR) (Dettwier et al., PLoS One, 2014, 9(11): e113540).

The FKBP DD-shield system has been used in cell lines, transgenic mice, protozoan Entamoeba histolytica, the flatworm Caenorhabditis elegans, the medaka, and transgenic xenografts to investigate the activity of a protein of interest and for iPSC reprogramming.

In addition, the destabilizing domain has been used for the conditional knock down/knock out of the target gene fused with the destabilizing domain. Park et al achieved this genomic engineering by CRISPR/Cas9-mediated homologous recombination and a donor template coding for a resistance cassette and the DD-tagged TCOF1 sequence.

More recently protein switches useful as biosensors as well as new chimeric antigen receptors and other small molecule stabilization frameworks have been disclosed.

One drawback of the FKBP/Shield-1 is that Shield-1 is a novel drug whose biodistribution is not fully characterized and it is not known to what extent Shield-1 crosses the blood-brain barrier.

Other DD ligand pairs include estrogen receptor domains which can be regulated by several estrogen receptor antagonists (Miyazaki et al., J Am Chem. Soc., 2012, 134(9): 3942-3945), and fluorescent destabilizing domain (FDD) derived from bilirubin-inducible fluorescent protein, UnaG. A FDD and its cognate ligand bilirubin (BR) can induce degradation of a protein fused to the FDD (Navarro et al., ACS Chem Biol., 2016, June 6, Epub). Other known DDs and their applications in protein stability include those described in U.S. Pat. Nos. 8,173,792 and 8,530,636, the contents of which are each incorporated herein by reference in their entirety.

In an orthogonal approach, the destabilizing domains of the bacterial dihydrofolate reductase (ecDHFR) were explored. Numerous inhibitors of DHFR have been developed as drugs and one such inhibitor Trimethoprim (TMP), inhibits ecDHFR much more potently than mammalian DHFR providing specificity to the interaction. Additionally, TMP is commercially available and has desirable pharmacological properties making this protein-ligand pair ideal for development for use as a biocircuit (Iwamoto, et al., Chem Biol. (2010) September 24; 17(9): 981-988).

The present disclosure expands upon the technology of tuning protein stability using novel destabilizing domains derived from estrogen receptor (ER). The destabilization and stabilization of a protein of interest, e.g., a transgene for gene therapy, can be controlled by ER mutant DDs having destabilizing or stabilizing properties and their ligands, e.g. Trimethoprim and Methotrexate specifically binding to such protein domains. The presence and/or absence of a small molecule ligand can tune the activity of a protein of interest that is genetically fused to the destabilizing domain.

### II. Compositions of the Disclosure

A variety of strategies that can directly control protein, e.g., a transgene, expression and function are available. The present disclosure provides novel protein domains displaying small molecule dependent stability. Such protein domains are called destabilizing domains (DDs). In the absence of its binding ligand, the DD causes degradation of a payload such as a protein of interest (POI) that is operably linked to the DD, while in the presence of its binding ligand, the fused DD and payload can be stabilized, and its stability is dose dependent.

According to the present disclosure, biocircuit systems are provided which comprise, at their core, at least one effector module system. Such effector module systems comprise at least one effector module having associated, or integral therewith, one or more stimulus response elements (SREs). The overall architecture of a biocircuit system of the disclosure is illustrated in FIG. 1 of International Publication No. WO2017180587 (the contents of which are herein incorporated by reference in their entirety), which shows an overview diagram of a biocircuit system of the disclosure. The biocircuit comprises a stimulus and at least one effector module responsive to a stimulus, where the response to the stimulus produces a signal or outcome. The effector module comprises at least one stimulus response element (SRE) and one payload. In particular, biocircuit systems and effector modules comprising the novel destabilizing domains discussed herein are provided. In some embodiments, the SRE is ER-derived SRE. In some embodiments, the effector module described herein may be a ER-derived SRE operably linked to a payload. FIG. 2 of International Publication No. WO2017180587 (the contents of which are herein incorporated by reference in their entirety) shows representative effector modules carrying one payload. The signal sequence (SS), SRE and payload may be located or positioned in various arrangements without (A to F) or with (G to Z, and AA to DD) a cleavage site. An optional linker may be inserted between each component of the effector module. FIG. 3 of International Publication No. WO2017180587 (the contents of which are herein incorporated by reference in their entirety) shows representative effector modules carrying two payloads without a cleavage site. The two payloads may be either directly linked to each other or separated.

As used herein, a “biocircuit” or “biocircuit system” is defined as a circuit within or useful in biologic systems comprising a stimulus and at least one effector module responsive to a stimulus, where the response to the stimulus produces at least one signal or outcome within, between, as an indicator of, or on a biologic system. Biologic systems are generally understood to be any cell, tissue, organ, organ system or organism, whether animal, plant, fungi, bacterial, or viral. It is also understood that biocircuits may be artificial circuits which employ the stimuli or effector modules taught by the present disclosure and effect signals or outcomes in acellular environments such as with diagnostic, reporter systems, devices, assays or kits. The artificial circuits may be associated with one or more electronic, magnetic, or radioactive components or parts. A much-detailed description of a biocircuit system can be found in International Publication No. WO2017180587 (the contents of which are herein incorporated by reference in their entirety).

In one aspect of the present disclosure, the biocircuit system is a DD biocircuit system.

As used herein, an “effector module” is a single or multi-component construct or complex comprising at least (a) one or more stimulus response elements (SREs) and (b) one or more payloads (i.e. proteins of interest (POIs). In the context of the present disclosure, the SRE is a DD.

As used herein a “payload” or “target payload” is defined as any protein or nucleic acid whose function is to be altered. Payloads may include any coding or non-coding gene or any protein or fragment thereof, or fusion constructs, or antibodies.

In some embodiments, the present disclosure provides biocircuit systems, effector modules and compositions comprising the DDs of the present disclosure. In one aspect, the biocircuit system is a DD biocircuit system.

Payloads are often associated with one or more SREs (e.g., DDs) and may be encoded alone or in combination with one or more DD in a polynucleotide of the disclosure. Payloads themselves may be altered (at the protein or nucleic acid level) thereby providing for an added layer of tenability of the effector module. For example, payloads may be engineered or designed to contain mutations, single or multiple, which affect the stability of the payload or its susceptibility to degradation, cleavage or trafficking. The combination of a DD which can have a spectrum of responses to a stimulus with a payload which is altered to exhibit a variety of responses or gradations of output signals, e.g., expression levels, produce biocircuits which are superior to those in the art. For example, mutations or substitutional designs such as those created for IL12 in WO2016048903 (specifically in Example 1 therein), the contents of which are incorporated herein by reference in their entirety, may be used in any protein payload in conjunction with a DD of the present disclosure to create dual tunable biocircuits. The ability to independently tune both the DD and the payload greatly increases the scope of uses of the effector modules of the present disclosure.

Effector modules may be designed to include one or more payloads, one or more DDs, one or more cleavage sites, one or more signal sequences, one or more tags, one or more targeting peptides, and one or more additional features including the presence or absence of one or more linkers. Representative effector module embodiments of the disclosure are illustrated in FIGS. 2-3 of International Publication No. WO2017180587 (the contents of which are herein incorporated by reference in their entirety). In some aspects, the DD can be positioned at the N-terminal end, or the C-terminal end, or internal of the effector module construct. Different components of an effector module such as DDs, payloads and additional features are organized linearly in one construct or are separately constructed in separate constructs.

Additionally, effector modules of the present disclosure may further comprise other regulatory moieties such as inducible promoters, enhancer sequences, microRNA sites, and/or microRNA targeting sites that provide flexibility on controlling the activity of the payload. The payloads of the present disclosure may be any natural proteins and their variants, or fusion polypeptides, antibodies and variants thereof, transgenes and therapeutic agents.

The stimulus of the biocircuit system may be, but is not limited to, a ligand, a small molecule, an environmental signal (e.g., pH, temperature, light and subcellular location), a peptide or a metabolite. In one aspect of the present disclosure, the stimulus is a ER DD binding ligand including methotrexate (MTX) and trimethoprim (TMP).

Polypeptides of DDs, biocircuit systems and effector modules comprising such DDs and payload constructs, other components, polynucleotides encoding these polypeptides and variants thereof, vectors comprising these polynucleotides, are provided in the present disclosure. The vector may be a plasmid or a viral vector including but not limited to a lentiviral vector, a retroviral vector, a recombinant AAV vector and oncolytic viral vector.

According to the present disclosure, biocircuit systems and effector modules of the disclosure can be used to regulate the expression and activity of a payload in response to the presence or absence of a ligand that specifically binds to the DD integrated within the biocircuit system and effector module.

In some aspects, DDs, effector modules and biocircuit systems of the disclosure may be used to regulate the expression, function and activity of a payload in a cell or a subject. The regulation refers to a lever of change of its expression, function and activity, by at least about 10%, or at least about 20%, or at least about 30%, or at least about 40%, or at least about 50%, or at least about 60%, or at least about 70%, or at least about 80%, or at least about 85%, or at least about 90%, or at least about 95%, or at least about 100%, or at least 20-30%, 20-40%, 20-50%, 20-60%, 20-70%, 20-80%, 20-90%, 20-95%, 20-100%, 30-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100%.

In some embodiments, the present disclosure provides methods for modulating protein, expression, function or level by measuring the stabilization ratio, destabilization ratio, and destabilizing mutation co-efficient. As used herein, the stabilization ratio may be defined as the ratio of expression, function or level of a protein of interest in response to the stimulus to the expression, function or level of the protein of interest in the absence of the stimulus specific to the SRE. In some aspects, the stabilization ratio is at least 1, such as by at least 1-10, 1-20, 1-30, 1-40, 1-50, 1-60, 1-70, 1-80, 1-90, 1-100, 20-30, 20-40, 20-50, 20-60, 20-70, 20-80, 20-90, 20-95, 20-100, 30-40, 30-50, 30-60, 30-70, 30-80, 30-90, 30-95, 30-100, 40-50, 40-60, 40-70, 40-80, 40-90, 40-95, 40-100, 50-60, 50-70, 50-80, 50-90, 50-95, 50-100, 60-70, 60-80, 60-90, 60-95, 60-100, 70-80, 70-90, 70-95, 70-100, 80-90, 80-95, 80-100, 90-95, 90-100 or 95-100. As used herein, the destabilization ratio may be defined as the ratio of expression, function or level of a protein of interest in the absence of the stimulus specific to the effector module to the expression, function or level of the protein of interest, that is expressed constitutively and in the absence of the stimulus specific to the SRE. As used herein “constitutively” refers to the expression, function or level a protein of interest that is not linked to an SRE or is linked to the wildtype protein from which the SRE is derived and is therefore expressed both in the presence and absence of the stimulus. As used herein, the destabilizing mutation co-efficient may be defined as the ratio of expression or level of a protein of interest that is appended to a DD, in the absence of the stimulus specific to the effector module to the expression, function or level of the protein that is appended to the wild type protein from which the DD is derived. In some aspects, the destabilization ratio and the destabilizing mutation co-efficient is at least 0, such as by at least 0.1, 0.2, 0.3, 0.4, 0.5, 0.6, 0.7, 0.8, 0.9, or at least, 0-0.1, 0-0.2, 0-0.3, 0-0.4, 0-0.5, 0-0.6, 0-0.7, 0-0.8, 0-0.9, 0.1-0.2, 0.1-0.3, 0.1-0.4, 0.1-0.5, 0.1-0.6, 0.1-0.7, 0.1-0.8, 0.1-0.9, 0.2-0.3, 0.2-0.4, 0.2-0.5, 0.2-0.6, 0.2-0.7, 0.2-0.8, 0.2-0.9, 0.3-0.4, 0.3-0.5, 0.3-0.6, 0.3-0.7, 0.3-0.8, 0.3-0.9, 0.4-0.5, 0.4-0.6, 0.4-0.7, 0.4-0.8, 0.4-0.9, 0.5-0.6, 0.5-0.7, 0.5-0.8, 0.5-0.9, 0.6-0.7, 0.6-0.8, 0.6-0.9, 0.7-0.8, 0.7-0.9 or 0.8-0.9.

The position of the payload with respect to the DD, within the SRE may be varied to achieve optimal DD regulation. In some embodiments, the payload may be fused to the N terminus of the DD. In another embodiment, the payload may be fused to the C terminus of the DDs. An optional start codon nucleotide sequence encoding for methionine may be added to the DD and/or payload. In some embodiments, effector modules of the present disclosure may include one or more degrons to tune expression. As used herein, a “degron” refers to a minimal sequence within a protein that is sufficient for the recognition and the degradation by the proteolytic system. An important property of degrons is that they are transferrable, that is, appending a degron to a sequence confers degradation upon the sequence. In some embodiments, the degron may be appended to the destabilizing domains, the payload or both. Incorporation of the degron within the effector module of the disclosure, confers additional protein instability to the effector module and may be used to minimize basal expression. In some embodiments, the degron may be an N-degron, a phospho degron, a heat inducible degron, a photosensitive degron, an oxygen dependent degron. As a non-limiting example, the degron may be an Ornithine decarboxylase degron as described by Takeuchi et al. (Takeuchi J et al. (2008). Biochem J. 2008 Mar. 1; 410(2):401-7; the contents of which are incorporated by reference in their entirety). Other examples of degrons useful in the present disclosure include degrons described in International patent publication Nos. WO2017004022, WO2016210343, and WO2011062962; the contents of each of which are incorporated by reference in their entirety.

In some embodiments, more than one biocircuit system may be used in combination to control various protein functions in the same cell or organism, each of which uses different DD and ligand pair and can be regulated separately.

In some embodiments, biocircuits of the disclosure may be modified to reduce their immunogenicity. Immunogenicity is the result of a complex series of responses to a substance that is perceived as foreign and may include the production of neutralizing and non-neutralizing antibodies, formation of immune complexes, complement activation, mast cell activation, inflammation, hypersensitivity responses, and anaphylaxis. Several factors can contribute to protein immunogenicity, including, but not limited to protein sequence, route and frequency of administration and patient population. In a preferred embodiment, protein engineering may be used to reduce the immunogenicity of the compositions of the disclosure. In some embodiments, modifications to reduce immunogenicity may include modifications that reduce binding of the processed peptides derived from the parent sequence to MHC proteins. For example, amino acid modifications may be engineered such that there are no or a minimal of number of immune epitopes that are predicted to bind with high affinity, to any prevalent MHC alleles. Several methods of identifying MHC binding epitopes of known protein sequences are known in the art and may be used to score epitopes in the compositions of the present disclosure. Such methods are disclosed in US Patent Publication No. US 20020119492, US20040230380, and US 20060148009; the contents of each of which are incorporated by reference in their entirety.

Epitope identification and subsequent sequence modification may be applied to reduce immunogenicity. The identification of immunogenic epitopes may be achieved either physically or computationally. Physical methods of epitope identification may include, for example, mass spectrometry and tissue culture/cellular techniques. Computational approaches that utilize information obtained on antigen processing, loading and display, structural and/or proteomic data toward identifying non-self-peptides that may result from antigen processing, and that are likely to have good binding characteristics in the groove of the MHC may also be utilized. One or more mutations may be introduced into the biocircuits of the disclosure directing the expression of the protein, to maintain its functionality while simultaneously rendering the identified epitope less or non-immunogenic.

In some embodiments, the endoplasmic reticulum associated degradation (ERAD) pathway may be used to optimize degradation of the payloads described herein e.g. secreted and membrane cargos. In one embodiment, the effector modules of the disclosure may have directed to the E3 ligases by using adaptor proteins or protein domains. The endoplasmic reticulum is endowed with a specialized machinery to ensure proteins deployed to the distal secretory pathway are correctly folded and assembled into native oligomeric complexes. Proteins failing to meet this conformational standard are degraded by the ERAD pathway, a process through which folding defective proteins are selected and ultimately degraded by the ubiquitin proteasome system. ERAD proceeds through four main steps involving substrate selection, dislocation across the endoplasmic reticulum membrane, covalent conjugation with polyubiquitin, and proteasome degradation. Any of these steps may be modulated to optimize the degradation of the payloads and the effector modules described herein. Protein adaptors within the endoplasmic reticulum membrane, link substrate recognition to the ERAD machinery (herein referred to as the “dislocon”), which causes the dislocation of the proteins from the endoplasmic reticulum. Non-limiting examples of protein adaptors that may be used to optimize ERAD pathway degradation include but are not limited to SEL1L (an adaptor that links glycan recognition to the dislocon), Erlins (intermembrane substrate adaptors), Insigs (client specific adaptors), F-Box proteins (act as adaptors for dislocated glycoproteins in the cytoplasm) and viral-encoded adaptors.

In some embodiments, protein modifications engineered into the structure of the compositions of the disclosure to interfere with antigen processing and peptide loading such as glycosylation and PEGylation, may also be useful in the present disclosure. Compositions of the disclosure may also be engineered to include non-classical amino acid sidechains to design less immunogenic compositions. Any of the methods discussed in International Patent Publication No. WO2005051975 for reducing immunogenicity may be useful in the present disclosure (the contents of which are incorporated by reference in their entirety).

In one embodiment, patients may also be stratified according to the immunogenic peptides presented by their immune cells and may be utilized as a parameter to determine suitable patient cohorts that may therapeutically benefit for the compositions of the disclosure.

In some embodiments, reduced immunogenicity may be achieved by limiting immunoproteasome processing. The proteasome is an important cellular protease that is found in two forms: the constitutive proteasome, which is expressed in all cell types and which contains active e.g. catalytic subunits and the immunoproteasome that is expressed in cell of the hematopoietic lineage, and which contains different active subunits termed low molecular weight proteins (LMP) namely LMP-2, LMP-7 and LMP-10. Immunoproteasomes exhibit altered peptidase activities and cleavage site preferences that result in more efficient liberation of many MHC class I epitopes. A well described function of the immunoproteasome is to generate peptides with hydrophobic C terminus that can be processed to fit in the groove of MHC class I molecules. Deol P et al. have shown that immunoproteasomes may lead to a frequent cleavage of specific peptide bonds and thereby to a faster appearance of a certain peptide on the surface of the antigen presenting cells; and enhanced peptide quantities (Deol P et al. (2007) J Immunol 178 (12) 7557-7562; the contents of which are incorporated herein reference in its entirety). This study indicates that reduced immunoproteasome processing may be accompanied by reduced immunogenicity. In some embodiments, immunogenicity of the compositions of the disclosure may be reduced by modifying the sequence encoding the compositions of the disclosure to prevent immunoproteasome processing. Biocircuits of the present disclosure may also be combined with immunoproteasome-selective inhibitors to achieve the same effects. Examples of inhibitors useful in the present disclosure include UK-101 (B1i selective compound), IPSI-001, ONX 0914 (PR-957), and PR-924 (IPSI).

### Destabilizing Domains (DDs)

As used herein, the term “destabilizing domains (DDs)” refers to protein domains that are unstable and degraded in the absence of ligand, but whose stability is rescued by binding to a high affinity cell-permeable ligand. The term destabilizing domain (DD) is interchangeable with the term drug responsive domain (DRD). Destabilizing domains (DDs) can be appended to a target protein of interest (POI) and can convey its destabilizing property to the protein of interest, causing protein degradation. The presence, absence or an amount of a small molecule ligand that binds to or interacts with the DD, can, upon such binding or interaction modulate the stability of the payload(s) and consequently the function of the payload. A protein domain with destabilizing property (e.g. a DD) is used in conjunction with a cell-permeable ligand to regulate any protein of interest when it is fused with the destabilizing domain. DDs render the attached protein of interest unstable in the absence of a DD-binding ligand such that the protein is rapidly degraded by the ubiquitin-proteasome system of the cell. However, when a specific small molecule ligand binds its intended DD as a ligand binding partner, the instability is reversed, and protein function is restored. The conditional nature of DD stability allows a rapid and non-perturbing switch from stable protein to unstable substrate for degradation. Moreover, its dependency on the concentration of its ligand further provides tunable control of degradation rates. Depending on the degree of binding and/or interaction the altered function of the payload may vary, hence providing a “tuning” of the payload function.

Due to its reversibility, specificity and the fast and easy regulation on protein level, the post-transcriptional tuning system provides a useful system for gene regulation. Furthermore, the regulation may be dose-dependent, thereby altering the protein-turnover rate to transform a short-lived or no detectable protein into a protein that functions for a precisely controlled period of time (Iwamoto et al., Chem. Biol. 2010, 17: 981-988).

In some embodiments, the desired characteristics of the DDs may include, but are not limited to, low protein levels in the absence of a ligand of the DD (i.e. low basal stability), large dynamic range, robust and predictable dose-response behavior, and rapid kinetics of degradation. Candidate DDs that bind to a desired ligand, but not endogenous molecules may be preferred.

Candidate destabilizing domain sequence identified from protein domains of known wildtype proteins (as a template) may be mutated to generate libraries of mutants based on the template candidate domain sequence. Mutagenesis strategies used to generate DD libraries may include site-directed mutagenesis e.g. by using structure guided information, or random mutagenesis e.g. using error-prone PCR, or a combination of both. In some embodiments, destabilizing domains identified using random mutagenesis may be used to identify structural properties of the candidate DDs that may be required for destabilization, which may then be used to further generate libraries of mutations using site directed mutagenesis.

In some embodiments, novel DDs may be identified by mutating one or more amino acids in the candidate destabilizing domain to an amino acid that is vicinal to the mutation site. As used herein a vicinal amino acid refers to an amino acid that is located 1, 2, 3, 4, 5 or more amino acids upstream or downstream of the mutation site in the linear sequence and/or the crystal structure of the candidate destabilizing domain. In some embodiments, the vicinal amino acid may be a conserved amino acid (with similar physicochemical properties as the amino acid at the mutation site), a semi conserved amino acid (e.g. negatively to positively charge amino acid) or a non-conserved amino acid (with different physicochemical properties than the amino acid at the mutation site).

In some embodiments, DD mutant libraries may be screened for mutations with altered, preferably higher binding affinity to the ligand, as compared to the wild type protein. DD libraries may also be screened using two or more ligands and DD mutations that are stabilized by some ligands but not others may be preferentially selected. DD mutations that bind preferentially to the ligand compared to a naturally occurring protein may also be selected. Such methods may be used to optimize ligand selection and ligand binding affinity of the DD. Additionally, such approaches can be used to minimize deleterious effects caused by off-target ligand binding.

In some embodiments, suitable DDs may be identified by screening mutant libraries using barcodes. Such methods may be used to detect, identify and quantify individual mutant clones within the heterogeneous mutant library. Each DD mutant within the library may have distinct barcode sequences (with respect to each other). In other instances, the polynucleotides can also have different barcode sequences with respect to 2, 3, 4, 5, 6, 7, 8, 9, 10 or more nucleic acid bases. Each DD mutant within the library may also comprise a plurality of barcode sequences. When used in plurality barcodes may be used such that each barcode is unique to any other barcode. Alternatively, each barcode used may not be unique, but the combination of barcodes used may create a unique sequence that can be individually tracked. The barcode sequence may be placed upstream of the SRE, downstream of the SRE, or in some instances may be placed within the SRE. DD mutants may be identified by barcodes using sequencing approaches such as Sanger sequencing, and next generation sequencing, but also by polymerase chain reaction and quantitative polymerase chain reaction. In some embodiments, polymerase chain reaction primers that amplify a different size product for each barcode may be used to identify each barcode on an agarose gel. In other instances, each barcode may have a unique quantitative polymerase chain reaction probe sequence that enables targeted amplification of each barcode.

Inventors of the present disclosure investigated several human proteins and identified novel human DDs which can confer its instability features to the fused payload and facilitate the rapid degradation of the fusion polypeptide in the absence of its ligand but stabilize the fused payload in response to the binding to its ligand. Specifically, the new DDs are derived from human ER protein.

### Human ER Mutants

In some embodiments, DDs of the disclosure may be derived from human estrogen receptor. In some embodiments, the DDs may be derived from the human estrogen receptor alpha (herein referred to as ER or hER), which is encoded by the gene ESR1. In one embodiment, the DD may be derived from the Uniprot ID: P03372.2 (SEQ ID NO. 1). In some embodiments, the DD may be derived from a region or a portion of the estrogen receptor. In one embodiment, the DD may be derived from the ligand binding domain of the estrogen receptor comprising amino acids 305-549 of SEQ ID NO. 1 (SEQ ID NO. 2; encoded by SEQ ID NO. 3).

ER is a nuclear hormone receptor that is involved in the regulation of eukaryotic genes responsible for cellular proliferation and differentiation. When ER binds to its cognate ligand, estrogen results in nuclear transactivation by direct binding to a palindromic estrogen response element (ERE) sequence or association with other DNA-binding transcription factors.

In some embodiments, DDs described herein may include one or mutations. In some embodiments, the mutations may render the DDs incapable of binding to its cognate ligand estrogen, such that the DD does not interfere with endogenous estrogen signaling.

In some aspects, mutations may be engineered into the DD such that it can bind to ligands that are unable to bind to the Wildtype ER protein (SEQ ID NO. 1). In one embodiment, the ER DD may comprise L384M, M421G, G521R mutations that allow binding to synthetic ligands.

In some embodiments, the DDs may include a Y537S mutations, which may destabilize the ER protein.

In some embodiments, mutations may be engineered in the ER protein to improve ligand binding. The binding of the ER to CMP1 may be limited by the following residues in ER L391, F404, M421, 1424, and L428. One or more of these residues may be mutated to improve binding to ER. Non-limiting mutation examples include L384M, M421G, and M421.

In some embodiments, DDs of the present disclosure may be identified by utilizing a cocktail of ER binding ligands. In other instances, the suitable DDs may be identified by screening first with one ER binding ligand and subsequently screening with a second ER binding ligand.

In some embodiments, the destabilizing domains of the disclosure may include wildtype nucleotide sequences which may be utilized to reduce the basal expression of the compositions of the disclosure. In some embodiments, protein molecules described herein can bind to their cognate mRNA and effectively repressing its translation. Alternatively, the nucleic acid sequence of the codons may be selected to alter translation rates. In some embodiments, amino acids identified as critical regulators of ER translation repression may be mutated to enhance translation rates.

The ER DD amino acid sequences and nucleotide sequences are provided in Table 1. The position of a mutation described in Table 1 is with respect to the full length ER protein (SEQ ID NO. 1).

TABLE 1
ER DDs

| | | | AA | NA |
|---|---|---|---|---|
| | | | SEQ | SEQ |
| ERDD- | | | ID | ID |
| ID | Mutations | AA sequence | NO. | NO. |
| ERDD-2 | ER (aa 305-549 | SLALSLTADQMVSALLDAEPPILYSEYDPTRPF | 4 | 5-7 |
| | of WT, L384M, | SEASMMGLLTNLADRELVHMINWAKRVPGFV | | |
| | M421G, G521R, | DLTLHDQVHLLECAWMEILMIGLVWRSMEHP | | |
| | Y537S) | GKLLFAPNLLLDRNQGKCVEGGVEIFDMLLAT | | |
| | | SSRFRMIMNLQGEEFVCLKSIILLNSGVYTFLSS | | |
| | | TLKSLEEKDHIHRVLDKITDTLIHLMAKAGLTL | | |
| | | QQQHQRLAQLLLILSHIRHMSNKRMEHLYSM | | |
| | | KCKNVVPLSDLLLEMLDAHRL | | |
| ERDD-1 | ER (aa 305-549 | SLALSLTADQMVSALLDAEPPILYSEYDPTRPF | 8 | 9-11 |
| | of WT, T371A, | SEASMMGLLTNLADRELVHMINWAKRVPGFV | | |
| | L384M, M421G, | DLALHDQVHLLECAWMEILMIGLVWRSMEHP | | |
| | N519S, G521R, | GKLLFAPNLLLDRNQGKCVEGGVEIFDMLLAT | | |
| | Y537S) | SSRFRMIMNLQGEEFVCLKSIILLNSGVYTFLSS | | |
| | | TLKSLEEKDHIHRVLDKITDTLIHLMAKAGLTL | | |
| | | QQQHQRLAQLLLILSHIRHMSSKRMEHLYSMK | | |
| | | CKNVVPLSDLLLEMLDAHRL | | |
| ERDD-3 | ER (aa 303-549 | RSSLALSLTADQMVSALLDAEPPILYSEYDPTR | 12 | — |
| | of WT, K303R, | PFSEASMIVIGLLTNLADRELVHMINWAKRVPG | | |
| | N3045, T371A, | FVDLALHDQVHLLECAWMEILMIGLVWRSME | | |
| | L384M, M421G, | HPGKLLFAPNLLLDRNQGKCVEGGVEIFDMLL | | |
| | N519S, G521R, | ATSSRFRMMNLQGEEFVCLKSIILLNSGVYTFL | | |
| | Y537S) | SSTLKSLEEKDHIHRVLDKITDTLIHLMAKAGL | | |
| | | TLQQQHQRLAQLLLILSHIRHMSSKRMEHLYS | | |
| | | MKCKNVVPLSDLLLEMLDAHRL | | |
| ERDD-6 | ER (aa 305-549 | SLALSLTADQMVSALLDAEPPILYSEYDPTGPF | 13 | 14 |
| | of WT, R335G, | SEASMMGLLTNLADRELVHMINWAKRVPGFV | | |
| | L384M, M421G, | DLTLHDQVHLLECAWMEILMIGLVWRSMEHP | | |
| | N519S, G521R, | GKLLFAPNLLLDRNQGKCVEGGVEIFDMLLAT | | |
| | Y537S) | SSRFRMMNLQGEEFVCLKSIILLNSGVYTFLSS | | |
| | | TLKSLEEKDHIHRVLDKITDTLIHLMAKAGLTL | | |
| | | QQQHQRLAQLLLILSHIRHMSSKRMEHLYSMK | | |
| | | CKNVVPLSDLLLEMLDAHRL | | |
| ERDD-7 | ER (aa 305-549 | SLALSLTADQMVSALLDAEPPILYSEYDPTGPF | 15 | 16 |
| | of WT, R335G, | SEASMMGLLTNLADRELVHMINWAKRVPGFV | | |
| | L384M, M421G, | DLTLHDQVHLLECAWMEILMIGLVWRSMEHP | | |
| | G521R, E523G, | GKLLFAPNLLLDRNQGKCVEGGVEIFDMLLAT | | |
| | Y537S, A546T) | SSRFRMMNLQGEEFVCLKSIILLNSGVYTFLSS | | |
| | | TLKSLEEKDHIHRVLDKITDTLIHLMAKAGLTL | | |
| | | QQQHQRLAQLLLILSHIRHMSNKRMGHLYSM | | |
| | | KCKNVVPLSDLLLEMLDTHRL | | |
| ERDD-8 | ER (aa 305-549 | SLALSLTADQMVSALLDAEPPILYSEYDPTRPF | 17 | 18 |
| | of WT, L384M; | SEASMMGLLTNLADRELVHMINWAKRVPGFV | | |
| | M421G; T431I; | DLTLHDQVHLLECAWMEILMIGLVWRSMEHP | | |
| | G521R, Y537S) | GKLLFAPNLLLDRNQGKCVEGGVEIFDMLLAI | | |
| | | SSRFRMIMNLQGEEFVCLKSIILLNSGVYTFLSS | | |
| | | TLKSLEEKDHIHRVLDKITDTLIHLMAKAGLTL | | |
| | | QQQHQRLAQLLLILSHIRHMSNKRMEHLYSM | | |
| | | KCKNVVPLSDLLLEMLDAHRL | | |
| ERDD-9 | ER (aa 305-549 | SLALSLTADQMVSALLDAEPPILYSEYDPTRPF | 19 | 20 |
| | of WT, L384M, | SEASMMGLLTNLADRELVHMINWAKRVPGFV | | |
| | N413D, M421G, | DLTLHDQVHLLECAWMEILMIGLVWRSMEHP | | |
| | G521R, Y537S) | GKLLFAPNLLLDRDQGKCVEGGVEIFDMLLAT | | |
| | | SSRFRMIMNLQGEEFVCLKSIILLNSGVYTFLSS | | |
| | | TLKSLEEKDHIHRVLDKITDTLIHLMAKAGLTL | | |
| | | QQQHQRLAQLLLILSHIRHMSNKRMEHLYSM | | |
| | | KCKNVVPLSDLLLEMLDAHRL | | |
| ERDD- | ER (aa 305-549 | SLALSLTADQMVSALLDAEPPILYSEYDPTRPF | 21 | 22 |
| 10 | of WT, L384M, | SEASMMGLLTNLADRELVHMINWAKRVPGFV | | |
| | M421G, N519S, | DLTLHDQVHLLECAWMEILMIGLVWRSMEHP | | |
| | G521R, Y537S) | GKLLFAPNLLLDRNQGKCVEGGVEIFDMLLAT | | |
| | | SSRFRMIMNLQGEEFVCLKSIILLNSGVYTFLSS | | |
| | | TLKSLEEKDHIHRVLDKITDTLIHLMAKAGLTL | | |
| | | QQQHQRLAQLLLILSHIRHMSSKRMEHLYSMK | | |
| | | CKNVVPLSDLLLEMLDAHRL | | |
| ERDD- | ER (aa 305-549 | SLALSLTADQMVSALLDAEPPILYSEYDPTRPF | 23 | 24 |
| 11 | of WT, L384M, | SEASMMGLLTNLADRELVHMINWAKRVPGFV | | |
| | M421G, Q502R, | DLTLHDQVHLLECAWMEILMIGLVWRSMEHP | | |
| | G521R, Y537S) | GKLLFAPNLLLDRNQGKCVEGGVEIFDMLLAT | | |
| | | SSRFRMIMNLQGEEFVCLKSIILLNSGVYTFLSS | | |
| | | TLKSLEEKDHIHRVLDKITDTLIHLMAKAGLTL | | |
| | | QQQHRRLAQLLLILSHIRHMSNKRMEHLYSMK | | |
| | | CKNVVPLSDLLLEMLDAHRL | | |
| ERDD- | ER (aa 305-549 | NLALSLTADQMVSALLDAEPPILYSEYDPTRPF | 25 | 26 |
| 12 | of WT, S305N, | SEASMMGLLTNLADRELVHMINWAKRVPGFV | | |
| | L384M, M421G, | DLTLHDQVHLLECAWMEILMIGLVWRSMEHP | | |
| | G442V, G521R, | GKLLFAPNLLLDRNQGKCVEGGVEIFDMLLAT | | |
| | Y537S) | SSRFRMIMNLQVEEFVCLKSIILLNSGVYTFLSS | | |
| | | TLKSLEEKDHIHRVLDKITDTLIHLMAKAGLTL | | |
| | | QQQHQRLAQLLLILSHIRHMSNKRMEHLYSM | | |
| | | KCKNVVPLSDLLLEMLDAHRL | | |

In some embodiments, the ER derived destabilizing domains may be derived from variants, and or isoforms of ER.

In some embodiments, the first amino acid from the destabilizing domain may be removed or substituted when fused to the linker region or payload. As a non-limiting example, the first amino acid is methionine (M) and it is removed from the destabilizing domain.

The amino acid sequences of the destabilizing domains encompassed in the disclosure have at least about 40%, 50% or 60%, 70% identity, preferably at least about 75% or 80% identity, more preferably at least about 85%, 86%, 87%, 88%, 89% or 90% identity, and further preferably at least about 91%, 92%, 93%, 94%, 95%, 96%, 97%, 98%, or 99% identity to the amino acid sequences described therein. Percent identity may be determined, for example, by comparing sequence information using the advanced BLAST computer program, including version Magic-BLAST 1.2.0, available from the National Institutes of Health. The BLAST program is based on the alignment method discussed in Karl and Altschul (1990) Proc. Natl. Acad. Sci USA, 87:2264-68 (the contents of which are incorporated by reference in their entirety).

In some embodiments, the DD mutations identified herein may be mapped back to the ER sequence to identify DD hotspots. DD hotspots as used herein refer to amino acids within the ER of SEQ ID NO. 1 whose mutation results in the “responsive” nature of the stimulus responsive element generated from ER. The DD characteristics may be improved by saturation mutagenesis, which involves mutating the amino acids at the hotspot position to any of the known amino acids, including, but not limited to lysine, aspartic acid, glutamic acid, glutamine, asparagine, histidine, serine, threonine, tyrosine, cysteine, methionine, tryptophan, alanine, isoleucine, leucine, phenylalanine, valine, proline, and glycine. In some embodiments, the Asparagine at position 413 of SEQ ID NO. 1 may be mutated to any of the known amino acids (Table 2). In some embodiments, the Glutamine at position 502 of SEQ ID NO. 1 may be mutated to any of the known amino acids (Table 3).

The position of the mutated amino acids listed in Table 2 and Table 3 is relative to the wildtype human ER of SEQ ID NO. 1.

TABLE 2
N413 ER mutants

| | | | AA | NA |
|---|---|---|---|---|
| | | | SEQ | SEQ |
| ERDD- | | | ID | ID |
| ID | Mutations | AA sequence | NO. | NO. |
| ERDD- | ER (aa | SLALSLTADQMVSALLDAEPPILYSEYDPTRPFSEAS | 27 | 28 |
| 13 | 305-549 of | MMGLLTNLADRELVHMINWAKRVPGFVDLTLHDQ | | |
| | WT, L384M, | VHLLECAWMEILMIGLVWRSMEHPGKLLFAPNLLL | | |
| | N413F, | DRFQGKCVEGGVEIFDMLLATSSRFRMMNLQGEEF | | |
| | M421G, | VCLKSIILLNSGVYTFLSSTLKSLEEKDHIHRVLDKIT | | |
| | G521R, | DTLIHLMAKAGLTLQQQHQRLAQLLLILSHIRHMSN | | |
| | Y537S) | KRMEHLYSMKCKNVVPLSDLLLEMLDAHRL | | |
| ERDD- | ER (aa | SLALSLTADQMVSALLDAEPPILYSEYDPTRPFSEAS | 29 | 30 |
| 14 | 305-549 of | MMGLLTNLADRELVHMINWAKRVPGFVDLTLHDQ | | |
| | WT, L384M, | VHLLECAWMEILMIGLVWRSMEHPGKLLFAPNLLL | | |
| | N413L, | DRLQGKCVEGGVEIFDMLLATSSRFRMIMNLQGEEF | | |
| | M421G, | VCLKSIILLNSGVYTFLSSTLKSLEEKDHIHRVLDKIT | | |
| | G521R, | DTLIHLMAKAGLTLQQQHQRLAQLLLILSHIRHMSN | | |
| | Y537S) | KRMEHLYSMKCKNVVPLSDLLLEMLDAHRL | | |
| ERDD- | ER (aa | SLALSLTADQMVSALLDAEPPILYSEYDPTRPFSEAS | 31 | 32 |
| 15 | 305-549 of | MMGLLTNLADRELVHMINWAKRVPGFVDLTLHDQ | | |
| | WT, L384M, | VHLLECAWMEILMIGLVWRSMEHPGKLLFAPNLLL | | |
| | N413Y, | DRYQGKCVEGGVEIFDMLLATSSRFRMMNLQGEEF | | |
| | M421G, | VCLKSIILLNSGVYTFLSSTLKSLEEKDHIHRVLDKIT | | |
| | G521R, | DTLIHLMAKAGLTLQQQHQRLAQLLLILSHIRHMSN | | |
| | Y537S) | KRMEHLYSMKCKNVVPLSDLLLEMLDAHRL | | |
| ERDD- | ER (aa | SLALSLTADQMVSALLDAEPPILYSEYDPTRPFSEAS | 33 | 34 |
| 16 | 305-549 of | MMGLLTNLADRELVHMINWAKRVPGFVDLTLHDQ | | |
| | WT, L384M, | VHLLECAWMEILMIGLVWRSMEHPGKLLFAPNLLL | | |
| | N413H, | DRHQGKCVEGGVEIFDMLLATSSRFRMMNLQGEEF | | |
| | M421G, | VCLKSIILLNSGVYTFLSSTLKSLEEKDHIHRVLDKIT | | |
| | G521R, | DTLIHLMAKAGLTLQQQHQRLAQLLLILSHIRHMSN | | |
| | Y537S) | KRMEHLYSMKCKNVVPLSDLLLEMLDAHRL | | |
| ERDD- | ER (aa | SLALSLTADQMVSALLDAEPPILYSEYDPTRPFSEAS | 35 | 36 |
| 17 | 305-549 of | MMGLLTNLADRELVHMINWAKRVPGFVDLTLHDQ | | |
| | WT, L384M, | VHLLECAWMEILMIGLVWRSMEHPGKLLFAPNLLL | | |
| | N413Q, | DRQQGKCVEGGVEIFDMLLATSSRFRMMNLQGEEF | | |
| | M421G, | VCLKSIILLNSGVYTFLSSTLKSLEEKDHIHRVLDKIT | | |
| | G521R, | DTLIHLMAKAGLTLQQQHQRLAQLLLILSHIRHMSN | | |
| | Y537S) | KRMEHLYSMKCKNVVPLSDLLLEMLDAHRL | | |
| ERDD- | ER (aa | SLALSLTADQMVSALLDAEPPILYSEYDPTRPFSEAS | 37 | 38 |
| 18 | 305-549 of | MMGLLTNLADRELVHMINWAKRVPGFVDLTLHDQ | | |
| | WT, L384M, | VHLLECAWMEILMIGLVWRSMEHPGKLLFAPNLLL | | |
| | N413I, | DRIQGKCVEGGVEIFDMLLATSSRFRMMNLQGEEF | | |
| | M421G, | VCLKSIILLNSGVYTFLSSTLKSLEEKDHIHRVLDKIT | | |
| | G521R, | DTLIHLMAKAGLTLQQQHQRLAQLLLILSHIRHMSN | | |
| | Y537S) | KRMEHLYSMKCKNVVPLSDLLLEMLDAHRL | | |
| ERDD- | ER (aa | SLALSLTADQMVSALLDAEPPILYSEYDPTRPFSEAS | 39 | 40 |
| 19 | 305-549 of | MMGLLTNLADRELVHMINWAKRVPGFVDLTLHDQ | | |
| | WT, L384M, | VHLLECAWMEILMIGLVWRSMEHPGKLLFAPNLLL | | |
| | N413M, | DRMQGKCVEGGVEIFDMLLATSSRFRMMNLQGEE | | |
| | M421G, | FVCLKSIILLNSGVYTFLSSTLKSLEEKDHIHRVLDKI | | |
| | G521R, | TDTLIHLMAKAGLTLQQQHQRLAQLLLILSHIRHMS | | |
| | Y537S) | NKRMEHLYSMKCKNVVPLSDLLLEMLDAHRL | | |
| ERDD- | ER (aa | SLALSLTADQMVSALLDAEPPILYSEYDPTRPFSEAS | 41 | 42 |
| 20 | 305-549 of | MMGLLTNLADRELVHMINWAKRVPGFVDLTLHDQ | | |
| | WT, L384M, | VHLLECAWMEILMIGLVWRSMEHPGKLLFAPNLLL | | |
| | N413K, | DRKQGKCVEGGVEIFDMLLATSSRFRMMNLQGEEF | | |
| | M421G, | VCLKSIILLNSGVYTFLSSTLKSLEEKDHIHRVLDKIT | | |
| | G521R, | DTLIHLMAKAGLTLQQQHQRLAQLLLILSHIRHMSN | | |
| | Y537S) | KRMEHLYSMKCKNVVPLSDLLLEMLDAHRL | | |
| ERDD- | ER (aa | SLALSLTADQMVSALLDAEPPILYSEYDPTRPFSEAS | 43 | 44 |
| 21 | 305-549 of | MMGLLTNLADRELVHMINWAKRVPGFVDLTLHDQ | | |
| | WT, L384M, | VHLLECAWMEILMIGLVWRSMEHPGKLLFAPNLLL | | |
| | N413V, | DRVQGKCVEGGVEIFDMLLATSSRFRMMNLQGEEF | | |
| | M421G, | VCLKSIILLNSGVYTFLSSTLKSLEEKDHIHRVLDKIT | | |
| | G521R, | DTLIHLMAKAGLTLQQQHQRLAQLLLILSHIRHMSN | | |
| | Y537S) | KRMEHLYSMKCKNVVPLSDLLLEMLDAHRL | | |
| ERDD- | ER (aa | SLALSLTADQMVSALLDAEPPILYSEYDPTRPFSEAS | 45 | 46 |
| 22 | 305-549 of | MMGLLTNLADRELVHMINWAKRVPGFVDLTLHDQ | | |
| | WT, L384M, | VHLLECAWMEILMIGLVWRSMEHPGKLLFAPNLLL | | |
| | N413S, | DRSQGKCVEGGVEIFDMLLATSSRFRMMNLQGEEF | | |
| | M421G, | VCLKSIILLNSGVYTFLSSTLKSLEEKDHIHRVLDKIT | | |
| | G521R, | DTLIHLMAKAGLTLQQQHQRLAQLLLILSHIRHMSN | | |
| | Y537S) | KRMEHLYSMKCKNVVPLSDLLLEMLDAHRL | | |
| ERDD- | ER (aa | SLALSLTADQMVSALLDAEPPILYSEYDPTRPFSEAS | 47 | 48 |
| 23 | 305-549 of | MMGLLTNLADRELVHMINWAKRVPGFVDLTLHDQ | | |
| | WT, L384M, | VHLLECAWMEILMIGLVWRSMEHPGKLLFAPNLLL | | |
| | N413C, | DRCQGKCVEGGVEIFDMLLATSSRFRMMNLQGEEF | | |
| | M421G, | VCLKSIILLNSGVYTFLSSTLKSLEEKDHIHRVLDKIT | | |
| | G521R, | DTLIHLMAKAGLTLQQQHQRLAQLLLILSHIRHMSN | | |
| | Y537S) | KRMEHLYSMKCKNVVPLSDLLLEMLDAHRL | | |
| ERDD- | ER (aa | SLALSLTADQMVSALLDAEPPILYSEYDPTRPFSEAS | 49 | 50 |
| 24 | 305-549 of | MMGLLTNLADRELVHMINWAKRVPGFVDLTLHDQ | | |
| | WT, L384M, | VHLLECAWMEILMIGLVWRSMEHPGKLLFAPNLLL | | |
| | N413W, | DRWQGKCVEGGVEIFDMLLATSSRFRMIMNLQGEE | | |
| | M421G, | FVCLKSIILLNSGVYTFLSSTLKSLEEKDHIHRVLDKI | | |
| | G521R, | TDTLIHLMAKAGLTLQQQHQRLAQLLLILSHIRHMS | | |
| | Y537S) | NKRMEHLYSMKCKNVVPLSDLLLEMLDAHRL | | |
| ERDD- | ER (aa | SLALSLTADQMVSALLDAEPPILYSEYDPTRPFSEAS | 51 | 52 |
| 25 | 305-549 of | MMGLLTNLADRELVHMINWAKRVPGFVDLTLHDQ | | |
| | WT, L384M, | VHLLECAWMEILMIGLVWRSMEHPGKLLFAPNLLL | | |
| | N413P, | DRPQGKCVEGGVEIFDMLLATSSRFRMMNLQGEEF | | |
| | M421G, | VCLKSIILLNSGVYTFLSSTLKSLEEKDHIHRVLDKIT | | |
| | G521R, | DTLIHLMAKAGLTLQQQHQRLAQLLLILSHIRHMSN | | |
| | Y537S) | KRMEHLYSMKCKNVVPLSDLLLEMLDAHRL | | |
| ERDD- | ER (aa | SLALSLTADQMVSALLDAEPPILYSEYDPTRPFSEAS | 53 | 54 |
| 26 | 305-549 of | MMGLLTNLADRELVHMINWAKRVPGFVDLTLHDQ | | |
| | WT, L384M, | VHLLECAWMEILMIGLVWRSMEHPGKLLFAPNLLL | | |
| | N413R, | DRRQGKCVEGGVEIFDMLLATSSRFRMMNLQGEEF | | |
| | M421G, | VCLKSIILLNSGVYTFLSSTLKSLEEKDHIHRVLDKIT | | |
| | G521R, | DTLIHLMAKAGLTLQQQHQRLAQLLLILSHIRHMSN | | |
| | Y537S) | KRMEHLYSMKCKNVVPLSDLLLEMLDAHRL | | |
| ERDD- | ER (aa | SLALSLTADQMVSALLDAEPPILYSEYDPTRPFSEAS | 55 | 56 |
| 27 | 305-549 of | MMGLLTNLADRELVHMINWAKRVPGFVDLTLHDQ | | |
| | WT, L384M, | VHLLECAWMEILMIGLVWRSMEHPGKLLFAPNLLL | | |
| | N413T, | DRTQGKCVEGGVEIFDMLLATSSRFRMIMNLQGEEF | | |
| | M421G, | VCLKSIILLNSGVYTFLSSTLKSLEEKDHIHRVLDKIT | | |
| | G521R, | DTLIHLMAKAGLTLQQQHQRLAQLLLILSHIRHMSN | | |
| | Y537S) | KRMEHLYSMKCKNVVPLSDLLLEMLDAHRL | | |
| ERDD- | ER (aa | SLALSLTADQMVSALLDAEPPILYSEYDPTRPFSEAS | 57 | 58 |
| 28 | 305-549 of | MMGLLTNLADRELVHMINWAKRVPGFVDLTLHDQ | | |
| | WT, L384M, | VHLLECAWMEILMIGLVWRSMEHPGKLLFAPNLLL | | |
| | N413A, | DRAQGKCVEGGVEIFDMLLATSSRFRMMNLQGEEF | | |
| | M421G, | VCLKSIILLNSGVYTFLSSTLKSLEEKDHIHRVLDKIT | | |
| | G521R, | DTLIHLMAKAGLTLQQQHQRLAQLLLILSHIRHMSN | | |
| | Y537S) | KRMEHLYSMKCKNVVPLSDLLLEMLDAHRL | | |
| ERDD- | ER (aa | SLALSLTADQMVSALLDAEPPILYSEYDPTRPFSEAS | 59 | 60 |
| 29 | 305-549 of | MMGLLTNLADRELVHMINWAKRVPGFVDLTLHDQ | | |
| | WT, L384M, | VHLLECAWMEILMIGLVWRSMEHPGKLLFAPNLLL | | |
| | N413E, | DREQGKCVEGGVEIFDMLLATSSRFRMMNLQGEEF | | |
| | M421G, | VCLKSIILLNSGVYTFLSSTLKSLEEKDHIHRVLDKIT | | |
| | G521R, | DTLIHLMAKAGLTLQQQHQRLAQLLLILSHIRHMSN | | |
| | Y537S) | KRMEHLYSMKCKNVVPLSDLLLEMLDAHRL | | |
| ERDD- | ER (aa | SLALSLTADQMVSALLDAEPPILYSEYDPTRPFSEAS | 61 | 62 |
| 30 | 305-549 of | MMGLLTNLADRELVHMINWAKRVPGFVDLTLHDQ | | |
| | WT, L384M, | VHLLECAWMEILMIGLVWRSMEHPGKLLFAPNLLL | | |
| | N413G, | DRGQGKCVEGGVEIFDMLLATSSRFRMMNLQGEEF | | |
| | M421G, | VCLKSIILLNSGVYTFLSSTLKSLEEKDHIHRVLDKIT | | |
| | G521R, | DTLIHLMAKAGLTLQQQHQRLAQLLLILSHIRHMSN | | |
| | Y537S) | KRMEHLYSMKCKNVVPLSDLLLEMLDAHRL | | |

TABLE 3
Q502 ER mutants

| | | | AA | NA |
|---|---|---|---|---|
| | | | SEQ | SEQ |
| ERDD- | | | ID | ID |
| ID | Mutations | AA sequence | NO. | NO. |
| ERDD- | ER (aa | SLALSLTADQMVSALLDAEPPILYSEYDPTRPFSEAS | 63 | 64 |
| 31 | 305-549 of WT, | MMGLLTNLADRELVHMINWAKRVPGFVDLTLHDQ | | |
| | L384M, | VHLLECAWMEILMIGLVWRSMEHPGKLLFAPNLLL | | |
| | M421G, | DRNQGKCVEGGVEIFDMLLATSSRFRMMNLQGEEF | | |
| | Q502F, | VCLKSIILLNSGVYTFLSSTLKSLEEKDHIHRVLDKIT | | |
| | G521R, | DTLIHLMAKAGLTLQQQHFRLAQLLLILSHIRHMSN | | |
| | Y537S) | KRMEHLYSMKCKNVVPLSDLLLEMLDAHRL | | |
| ERDD- | ER (aa | SLALSLTADQMVSALLDAEPPILYSEYDPTRPFSEAS | 65 | 66 |
| 32 | 305-549 of WT, | MMGLLTNLADRELVHMINWAKRVPGFVDLTLHDQ | | |
| | L384M, | VHLLECAWMEILMIGLVWRSMEHPGKLLFAPNLLL | | |
| | M421G, | DRNQGKCVEGGVEIFDMLLATSSRFRMMNLQGEEF | | |
| | Q502L, | VCLKSIILLNSGVYTFLSSTLKSLEEKDHIHRVLDKIT | | |
| | G521R, | DTLIHLMAKAGLTLQQQHLRLAQLLLILSHIRHMSN | | |
| | Y537S) | KRMEHLYSMKCKNVVPLSDLLLEMLDAHRL | | |
| ERDD- | ER (aa | SLALSLTADQMVSALLDAEPPILYSEYDPTRPFSEAS | 67 | 68 |
| 33 | 305-549 of WT, | MMGLLTNLADRELVHMINWAKRVPGFVDLTLHDQ | | |
| | L384M, | VHLLECAWMEILMIGLVWRSMEHPGKLLFAPNLLL | | |
| | M421G, | DRNQGKCVEGGVEIFDMLLATSSRFRMMNLQGEEF | | |
| | Q502Y, | VCLKSIILLNSGVYTFLSSTLKSLEEKDHIHRVLDKIT | | |
| | G521R, | DTLIHLMAKAGLTLQQQHYRLAQLLLILSHIRHMSN | | |
| | Y537S) | KRMEHLYSMKCKNVVPLSDLLLEMLDAHRL | | |
| ERDD- | ER (aa | SLALSLTADQMVSALLDAEPPILYSEYDPTRPFSEAS | 69 | 70 |
| 34 | 305-549 of WT, | MMGLLTNLADRELVHMINWAKRVPGFVDLTLHDQ | | |
| | L384M, | VHLLECAWMEILMIGLVWRSMEHPGKLLFAPNLLL | | |
| | M421G, | DRNQGKCVEGGVEIFDMLLATSSRFRMMNLQGEEF | | |
| | Q502H, | VCLKSIILLNSGVYTFLSSTLKSLEEKDHIHRVLDKIT | | |
| | G521R, | DTLIHLMAKAGLTLQQQHHRLAQLLLILSHIRHMSN | | |
| | Y537S) | KRMEHLYSMKCKNVVPLSDLLLEMLDAHRL | | |
| ERDD- | ER (aa | SLALSLTADQMVSALLDAEPPILYSEYDPTRPFSEAS | 71 | 72 |
| 35 | 305-549 of WT, | MMGLLTNLADRELVHMINWAKRVPGFVDLTLHDQ | | |
| | L384M, | VHLLECAWMEILMIGLVWRSMEHPGKLLFAPNLLL | | |
| | M421G, | DRNQGKCVEGGVEIFDMLLATSSRFRMMNLQGEEF | | |
| | Q502I, | VCLKSIILLNSGVYTFLSSTLKSLEEKDHIHRVLDKIT | | |
| | G521R, | DTLIHLMAKAGLTLQQQHIRLAQLLLILSHIRHMSN | | |
| | Y537S) | KRMEHLYSMKCKNVVPLSDLLLEMLDAHRL | | |
| ERDD- | ER (aa | SLALSLTADQMVSALLDAEPPILYSEYDPTRPFSEAS | 73 | 74 |
| 36 | 305-549 of WT, | MMGLLTNLADRELVHMINWAKRVPGFVDLTLHDQ | | |
| | L384M, | VHLLECAWMEILMIGLVWRSMEHPGKLLFAPNLLL | | |
| | M421G, | DRNQGKCVEGGVEIFDMLLATSSRFRMMNLQGEEF | | |
| | Q502M, | VCLKSIILLNSGVYTFLSSTLKSLEEKDHIHRVLDKIT | | |
| | G521R, | DTLIHLMAKAGLTLQQQHMRLAQLLLILSHIRHMS | | |
| | Y537S) | NKRMEHLYSMKCKNVVPLSDLLLEMLDAHRL | | |
| ERDD- | ER (aa | SLALSLTADQMVSALLDAEPPILYSEYDPTRPFSEAS | 75 | 76 |
| 37 | 305-549 of WT, | MMGLLTNLADRELVHMINWAKRVPGFVDLTLHDQ | | |
| | L384M, | VHLLECAWMEILMIGLVWRSMEHPGKLLFAPNLLL | | |
| | M421G, | DRNQGKCVEGGVEIFDMLLATSSRFRMMNLQGEEF | | |
| | Q502N, | VCLKSIILLNSGVYTFLSSTLKSLEEKDHIHRVLDKIT | | |
| | G521R, | DTLIHLMAKAGLTLQQQHNRLAQLLLILSHIRHMSN | | |
| | Y537S) | KRMEHLYSMKCKNVVPLSDLLLEMLDAHRL | | |
| ERDD- | ER (aa | SLALSLTADQMVSALLDAEPPILYSEYDPTRPFSEAS | 77 | 78 |
| 38 | 305-549 of WT, | MMGLLTNLADRELVHMINWAKRVPGFVDLTLHDQ | | |
| | L384M, | VHLLECAWMEILMIGLVWRSMEHPGKLLFAPNLLL | | |
| | M421G, | DRNQGKCVEGGVEIFDMLLATSSRFRMMNLQGEEF | | |
| | Q502K, | VCLKSIILLNSGVYTFLSSTLKSLEEKDHIHRVLDKIT | | |
| | G521R, | DTLIHLMAKAGLTLQQQHKRLAQLLLILSHIRHMSN | | |
| | Y537S) | KRMEHLYSMKCKNVVPLSDLLLEMLDAHRL | | |
| ERDD- | ER (aa | SLALSLTADQMVSALLDAEPPILYSEYDPTRPFSEAS | 79 | 80 |
| 39 | 305-549 of WT, | MMGLLTNLADRELVHMINWAKRVPGFVDLTLHDQ | | |
| | L384M, | VHLLECAWMEILMIGLVWRSMEHPGKLLFAPNLLL | | |
| | M421G, | DRNQGKCVEGGVEIFDMLLATSSRFRMMNLQGEEF | | |
| | Q502V, | VCLKSIILLNSGVYTFLSSTLKSLEEKDHIHRVLDKIT | | |
| | G521R, | DTLIHLMAKAGLTLQQQHVRLAQLLLILSHIRHMSN | | |
| | Y537S) | KRMEHLYSMKCKNVVPLSDLLLEMLDAHRL | | |
| ERDD- | ER (aa | SLALSLTADQMVSALLDAEPPILYSEYDPTRPFSEAS | 81 | 82 |
| 40 | 305-549 of WT, | MMGLLTNLADRELVHMINWAKRVPGFVDLTLHDQ | | |
| | L384M, | VHLLECAWMEILMIGLVWRSMEHPGKLLFAPNLLL | | |
| | M421G, | DRNQGKCVEGGVEIFDMLLATSSRFRMMNLQGEEF | | |
| | Q502S, | VCLKSIILLNSGVYTFLSSTLKSLEEKDHIHRVLDKIT | | |
| | G521R, | DTLIHLMAKAGLTLQQQHSRLAQLLLILSHIRHMSN | | |
| | Y537S) | KRMEHLYSMKCKNVVPLSDLLLEMLDAHRL | | |
| ERDD- | ER (aa | SLALSLTADQMVSALLDAEPPILYSEYDPTRPFSEAS | 83 | 84 |
| 41 | 305-549 of WT, | MMGLLTNLADRELVHMINWAKRVPGFVDLTLHDQ | | |
| | L384M, | VHLLECAWMEILMIGLVWRSMEHPGKLLFAPNLLL | | |
| | M421G, | DRNQGKCVEGGVEIFDMLLATSSRFRMMNLQGEEF | | |
| | Q502C, | VCLKSIILLNSGVYTFLSSTLKSLEEKDHIHRVLDKIT | | |
| | G521R, | DTLIHLMAKAGLTLQQQHCRLAQLLLILSHIRHMSN | | |
| | Y537S) | KRMEHLYSMKCKNVVPLSDLLLEMLDAHRL | | |
| ERDD- | ER (aa | SLALSLTADQMVSALLDAEPPILYSEYDPTRPFSEAS | 85 | 86 |
| 42 | 305-549 of WT, | MMGLLTNLADRELVHMINWAKRVPGFVDLTLHDQ | | |
| | L384M, | VHLLECAWMEILMIGLVWRSMEHPGKLLFAPNLLL | | |
| | M421G, | DRNQGKCVEGGVEIFDMLLATSSRFRMMNLQGEEF | | |
| | Q502W, | VCLKSIILLNSGVYTFLSSTLKSLEEKDHIHRVLDKIT | | |
| | G521R, | DTLIHLMAKAGLTLQQQHWRLAQLLLILSHIRHMS | | |
| | Y537S) | NKRMEHLYSMKCKNVVPLSDLLLEMLDAHRL | | |
| ERDD- | ER (aa | SLALSLTADQMVSALLDAEPPILYSEYDPTRPFSEAS | 87 | 88 |
| 43 | 305-549 of WT, | MMGLLTNLADRELVHMINWAKRVPGFVDLTLHDQ | | |
| | L384M, | VHLLECAWMEILMIGLVWRSMEHPGKLLFAPNLLL | | |
| | M421G, | DRNQGKCVEGGVEIFDMLLATSSRFRMMNLQGEEF | | |
| | Q502P, | VCLKSIILLNSGVYTFLSSTLKSLEEKDHIHRVLDKIT | | |
| | G521R, | DTLIHLMAKAGLTLQQQHPRLAQLLLILSHIRHMSN | | |
| | Y537S) | KRMEHLYSMKCKNVVPLSDLLLEMLDAHRL | | |
| ERDD- | ER (aa | SLALSLTADQMVSALLDAEPPILYSEYDPTRPFSEAS | 89 | 90 |
| 44 | 305-549 of WT, | MMGLLTNLADRELVHMINWAKRVPGFVDLTLHDQ | | |
| | L384M, | VHLLECAWMEILMIGLVWRSMEHPGKLLFAPNLLL | | |
| | M421G, | DRNQGKCVEGGVEIFDMLLATSSRFRMMNLQGEEF | | |
| | Q502T, | VCLKSIILLNSGVYTFLSSTLKSLEEKDHIHRVLDKIT | | |
| | G521R, | DTLIHLMAKAGLTLQQQHTRLAQLLLILSHIRHMSN | | |
| | Y537S) | KRMEHLYSMKCKNVVPLSDLLLEMLDAHRL | | |
| ERDD- | ER (aa | SLALSLTADQMVSALLDAEPPILYSEYDPTRPFSEAS | 91 | 92 |
| 45 | 305-549 of WT, | MMGLLTNLADRELVHMINWAKRVPGFVDLTLHDQ | | |
| | L384M, | VHLLECAWMEILMIGLVWRSMEHPGKLLFAPNLLL | | |
| | M421G, | DRNQGKCVEGGVEIFDMLLATSSRFRMMNLQGEEF | | |
| | Q502A, | VCLKSIILLNSGVYTFLSSTLKSLEEKDHIHRVLDKIT | | |
| | G521R, | DTLIHLMAKAGLTLQQQHARLAQLLLILSHIRHMSN | | |
| | Y537S) | KRMEHLYSMKCKNVVPLSDLLLEMLDAHRL | | |
| ERDD- | ER (aa | SLALSLTADQMVSALLDAEPPILYSEYDPTRPFSEAS | 93 | 94 |
| 46 | 305-549 of WT, | MMGLLTNLADRELVHMINWAKRVPGFVDLTLHDQ | | |
| | L384M, | VHLLECAWMEILMIGLVWRSMEHPGKLLFAPNLLL | | |
| | M421G, | DRNQGKCVEGGVEIFDMLLATSSRFRMMNLQGEEF | | |
| | Q502D, | VCLKSIILLNSGVYTFLSSTLKSLEEKDHIHRVLDKIT | | |
| | G521R, | DTLIHLMAKAGLTLQQQHDRLAQLLLILSHIRHMSN | | |
| | Y537S) | KRMEHLYSMKCKNVVPLSDLLLEMLDAHRL | | |
| ERDD- | ER (aa | SLALSLTADQMVSALLDAEPPILYSEYDPTRPFSEAS | 95 | 96 |
| 47 | 305-549 of WT, | MMGLLTNLADRELVHMINWAKRVPGFVDLTLHDQ | | |
| | L384M, | VHLLECAWMEILMIGLVWRSMEHPGKLLFAPNLLL | | |
| | M421G, | DRNQGKCVEGGVEIFDMLLATSSRFRMMNLQGEEF | | |
| | Q502E, | VCLKSIILLNSGVYTFLSSTLKSLEEKDHIHRVLDKIT | | |
| | G521R, | DTLIHLMAKAGLTLQQQHERLAQLLLILSHIRHMSN | | |
| | Y537S) | KRMEHLYSMKCKNVVPLSDLLLEMLDAHRL | | |
| ERDD- | ER (aa | SLALSLTADQMVSALLDAEPPILYSEYDPTRPFSEAS | 97 | 98 |
| 48 | 305-549 of WT, | MMGLLTNLADRELVHMINWAKRVPGFVDLTLHDQ | | |
| | L384M, | VHLLECAWMEILMIGLVWRSMEHPGKLLFAPNLLL | | |
| | M421G, | DRNQGKCVEGGVEIFDMLLATSSRFRMMNLQGEEF | | |
| | Q502G, | VCLKSIILLNSGVYTFLSSTLKSLEEKDHIHRVLDKIT | | |
| | G521R, | DTLIHLMAKAGLTLQQQHGRLAQLLLILSHIRHMSN | | |
| | Y537S) | KRMEHLYSMKCKNVVPLSDLLLEMLDAHRL | | |

In some embodiments. ER destabilizing mutants may be discovered by random mutagenesis of the wildtype human ER using error prone polymerase chain reaction (PCR). The destabilization of the mutants in the absence of its binding ligand may be tested. Binding to ER ligands, Bazedoxifene to human ER may be tested to characterize ligand dependent stabilization.

In some embodiments, the DDs may be derived from ER by mutating one or more amino acids residues between positions 305-315, 315-325, 325-335, 335-345, 345-355, 355-365, 365-375, 375-385, 385-395, 395-405, 405-415, 415-425, 425-435, 435-445, 445-455, 455-465, 465-475, 475-485, 485-495, 495-505, 505-515, 515-525, 525-535, 535-545, and 545-555 of human ER wild type protein (SEQ ID NO. 1). In some embodiments, the mutation may be a conserved (with similar physicochemical properties as the amino acid at the mutation site), a semi conserved (e.g. negatively to positively charge amino acid) or a non-conserved (amino acid with different physicochemical properties than the amino acid at the mutation site). Regions or portions or domains of wild type proteins may be utilized as SREs/DDs in whole or in part. They may be combined or rearranged to create new peptides, proteins, regions or domains of which any may be used as SREs/DDs or the starting point for the design of further SREs and/or DDs.

In some embodiments, variant libraries may be generated by methods known in the art. In one embodiment, VariantFind™ may be used to generate variant library. The VariantFind™ platform is a series of multiplex PCRs that mutates multiple amino acid positions simultaneously. Desired mutations are directly encoded by oligonucleotides, providing high control and specificity during the mutagenesis process. These oligonucleotides are combined in a series of sequential PCRs that result in a ready-to-clone DNA library encoding all desired mutations. In one embodiment, any of the codons in the polynucleotides of the disclosure may be altered by saturation mutagenesis. In another embodiment, a ruleset for amino acid changes may be used to mutate select amino acids of the DDs. In one embodiment, the rule may be the mutation of all Arginine residues in the DD to alanine, lysine and/or leucine. In other embodiments, all phenyl alanine residues in the DD may be mutated to alanine, leucine and/or threonine.

The destabilization domains described herein may also include amino acid and nucleotide substitutions that do not affect stability, including conservative, non-conservative substitutions and or polymorphisms.

In some embodiments, DD mutations that do not inhibit ligand binding may be preferentially selected. In some embodiments, ligand binding may be improved by mutation of residues in ER.

In one embodiment, the ER derived DD may be truncated and the smallest ER based DD may be identified. In some embodiments, ER DDs described herein may also be fragments of the above destabilizing domains, including fragments containing variant amino acid sequences. Preferred fragments are unstable in the absence of the stimulus and stabilized upon addition of the stimulus. Preferred fragments retain the ability to interact with the stimulus with similar efficiency as the DDs described herein.

In some embodiments, the ER mutant may comprise one or more mutations in an amino acid at the mutation site being identical to one or more vicinal amino acids. The vicinal amino acid may be selected from, but not limited to one, two, three, four, and five amino acids, upstream or downstream from the mutation.

In some embodiments, the ER-derived SRE of the effector module may exhibit both a destabilization ratio between 0 and 0.09 and a destabilizing mutation co-efficient between 0.09. The destabilization ratio may comprise the ratio of expression, function or level of the payload in the absence of the stimulus specific to the ER-derived SRE to the expression, function or level of the payload that is expressed constitutively in the absence of the same stimulus. The destabilizing mutation co-efficient may comprise the ratio of expression, function or level of the payload when operably linked to the ER-derived SRE, in the absence of the stimulus specific to the ER-derived SRE; to the expression, function or level of the payload when operably linked to the wildtype protein from which the ER-derived SRE is derived and in the absence of the same stimulus. In one aspect, the ER-derived SRE of the effector module may stabilize the payload by a stabilization ratio of 1 or more. The stabilization ratio may be the ratio of expression, function or level of the payload in the presence of the stimulus to the expression, function or level of the payload in the absence of the stimulus.

### Protein to Protein Interactions

In some embodiments, the stimulus response element may be destabilized by the stimulus. In some embodiments, SREs may be derived from protein complexes that comprise at least one protein-protein interaction. In other aspects, the SRE may form a protein-protein interaction with a natural protein within the cell. Protein complexes reduce the exposure of the constituent proteins to the risk of undesired oligomerization by reducing the concentration of the free monomeric state. Payloads appended to such SREs may stabilized in the absence of the stimulus. In some aspects, the stimulus may be a small molecule that is capable of interrupting or disrupting the protein-protein interactions related to the SRE. In such instances, addition of the stimulus, results in the reduced expression and/or function of the payload. In some embodiments, stimuli that induce conformational change of the SRE may be utilized. In one aspect, the SRE may be stabilized by the conformational change. In another aspect, the SRE may be destabilized by the conformational change. The stimuli may also be small molecules that disrupt post translational modification of SREs which may result in the disruption of the protein-protein interaction related to the SRE. In some embodiments, SREs may be identified using protein interactomic techniques known in the art. Such methods may enable the identification of protein interactions that are therapeutically relevant. Any of the large-scale quantitative proteomics methods described in International Patent Publication NOs. WO2017210427A1, WO2016196994A9, and WO2014200987A3 may be useful in the present disclosure (the contents of each of which are incorporated by reference in their entirety).

### Stimulus

Biocircuits of the disclosure are triggered by one or more stimuli. Stimuli may be selected from a ligand, an externally added or endogenous metabolite, the presence or absence of a defined ligand, pH, temperature, light, ionic strength, radioactivity, cellular location, subject site, microenvironment, the presence or the concentration of one or more metal ions.

In some embodiments, the stimulus is a ligand. Ligands may be nucleic acid-based, protein-based, lipid based, organic, inorganic or any combination of the foregoing. In some embodiments, the ligand is selected from the group consisting of a protein, peptide, nucleic acid, lipid, lipid derivative, sterol, steroid, metabolite derivative and a small molecule. In some embodiments, the stimulus is a small molecule. In some embodiments, the small molecules are cell permeable. Ligands useful in the present disclosure include without limitation, any of those taught in Table 2 of co-pending commonly owned U.S. Ser. No. 62/320,864 filed on Apr. 11, 2016, 62/466,596 filed Mar. 3, 2017 and the International Publication WO2017/180587, the contents of each of which are incorporated herein by reference in their entirety. In some embodiments, the small molecules are FDA-approved, safe and orally administered.

In some embodiments, the ligand binds to ER. Ligands may be agonists or antagonists. In some embodiments, the ligand binds to and inhibits ER function and is herein referred to as a ER inhibitor. In some embodiments, the ligand may be a selective inhibitor of human ER. Ligands of the disclosure may also be selective inhibitors of ER of other species. Ligands specific to other ER may be modified to improve binding to ER.

Ligands may be ER agonists such as but not limited to endogenous estrogen 17b-estradiol (E2) and the synthetic nonsteroidal estrogen diethylstilbestrol (DES). In some embodiments. The ligands may be ER antagonists, such as ICI-164,384, RU486, tamoxifen, 4-hydroxytamoxifen (4-OHT), fulvestrant, oremifene, lasofoxifene, clomifene, femarelle and ormeloxifene and raloxifene (RAL).

In some embodiments, the stimulus of the current disclosure may be ER antagonists such as, but not limited to Bazedoxifene and/or Raloxifene. In some embodiments, the ER antagonist may be Duavee, which is Bazedoxifene conjugated with estrogen. In one embodiment, the ER antagonist may be administered at clinically approved doses. Non limiting examples include Duavee, which may be administered at 20 mg (daily) and Raloxifene may be administered at 60 mg (daily).

In some embodiments, synthetic ligands CMP1, CMP2, CMP3, CMP4, CMP5, CMP6, CMP7, and CMP8 described by Gallinari et al. (2005) Chem. & Biol., Vol. 12, 883-893 AND Miyazaki et al. (2012) J Am Chem Soc. March 7; 134(9):3942-5 (the contents of each of which are herein incorporated by reference in their entirety).

Ligands may also be selected from but not limited to 2,3-Diaryl Isoquinolinone Bisphenol A, bisphenol AF, HPTE, CMPD3, CMPD6, CMPD9, Hydrazide derivatives, Fluoren-3-one derivatives, Triphenylethylene Coumarin, 1,1,2-Triarylolefine derivatives, aptamer modulators of estrogen receptors, Carborane analogues, pyrazole-based ligands (propyl-pyrazole-triol (PPT) and methyl-piperidino-pyrazole (MPP)).

In some embodiments, ligands of the present disclosure may be polyglutamate or non polyglutamylatable. Like naturally occurring folates, polyglutamatable folates also contain a glutamic acid residue and therefore undergo intracellular polyglutamylation. In contrast, non-polyglutamatable antifolates are devoid of a glutamate residue and thus are not available for polyglutamylation. In some embodiments, polyglutamylatable ligands may be preferred to increase intracellular retention as they can no longer be exported out of the cell. In other embodiments, non polyglutamylatable ligands may be preferred to decrease intracellular retention.

In one embodiment, the ligands of the present disclosure may be FDA approved ligands capable of binding to the specific DDs or target regions within the DDs. In other embodiments, FDA approved ligands may be used to screen potential binders in the human protein. DDs may be designed based on the positive hits from the screen using the portion of the protein that binds to the ligand. In one embodiment, proteins that bind to FDA approved ligands as off target interactions may be used to design DDs of the present disclosure.

In some embodiments, ligands include Bazedoxifene-derived ligands containing portions of the ligand known to mediate binding to ER. Ligands may also be modified to reduce off-target binding to other folate metabolism enzymes and increase specific binding to ER derived DDs.

In some embodiments, the ligand selection is determined by the magnitude and duration of expression of the effector modules of the disclosure using the PK parameters. In some embodiments, high levels of expression of the payload for a short duration of time may be desired. In some embodiments, high levels of expression of the payload may be desired for a long duration. In some embodiments, low levels of expression of the payload may be desired for a long duration of time. In some embodiments, low levels of expression for a short duration of time may be desired. In such instances, TMP may be used as the ligand.

Ligands may also be selected from the analysis of the dependence of a known ER ligand on its molecular/chemical structure, through Structure Activity Relationships (SAR) study. Any of the methods related to SAR, known in art may be utilized to identify stabilizing ligands of the disclosure. SAR may be utilized to improve properties of the ligand such as specificity, potency, pharmacokinetics, bioavailability, and safety. SAR analysis of known ER ligands may also be combined with computational strategies and the high-resolution X ray structures of ER complexed with ligands may be used to develop compounds that can fit these criteria.

In one embodiment, the ligand may not be capable of suppressing the immune system i.e. the ligand may be non-immunosuppressive.

### Payloads

According to the present disclosure, payloads can be any natural protein in an organism genome, a fusion polypeptide, an antibody, or variants, mutants and derivatives thereof

### 1. Protein of Interest

In some embodiments, payloads of the disclosure may be a natural protein in an organism genome, or variants, mutants, derivatives thereof. The natural protein may be from, for example, a mammalian organism, a bacterium, and a virus.

In one example, the payload may be a protein of interest, or a polypeptide from human genome.

In some embodiments, the payload of the present disclosure may be cardiac lineage specification factors such as eomesodermin (EOMES), a T-box transcription factor; WNT signaling pathway components such as WNT3 and WNT 3A. EOMES is crucially required for the development of the heart. Cardiomyocyte programming by EOMES involves autocrine activation of the canonical WNT signaling pathway and vice versa. Under conditions that are conducive to promoting cardiac lineage, WNT signaling activates EOMES and EOMES in turn promotes WNT signaling creating a self-sustaining loop that promotes the cardiac lineage. An activation loop that is too weak or too strong promotes non-cardiac fates such as endodermal and other mesodermal fates respectively. The DDs of the present disclosure may be used to tune EOMES and WNT payload levels to generate an activation loop that initiate and/or sustain cardiac specification during gastrulation.

In some embodiments, the payload may be a fusion protein comprising any of the immunotherapeutic agents described and ubiquitin. Within the fusion protein, the ubiquitin may be positioned at the N terminus and the immunotherapeutic agent may be positioned at the C terminus. In one aspect, the immunotherapeutic agent may itself be a fusion protein and the ubiquitin may be located in between the proteins that are fused. The payloads may include a single ubiquitin protein or a chain of ubiquitin proteins. The ubiquitin protein may be linked to the immunotherapeutic agent through a single amino acid. The selection of the single amino acid may depend on the desired half-life of the fusion protein. In one embodiment, the immunotherapeutic agent may be IL12.

### 2. Antibodies, Antibody Fragments and Variants

In some embodiments, payloads of the disclosure may be an antibody or fragments thereof. Antibodies useful in this method include without limitation, any of those taught in co-pending commonly owned U.S. Provisional Patent Application No. 62/320,864 filed on Apr. 11, 2016, or in U.S. Provisional Application No. 62/466,596 filed Mar. 3, 2017 and the International Publication WO2017/180587, the contents of each of which are incorporated herein by reference in their entirety.

The antibody may be an intact antibody, an antibody light chain, antibody heavy chain, an antibody fragment, an antibody variant, or an antibody derivative.

For the purposes herein, an “antibody” may comprise a heavy and light variable domain as well as an Fc region. As used herein, the term “native antibody” refers to a usually heterotetrameric glycoprotein of about 150,000 Daltons, composed of two identical light (L) chains and two identical heavy (H) chains. Genes encoding antibody heavy and light chains are known and segments making up each have been well characterized and described (Matsuda et al., The Journal of Experimental Medicine. 1998, 188(11): 2151-62 and Li et al., Blood, 2004, 103(12): 4602-4609; the content of each of which are herein incorporated by reference in their entirety). Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (VH) followed by a number of constant domains. Each light chain has a variable domain at one end (VL) and a constant domain at its other end; the constant domain of the light chain is aligned with the first constant domain of the heavy chain, and the light chain variable domain is aligned with the variable domain of the heavy chain.

As used herein, the term “variable domain” refers to specific antibody domains found on both the antibody heavy and light chains that differ extensively in sequence among antibodies and are used in the binding and specificity of each particular antibody for its particular antigen. Variable domains comprise hypervariable regions. As used herein, the term “hypervariable region” refers to a region within a variable domain comprising amino acid residues responsible for antigen binding. The amino acids present within the hypervariable regions determine the structure of the complementarity determining regions (CDRs) that become part of the antigen-binding site of the antibody. As used herein, the term “CDR” refers to a region of an antibody comprising a structure that is complimentary to its target antigen or epitope. Other portions of the variable domain, not interacting with the antigen, are referred to as framework (FW) regions. The antigen-binding site (also known as the antigen combining site or paratope) comprises the amino acid residues necessary to interact with a particular antigen. The exact residues making up the antigen-binding site are typically elucidated by co-crystallography with bound antigen, however computational assessments can also be used based on comparisons with other antibodies (Strohl, W. R. Therapeutic Antibody Engineering. Woodhead Publishing, Philadelphia Pa. 2012. Ch. 3, p47-54, the contents of which are herein incorporated by reference in their entirety). Determining residues making up CDRs may include the use of numbering schemes including, but not limited to, those taught by Kabat (Wu et al., JEM, 1970, 132(2):211-250 and Johnson et al., Nucleic Acids Res. 2000, 28(1): 214-218, the contents of each of which are herein incorporated by reference in their entirety), Chothia (Chothia and Lesk, J. Mol. Biol. 1987, 196, 901, Chothia et al., Nature, 1989, 342, 877, and Al-Lazikani et al., J. Mol. Biol. 1997, 273(4): 927-948, the contents of each of which are herein incorporated by reference in their entirety), Lefranc (Lefranc et al., Immunome Res. 2005, 1:3) and Honegger (Honegger and Pluckthun, J. Mol. Biol. 2001, 309(3): 657-70, the contents of which are herein incorporated by reference in their entirety).

VH and VL domains have three CDRs each. VL CDRs are referred to herein as CDR-L1, CDR-L2 and CDR-L3, in order of occurrence when moving from N- to C-terminus along the variable domain polypeptide. VH CDRs are referred to herein as CDR-H1, CDR-H2 and CDR-H3, in order of occurrence when moving from N- to C-terminus along the variable domain polypeptide. Each of CDRs have favored canonical structures with the exception of the CDR-H3, which comprises amino acid sequences that may be highly variable in sequence and length between antibodies resulting in a variety of three-dimensional structures in antigen-binding domains (Nikoloudis, et al., Peer J. 2014, 2: e456). In some cases, CDR-H3s may be analyzed among a panel of related antibodies to assess antibody diversity. Various methods of determining CDR sequences are known in the art and may be applied to known antibody sequences (Strohl, W. R. Therapeutic Antibody Engineering. Woodhead Publishing, Philadelphia Pa. 2012. Ch. 3, p47-54, the contents of which are herein incorporated by reference in their entirety).

As used herein, the term “light chain” refers to a component of an antibody from any vertebrate species assigned to one of two clearly distinct types, called kappa and lambda based on amino acid sequences of constant domains. Depending on the amino acid sequence of the constant domain of their heavy chains, antibodies can be assigned to different classes. There are five major classes of intact antibodies: IgA, IgD, IgE, IgG, and IgM, and several of these may be further divided into subclasses (isotypes), e.g., IgG1, IgG2, IgG3, IgG4, IgA, and IgA2.

In some embodiments, the payload maybe a monoclonal antibody. As used herein, the term “monoclonal antibody” refers to an antibody obtained from a population of substantially homogeneous cells (or clones), i.e., the individual antibodies comprising the population are identical and/or bind the same epitope, except for possible variants that may arise during production of the monoclonal antibodies, such variants generally being present in minor amounts. In contrast to polyclonal antibody preparations that typically include different antibodies directed against different determinants (epitopes), each monoclonal antibody is directed against a single determinant on the antigen.

The modifier “monoclonal” indicates the character of the antibody as being obtained from a substantially homogeneous population of antibodies and is not to be construed as requiring production of the antibody by any particular method. The monoclonal antibodies herein include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies.

In one embodiment, the payload of the present disclosure may be a humanized antibody. As used herein, the term “humanized antibody” refers to a chimeric antibody comprising a minimal portion from one or more non-human (e.g., murine) antibody source(s) with the remainder derived from one or more human immunoglobulin sources. For the most part, humanized antibodies are human immunoglobulins (recipient antibody) in which residues from the hypervariable region from an antibody of the recipient are replaced by residues from the hypervariable region from an antibody of a non-human species (donor antibody) such as mouse, rat, rabbit or nonhuman primate having the desired specificity, affinity, and/or capacity. In one embodiment, the antibody may be a humanized full-length antibody. As a non-limiting example, the antibody may have been humanized using the methods taught in US Patent Publication NO. US20130303399, the contents of which are herein incorporated by reference in its entirety.

In one embodiment, the antibody may comprise a modified Fc region. As a non-limiting example, the modified Fc region may be made by the methods or may be any of the regions described in US Patent Publication NO. US20150065690, the contents of which are herein incorporated by reference in its entirety.

As used herein, the term “antibody variant” refers to a modified antibody (in relation to a native or starting antibody) or a biomolecule resembling a native or starting antibody in structure and/or function (e.g., an antibody mimetic). Antibody variants may be altered in their amino acid sequence, composition or structure as compared to a native antibody. Antibody variants may include, but are not limited to, antibodies with altered isotypes (e.g., IgA, IgD, IgE, IgG1, IgG2, IgG3, IgG4, or IgM), humanized variants, optimized variants, multispecific antibody variants (e.g., bispecific variants), and antibody fragments.

In some embodiments, antibody fragments and variants may comprise antigen binding regions from intact antibodies. Examples of antibody fragments and variants may include, but are not limited to Fab, Fab′, F(ab′)2, and Fv fragments; diabodies; linear antibodies; single-chain antibody molecules such as single chain variable fragment (scFv); dimeric single-chain variable fragment (di-scFv), single domain antibody (sdAb) and multispecific antibodies formed from antibody fragments. Papain digestion of antibodies produces two identical antigen-binding fragments, called “Fab” fragments, each with a single antigen-binding site. Also produced is a residual “Fc” fragment, whose name reflects its ability to crystallize readily. Pepsin treatment yields an F(ab′)2 fragment that has two antigen-binding sites and is still capable of cross-linking antigen. Pharmaceutical compositions, biocircuits, biocircuit components, effector modules including their SREs or payloads of the present disclosure may comprise one or more of these fragments.

As used herein, the term “Fv” refers to an antibody fragment comprising the minimum fragment on an antibody needed to form a complete antigen-binding site. These regions consist of a dimer of one heavy chain and one light chain variable domain in tight, non-covalent association. Fv fragments can be generated by proteolytic cleavage but are largely unstable. Recombinant methods are known in the art for generating stable Fv fragments, typically through insertion of a flexible linker between the light chain variable domain and the heavy chain variable domain (to form a single chain Fv (scFv) or through the introduction of a disulfide bridge between heavy and light chain variable domains (Strohl, W. R. Therapeutic Antibody Engineering. Woodhead Publishing, Philadelphia Pa. 2012. Ch. 3, p 46-47, the contents of which are herein incorporated by reference in their entirety).

As used herein, the term “single chain Fv” or “scFv” refers to a fusion protein of VH and VL antibody domains, wherein these domains are linked together into a single polypeptide chain by a flexible peptide linker. In some embodiments, the Fv polypeptide linker enables the scFv to form the desired structure for antigen binding. In some embodiments, scFvs are utilized in conjunction with phage display, yeast display or other display methods where they may be expressed in association with a surface member (e.g. phage coat protein) and used in the identification of high affinity peptides for a given antigen.

Using molecular genetics, two scFvs can be engineered in tandem into a single polypeptide, separated by a linker domain, called a “tandem scFv” (tascFv). Construction of a tascFv with genes for two different scFvs yields a “bispecific single-chain variable fragments” (bis-scFvs). Only two tascFvs have been developed clinically by commercial firms; both are bispecific agents in active early phase development by Micromet for oncologic indications and are described as “Bispecific T-cell Engagers (BiTE).” Blinatumomab is an anti-CD19/anti-CD3 bispecific tascFv that potentiates T-cell responses to B-cell non-Hodgkin lymphoma in Phase 2. MT110 is an anti-EP-CAM/anti-CD3 bispecific tascFv that potentiates T-cell responses to solid tumors in Phase 1. Bispecific, tetravalent “TandAbs” are also being researched by Affimed (Nelson, A. L., MAbs., 2010, January-February; 2(1):77-83). maxibodies (bivalent scFv fused to the amino terminus of the Fc (CH2-CH3 domains) of IgG may also be included.

As used herein, the term “bispecific antibody” refers to an antibody capable of binding two different antigens. Such antibodies typically comprise regions from at least two different antibodies. Bispecific antibodies may include any of those described in Riethmuller, G. Cancer Immunity. 2012, 12:12-18, Marvin et al., 2005. Acta Pharmacologica Sinica. 2005, 26(6): 649-658 and Schaefer et al., PNAS. 2011, 108(27):11187-11192, the contents of each of which are herein incorporated by reference in their entirety. In some aspects, bispecific antibodies may be trifunctional antibodies (3funct) and BiTE (bi-specific T cell engager).

As used herein, the term “diabody” refers to a small antibody fragment with two antigen-binding sites. Diabodies are functional bispecific single-chain antibodies (bscAb). Diabodies comprise a heavy chain variable domain VH connected to a light chain variable domain VL in the same polypeptide chain. By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al. (Hollinger, P. et al., “Diabodies”: Small bivalent and bispecific antibody fragments. PNAS, 1993. 90: 6444-6448); the contents of each of which are incorporated herein by reference in their entirety.

The term “intrabody” refers to a form of antibody that is not secreted from a cell in which it is produced, but instead targets one or more intracellular proteins. Intrabodies may be used to affect a multitude of cellular processes including, but not limited to intracellular trafficking, transcription, translation, metabolic processes, proliferative signaling and cell division. In some embodiments, methods of the present disclosure may include intrabody-based therapies. In some such embodiments, variable domain sequences and/or CDR sequences disclosed herein may be incorporated into one or more constructs for intrabody-based therapy.

In some embodiments, antibody variants may be antibody mimetics. As used herein, the term “antibody mimetic” refers to any molecule which mimics the function or effect of an antibody and which binds specifically and with high affinity to their molecular targets. In some embodiments, antibody mimetics may be monobodies, designed to incorporate the fibronectin type III domain (Fn3) as a protein scaffold (U.S. Pat. Nos. 6,673,901; 6,348,584). In some embodiments, antibody mimetics may be those known in the art including, but are not limited to affibody molecules, affilins, affitins, anticalins, avimers, Centyrins, DARPINS™, Fynomers and Kunitz and domain peptides. In other embodiments, antibody mimetics may include one or more non-peptide regions.

In some embodiments, antibody variants may be multispecific antibodies that bind more than one epitope. As used herein, the terms “multibody” or “multispecific antibody” refer to an antibody wherein two or more variable regions bind to different epitopes. The epitopes may be on the same or different targets. In one embodiment, the multispecific antibody may be generated and optimized by the methods described in International Patent Publication NO. WO2011109726 and US Patent Publication NO. US20150252119, the contents of which each of which are herein incorporated by reference in their entirety. These antibodies are able to bind to multiple antigens with high specificity and high affinity.

In certain embodiments, a multi-specific antibody is a “bispecific antibody” which recognizes two different epitopes on the same or different antigens. In one aspect, bispecific antibodies are capable of binding two different antigens. Such antibodies typically comprise antigen-binding regions from at least two different antibodies. For example, a bispecific monoclonal antibody (BsMAb, BsAb) is an artificial protein composed of fragments of two different monoclonal antibodies, thus allowing the BsAb to bind to two different types of antigen. Bispecific antibody frameworks may include any of those described in Riethmuller, G., 2012. Cancer Immunity, 2012, 12:12-18; Marvin et al., Acta Pharmacologica Sinica. 2005, 26(6):649-658; and Schaefer et al., PNAS. 2011, 108(27): 11187-11192, the contents of each of which are herein incorporated by reference in their entirety. New generations of BsMAb, called “trifunctional bispecific” antibodies, have been developed. These consist of two heavy and two light chains, one each from two different antibodies, where the two Fab regions (the arms) are directed against two antigens, and the Fc region (the foot) comprises the two heavy chains and forms the third binding site.

In certain embodiments, antibody variants may be antibodies comprising a single antigen-binding domain. These molecules are extremely small, with molecular weights approximately one-tenth of those observed for full-sized mAbs. Further antibodies may include “nobodies” derived from the antigen-binding variable heavy chain regions (VHHs) of heavy chain antibodies found in camels and llamas, which lack light chains (Nelson, A. L., MAbs. 2010. January-February; 2(1):77-83).

In some embodiments, the antibody may be “miniaturized”. Among the best examples of mAb miniaturization are the small modular immunopharmaceuticals (SMIPs) from Trubion Pharmaceuticals. These molecules, which can be monovalent or bivalent, are recombinant single-chain molecules containing one VL, one VH antigen-binding domain, and one or two constant “effector” domains, all connected by linker domains. Presumably, such a molecule might offer the advantages of increased tissue or tumor penetration claimed by fragments while retaining the immune effector functions conferred by constant domains. At least three “miniaturized” SMIPs have entered clinical development. TRU-015, an anti-CD20 SMIP developed in collaboration with Wyeth, is the most advanced project, having progressed to Phase 2 for rheumatoid arthritis (RA). Earlier attempts in systemic lupus erythematosus (SLE) and B cell lymphomas were ultimately discontinued. Trubion and Facet Biotechnology are collaborating in the development of TRU-016, an anti-CD37 SMIP, for the treatment of CLL and other lymphoid neoplasias, a project that has reached Phase 2. Wyeth has licensed the anti-CD20 SMIP SBI-087 for the treatment of autoimmune diseases, including RA, SLE and possibly multiple sclerosis, although these projects remain in the earliest stages of clinical testing. (Nelson, A. L., MAbs, 2010. January-February; 2(1):77-83).

One example of miniaturized antibodies is called “unibody” in which the hinge region has been removed from IgG4 molecules. While IgG4 molecules are unstable and can exchange light-heavy chain heterodimers with one another, deletion of the hinge region prevents heavy chain-heavy chain pairing entirely, leaving highly specific monovalent light/heavy heterodimers, while retaining the Fc region to ensure stability and half-life in vivo. This configuration may minimize the risk of immune activation or oncogenic growth, as IgG4 interacts poorly with FcRs and monovalent unibodies fail to promote intracellular signaling complex formation (see, e.g., Nelson, A. L., MAbs, 2010. January-February; 2(1):77-83).

In some embodiments, antibody variants may include single-domain antibodies (sdAbs, or nanobodies) which are antibody fragment consisting of a single monomeric variable antibody domain. Like a whole antibody, it is able to bind selectively to a specific antigen. In one aspect, a sdAb may be a “Camel Ig or “camelid VHH”. As used herein, the term “camel Ig” refers to the smallest known antigen-binding unit of a heavy chain antibody (Koch-Nolte, et al, FASEB J., 2007, 21: 3490-3498). A “heavy chain antibody” or a “camelid antibody” refers to an antibody that contains two VH domains and no light chains (Riechmann L. et al, J. Immunol. Methods, 1999, 231: 25-38; International patent publication NOs. WO1994/04678 and WO1994/025591; and U.S. Pat. No. 6,005,079). In another aspect, an sdAb may be a “immunoglobulin new antigen receptor” (IgNAR). As used herein, the term “immunoglobulin new antigen receptor” refers to class of antibodies from the shark immune repertoire that consist of homodimers of one variable new antigen receptor (VNAR) domain and five constant new antigen receptor (CNAR) domains. IgNARs represent some of the smallest known immunoglobulin-based protein scaffolds and are highly stable and possess efficient binding characteristics. The inherent stability can be attributed to both (i) the underlying Ig scaffold, which presents a considerable number of charged and hydrophilic surface exposed residues compared to the conventional antibody VH and VL domains found in murine antibodies; and (ii) stabilizing structural features in the complementary determining region (CDR) loops including inter-loop disulfide bridges, and patterns of intra-loop hydrogen bonds.

In some embodiments, antibody variants may include intrabodies. Intrabodies are a form of antibody that is not secreted from a cell in which it is produced, but instead targets one or more intracellular proteins. Intrabodies are expressed and function intracellularly and may be used to affect a multitude of cellular processes including, but not limited to intracellular trafficking, transcription, translation, metabolic processes, proliferative signaling and cell division. In some embodiments, methods described herein include intrabody-based therapies. In some such embodiments, variable domain sequences and/or CDR sequences disclosed herein are incorporated into one or more constructs for intrabody-based therapy. For example, intrabodies may target one or more glycated intracellular proteins or may modulate the interaction between one or more glycated intracellular proteins and an alternative protein.

The intracellular expression of intrabodies in different compartments of mammalian cells allows blocking or modulation of the function of endogenous molecules (Biocca, et al., EMBO J. 1990, 9: 101-108; Colby et al., Proc. Natl. Acad. Sci. U.S.A. 2004, 101: 17616-17621). Intrabodies can alter protein folding, protein-protein, protein-DNA, protein-RNA interactions and protein modification. They can induce a phenotypic knockout and work as neutralizing agents by direct binding to the target antigen, by diverting its intracellular trafficking or by inhibiting its association with binding partners. With high specificity and affinity to target antigens, intrabodies have advantages to block certain binding interactions of a particular target molecule, while sparing others.

Sequences from donor antibodies may be used to develop intrabodies. Intrabodies are often recombinantly expressed as single domain fragments such as isolated VH and VL domains or as a single chain variable fragment (scFv) antibody within the cell. For example, intrabodies are often expressed as a single polypeptide to form a single chain antibody comprising the variable domains of the heavy and light chains joined by a flexible linker polypeptide. Intrabodies typically lack disulfide bonds and are capable of modulating the expression or activity of target genes through their specific binding activity. Single chain intrabodies are often expressed from a recombinant nucleic acid molecule and engineered to be retained intracellularly (e.g., retained in the cytoplasm, endoplasmic reticulum, or periplasm). Intrabodies may be produced using methods known in the art, such as those disclosed and reviewed in: (Marasco et al., PNAS, 1993, 90: 7889-7893; Chen et al., Hum. Gene Ther. 1994, 5:595-601; Chen et al., 1994, PNAS, 91: 5932-5936; Maciejewski et al., 1995, Nature Med., 1: 667-673; Marasco, 1995, Immunotech, 1: 1-19; Mhashilkar, et al., 1995, EMBO J. 14: 1542-51; Chen et al., 1996, Hum. Gene Therap., 7: 1515-1525; Marasco, Gene Ther. 4:11-15, 1997; Rondon and Marasco, 1997, Annu. Rev. Microbiol. 51:257-283; Cohen, et al., 1998, Oncogene 17:2445-56; Proba et al., 1998, J. Mol. Biol. 275:245-253; Cohen et al., 1998, Oncogene 17:2445-2456; Hassanzadeh, et al., 1998, FEBS Lett. 437:81-6; Richardson et al., 1998, Gene Ther. 5:635-44; Ohage and Steipe, 1999, J. Mol. Biol. 291:1119-1128; Ohage et al., 1999, J. Mol. Biol. 291:1129-1134; Wirtz and Steipe, 1999, Protein Sci. 8:2245-2250; Zhu et al., 1999, J. Immunol. Methods 231:207-222; Arafat et al., 2000, Cancer Gene Ther. 7:1250-6; der Maur et al., 2002, J. Biol. Chem. 277:45075-85; Mhashilkar et al., 2002, Gene Ther. 9:307-19; and Wheeler et al., 2003, FASEB J. 17: 1733-5; and references cited therein).

In certain embodiments, antibody variants may include biosynthetic antibodies as described in U.S. Pat. No. 5,091,513, the contents of which are herein incorporated by reference in their entirety. Such antibody may include one or more sequences of amino acids constituting a region which behaves as a biosynthetic antibody binding site (BABS). The sites comprise 1) non-covalently associated or disulfide bonded synthetic VH and VL dimers, 2) VH-VL or VL-VH single chains wherein the VH and VL are attached by a polypeptide linker, or 3) individuals VH or VL domains. The binding domains comprise linked CDR and FR regions, which may be derived from separate immunoglobulins. The biosynthetic antibodies may also include other polypeptide sequences which function, e.g., as an enzyme, toxin, binding site, or site of attachment to an immobilization media or radioactive atom. Methods are disclosed for producing the biosynthetic antibodies, for designing BABS having any specificity that can be elicited by in vivo generation of antibody, and for producing analogs thereof.

In some embodiments, antibody variants may include antibodies with antibody acceptor frameworks taught in U.S. Pat. No. 8,399,625. Such antibody acceptor frameworks may be particularly well suited accepting CDRs from an antibody of interest.

In one embodiment, the antibody may be a conditionally active biologic protein. An antibody may be used to generate a conditionally active biologic protein which are reversibly or irreversibly inactivated at the wild type normal physiological conditions as well as to such conditionally active biologic proteins and uses of such conditional active biologic proteins are provided. Such methods and conditionally active proteins are taught in, for example, International Publication Nos. WO2015175375 and WO2016036916 and US Patent Publication No. US20140378660, the contents of each of which are incorporated herein by reference in their entirety.

The preparation of antibodies, whether monoclonal or polyclonal, is known in the art. Techniques for the production of antibodies are well known in the art and described, e.g. in Harlow and Lane “Antibodies, A Laboratory Manual”, Cold Spring Harbor Laboratory Press, 1988; Harlow and Lane “Using Antibodies: A Laboratory Manual” Cold Spring Harbor Laboratory Press, 1999 and “Therapeutic Antibody Engineering: Current and Future Advances Driving the Strongest Growth Area in the Pharmaceutical Industry” Woodhead Publishing, 2012.

The antibodies and fragments and variants thereof as described herein can be produced using recombinant polynucleotides. In one embodiment, the polynucleotides have a modular design to encode at least one of the antibodies, fragments or variants thereof. As a non-limiting example, the polynucleotide construct may encode any of the following designs: (1) the heavy chain of an antibody, (2) the light chain of an antibody, (3) the heavy and light chain of the antibody, (4) the heavy chain and light chain separated by a linker, (5) the VH1, CH1, CH2, CH3 domains, a linker and the light chain or (6) the VH1, CH1, CH2, CH3 domains, VL region, and the light chain. Any of these designs may also comprise optional linkers between any domain and region. The polynucleotides of the present disclosure may be engineered to produce any standard class of immunoglobulins using an antibody described herein or any of its component parts as a starting molecule.

In some embodiments, antibody payloads of the present disclosure may be therapeutic antibodies. As non-limiting examples, antibodies and fragments and variants thereof may be specific to tumor associated antigens, or tumor specific antigens, or pathogen antigens. In some aspects, antibodies may be blocking antibodies (also referred to as antagonistic antibodies), for example, blocking antibodies against PD-1, PD-L1, PD-L2, CTLA-4 and other inhibitory molecules. In other aspects, antibodies may be agonist antibodies such as agonistic antibodies specific to stimulatory molecules, e.g., 4-1BB (CD137), OX40 (CD134), CD40, GITR and CD27.

Other exemplary therapeutic antibodies may include, but are not limited to, Abagovomab, Abcxmab, Abituzumab, Abrilumab, Actoxumab, Adalimumab, Adecatumumab, Afasevikumab, Afelimomab, Afutuzumab, Alacizumab, Alemtuzumab, Alirocumab, Altumomab, Amatuximab, Anetumab, Anifrolumab, Apolizumab, Arcitumomab, Ascrinvacumab, Aselizumab, Atezolizumab, Atinumab, Atlizumab, Atorolimumab, Avelumab, Bapineuzumab, Basiliximab, Bavituximab, Bectumomab, Begelomab, Belimumab, Benralizumab, Bertilimumab, Besilesomab, Bevacizumab, Bezlotoxumab, Biciromab, Bimagrumab, Bimekizumab, Bivatuzumab, Bleselumab, Blinatumomab, Blinatumomab, Blosozumab, Bococizumab, Brentuximab, Briaknumab, Brodalumab, Brolucizumab, Brontictuzumab, Cabiralizumab, Canakinumab, Cantuzumab, Caplacizumab, Capromab, Carlumab, Carotuximab, Catumaxomab, cBR96-doxorubicin immunoconjugate, Cedelizumab, Cergutuzumab, Certolizumab pegol, Cetuximab, Citatuzumab, Cixutumumab, Clazakizumab, Clenoliximab, Clivatuzumab, Codrituzumab, Coltuximab, Contatumumab, Concizumab, Crenezumab, Crotedumab, CR6261, Dacetumab, Daclizumab, Dalotuzumab, Dapirolizumab pegol, Daratumumab, Dectrekumab, Demcizumab, Denintuzumab, Denosumab, Derlotuximab biotin, Detumomab, Dinutuximab, Diridavumab, Domagrozumab, Dorlimomab aritox, Drozitumab, Duligotumab, Dupilumab, Durvalumab, Dusigitumab, Ecromeximab, Eculizumab, Edobacomab, Edrecolomab, Efalizumab, Efungumab, Eldelumab, Elgemtumab, Elotuzumab, Elsilimomab, Emactuzumab, Emibetuzumab, Emicizumab, Enavatuzumab, Enfortumab vedotin, Enlimomab pegol, Enoblituzumab, Enokizumab, Enoticumab, Ensituximab, Epitumomab cituxetan, Epratuzumab, Erlizumab, Ertumaxomab, Etaracizumab, Etrolizumab, Evinacumab, Evolocumab, Exbivirumab, Fanolesomab, Faralimomab, Farletuzumab, Fasinumab, FBTA05, Felvizumab, Fezakinumab, Fibatuzumab, Ficlatuzumab, Figitumumab, Firivumab, Flanvotumab, Fletikumab, Fontolizumab, Foralumab, Foravirumab, Fresolimumab, Fulranumab, Futuximab, Galcanezumab, Galiximab, Ganitumab, Gantenerumab, Gavilimomab, Gemtuzumab ozogamicin, Gevokizumab, Girentuximab, Glembatumumab vedotin, Golimumab, Gomiliximab, Guselkumab, Ibalizumab, Ibritumomab tituxetan, icrucumab, Idarucizumab, Igovomab, IMAB362, Imalumab, Imciromab, Imgatuzumab, Inclacumab, Indatuximab, Indusatumab, Inebilizumab, Infliximab, Intetumumab, Inolimomab, Inotuzumab, Ipilimumab, Iratumumab, Isatuximab, Itolizumab, Ixekizumab, Keliximab, Labetuzumab, Lambrolizumab, Lampalizumab, Lanadelumab, Landogrozumab, Laprituximab, Lebrikizumab, Lemalesomab, Lendalizumab, Lenzilumab, Lerdelimumab, Lexatumumab, Libivirumab, Lifastuzumab, Ligelizumab, Lilotomab, Lintuzumab, Lirilumab, Lodelcizumab, Lokivetmab, Lorvotuzumab, Lucatumumab, Lulizumab pegol, Lumiliximab, Lumretuzumab, Mapatumumab, Margetuximab, Maslimomab, Mavrilimumab, Matuzumab, Mepolizumab, Metelimumab, Milatuzumab, Minretumomab, Mirvetuximab, Mitumomab, Mogamulizumab, Monalizumab, Morolimumab, Motavizumab, Moxetumomab pasudotox, Muromonab-CD3, nacolomab tafenatox, Namilumab, naptumomab, naratuximab, Narnatumab, Natalizumab, Navicixizumab, Navivumab, Nebacumab, Necitumumab, Nemolizumab, Nerelimomab, Nesvacumab, Nimotuzumab, Nivolumab, Nofetumomab, Obiltoxaximab, Obinutuzumab, Ocaratuzumab, Ocrelizumab, Odulimomab, Ofatumumab, Olaratumab, Olaratumab, Olokizumab, Omalizumab, Onartuzumab, Ontuxizumab, Opicinumab, Oportuzumab monatox, Oregovomab, Orticumab, Otelixizumab, Otlertuzumab, Oxelumab, Ozanezumab, Ozoralizumab, Pagibaximab, Palivizumab, Pamrevlumab, Panitumumab, Pankomab, Panobacumab, Parsatuzumab, Pascolizumab, Pasotuxizumab, Pateclizumab, Patritumab, Pembrolizumab, Pemtumomab, Perakizumab, Pertuzumab, Pexelizumab, Pidilizumab, Pinatuzumab, Pintumomab, Placulumab, Plozalizumab, Pogalizumab, Polatuzumab, Ponezumab, Prezalizumab, Priliximab, Pritoxaximab, Pritumumab, PRO 140, Quilizumab, Racotumomab, Radretumab, Rafivirumab, Ralpancizumab, Ramucirumab, Ranibizumab, Raxibacumab, Refanezumab, Regavirumab, Reslizumab, Rilotumumab, Rinucumab, Risankizumab, Rituximab, Rivabazumab pegol, Robatumumab, Roledumab, Romosozumab, Rontalizumab, Rovalpituzumab, Rovelizumab, Ruplizumab, Sacituzumab, Samalizumab, Sapelizumab, Sarilumab, Satumomab pendetide, Secukinumab, Seribantumab, Setoxaximab, Sevirumab, Sibrotuzumab, SGN-CD19A, SGN-CD33A, Sifalimumab, Siltuximab, Simtuzumab, Siplizumab, Sirukumab, Sofituzumab vedotin, Solanezumab, Solitomab, Sonepcizumab, Sontuzumab, Stamulumab, Sulesomab, Suvizumab, tabalumab, Tacatuzumab, Tadocizumab, Talizumab, Tamtuvetmab, Tanezumab, Taplitumomab, Tarextumab, Tefibazumab, Telimomab aritox, Tenatumomab, Teneliximab, Teplizumab, Teprotumumab, Tesidolumab, Tetulomab, Tezepelumab, TGN1412, Ticilimumab, Tildrakizumab, Tigatuzumab, Timolumab, Tisotumab vedotin, TNX-650, Tocilizumab, Toralizumab, Tosatoxumab, Tositumomab, Tovetumab, Tralokinumab, Trastuzumab, TRBS07, Tregalizumab, Tremelimumab, Trevogrumab, Tucotuzumab, Tuvirumab, Ublituximab, Ulcocuplumab, Urelumab, Urtoxazumab, Ustekinumab, Vadastuximab talirine, Vandortuzumab vedotin, Vantictumab, Vanucizumab, Vapaliximab, Varlilumab, Vatelizumab, Vedolizumab, Veltuzumab, Vepalimomab, Vesencumab, Visilizumab, Vobarilizumab, Volociximab, Vorsetuzumab, Votumumab, Xentuzumab, Zalutumumab, Zanolimumab, Zatuximab, Ziralimumab and Zolimomab aritox.

Bicistronic and/or Pseudo-Bicistronic Antibody Payloads

According to the present disclosure, a bicistronic payload is a polynucleotide encoding a two-protein chain antibody on a single polynucleotide strand. A pseudo-bicistronic payload is a polynucleotide encoding a single chain antibody discontinuously on a single polynucleotide strand. For bicistronic payloads, the encoded two strands or two portions/regions and/or domains (as is the case with pseudo-bicistronic) are separated by at least one nucleotide not encoding the strands or domains. More often the separation comprises a cleavage signal or site or a non-coding region of nucleotides. Such cleavage sites include, for example, furin cleavage sites encoded as an “RKR” site, or a modified furin cleavage site in the resultant polypeptide or any of those taught herein.

According to the present disclosure, a single domain payload comprises one or two polynucleotides encoding a single monomeric variable antibody domain. Typically, single domain antibodies comprise one variable domain (VH) of a heavy-chain antibody.

According to the present disclosure, a single chain Fv payloads is a polynucleotide encoding at least two coding regions and a linker region. The scFv payload may encode a fusion protein of the variable regions of the heavy (VH) and light chains (VL) of immunoglobulins, connected with a short linker peptide of ten to about 25 amino acids. The linker is usually rich in glycine for flexibility, as well as serine or threonine for solubility, and can either connect the N-terminus of the VH with the C-terminus of the VL, or vice versa. Other linkers include those known in the art and disclosed herein.

According to the present disclosure, a bispecific payload is a polynucleotide encoding portions or regions of two different antibodies. Bispecific payloads encode polypeptides which may bind two different antigens. Polynucleotides of the present disclosure may also encode trispecific antibodies having an affinity for three antigens.

### 3. Therapeutic Agents

In some embodiments, payloads of the present disclosure may be a therapeutic agent, such as a cancer therapeutic agent, an immunotherapeutic agent, an anti-pathogen agent or a gene therapy agent. In some aspects, the immunotherapeutic agent may be a TCR receptor, a chimeric antigen receptor (CAR), a chimeric switch receptor, an antagonist of a co-inhibitory molecule, an agonist of a co-stimulatory molecule, a cytokine, a cytokine receptor, a chemokine, a chemokine receptor, a metabolic factor, a homing receptor and a safety switch.

As used herein, the term “chimeric antigen receptor (CAR)” refers to a synthetic receptor that mimics TCR on the surface of T cells. In general, a CAR is composed of an extracellular targeting domain, a transmembrane domain/region and an intracellular signaling/activation domain. Cells such as T cells engineered to express a CAR can be redirected to attack target cells that express a molecule which can be recognized by the targeting moiety of the CAR. In a standard CAR receptor, the components: the extracellular targeting domain, transmembrane domain and intracellular signaling/activation domain, are linearly constructed as a single fusion protein. The extracellular region comprises a targeting domain/moiety (e.g., a scFv) that recognizes a specific tumor antigen or other tumor cell-surface molecules. The intracellular region may contain a signaling domain of TCR complex (e.g., the signal region of CD3ζ), and/or one or more costimulatory signaling domains, such as those from CD28, 4-1BB (CD137) and OX-40 (CD134). For example, a “first-generation CAR” only has the CD3ζ signaling domain, whereas in an effort to augment T-cell persistence and proliferation, costimulatory intracellular domains are added, giving rise to second generation CARs having a CD3ζ signal domain plus one costimulatory signaling domain, and third generation CARs having CD3ζ signal domain plus two or more costimulatory signaling domains. A CAR, when expressed by a T cell, endows the T cell with antigen specificity determined by the extracellular targeting moiety of the CAR. Recently, it is also desirable to add one or more elements such as homing and suicide genes to develop a more competent and safer architecture of CAR, so called the fourth-generation CAR.

A CAR may be capable of binding to a tumor specific antigen selected from 5T4, 707-AP, A33, AFP (α-fetoprotein), AKAP-4 (A kinase anchor protein 4), ALK, α5β1-integrin, androgen receptor, annexin II, alpha-actinin-4, ART-4, B1, B7H3, B7H4, BAGE (B melanoma antigen), BCMA, BCR-ABL fusion protein, beta-catenin, BKT-antigen, BTAA, CA-I (carbonic anhydrase I), CA50 (cancer antigen 50), CA125, CA15-3, CA195, CA242, cairetinin, CAIX (carbonic anhydrase), CAMEL (cytotoxic T-lymphocyte recognized antigen on melanoma), CAM43, CAP-1, Caspase-8/m, CD4, CD5, CD7, CD19, CD20, CD22, CD23, CD25, CD27/m, CD28, CD30, CD33, CD34, CD36, CD38, CD40/CD154, CD41, CD44v6, CD44v7/8, CD45, CD49f, CD56, CD68\KP1, CD74, CD79a/CD79b, CD103, CD123, CD133, CD138, CD171, cdc27/m, CDK4 (cyclin dependent kinase 4), CDKN2A, CDS, CEA (carcinoembryonic antigen), CEACAM5, CEACAM6, chromogranin, c-Met, c-Myc, coa-1, CSAp, CT7, CT10, cyclophilin B, cyclin B1, cytoplasmic tyrosine kinases, cytokeratin, DAM-10, DAM-6, dek-can fusion protein, desmin, DEPDC1 (DEP domain containing 1), E2A-PRL, EBNA, EGF-R (epidermal growth factor receptor), EGP-1 (epithelial glycoprotein-1) (TROP-2), EGP-2, EGP-40, EGFR (epidermal growth factor receptor), EGFRvIII, EF-2, ELF2M, EMMPRIN, EpCAM (epithelial cell adhesion molecule), EphA2, Epstein Barr virus antigens, Erb (ErbB1; ErbB3; ErbB4), ETA (epithelial tumor antigen), ETV6-AML1 fusion protein, FAP (fibroblast activation protein), FBP (folate-binding protein), FGF-5, folate receptor α, FOS related antigen 1, fucosyl GM1, G250, GAGE (GAGE-1; GAGE-2), galactin, GD2 (ganglioside), GD3, GFAP (glial fibrillary acidic protein), GM2 (oncofetal antigen-immunogenic-1; OFA-I-1), GnT-V, Gp100, H4-RET, HAGE (helicase antigen), HER-2/neu, HIFs (hypoxia inducible factors), HIF-1α, HIF-2α, HLA-A2, HLA-A*0201-R170I, HLA-A11, HMWMAA, Hom/Mel-40, HSP70-2M (Heat shock protein 70), HST-2, HTgp-175, hTERT (or hTRT), human papillomavirus-E6/human papillomavirus-E7 and E6, iCE (immune-capture EIA), IGF-1R, IGH-IGK, IL2R, IL5, ILK (integrin-linked kinase), IMP3 (insulin-like growth factor II mRNA-binding protein 3), IRF4 (interferon regulatory factor 4), KDR (kinase insert domain receptor), KIAA0205, KRAB-zinc finger protein (KID)-3; KID31, KSA (17-1A), K-ras, LAGE, LCK, LDLR/FUT (LDLR-fucosyltransferaseAS fusion protein), LeY (Lewis Y), MAD-CT-1, MAGE (tyrosinase, melanoma-associated antigen) (MAGE-1; MAGE-3), melan-A tumor antigen (MART), MART-2/Ski, MC1R (melanocortin 1 receptor), MDM2, mesothelin, MPHOSPH1, MSA (muscle-specific actin), mTOR (mammalian targets of rapamycin), MUC-1, MUC-2, MUM-1 (melanoma associated antigen (mutated) 1), MUM-2, MUM-3, Myosin/m, MYL-RAR, NA88-A, N-acetylglucosaminyltransferase, neo-PAP, NF-KB (nuclear factor-kappa B), neurofilament, NSE (neuron-specific enolase), Notch receptors, NuMa, N-Ras, NY-BR-1, NY-CO-1, NY-ESO-1, Oncostatin M, OS-9, OY-TES1, p53 mutants, p190 minor bcr-abl, p15(58), p185erbB2, p180erbB-3, PAGE (prostate associated gene), PAP (prostatic acid phosphatase), PAX3, PAX5, PDGFR (platelet derived growth factor receptor), cytochrome P450 involved in piperidine and pyrrolidine utilization (PIPA), Pml-RAR alpha fusion protein, PR-3 (proteinase 3), PSA (prostate specific antigen), PSM, PSMA (Prostate stem cell antigen), PRAME (preferentially expressed antigen of melanoma), PTPRK, RAGE (renal tumor antigen), Raf (A-Raf, B-Raf and C-Raf), Ras, receptor tyrosine kinases, RCAS1, RGSS, ROR1 (receptor tyrosine kinase-like orphan receptor 1), RU1, RU2, SAGE, SART-1, SART-3, SCP-1, SDCCAG16, SP-17 (sperm protein 17), src-family, SSX (synovial sarcoma X breakpoint)-1, SSX-2 (HOM-MEL-40), SSX-3, SSX-4, SSX-5, STAT-3, STAT-5, STAT-6, STEAD, STn, survivin, syk-ZAP70, TA-90 (Mac-2 binding protein\cyclophilin C-associated protein), TAAL6, TACSTD1 (tumor associated calcium signal transducer 1), TACSTD2, TAG-72-4, TAGE, TARP (T cell receptor gamma alternate reading frame protein), TEL/AML1 fusion protein, TEM1, TEM8 (endosialin or CD248), TGFβ, TIE2, TLP, TMPRSS2 ETS fusion gene, TNF-receptor (TNF-α receptor, TNF-β receptor; or TNF-γ receptor), transferrin receptor, TPS, TRP-1 (tyrosine related protein 1), TRP-2, TRP-2/INT2, TSP-180, VEGF receptor, WNT, WT-1 (Wilm's tumor antigen) and XAGE.

Exemplary CAR constructs may include a CAR targeting mesothelin (U.S. Pat. Nos. 9,272,002 and 9,359,447); EGFRvIII specific CARs in U.S. Pat. No. 9,266,960; anti-TAG CARs in U.S. Pat. No. 9,233,125; CD19 CARs in US Patent Publication NO. 2016/014533; CD19 CAR having the amino acid sequence of SEQ ID NO. 24 of U.S. Pat. No. 9,328,156; CD19 CARs in U.S. Pat. Nos. 8,911,993, 8,975,071, 9,101,584, 9,102,760, and 9,102,761; BCMA (CD269) specific CARs disclosed in International Patent Publication NOs. WO2016/014565 and WO2016/014789; CLL-1 (C-type lectin-like molecule 1) CARs comprising the amino acid sequences of SEQ ID NOs. 99, 96, 100, 101, 102, 91, 92, 93, 94, 95, 97, 98, 103, and 197 disclosed in International Patent Publication NO. WO2016/014535; CD33 specific CARs comprising the amino acid sequences of SEQ ID NOs. 48-56 in International Patent Publication NO. WO2016/014576; CD33 specific CARs comprising the amino acid sequences of SEQ ID NOs. 19-22, 27-30 and 35-38 in International Patent Publication NO. WO2015/150526; CD37 specific CARs encoded by the nucleic acids of SEQ ID NOs. 1-5 in US patent publication NO. US2015/0329640; GPC3 CAR (International patent publication NO. WO2016/036973), GFRalpha 4 CARs having the amino acid sequences of SEQ ID NOs. 85, 86, 90, 92, 94, 96, 98, 100, 102, and 104 in International Patent Publication NO. WO2016/025880; CD123 CARs comprising the amino acid sequences of SEQ ID NOs. 98, 99, 100 and 101 in International Patent Publication NOs. WO2016/028896; CD123 specific multi-chain CARs in International Patent Publication NO. WO2015/193406; ROR-1 specific CARs comprising the amino acid sequences of SEQ ID NOs. 93, 95 and 117 in International Patent Publication NO. WO2016/016344; ROR-1 specific multi-chain CARs in International patent publication NO. WO2016/016343; trophoblast glycoprotein (5T4, TPBG) specific CARs comprising the amino acid sequences of SEQ ID NOs. 21, 27, 33, 39, 23, 29, 34, 41, 19, 25, 31, 37, 20, 26, 32, 38, 22, 28, 34, 40, 24, 30, 36 and 42 in International Patent Publication NO. WO2016/034666; EGFRvIII specific CARs comprising the amino acid sequences of SEQ ID NOs. 15, 17, 24, 25, 26 and 27 in International Patent Publication NO. WO2016016341; a TEM 8 CAR comprising the amino acid sequence of SEQ ID NO. 1 in International Patent Publication NO. WO2014164544, a TEMl CAR comprising the amino acid sequence of SEQ 1D NO. 2 in International Patent Publication NO. WO2014164544; GPC-3 CAR having the amino acid sequences of SEQ ID NOs. 3 and 26 in international Patent Publication NO. WO2016/049459; a chondroitin sulfate proteoglycan-4 (CSPG4) CAR in International Patent Publication NO. WO2015/080981; Kappa/lambda CARs in International Patent Publication NO. WO2015/164739; the contents of each of which are incorporated herein by reference in their entirety.

In some embodiments, the chimeric antigen receptors described herein may include CD3 zeta domains altered to tune CAR activity. The CD3 zeta domains may include one or more mutations in the immunoreceptor tyrosine-based activation motifs (ITAMs). In one aspect, the tyrosine residues within the ITAMs may be mutated resulting in reduced phosphorylation and limited downstream signaling. In some embodiments, one or more of the ITAMs may be deleted from the CD3 zeta domain. In one aspect, the CD3 zeta may include one ITAM. Any of the CARs and CD3 zeta domains described by Feucht et al. 2019 may be used herein (Calibration of CAR activation potential directs alternative T cell fates and therapeutic potency. Nature Medicine 25, 82-88 (2019); the contents of which are herein incorporated by reference in their entirety).

In some embodiments, the CAR constructs may include CAIX (carboxy-anhydrase-IX (CAIX) specific CAR (Lamers et al., Biochem Soc Trans, 2016, 44(3): 951-959), HIV-1 specific CAR (Ali et al., J Virol., 2016, May 25, pii: JVI.00805-16), CD20 specific CAR (Rufener et al., Cancer Immunol. Res., 2016, 4(6): 509-519), a CD20/CD19 bispecific CAR (Zah et al., Cancer Immunol Res., 2016, 4(6): 498-508), and EGFR specific CARs; the contents of each of which are incorporated herein by reference in their entirety.

In some embodiments, effector modules that include ER derived SREs and CD19 CAR payloads may have a sequence described in International Patent Publication WO2017181119, herein incorporated by reference in its entirety, such as but not limited to SEQ ID NO. 897; 900; 902; 905; 907; 910; 912; 915; 917; 920; 922; 925; 927; 930; 932; 935; 937; 940; 942; 945; 947; 950; 952 of WO2017181119 or variants thereof.

In some embodiments, the CD19 CAR tertiary structure may be modified to improve safety profile of CD19 CART cell therapy using tertiary-structure-prediction program (Phrye2) (Kelley et al. 2009. Nat. Protoc. 4, 363-371. CAR with improved safety profile may include but are not limited to those with reduced ability to produce cytokines. In some embodiments, the CARs with improved safety profile may include the CD19-BBz CAR variant contains an 86-amino-acid fragment from human CD8a, comprising a longer extracellular-domain fragment (55 amino acids versus 45 amino acids in the CD19-BBz (prototype) and a longer intracellular sequence (7 amino acids versus 3 amino acids in CD19-BBz (prototype/Kymriahl™) as described by Ying et al. 2019 Nat Med. doi: 10.1038/s41591-019-0421-7 (the contents of which are herein incorporated by reference in their entirety). In another aspect, the payloads of the present disclosure may be the CD19-BBz(86) variant CAR co-expressed with truncated, nonfunctional epidermal growth factor receptor (tEGFR) as described by Ying et al. 2019.

In one embodiment, the payload is a CD19 specific CAR operably linked to human ER DD. Table 4 provides CD19 CAR components. In some embodiments, the payloads may be CAR expressed in tandem with IL12. Table 5 provides the CD19-IL12 CAR constructs, Table 6 provides the CD19 CAR constructs. In Table 5 and Table 6, * indicates translation of the stop codon.

TABLE 4
CD19 CAR and Tandem Construct Components

| | | AA | NA |
|---|---|---|---|
| | | SEQ | SEQ |
| | | ID | ID |
| Description | AA sequence | NO. | NO. |
| CD8α leader | MALPVTALLLPLALLLHAARP | 99 | 100; |
| | | | 367 |
| CD8α leader 2 | ALPVTALLLPLALLLHAARP | 408 | 409 |
| 1L12B (p40) signal | MCHQQLVISWFSLVFLASPLVA | 101 | 102 |
| sequence | | | |
| CD19 scFv | DIQMTQTTSSLSASLGDRVTISCRASQDISKYLNWYQQ | 103 | 104 |
| | KPDGTVKLLIYHTSRLHSGVPSRFSGSGSGTDYSLTISN | | |
| | LEQEDIATYFCQQGNTLPYTFGGGTKLEITGGGGSGGG | | |
| | GSGGGGSEVKLQESGPGLVAPSQSLSVTCTVSGVSLPD | | |
| | YGVSWIRQPPRKGLEWLGVIWGSETTYYNSALKSRLTI | | |
| | IKDNSKSQVFLKMNSLQTDDTAIYYCAKHYYYGGSYA | | |
| | MDYWGQGTSVTVSS | | |
| CD19 scFv2 | EIVMTQSPATLSLSPGERATLSCRASQDISKYLNWYQQ | 368 | 369 |
| | KPGQAPRLLIYHTSRLHSGIPARFSGSGSGTDYTLTISSL | | |
| | QPEDFAVYFCQQGNTLPYTFGQGTKLEIKGGGGSGGG | | |
| | GSGGGGSQVQLQESGPGLVKPSETLSLTCTVSGVSLPD | | |
| | YGVSWIRQPPGKGLEWIGVIWGSETTYYSSSLKSRVTIS | | |
| | KDNSKNQVSLKLSSVTAADTAVYYCAKHYYYGGSYA | | |
| | MDYWGQGTLVTVSS | | |
| CD8 Hinge and | TTTPAPRPPTPAPTIASQPLSLRPEACRPAAGGAVHTRG | 105 | 106; |
| TM | LDFACDIYIWAPLAGTCGVLLLSLVITLYC | | 370 |
| 4-1BB intracellular | KRGRKKLLYIFKQPFMRPVQTTQEEDGCSCRFPEEEEG | 107 | 108; |
| signaling domain | GCEL | | 371 |
| CD3 zeta signaling | RVKFSRSADAPAYKQGQNQLYNELNLGRREEYDVLD | 109 | 110; |
| domain | KRRGRDPEMGGKPRRKNPQEGLYNELQKDKMAEAYS | | 372 |
| | EIGMKGERRRGKGHDGLYQGLSTATKDTYDALHMQA | | |
| | LPPR | | |
| IL12B (p40) | IWELKKDVYVVELDWYPDAPGEMVVLTCDTPEEDGIT | 111 | 112 |
| (23-328 of WT) | WTLDQSSEVLGSGKTLTIQVKEFGDAGQYTCHKGGEV | | |
| | LSHSLLLLHKKEDGIWSTDILKDQKEPKNKTFLRCEAK | | |
| | NYSGRFTCWWLTTISTDLTFSVKSSRGSSDPQGVTCGA | | |
| | ATLSAERVRGDNKEYEYSVECQEDSACPAAEESLPIEV | | |
| | MVDAVHKLKYENYTSSFFIRDIIKPDPPKNLQLKPLKNS | | |
| | RQVEVSWEYPDTWSTPHSYFSLTFCVQVQGKSKREKK | | |
| | DRVFTDKTSATVICRKNASISVRAQDRYYSSSWSEWAS | | |
| | VPCS | | |
| IL12A (p35) | RNLPVATPDPGMFPCLHHSQNLLRAVSNMLQKARQTL | 113 | 114-115 |
| (57-253 of WT) | EFYPCTSEEIDHEDITKDKTSTVEACLPLELTKNESCLN | | |
| | SRETSFITNGSCLASRKTSFMNIALCLSSIYEDLKMYQV | | |
| | EFKTMNAKLLMDPKRQIFLDQNMLAVIDELMQALNFN | | |
| | SETVPQKSSLEEPDFYKTKIKLCILLHAFRIRAVTIDRV | | |
| | MSYLNAS | | |
| Furin cleavage | RTKR | 116 | 117 |
| site 1 | | | |
| Modified Furin | ESRRVRRNKRSK | 118 | 119 |
| P2A cleavage site | ATNFSLLKQAGDVEENPGP | 120 | 121 |
| Spacer | — | — | 122 |
| Spacer | — | — | 123 |
| IRES | — | — | 124 |
| Linker ((G4S)3 | GGGGSGGGGSGGGGS | 125 | 126 |
| Flexible G/S rich | GS | | |
| linker | | | |

TABLE 5
CD19-IL12 Tandem CAR Constructs

| | | | AA | NA |
|---|---|---|---|---|
| | | | SEQ | SEQ |
| | | | ID | ID |
| Construct ID | Description | AA sequence | NO. | NO. |
| OT-001640 (Full | Full | — | — | 127 |
| Construct: CD8α | Construct | | | |
| leader sequence; | Encoded | MALPVTALLLPLALLLHAARPDIQMTQTTSSLSASL | 128 | 129 |
| CD19 scFv; | Protein 1 | GDRVTISCRASQDISKYLNWYQQKPDGTVKLLIYH | | |
| CD8α Hinge-TM; | | TSRLHSGVPSRFSGSGSGTDYSLTISNLEQEDIATYF | | |
| 4-1BB signaling | | CQQGNTLPYTFGGGTKLEITGGGGSGGGGSGGGGS | | |
| domain; CD3 zeta | | EVKLQESGPGLVAPSQSLSVTCTVSGVSLPDYGVS | | |
| signaling domain; | | WIRQPPRKGLEWLGVIWGSETTYYNSALKSRLTIIK | | |
| stop; Spacer; | | DNSKSQVFLKMNSLQTDDTAIYYCAKHYYYGGSY | | |
| IRES; Spacer; | | AMDYWGQGTSVTVSSTTTPAPRPPTPAPTIASQPLS | | |
| (IL12B (p40) | | LRPEACRPAAGGAVHTRGLDFACDIYIWAPLAGTC | | |
| signal sequence; | | GVLLLSLVITLYCKRGRKKLLYIFKQPFMRPVQTTQ | | |
| IL12B (p40) | | EEDGCSCRFPEEEEGGCELRVKFSRSADAPAYKQG | | |
| (23-328 of WT); | | QNQLYNELNLGRREEYDVLDKRRGRDPEMGGKPR | | |
| Linker ((G4S)3; | | RKNPQEGLYNELQKDKMAEAYSEIGMKGERRRGK | | |
| IL12A (p35) | | GHDGLYQGLSTATKDTYDALHMQALPPR* | | |
| (57-253 of WT); | Encoded | MCHQQLVISWFSLVFLASPLVAIWELKKDVYVVEL | 130 | 131 |
| Linker (GS); ER | Protein 2 | DWYPDAPGEMVVLTCDTPEEDGITWTLDQSSEVL | | |
| (aa 305-549 of | | GSGKTLTIQVKEFGDAGQYTCHKGGEVLSHSLLLL | | |
| WT, R335G, | | HKKEDGIWSTDILKDQKEPKNKTFLRCEAKNYSGR | | |
| L384M, M421G, | | FTCWWLTTISTDLTFSVKSSRGSSDPQGVTCGAATL | | |
| G521R, E523G, | | SAERVRGDNKEYEYSVECQEDSACPAAEESLPIEV | | |
| Y537S, A546T); | | MVDAVHKLKYENYTSSFFIRDIIKPDPPKNLQLKPL | | |
| stop) | | KNSRQVEVSWEYPDTWSTPHSYFSLTFCVQVQGKS | | |
| | | KREKKDRVFTDKTSATVICRKNASISVRAQDRYYS | | |
| | | SSWSEWASVPCSGGGGSGGGGSGGGGSRNLPVAT | | |
| | | PDPGMFPCLHHSQNLLRAVSNMLQKARQTLEFYPC | | |
| | | TSEEIDHEDITKDKTSTVEACLPLELTKNESCLNSRE | | |
| | | TSFITNGSCLASRKTSFMMALCLSSIYEDLKMYQVE | | |
| | | FKTMNAKLLMDPKRQIFLDQNMLAVIDELMQALN | | |
| | | FNSETVPQKSSLEEPDFYKTKIKLCILLHAFRIRAVTI | | |
| | | DRVMSYLNASGSSLALSLTADQMVSALLDAEPPIL | | |
| | | YSEYDPTGPFSEASMMGLLTNLADRELVHMINWA | | |
| | | KRVPGFVDLTLHDQVHLLECAWMEILMIGLVWRS | | |
| | | MEHPGKLLFAPNLLLDRNQGKCVEGGVEIHMLL | | |
| | | ATSSRFRMMNLQGEEFVCLKSIILLNSGVYTFLSST | | |
| | | LKSLEEKDHIHRVLDKITDTLIHLMAKAGLTLQQQ | | |
| | | HQRLAQLLLILSHIRHMSNKRMGHLYSMKCKNVV | | |
| | | PLSDLLLEMLDTHRL* | | |
| OT-001641 (Full | Full | — | — | 132 |
| Construct: CD8α | Construct | | | |
| leader sequence; | Encoded | MALPVTALLLPLALLLHAARPDIQMTQTTSSLSASL | 128 | 129 |
| CD19 scFv; | Protein 1 | GDRVTISCRASQDISKYLNWYQQKPDGTVKLLIYH | | |
| CD8α Hinge-TM; | | TSRLHSGVPSRFSGSGSGTDYSLTISNLEQEDIATYF | | |
| 4-1BB signaling | | CQQGNTLPYTFGGGTKLEITGGGGSGGGGSGGGGS | | |
| domain; CD3 zeta | | EVKLQESGPGLVAPSQSLSVTCTVSGVSLPDYGVS | | |
| signaling domain; | | WIRQPPRKGLEWLGVIWGSETTYYNSALKSRLTIIK | | |
| stop; Spacer; | | DNSKSQVFLKMNSLQTDDTAIYYCAKHYYYGGSY | | |
| IRES; Spacer; | | AMDYWGQGTSVTVSSTTTPAPRPPTPAPTIASQPLS | | |
| (IL12B (p40) | | LRPEACRPAAGGAVHTRGLDFACDIYIWAPLAGTC | | |
| signal sequence; | | GVLLLSLVITLYCKRGRKKLLYIFKQPFMRPVQTTQ | | |
| IL12B (p40) | | EEDGCSCRFPEEEEGGCELRVKFSRSADAPAYKQG | | |
| (23-328 of WT); | | QNQLYNELNLGRREEYDVLDKRRGRDPEMGGKPR | | |
| Linker ((G4S)3; | | RKNPQEGLYNELQKDKMAEAYSEIGMKGERRRGK | | |
| IL12A (p35) | | GHDGLYQGLSTATKDTYDALHMQALPPR* | | |
| (57-253 of WT); | Encoded | MCHQQLVISWFSLVFLASPLVAIWELKKDVYVVEL | 133 | 134 |
| Linker (GS); ER | Protein 2 | DWYPDAPGEMVVLTCDTPEEDGITWTLDQSSEVL | | |
| (aa 305-549 of | | GSGKTLTIQVKEFGDAGQYTCHKGGEVLSHSLLLL | | |
| WT, L384M; | | HKKEDGIWSTDILKDQKEPKNKTFLRCEAKNYSGR | | |
| M421G; T431I; | | FTCWWLTTISTDLTFSVKSSRGSSDPQGVTCGAATL | | |
| G521R,Y537S); | | SAERVRGDNKEYEYSVECQEDSACPAAEESLPIEV | | |
| stop) | | MVDAVHKLKYENYTSSFFIRDIIKPDPPKNLQLKPL | | |
| | | KNSRQVEVSWEYPDTWSTPHSYFSLTFCVQVQGKS | | |
| | | KREKKDRVFTDKTSATVICRKNASISVRAQDRYYS | | |
| | | SSWSEWASVPCSGGGGSGGGGSGGGGSRNLPVAT | | |
| | | PDPGMFPCLHHSQNLLRAVSNMLQKARQTLEFYPC | | |
| | | TSEEIDHEDITKDKTSTVEACLPLELTKNESCLNSRE | | |
| | | TSFITNGSCLASRKTSFMMALCLSSIYEDLKMYQVE | | |
| | | FKTMNAKLLMDPKRQIFLDQNMLAVIDELMQALN | | |
| | | FNSETVPQKSSLEEPDFYKTKIKLCILLHAFRIRAVTI | | |
| | | DRVMSYLNASGSSLALSLTADQMVSALLDAEPPIL | | |
| | | YSEYDPTRPFSEASMMGLLTNLADRELVHMINWA | | |
| | | KRVPGFVDLTLHDQVHLLECAWMEILMIGLVWRS | | |
| | | MEHPGKLLFAPNLLLDRNQGKCVEGGVEIHMLL | | |
| | | AISSRFRMMNLQGEEFVCLKSIILLNSGVYTFLSSTL | | |
| | | KSLEEKDHIHRVLDKITDTLIHLMAKAGLTLQQQH | | |
| | | QRLAQLLLILSHIRHMSNKRMEHLYSMKCKNVVPL | | |
| | | SDLLLEMLDAHRL* | | |
| OT-001642 (Full | Full | — | — | 135 |
| Construct: CD8α | Construct | | | |
| leader sequence; | Encoded | MALPVTALLLPLALLLHAARPDIQMTQTTSSLSASL | 128 | 129 |
| CD19 scFv; | Protein 1 | GDRVTISCRASQDISKYLNWYQQKPDGTVKLLIYH | | |
| CD8α Hinge-TM; | | TSRLHSGVPSRFSGSGSGTDYSLTISNLEQEDIATYF | | |
| 4-1BB signaling | | CQQGNTLPYTFGGGTKLEITGGGGSGGGGSGGGGS | | |
| domain; CD3 zeta | | EVKLQESGPGLVAPSQSLSVTCTVSGVSLPDYGVS | | |
| signaling domain; | | WIRQPPRKGLEWLGVIWGSETTYYNSALKSRLTIIK | | |
| stop; Spacer; | | DNSKSQVFLKMNSLQTDDTAIYYCAKHYYYGGSY | | |
| IRES; Spacer; | | AMDYWGQGTSVTVSSTTTPAPRPPTPAPTIASQPLS | | |
| (IL12B (p40) | | LRPEACRPAAGGAVHTRGLDFACDIYIWAPLAGTC | | |
| signal sequence; | | GVLLLSLVITLYCKRGRKKLLYIFKQPFMRPVQTTQ | | |
| IL12B (p40) | | EEDGCSCRFPEEEEGGCELRVKFSRSADAPAYKQG | | |
| (23-328 of WT); | | QNQLYNELNLGRREEYDVLDKRRGRDPEMGGKPR | | |
| Linker ((G4S)3; | | RKNPQEGLYNELQKDKMAEAYSEIGMKGERRRGK | | |
| IL12A (p35) | | GHDGLYQGLSTATKDTYDALHMQALPPR* | | |
| (57-253 of WT); | Encoded | MCHQQLVISWFSLVFLASPLVAIWELKKDVYVVEL | 136 | 137 |
| Linker (GS); | Protein 2 | DWYPDAPGEMVVLTCDTPEEDGITWTLDQSSEVL | | |
| ER(aa 305-549 of | | GSGKTLTIQVKEFGDAGQYTCHKGGEVLSHSLLLL | | |
| WT, L384M, | | HKKEDGIWSTDILKDQKEPKNKTFLRCEAKNYSGR | | |
| N413D , M421G, | | FTCWWLTTISTDLTFSVKSSRGSSDPQGVTCGAATL | | |
| G521R, Y537S); | | SAERVRGDNKEYEYSVECQEDSACPAAEESLPIEV | | |
| stop) | | MVDAVHKLKYENYTSSFFIRDIIKPDPPKNLQLKPL | | |
| | | KNSRQVEVSWEYPDTWSTPHSYFSLTFCVQVQGKS | | |
| | | KREKKDRVFTDKTSATVICRKNASISVRAQDRYYS | | |
| | | SSWSEWASVPCSGGGGSGGGGSGGGGSRNLPVAT | | |
| | | PDPGMFPCLHHSQNLLRAVSNMLQKARQTLEFYPC | | |
| | | TSEEIDHEDITKDKTSTVEACLPLELTKNESCLNSRE | | |
| | | TSFITNGSCLASRKTSFMMALCLSSIYEDLKMYQVE | | |
| | | FKTMNAKLLMDPKRQIFLDQNMLAVIDELMQALN | | |
| | | FNSETVPQKSSLEEPDFYKTKIKLCILLHAFRIRAVTI | | |
| | | DRVMSYLNASGSSLALSLTADQMVSALLDAEPPIL | | |
| | | YSEYDPTRPFSEASMMGLLTNLADRELVHMINWA | | |
| | | KRVPGFVDLTLHDQVHLLECAWMEILMIGLVWRS | | |
| | | MEHPGKLLFAPNLLLDRDQGKCVEGGVEIHMLL | | |
| | | ATSSRFRMMNLQGEEFVCLKSIILLNSGVYTFLSST | | |
| | | LKSLEEKDHIHRVLDKITDTLIHLMAKAGLTLQQQ | | |
| | | HQRLAQLLLILSHIRHMSNKRMEHLYSMKCKNVV | | |
| | | PLSDLLLEMLDAHRL* | | |
| OT-001643 (Full | Full | — | — | 138 |
| Construct: CD8α | Construct | | | |
| leader sequence; | Encoded | MALPVTALLLPLALLLHAARPDIQMTQTTSSLSASL | 128 | 129 |
| CD19 scFv; | Protein 1 | GDRVTISCRASQDISKYLNWYQQKPDGTVKLLIYH | | |
| CD8α Hinge-TM; | | TSRLHSGVPSRFSGSGSGTDYSLTISNLEQEDIATYF | | |
| 4-1BB signaling | | CQQGNTLPYTFGGGTKLEITGGGGSGGGGSGGGGS | | |
| domain; CD3 zeta | | EVKLQESGPGLVAPSQSLSVTCTVSGVSLPDYGVS | | |
| signaling domain; | | WIRQPPRKGLEWLGVIWGSETTYYNSALKSRLTIIK | | |
| stop; Spacer; | | DNSKSQVFLKMNSLQTDDTAIYYCAKHYYYGGSY | | |
| IRES; Spacer; | | AMDYWGQGTSVTVSSTTTPAPRPPTPAPTIASQPLS | | |
| (IL12B (p40) | | LRPEACRPAAGGAVHTRGLDFACDIYIWAPLAGTC | | |
| signal sequence; | | GVLLLSLVITLYCKRGRKKLLYIFKQPFMRPVQTTQ | | |
| IL12B (p40) | | EEDGCSCRFPEEEEGGCELRVKFSRSADAPAYKQG | | |
| (23-328 of WT); | | QNQLYNELNLGRREEYDVLDKRRGRDPEMGGKPR | | |
| Linker ((G4S)3; | | RKNPQEGLYNELQKDKMAEAYSEIGMKGERRRGK | | |
| IL12A (p35) | | GHDGLYQGLSTATKDTYDALHMQALPPR* | | |
| (57-253 of WT); | Encoded | MCHQQLVISWFSLVFLASPLVAIWELKKDVYVVEL | 139 | 140 |
| Linker (GS); ER | Protein 2 | DWYPDAPGEMVVLTCDTPEEDGITWTLDQSSEVL | | |
| (aa 305-549 of | | GSGKTLTIQVKEFGDAGQYTCHKGGEVLSHSLLLL | | |
| WT, L384M, | | HKKEDGIWSTDILKDQKEPKNKTFLRCEAKNYSGR | | |
| M421G, N519S, | | FTCWWLTTISTDLTFSVKSSRGSSDPQGVTCGAATL | | |
| G521R, Y537S); | | SAERVRGDNKEYEYSVECQEDSACPAAEESLPIEV | | |
| stop) | | MVDAVHKLKYENYTSSFFIRDIIKPDPPKNLQLKPL | | |
| | | KNSRQVEVSWEYPDTWSTPHSYFSLTFCVQVQGKS | | |
| | | KREKKDRVFTDKTSATVICRKNASISVRAQDRYYS | | |
| | | SSWSEWASVPCSGGGGSGGGGSGGGGSRNLPVAT | | |
| | | PDPGMFPCLHHSQNLLRAVSNMLQKARQTLEFYPC | | |
| | | TSEEIDHEDITKDKTSTVEACLPLELTKNESCLNSRE | | |
| | | TSFITNGSCLASRKTSFMMALCLSSIYEDLKMYQVE | | |
| | | FKTMNAKLLMDPKRQIFLDQNMLAVIDELMQALN | | |
| | | FNSETVPQKSSLEEPDFYKTKIKLCILLHAFRIRAVTI | | |
| | | DRVMSYLNASGSSLALSLTADQMVSALLDAEPPIL | | |
| | | YSEYDPTRPFSEASMMGLLTNLADRELVHMINWA | | |
| | | KRVPGFVDLTLHDQVHLLECAWMEILMIGLVWRS | | |
| | | MEHPGKLLFAPNLLLDRNQGKCVEGGVEIFDMLL | | |
| | | ATSSRFRMMNLQGEEFVCLKSIILLNSGVYTFLSST | | |
| | | LKSLEEKDHIHRVLDKITDTLIHLMAKAGLTLQQQ | | |
| | | HQRLAQLLLILSHIRHMSSKRMEHLYSMKCKNVVP | | |
| | | LSDLLLEMLDAHRL* | | |
| OT-001644 (Full | Full | — | — | 141 |
| Construct: CD8α | Construct | | | |
| leader sequence; | Encoded | MALPVTALLLPLALLLHAARPDIQMTQTTSSLSASL | 128 | 129 |
| CD19 scFv; | Protein 1 | GDRVTISCRASQDISKYLNWYQQKPDGTVKLLIYH | | |
| CD8α Hinge-TM; | | TSRLHSGVPSRFSGSGSGTDYSLTISNLEQEDIATYF | | |
| 4-1BB signaling | | CQQGNTLPYTFGGGTKLEITGGGGSGGGGSGGGGS | | |
| domain; CD3 zeta | | EVKLQESGPGLVAPSQSLSVTCTVSGVSLPDYGVS | | |
| signaling domain; | | WIRQPPRKGLEWLGVIWGSETTYYNSALKSRLTIIK | | |
| stop; Spacer; | | DNSKSQVFLKMNSLQTDDTAIYYCAKHYYYGGSY | | |
| IRES; Spacer; | | AMDYWGQGTSVTVSSTTTPAPRPPTPAPTIASQPLS | | |
| (IL12B (p40) | | LRPEACRPAAGGAVHTRGLDFACDIYIWAPLAGTC | | |
| signal sequence; | | GVLLLSLVITLYCKRGRKKLLYIFKQPFMRPVQTTQ | | |
| IL12B (p40) | | EEDGCSCRFPEEEEGGCELRVKFSRSADAPAYKQG | | |
| (23-328 of WT); | | QNQLYNELNLGRREEYDVLDKRRGRDPEMGGKPR | | |
| Linker ((G4S)3; | | RKNPQEGLYNELQKDKMAEAYSEIGMKGERRRGK | | |
| IL12A (p35) | | GHDGLYQGLSTATKDTYDALHMQALPPR* | | |
| (57-253 of WT); | Encoded | MCHQQLVISWFSLVFLASPLVAIWELKKDVYVVEL | 142 | 143 |
| Linker (GS); ER | Protein 2 | DWYPDAPGEMVVLTCDTPEEDGITWTLDQSSEVL | | |
| (305-549 of WT, | | GSGKTLTIQVKEFGDAGQYTCHKGGEVLSHSLLLL | | |
| L384M, M421G, | | HKKEDGIWSTDILKDQKEPKNKTFLRCEAKNYSGR | | |
| Q502R, G521R, | | FTCWWLTTISTDLTFSVKSSRGSSDPQGVTCGAATL | | |
| Y537S); stop) | | SAERVRGDNKEYEYSVECQEDSACPAAEESLPIEV | | |
| | | MVDAVHKLKYENYTSSFFIRDIIKPDPPKNLQLKPL | | |
| | | KNSRQVEVSWEYPDTWSTPHSYFSLTFCVQVQGKS | | |
| | | KREKKDRVFTDKTSATVICRKNASISVRAQDRYYS | | |
| | | SSWSEWASVPCSGGGGSGGGGSGGGGSRNLPVAT | | |
| | | PDPGMFPCLHHSQNLLRAVSNMLQKARQTLEFYPC | | |
| | | TSEEIDHEDITKDKTSTVEACLPLELTKNESCLNSRE | | |
| | | TSFITNGSCLASRKTSFMMALCLSSIYEDLKMYQVE | | |
| | | FKTMNAKLLMDPKRQIFLDQNMLAVIDELMQALN | | |
| | | FNSETVPQKSSLEEPDFYKTKIKLCILLHAFRIRAVTI | | |
| | | DRVMSYLNASGSSLALSLTADQMVSALLDAEPPIL | | |
| | | YSEYDPTRPFSEASMMGLLTNLADRELVHMINWA | | |
| | | KRVPGFVDLTLHDQVHLLECAWMEILMIGLVWRS | | |
| | | MEHPGKLLFAPNLLLDRNQGKCVEGGVEIHMLL | | |
| | | ATSSRFRMMNLQGEEFVCLKSIILLNSGVYTFLSST | | |
| | | LKSLEEKDHIHRVLDKITDTLIHLMAKAGLTLQQQ | | |
| | | HRRLAQLLLILSHIRHMSNKRMEHLYSMKCKNVVP | | |
| | | LSDLLLEMLDAHRL* | | |
| OT-001645 (Full | Full | — | — | 144 |
| Construct: CD8α | Construct | | | |
| leader sequence; | Encoded | MALPVTALLLPLALLLHAARPDIQMTQTTSSLSASL | 128 | 129 |
| CD19 scFv; | Protein 1 | GDRVTISCRASQDISKYLNWYQQKPDGTVKLLIYH | | |
| CD8α Hinge-TM; | | TSRLHSGVPSRFSGSGSGTDYSLTISNLEQEDIATYF | | |
| 4-1BB signaling | | CQQGNTLPYTFGGGTKLEITGGGGSGGGGSGGGGS | | |
| domain; CD3 zeta | | EVKLQESGPGLVAPSQSLSVTCTVSGVSLPDYGVS | | |
| signaling domain; | | WIRQPPRKGLEWLGVIWGSETTYYNSALKSRLTIIK | | |
| stop; Spacer; | | DNSKSQVFLKMNSLQTDDTAIYYCAKHYYYGGSY | | |
| IRES; Spacer; | | AMDYWGQGTSVTVSSTTTPAPRPPTPAPTIASQPLS | | |
| (IL12B (p40) | | LRPEACRPAAGGAVHTRGLDFACDIYIWAPLAGTC | | |
| signal sequence; | | GVLLLSLVITLYCKRGRKKLLYIFKQPFMRPVQTTQ | | |
| IL12B (p40) | | EEDGCSCRFPEEEEGGCELRVKFSRSADAPAYKQG | | |
| (23-328 of WT); | | QNQLYNELNLGRREEYDVLDKRRGRDPEMGGKPR | | |
| Linker ((G4S)3; | | RKNPQEGLYNELQKDKMAEAYSEIGMKGERRRGK | | |
| IL12A (p35) | | GHDGLYQGLSTATKDTYDALHMQALPPR* | | |
| (57-253 of WT); | Encoded | MCHQQLVISWFSLVFLASPLVAIWELKKDVYVVEL | 145 | 146 |
| Linker (GS); ER | Protein 2 | DWYPDAPGEMVVLTCDTPEEDGITWTLDQSSEVL | | |
| (aa 305-549 of | | GSGKTLTIQVKEFGDAGQYTCHKGGEVLSHSLLLL | | |
| WT, T371A, | | HKKEDGIWSTDILKDQKEPKNKTFLRCEAKNYSGR | | |
| L384M, M421G, | | FTCWWLTTISTDLTFSVKSSRGSSDPQGVTCGAATL | | |
| N519S, G521R, | | SAERVRGDNKEYEYSVECQEDSACPAAEESLPIEV | | |
| Y537S); stop) | | MVDAVHKLKYENYTSSFFIRDIIKPDPPKNLQLKPL | | |
| | | KNSRQVEVSWEYPDTWSTPHSYFSLTFCVQVQGKS | | |
| | | KREKKDRVFTDKTSATVICRKNASISVRAQDRYYS | | |
| | | SSWSEWASVPCSGGGGSGGGGSGGGGSRNLPVAT | | |
| | | PDPGMFPCLHHSQNLLRAVSNMLQKARQTLEFYPC | | |
| | | TSEEIDHEDITKDKTSTVEACLPLELTKNESCLNSRE | | |
| | | TSFITNGSCLASRKTSFMMALCLSSIYEDLKMYQVE | | |
| | | FKTMNAKLLMDPKRQIFLDQNMLAVIDELMQALN | | |
| | | FNSETVPQKSSLEEPDFYKTKIKLCILLHAFRIRAVTI | | |
| | | DRVMSYLNASGSSLALSLTADQMVSALLDAEPPIL | | |
| | | YSEYDPTRPFSEASMMGLLTNLADRELVHMINWA | | |
| | | KRVPGFVDLALHDQVHLLECAWMEILMIGLVWRS | | |
| | | MEHPGKLLFAPNLLLDRNQGKCVEGGVEIHMLL | | |
| | | ATSSRFRMMNLQGEEFVCLKSIILLNSGVYTFLSST | | |
| | | LKSLEEKDHIHRVLDKITDTLIHLMAKAGLTLQQQ | | |
| | | HQRLAQLLLILSHIRHMSSKRMEHLYSMKCKNVVP | | |
| | | LSDLLLEMLDAHRL* | | |
| OT-001646 (Full | Full | — | — | 147 |
| Construct: CD8α | Construct | | | |
| leader sequence; | Encoded | MALPVTALLLPLALLLHAARPDIQMTQTTSSLSASL | 128 | 129 |
| CD19 scFv; | Protein 1 | GDRVTISCRASQDISKYLNWYQQKPDGTVKLLIYH | | |
| CD8α Hinge-TM; | | TSRLHSGVPSRFSGSGSGTDYSLTISNLEQEDIATYF | | |
| 4-1BB signaling | | CQQGNTLPYTFGGGTKLEITGGGGSGGGGSGGGGS | | |
| domain; CD3 zeta | | EVKLQESGPGLVAPSQSLSVTCTVSGVSLPDYGVS | | |
| signaling domain; | | WIRQPPRKGLEWLGVIWGSETTYYNSALKSRLTIIK | | |
| stop; Spacer; | | DNSKSQVFLKMNSLQTDDTAIYYCAKHYYYGGSY | | |
| IRES; Spacer; | | AMDYWGQGTSVTVSSTTTPAPRPPTPAPTIASQPLS | | |
| (IL12B (p40) | | LRPEACRPAAGGAVHTRGLDFACDIYIWAPLAGTC | | |
| signal sequence; | | GVLLLSLVITLYCKRGRKKLLYIFKQPFMRPVQTTQ | | |
| IL12B (p40) | | EEDGCSCRFPEEEEGGCELRVKFSRSADAPAYKQG | | |
| (23-328 of WT); | | QNQLYNELNLGRREEYDVLDKRRGRDPEMGGKPR | | |
| Linker ((G4S)3; | | RKNPQEGLYNELQKDKMAEAYSEIGMKGERRRGK | | |
| IL12A (p35) | | GHDGLYQGLSTATKDTYDALHMQALPPR* | | |
| (57-253 of WT); | Encoded | MCHQQLVISWFSLVFLASPLVAIWELKKDVYVVEL | 148 | 149 |
| Linker (GS); ER | Protein 2 | DWYPDAPGEMVVLTCDTPEEDGITWTLDQSSEVL | | |
| (aa 305-549 of | | GSGKTLTIQVKEFGDAGQYTCHKGGEVLSHSLLLL | | |
| WT, S305N, | | HKKEDGIWSTDILKDQKEPKNKTFLRCEAKNYSGR | | |
| L384M, M421G, | | FTCWWLTTISTDLTFSVKSSRGSSDPQGVTCGAATL | | |
| G442V, G521R, | | SAERVRGDNKEYEYSVECQEDSACPAAEESLPIEV | | |
| Y537S); stop) | | MVDAVHKLKYENYTSSFFIRDIIKPDPPKNLQLKPL | | |
| | | KNSRQVEVSWEYPDTWSTPHSYFSLTFCVQVQGKS | | |
| | | KREKKDRVFTDKTSATVICRKNASISVRAQDRYYS | | |
| | | SSWSEWASVPCSGGGGSGGGGSGGGGSRNLPVAT | | |
| | | PDPGMFPCLHHSQNLLRAVSNMLQKARQTLEFYPC | | |
| | | TSEEIDHEDITKDKTSTVEACLPLELTKNESCLNSRE | | |
| | | TSFITNGSCLASRKTSFMMALCLSSIYEDLKMYQVE | | |
| | | FKTMNAKLLMDPKRQIFLDQNMLAVIDELMQALN | | |
| | | FNSETVPQKSSLEEPDFYKTKIKLCILLHAFRIRAVTI | | |
| | | DRVMSYLNASGSNLALSLTADQMVSALLDAEPPIL | | |
| | | YSEYDPTRPFSEASMMGLLTNLADRELVHMINWA | | |
| | | KRVPGFVDLTLHDQVHLLECAWMEILMIGLVWRS | | |
| | | MEHPGKLLFAPNLLLDRNQGKCVEGGVEIFDMLL | | |
| | | ATSSRFRMMNLQVEEFVCLKSIILLNSGVYTFLSST | | |
| | | LKSLEEKDHIHRVLDKITDTLIHLMAKAGLTLQQQ | | |
| | | HQRLAQLLLILSHIRHMSNKRMEHLYSMKCKNVV | | |
| | | PLSDLLLEMLDAHRL* | | |
| OT-001647 (Full | Full | — | — | 150 |
| Construct: CD8α | Construct | | | |
| leader sequence; | Encoded | MALPVTALLLPLALLLHAARPDIQMTQTTSSLSASL | 128 | 129 |
| CD19 scFv; | Protein 1 | GDRVTISCRASQDISKYLNWYQQKPDGTVKLLIYH | | |
| CD8α Hinge-TM; | | TSRLHSGVPSRFSGSGSGTDYSLTISNLEQEDIATYF | | |
| 4-1BB signaling | | CQQGNTLPYTFGGGTKLEITGGGGSGGGGSGGGGS | | |
| domain; CD3 zeta | | EVKLQESGPGLVAPSQSLSVTCTVSGVSLPDYGVS | | |
| signaling domain; | | WIRQPPRKGLEWLGVIWGSETTYYNSALKSRLTIIK | | |
| stop; Spacer; | | DNSKSQVFLKMNSLQTDDTAIYYCAKHYYYGGSY | | |
| IRES; Spacer; | | AMDYWGQGTSVTVSSTTTPAPRPPTPAPTIASQPLS | | |
| (IL12B (p40) | | LRPEACRPAAGGAVHTRGLDFACDIYIWAPLAGTC | | |
| signal sequence; | | GVLLLSLVITLYCKRGRKKLLYIFKQPFMRPVQTTQ | | |
| IL12B (p40) | | EEDGCSCRFPEEEEGGCELRVKFSRSADAPAYKQG | | |
| (23-328 of WT); | | QNQLYNELNLGRREEYDVLDKRRGRDPEMGGKPR | | |
| Linker ((G4S)3; | | RKNPQEGLYNELQKDKMAEAYSEIGMKGERRRGK | | |
| IL12A (p35) | | GHDGLYQGLSTATKDTYDALHMQALPPR* | | |
| (57-253 of WT); | Encoded | MCHQQLVISWFSLVFLASPLVAIWELKKDVYVVEL | 151 | 152 |
| Linker (GS); ER | Protein 2 | DWYPDAPGEMVVLTCDTPEEDGITWTLDQSSEVL | | |
| (aa 305-549 of | | GSGKTLTIQVKEFGDAGQYTCHKGGEVLSHSLLLL | | |
| WT, R335G, | | HKKEDGIWSTDILKDQKEPKNKTFLRCEAKNYSGR | | |
| L384M, M421G, | | FTCWWLTTISTDLTFSVKSSRGSSDPQGVTCGAATL | | |
| N519S, G521R, | | SAERVRGDNKEYEYSVECQEDSACPAAEESLPIEV | | |
| Y537S); stop) | | MVDAVHKLKYENYTSSFFIRDIIKPDPPKNLQLKPL | | |
| | | KNSRQVEVSWEYPDTWSTPHSYFSLTFCVQVQGKS | | |
| | | KREKKDRVFTDKTSATVICRKNASISVRAQDRYYS | | |
| | | SSWSEWASVPCSGGGGSGGGGSGGGGSRNLPVAT | | |
| | | PDPGMFPCLHHSQNLLRAVSNMLQKARQTLEFYPC | | |
| | | TSEEIDHEDITKDKTSTVEACLPLELTKNESCLNSRE | | |
| | | TSFITNGSCLASRKTSFMMALCLSSIYEDLKMYQVE | | |
| | | FKTMNAKLLMDPKRQIFLDQNMLAVIDELMQALN | | |
| | | FNSETVPQKSSLEEPDFYKTKIKLCILLHAFRIRAVTI | | |
| | | DRVMSYLNASGSSLALSLTADQMVSALLDAEPPIL | | |
| | | YSEYDPTGPFSEASMMGLLTNLADRELVHMINWA | | |
| | | KRVPGFVDLTLHDQVHLLECAWMEILMIGLVWRS | | |
| | | MEHPGKLLFAPNLLLDRNQGKCVEGGVEIHMLL | | |
| | | ATSSRFRMMNLQGEEFVCLKSIILLNSGVYTFLSST | | |
| | | LKSLEEKDHIHRVLDKITDTLIHLMAKAGLTLQQQ | | |
| | | HQRLAQLLLILSHIRHMSSKRMEHLYSMKCKNVVP | | |
| | | LSDLLLEMLDAHRL* | | |
| OT-001648 (Full | Full | — | — | 153 |
| Construct: CD8α | Construct | | | |
| leader sequence; | Encoded | MALPVTALLLPLALLLHAARPDIQMTQTTSSLSASL | 128 | 129 |
| CD19 scFv; | Protein 1 | GDRVTISCRASQDISKYLNWYQQKPDGTVKLLIYH | | |
| CD8α Hinge-TM; | | TSRLHSGVPSRFSGSGSGTDYSLTISNLEQEDIATYF | | |
| 4-1BB signaling | | CQQGNTLPYTFGGGTKLEITGGGGSGGGGSGGGGS | | |
| domain; CD3 zeta | | EVKLQESGPGLVAPSQSLSVTCTVSGVSLPDYGVS | | |
| signaling domain; | | WIRQPPRKGLEWLGVIWGSETTYYNSALKSRLTIIK | | |
| stop; Spacer; | | DNSKSQVFLKMNSLQTDDTAIYYCAKHYYYGGSY | | |
| IRES; Spacer; | | AMDYWGQGTSVTVSSTTTPAPRPPTPAPTIASQPLS | | |
| IL12B (p40) | | LRPEACRPAAGGAVHTRGLDFACDIYIWAPLAGTC | | |
| signal sequence; | | GVLLLSLVITLYCKRGRKKLLYIFKQPFMRPVQTTQ | | |
| IL12B (p40) | | EEDGCSCRFPEEEEGGCELRVKFSRSADAPAYKQG | | |
| (23-328 of WT); | | QNQLYNELNLGRREEYDVLDKRRGRDPEMGGKPR | | |
| Linker ((G4S)3; | | RKNPQEGLYNELQKDKMAEAYSEIGMKGERRRGK | | |
| IL12A (p35) | | GHDGLYQGLSTATKDTYDALHMQALPPR* | | |
| (57-253 of WT); | Encoded | MCHQQLVISWFSLVFLASPLVAIWELKKDVYVVEL | 154 | 155 |
| Linker (GS); ER | Protein 2 | DWYPDAPGEMVVLTCDTPEEDGITWTLDQSSEVL | | |
| (aa 305-549 of | | GSGKTLTIQVKEFGDAGQYTCHKGGEVLSHSLLLL | | |
| WT, L384M, | | HKKEDGIWSTDILKDQKEPKNKTFLRCEAKNYSGR | | |
| M421G, G521R, | | FTCWWLTTISTDLTFSVKSSRGSSDPQGVTCGAATL | | |
| Y537S); stop) | | SAERVRGDNKEYEYSVECQEDSACPAAEESLPIEV | | |
| | | MVDAVHKLKYENYTSSFFIRDIIKPDPPKNLQLKPL | | |
| | | KNSRQVEVSWEYPDTWSTPHSYFSLTFCVQVQGKS | | |
| | | KREKKDRVFTDKTSATVICRKNASISVRAQDRYYS | | |
| | | SSWSEWASVPCSGGGGSGGGGSGGGGSRNLPVAT | | |
| | | PDPGMFPCLHHSQNLLRAVSNMLQKARQTLEFYPC | | |
| | | TSEEIDHEDITKDKTSTVEACLPLELTKNESCLNSRE | | |
| | | TSFITNGSCLASRKTSFMMALCLSSIYEDLKMYQVE | | |
| | | FKTMNAKLLMDPKRQIFLDQNMLAVIDELMQALN | | |
| | | FNSETVPQKSSLEEPDFYKTKIKLCILLHAFRIRAVTI | | |
| | | DRVMSYLNASGSSLALSLTADQMVSALLDAEPPIL | | |
| | | YSEYDPTRPFSEASMMGLLTNLADRELVHMINWA | | |
| | | KRVPGFVDLTLHDQVHLLECAWMEILMIGLVWRS | | |
| | | MEHPGKLLFAPNLLLDRNQGKCVEGGVEIHMLL | | |
| | | ATSSRFRMMNLQGEEFVCLKSIILLNSGVYTFLSST | | |
| | | LKSLEEKDHIHRVLDKITDTLIHLMAKAGLTLQQQ | | |
| | | HQRLAQLLLILSHIRHMSNKRMEHLYSMKCKNVV | | |
| | | PLSDLLLEMLDAHRL* | | |
| OT-001736 (Full | Full | MALPVTALLLPLALLLHAARPDIQMTQTTSSLSASL | 156 | 157 |
| Construct: CD8α | Construct | GDRVTISCRASQDISKYLNWYQQKPDGTVKLLIYH | | |
| leader sequence; | | TSRLHSGVPSRFSGSGSGTDYSLTISNLEQEDIATYF | | |
| CD19 scFv; | | CQQGNTLPYTFGGGTKLEITGGGGSGGGGSGGGGS | | |
| CD8α Hinge-TM; | | EVKLQESGPGLVAPSQSLSVTCTVSGVSLPDYGVS | | |
| 4-1BB signaling | | WIRQPPRKGLEWLGVIWGSETTYYNSALKSRLTIIK | | |
| domain; CD3 zeta | | DNSKSQVFLKMNSLQTDDTAIYYCAKHYYYGGSY | | |
| signaling domain; | | AMDYWGQGTSVTVSSTTTPAPRPPTPAPTIASQPLS | | |
| Linker (GS); P2A | | LRPEACRPAAGGAVHTRGLDFACDIYIWAPLAGTC | | |
| cleavage site; | | GVLLLSLVITLYCKRGRKKLLYIFKQPFMRPVQTTQ | | |
| IL12B (p40) | | EEDGCSCRFPEEEEGGCELRVKFSRSADAPAYKQG | | |
| signal sequence; | | QNQLYNELNLGRREEYDVLDKRRGRDPEMGGKPR | | |
| IL12B (p40) | | RKNPQEGLYNELQKDKMAEAYSEIGMKGERRRGK | | |
| (23-328 of WT); | | GHDGLYQGLSTATKDTYDALHMQALPPRGSATNF | | |
| Linker ((G4S)3; | | SLLKQAGDVEENPGPMCHQQLVISWFSLVFLASPL | | |
| IL12A (p35) | | VAIWELKKDVYVVELDWYPDAPGEMVVLTCDTPE | | |
| (57-253 of WT); | | EDGITWTLDQSSEVLGSGKTLTIQVKEFGDAGQYT | | |
| Linker (GS); | | CHKGGEVLSHSLLLLHKKEDGIWSTDILKDQKEPK | | |
| ER (aa 305-549 of | | NKTFLRCEAKNYSGRFTCWWLTTISTDLTFSVKSS | | |
| WT, L384M, | | RGSSDPQGVTCGAATLSAERVRGDNKEYEYSVEC | | |
| N413D , M421G, | | QEDSACPAAEESLPIEVMVDAVHKLKYENYTSSFFI | | |
| G521R, Y537S); | | RDIIKPDPPKNLQLKPLKNSRQVEVSWEYPDTWSTP | | |
| stop) | | HSYFSLTFCVQVQGKSKREKKDRVFTDKTSATVIC | | |
| | | RKNASISVRAQDRYYSSSWSEWASVPCSGGGGSG | | |
| | | GGGSGGGGSRNLPVATPDPGMFPCLHHSQNLLRA | | |
| | | VSNMLQKARQTLEFYPCTSEEIDHEDITKDKTSTVE | | |
| | | ACLPLELTKNESCLNSRETSFITNGSCLASRKTSFM | | |
| | | MALCLSSIYEDLKMYQVEFKTMNAKLLMDPKRQI | | |
| | | FLDQNMLAVIDELMQALNFNSETVPQKSSLEEPDF | | |
| | | YKTKIKLCILLHAFRIRAVTIDRVMSYLNASGSSLA | | |
| | | LSLTADQMVSALLDAEPPILYSEYDPTRPFSEASMM | | |
| | | GLLTNLADRELVHMINWAKRVPGFVDLTLHDQVH | | |
| | | LLECAWMEILMIGLVWRSMEHPGKLLFAPNLLLDR | | |
| | | DQGKCVEGGVEIFDMLLATSSRFRMMNLQGEEFV | | |
| | | CLKSIILLNSGVYTFLSSTLKSLEEKDHIHRVLDKIT | | |
| | | DTLIHLMAKAGLTLQQQHQRLAQLLLILSHIRHMS | | |
| | | NKRMEHLYSMKCKNVVPLSDLLLEMLDAHRL* | | |
| OT-001649 | Full | MALPVTALLLPLALLLHAARPDIQMTQTTSSLSASL | 373 | 374 |
| (CD8α leader | Construct | GDRVTISCRASQDISKYLNWYQQKPDGTVKLLIYH | | |
| sequence; CD19 | | TSRLHSGVPSRFSGSGSGTDYSLTISNLEQEDIATYF | | |
| scFv; CD8α | | CQQGNTLPYTFGGGTKLEITGGGGSGGGGSGGGGS | | |
| Hinge-TM; 4- | | EVKLQESGPGLVAPSQSLSVTCTVSGVSLPDYGVS | | |
| 1BB signaling | | WIRQPPRKGLEWLGVIWGSETTYYNSALKSRLTIIK | | |
| domain; CD3 zeta | | DNSKSQVFLKMNSLQTDDTAIYYCAKHYYYGGSY | | |
| signaling domain; | | AMDYWGQGTSVTVSSTTTPAPRPPTPAPTIASQPLS | | |
| Linker (GS); P2A | | LRPEACRPAAGGAVHTRGLDFACDIYIWAPLAGTC | | |
| cleavage site; | | GVLLLSLVITLYCKRGRKKLLYIFKQPFMRPVQTTQ | | |
| IL12B (p40) | | EEDGCSCRFPEEEEGGCELRVKFSRSADAPAYKQG | | |
| signal sequence; | | QNQLYNELNLGRREEYDVLDKRRGRDPEMGGKPR | | |
| IL12B (p40) | | RKNPQEGLYNELQKDKMAEAYSEIGMKGERRRGK | | |
| (23-328 of WT); | | GHDGLYQGLSTATKDTYDALHMQALPPRGSATNF | | |
| Linker ((G4S)3; | | SLLKQAGDVEENPGPMCHQQLVISWFSLVFLASPL | | |
| IL12A (p35) | | VAIWELKKDVYVVELDWYPDAPGEMVVLTCDTPE | | |
| (57-253 of WT); | | EDGITWTLDQSSEVLGSGKTLTIQVKEFGDAGQYT | | |
| Linker (GS); ER | | CHKGGEVLSHSLLLLHKKEDGIWSTDILKDQKEPK | | |
| (aa 305-549 of | | NKTFLRCEAKNYSGRFTCWWLTTISTDLTFSVKSSR | | |
| WT, L384M, | | GSSDPQGVTCGAATLSAERVRGDNKEYEYSVECQE | | |
| N413T, M421G, | | DSACPAAEESLPIEVMVDAVHKLKYENYTSSFFIRD | | |
| G521R, Y537S); | | IIKPDPPKNLQLKPLKNSRQVEVSWEYPDTWSTPHS | | |
| stop) | | YFSLTFCVQVQGKSKREKKDRVFTDKTSATVICRK | | |
| | | NASISVRAQDRYYSSSWSEWASVPCSGGGGSGGGG | | |
| | | SGGGGSRNLPVATPDPGMFPCLHHSQNLLRAVSNM | | |
| | | LQKARQTLEFYPCTSEEIDHEDITKDKTSTVEACLPL | | |
| | | ELTKNESCLNSRETSFITNGSCLASRKTSFMMALCL | | |
| | | SSIYEDLKMYQVEFKTMNAKLLMDPKRQIFLDQN | | |
| | | MLAVIDELMQALNFNSETVPQKSSLEEPDFYKTKIK | | |
| | | LCILLHAFRIRAVTIDRVMSYLNASGSSLALSLTAD | | |
| | | QMVSALLDAEPPILYSEYDPTRPFSEASMMGLLTNL | | |
| | | ADRELVHMINWAKRVPGFVDLTLHDQVHLLECAW | | |
| | | MEILMIGLVWRSMEHPGKLLFAPNLLLDRTQGKCV | | |
| | | EGGVEIFDMLLATSSRFRMMNLQGEEFVCLKSIILL | | |
| | | NSGVYTFLSSTLKSLEEKDHIHRVLDKITDTLIHLM | | |
| | | AKAGLTLQQQHQRLAQLLLILSHIRHMSNKRMEHL | | |
| | | YSMKCKNVVPLSDLLLEMLDAHRL* | | |
| OT-001651 | Full | MALPVTALLLPLALLLHAARPDIQMTQTTSSLSASL | 426 | 427 |
| (CD8a leader; | construct | GDRVTISCRASQDISKYLNWYQQKPDGTVKLLIYH | | |
| CD19 scFv; CD8α | | TSRLHSGVPSRFSGSGSGTDYSLTISNLEQEDIATYF | | |
| hinge-TM; 4-1BB | | CQQGNTLPYTFGGGTKLEITGGGGSGGGGSGGGGS | | |
| intracellular | | EVKLQESGPGLVAPSQSLSVTCTVSGVSLPDYGVS | | |
| signaling domain; | | WIRQPPRKGLEWLGVIWGSETTYYNSALKSRLTIIK | | |
| CD3 zeta | | DNSKSQVFLKMNSLQTDDTAIYYCAKHYYYGGSY | | |
| signaling domain; | | AMDYWGQGTSVTVSSTTTPAPRPPTPAPTIASQPLS | | |
| Linker (GS); P2A | | LRPEACRPAAGGAVHTRGLDFACDIYIWAPLAGTC | | |
| cleavage site; | | GVLLLSLVITLYCKRGRKKLLYIFKQPFMRPVQTTQ | | |
| Interleukin-12 | | EEDGCSCRFPEEEEGGCELRVKFSRSADAPAYKQG | | |
| subunit beta (p40) | | QNQLYNELNLGRREEYDVLDKRRGRDPEMGGKPR | | |
| leader; IL12B | | RKNPQEGLYNELQKDKMAEAYSEIGMKGERRRGK | | |
| (p40) (23-328 of | | GHDGLYQGLSTATKDTYDALHMQALPPRGSATNF | | |
| WT); Linker | | SLLKQAGDVEENPGPMCHQQLVISWFSLVFLASPL | | |
| ((G4S)3); IL12A | | VAIWELKKDVYVVELDWYPDAPGEMVVLTCDTPE | | |
| (p35) (57-253 of | | EDGITWTLDQSSEVLGSGKTLTIQVKEFGDAGQYT | | |
| WT); Linker | | CHKGGEVLSHSLLLLHKKEDGIWSTDILKDQKEPK | | |
| (GS); ER (aa | | NKTFLRCEAKNYSGRFTCWWLTTISTDLTFSVKSSR | | |
| 305-549 of WT, | | GSSDPQGVTCGAATLSAERVRGDNKEYEYSVECQE | | |
| L384M, M421G, | | DSACPAAEESLPIEVMVDAVHKLKYENYTSSFFIRD | | |
| Q502H, G521R, | | IIKPDPPKNLQLKPLKNSRQVEVSWEYPDTWSTPHS | | |
| Y537S); stop) | | YFSLTFCVQVQGKSKREKKDRVFTDKTSATVICRK | | |
| | | NASISVRAQDRYYSSSWSEWASVPCSGGGGSGGGG | | |
| | | SGGGGSRNLPVATPDPGMFPCLHHSQNLLRAVSNM | | |
| | | LQKARQTLEFYPCTSEEIDHEDITKDKTSTVEACLPL | | |
| | | ELTKNESCLNSRETSFITNGSCLASRKTSFMMALCL | | |
| | | SSIYEDLKMYQVEFKTMNAKLLMDPKRQIFLDQN | | |
| | | MLAVIDELMQALNFNSETVPQKSSLEEPDFYKTKIK | | |
| | | LCILLHAFRIRAVTIDRVMSYLNASGSSLALSLTAD | | |
| | | QMVSALLDAEPPILYSEYDPTRPFSEASMMGLLTNL | | |
| | | ADRELVHMINWAKRVPGFVDLTLHDQVHLLECAW | | |
| | | MEILMIGLVWRSMEHPGKLLFAPNLLLDRNQGKCV | | |
| | | EGGVEIFDMLLATSSRFRMMNLQGEEFVCLKSIILL | | |
| | | NSGVYTFLSSTLKSLEEKDHIHRVLDKITDTLIHLM | | |
| | | AKAGLTLQQQHHRLAQLLLILSHIRHMSNKRMEHL | | |
| | | YSMKCKNVVPLSDLLLEMLDAHRL* | | |
| OT-001652 | Full | MALPVTALLLPLALLLHAARPDIQMTQTTSSLSASL | 375 | 376 |
| (CD8α leader | Construct | GDRVTISCRASQDISKYLNWYQQKPDGTVKLLIYH | | |
| sequence; CD19 | | TSRLHSGVPSRFSGSGSGTDYSLTISNLEQEDIATYF | | |
| scFv; CD8α | | CQQGNTLPYTFGGGTKLEITGGGGSGGGGSGGGGS | | |
| Hinge-TM; 4- | | EVKLQESGPGLVAPSQSLSVTCTVSGVSLPDYGVS | | |
| 1BB signaling | | WIRQPPRKGLEWLGVIWGSETTYYNSALKSRLTIIK | | |
| domain; CD3 zeta | | DNSKSQVFLKMNSLQTDDTAIYYCAKHYYYGGSY | | |
| signaling domain; | | AMDYWGQGTSVTVSSTTTPAPRPPTPAPTIASQPLS | | |
| Linker (GS); P2A | | LRPEACRPAAGGAVHTRGLDFACDIYIWAPLAGTC | | |
| cleavage site; | | GVLLLSLVITLYCKRGRKKLLYIFKQPFMRPVQTTQ | | |
| IL12B (p40) | | EEDGCSCRFPEEEEGGCELRVKFSRSADAPAYKQG | | |
| signal sequence; | | QNQLYNELNLGRREEYDVLDKRRGRDPEMGGKPR | | |
| IL12B (p40) | | RKNPQEGLYNELQKDKMAEAYSEIGMKGERRRGK | | |
| (23-328 of WT); | | GHDGLYQGLSTATKDTYDALHMQALPPRGSATNF | | |
| Linker ((G4S)3; | | SLLKQAGDVEENPGPMCHQQLVISWFSLVFLASPL | | |
| IL12A (p35) | | VAIWELKKDVYVVELDWYPDAPGEMVVLTCDTPE | | |
| (57-253 of WT); | | EDGITWTLDQSSEVLGSGKTLTIQVKEFGDAGQYT | | |
| Linker (GS); ER | | CHKGGEVLSHSLLLLHKKEDGIWSTDILKDQKEPK | | |
| (aa 305-549 of | | NKTFLRCEAKNYSGRFTCWWLTTISTDLTFSVKSSR | | |
| WT, L384M, | | GSSDPQGVTCGAATLSAERVRGDNKEYEYSVECQE | | |
| M421G, Q502D, | | DSACPAAEESLPIEVMVDAVHKLKYENYTSSFFIRD | | |
| G521R, Y537S); | | IIKPDPPKNLQLKPLKNSRQVEVSWEYPDTWSTPHS | | |
| stop) | | YFSLTFCVQVQGKSKREKKDRVFTDKTSATVICRK | | |
| | | NASISVRAQDRYYSSSWSEWASVPCSGGGGSGGGG | | |
| | | SGGGGSRNLPVATPDPGMFPCLHHSQNLLRAVSNM | | |
| | | LQKARQTLEFYPCTSEEIDHEDITKDKTSTVEACLPL | | |
| | | ELTKNESCLNSRETSFITNGSCLASRKTSFMMALCL | | |
| | | SSIYEDLKMYQVEFKTMNAKLLMDPKRQIFLDQN | | |
| | | MLAVIDELMQALNFNSETVPQKSSLEEPDFYKTKIK | | |
| | | LCILLHAFRIRAVTIDRVMSYLNASGSSLALSLTAD | | |
| | | QMVSALLDAEPPILYSEYDPTRPFSEASMMGLLTNL | | |
| | | ADRELVHMINWAKRVPGFVDLTLHDQVHLLECAW | | |
| | | MEILMIGLVWRSMEHPGKLLFAPNLLLDRNQGKCV | | |
| | | EGGVEIFDMLLATSSRFRMMNLQGEEFVCLKSIILL | | |
| | | NSGVYTFLSSTLKSLEEKDHIHRVLDKITDTLIHLM | | |
| | | AKAGLTLQQQHDRLAQLLLILSHIRHMSNKRMEHL | | |
| | | YSMKCKNVVPLSDLLLEMLDAHRL* | | |
| OT-001653 | Full | MALPVTALLLPLALLLHAARPDIQMTQTTSSLSASL | 377 | 378 |
| (CD8α leader | Construct | GDRVTISCRASQDISKYLNWYQQKPDGTVKLLIYH | | |
| sequence; CD19 | | TSRLHSGVPSRFSGSGSGTDYSLTISNLEQEDIATYF | | |
| scFv; CD8α | | CQQGNTLPYTFGGGTKLEITGGGGSGGGGSGGGGS | | |
| Hinge-TM; 4- | | EVKLQESGPGLVAPSQSLSVTCTVSGVSLPDYGVS | | |
| 1BB signaling | | WIRQPPRKGLEWLGVIWGSETTYYNSALKSRLTIIK | | |
| domain; CD3 zeta | | DNSKSQVFLKMNSLQTDDTAIYYCAKHYYYGGSY | | |
| signaling domain; | | AMDYWGQGTSVTVSSTTTPAPRPPTPAPTIASQPLS | | |
| Linker (GS); P2A | | LRPEACRPAAGGAVHTRGLDFACDIYIWAPLAGTC | | |
| cleavage site; | | GVLLLSLVITLYCKRGRKKLLYIFKQPFMRPVQTTQ | | |
| IL12B (p40) | | EEDGCSCRFPEEEEGGCELRVKFSRSADAPAYKQG | | |
| signal sequence; | | QNQLYNELNLGRREEYDVLDKRRGRDPEMGGKPR | | |
| IL12B (p40) | | RKNPQEGLYNELQKDKMAEAYSEIGMKGERRRGK | | |
| (23-328 of WT); | | GHDGLYQGLSTATKDTYDALHMQALPPRGSATNF | | |
| Linker ((G4S)3; | | SLLKQAGDVEENPGPMCHQQLVISWFSLVFLASPL | | |
| IL12A (p35) | | VAIWELKKDVYVVELDWYPDAPGEMVVLTCDTPE | | |
| (57-253 of WT); | | EDGITWTLDQSSEVLGSGKTLTIQVKEFGDAGQYT | | |
| Linker (GS); ER | | CHKGGEVLSHSLLLLHKKEDGIWSTDILKDQKEPK | | |
| (aa 305-549 of | | NKTFLRCEAKNYSGRFTCWWLTTISTDLTFSVKSSR | | |
| WT, L384M, | | GSSDPQGVTCGAATLSAERVRGDNKEYEYSVECQE | | |
| M421G, Q502R, | | DSACPAAEESLPIEVMVDAVHKLKYENYTSSFFIRD | | |
| G521R, Y537S); | | IIKPDPPKNLQLKPLKNSRQVEVSWEYPDTWSTPHS | | |
| stop) | | YFSLTFCVQVQGKSKREKKDRVFTDKTSATVICRK | | |
| | | NASISVRAQDRYYSSSWSEWASVPCSGGGGSGGGG | | |
| | | SGGGGSRNLPVATPDPGMFPCLHHSQNLLRAVSNM | | |
| | | LQKARQTLEFYPCTSEEIDHEDITKDKTSTVEACLPL | | |
| | | ELTKNESCLNSRETSFITNGSCLASRKTSFMMALCL | | |
| | | SSIYEDLKMYQVEFKTMNAKLLMDPKRQIFLDQN | | |
| | | MLAVIDELMQALNFNSETVPQKSSLEEPDFYKTKIK | | |
| | | LCILLHAFRIRAVTIDRVMSYLNASGSSLALSLTAD | | |
| | | QMVSALLDAEPPILYSEYDPTRPFSEASMMGLLTNL | | |
| | | ADRELVHMINWAKRVPGFVDLTLHDQVHLLECAW | | |
| | | MEILMIGLVWRSMEHPGKLLFAPNLLLDRNQGKCV | | |
| | | EGGVEIFDMLLATSSRFRMMNLQGEEFVCLKSIILL | | |
| | | NSGVYTFLSSTLKSLEEKDHIHRVLDKITDTLIHLM | | |
| | | AKAGLTLQQQHRRLAQLLLILSHIRHMSNKRMEHL | | |
| | | YSMKCKNVVPLSDLLLEMLDAHRL* | | |
| OT-001356 (Full | Full | — | — | 379 |
| Construct: CD8a | Construct | | | |
| leader; CD19 | Encoded | MALPVTALLLPLALLLHAARPDIQMTQTTSSLSASL | 380 | 381 |
| scFv; CD8a Hinge | Protein 1 | GDRVTISCRASQDISKYLNWYQQKPDGTVKLLIYH | | |
| and | | TSRLHSGVPSRFSGSGSGTDYSLTISNLEQEDIATYF | | |
| Transmembrane | | CQQGNTLPYTFGGGTKLEITGGGGSGGGGSGGGGS | | |
| Domain; 4-1BB | | EVKLQESGPGLVAPSQSLSVTCTVSGVSLPDYGVS | | |
| intracellular | | WIRQPPRKGLEWLGVIWGSETTYYNSALKSRLTIIK | | |
| domain; CD3 zeta | | DNSKSQVFLKMNSLQTDDTAIYYCAKHYYYGGSY | | |
| signaling domain; | | AMDYWGQGTSVTVSSTTTPAPRPPTPAPTIASQPLS | | |
| stop; spacer; | | LRPEACRPAAGGAVHTRGLDFACDIYIWAPLAGTC | | |
| IRES; spacer; p40 | | GVLLLSLVITLYCKRGRKKLLYIFKQPFMRPVQTTQ | | |
| signal sequence; | | EEDGCSCRFPEEEEGGCELRVKFSRSADAPAYKQG | | |
| p40 (23-328 of | | QNQLYNELNLGRREEYDVLDKRRGRDPEMGGKPR | | |
| WT); Linker | | RKNPQEGLYNELQKDKMAEAYSEIGMKGERRRGK | | |
| ((G4S)3); p35 | | GHDGLYQGLSTATKDTYDALHMQALPPR* | | |
| (57-253 of WT); | Encoded | MCHQQLVISWFSLVFLASPLVAIWELKKDVYVVEL | 382 | 383 |
| BamHI (GS); | Protein 2 | DWYPDAPGEMVVLTCDTPEEDGITWTLDQSSEVLG | | |
| stop) (Encoded | | SGKTLTIQVKEFGDAGQYTCHKGGEVLSHSLLLLH | | |
| Protein 1: CD8a | | KKEDGIWSTDILKDQKEPKNKTFLRCEAKNYSGRF | | |
| leader; CD19 | | TCWWLTTISTDLTFSVKSSRGSSDPQGVTCGAATLS | | |
| scFv; CD8a Hinge | | AERVRGDNKEYEYSVECQEDSACPAAEESLPIEVM | | |
| and | | VDAVHKLKYENYTSSHFFIRDIIKPDPPKNLQLKPLK | | |
| Transmembrane | | NSRQVEVSWEYPDTWSTPHSYFSLTFCVQVQGKSK | | |
| Domain; 4-1BB | | REKKDRVFTDKTSATVICRKNASISVRAQDRYYSSS | | |
| intracellular | | WSEWASVPCSGGGGSGGGGSGGGGSRNLPVATPD | | |
| domain; CD3 zeta | | PGMFPCLHHSQNLLRAVSNMLQKARQTLEFYPCTS | | |
| signaling domain; | | EEIDHEDITKDKTSTVEACLPLELTKNESCLNSRETS | | |
| stop) (Encoded | | FITNGSCLASRKTSFMMALCLSSIYEDLKMYQVEFK | | |
| Protein 2: p40 | | TMNAKLLMDPKRQIFLDQNMLAVIDELMQALNFN | | |
| signal sequence; | | SETVPQKSSLEEPDFYKTKIKLCILLHAFRIRAVTIDR | | |
| p40 (23-328 of | | VMSYLNASGS* | | |
| WT); Linker | | | | |
| ((G4S)3); p35 | | | | |
| (57-253 of WT); | | | | |
| BamHI (GS); | | | | |
| stop) | | | | |
| OT-001357 | Full | MALPVTALLLPLALLLHAARPDIQMTQTTSSLSASL | 384 | 385 |
| (CD8a leader; | Construct | GDRVTISCRASQDISKYLNWYQQKPDGTVKLLIYH | | |
| CD19 scFv; CD8a | | TSRLHSGVPSRFSGSGSGTDYSLTISNLEQEDIATYF | | |
| Hinge and | | CQQGNTLPYTFGGGTKLEITGGGGSGGGGSGGGGS | | |
| Transmembrane | | EVKLQESGPGLVAPSQSLSVTCTVSGVSLPDYGVS | | |
| Domain; 4-1BB | | WIRQPPRKGLEWLGVIWGSETTYYNSALKSRLTIIK | | |
| intracellular | | DNSKSQVFLKMNSLQTDDTAIYYCAKHYYYGGSY | | |
| domain; CD3 zeta | | AMDYWGQGTSVTVSSTTTPAPRPPTPAPTIASQPLS | | |
| signaling domain; | | LRPEACRPAAGGAVHTRGLDFACDIYIWAPLAGTC | | |
| BamHI (GS); P2A | | GVLLLSLVITLYCKRGRKKLLYIFKQPFMRPVQTTQ | | |
| Cleavage Site; | | EEDGCSCRFPEEEEGGCELRVKFSRSADAPAYKQG | | |
| p40 signal | | QNQLYNELNLGRREEYDVLDKRRGRDPEMGGKPR | | |
| sequence; p40 | | RKNPQEGLYNELQKDKMAEAYSEIGMKGERRRGK | | |
| (23-328 of WT); | | GHDGLYQGLSTATKDTYDALHMQALPPRGSATNF | | |
| Linker ((G4S)3); | | SLLKQAGDVEENPGPMCHQQLVISWFSLVFLASPL | | |
| p35 (57-253 of | | VAIWELKKDVYVVELDWYPDAPGEMVVLTCDTPE | | |
| WT); BamHI | | EDGITWTLDQSSEVLGSGKTLTIQVKEFGDAGQYT | | |
| (GS); stop)) | | CHKGGEVLSHSLLLLHKKEDGIWSTDILKDQKEPK | | |
| | | NKTFLRCEAKNYSGRFTCWWLTTISTDLTFSVKSSR | | |
| | | GSSDPQGVTCGAATLSAERVRGDNKEYEYSVECQE | | |
| | | DSACPAAEESLPIEVMVDAVHKLKYENYTSSFFIRD | | |
| | | IIKPDPPKNLQLKPLKNSRQVEVSWEYPDTWSTPHS | | |
| | | YFSLTFCVQVQGKSKREKKDRVFTDKTSATVICRK | | |
| | | NASISVRAQDRYYSSSWSEWASVPCSGGGGSGGGG | | |
| | | SGGGGSRNLPVATPDPGMFPCLHHSQNLLRAVSNM | | |
| | | LQKARQTLEFYPCTSEEIDHEDITKDKTSTVEACLPL | | |
| | | ELTKNESCLNSRETSFITNGSCLASRKTSFMMALCL | | |
| | | SSIYEDLKMYQVEFKTMNAKLLMDPKRQIFLDQN | | |
| | | MLAVIDELMQALNFNSETVPQKSSLEEPDFYKTKIK | | |
| | | LCILLHAFRIRAVTIDRVMSYLNASGS* | | |

TABLE 6
CD19-CAR Constructs

| | | AA | NA |
|---|---|---|---|
| | | SEQ | SEQ |
| Construct ID | AA sequence | ID NO. | ID NO. |
| OT-001407 (CD8a | MALPVTALLLPLALLLHAARPDIQMTQTTSSLSASLGDRVTIS | 386 | 387 |
| leader; CD19 | CRASQDISKYLNWYQQKPDGTVKLLIYHTSRLHSGVPSRFSG | | |
| scFV; CD8a-Tm; | SGSGTDYSLTISNLEQEDIATYFCQQGNTLPYTFGGGTKLEIT | | |
| 41BB ; CD3 zeta; | GGGGSGGGGSGGGGSEVKLQESGPGLVAPSQSLSVTCTVSG | | |
| stop) | VSLPDYGVSWIRQPPRKGLEWLGVIWGSETTYYNSALKSRLT | | |
| | IIKDNSKSQVFLKMNSLQTDDTAIYYCAKHYYYGGSYAMDY | | |
| | WGQGTSVTVSSTTTPAPRPPTPAPTIASQPLSLRPEACRPAAG | | |
| | GAVHTRGLDFACDIYIWAPLAGTCGVLLLSLVITLYCKRGRK | | |
| | KLLYIFKQPFMRPVQTTQEEDGCSCRFPEEEEGGCELRVKFSR | | |
| | SADAPAYKQGQNQLYNELNLGRREEYDVLDKRRGRDPEMG | | |
| | GKPRRKNPQEGLYNELQKDKMAEAYSEIGMKGERRRGKGH | | |
| | DGLYQGLSTATKDTYDALHMQALPPR* | | |
| OT-001790 (CD8α | MALPVTALLLPLALLLHAARPDIQMTQTTSSLSASLGDRVTIS | 388 | 389 |
| leader sequence; | CRASQDISKYLNWYQQKPDGTVKLLIYHTSRLHSGVPSRFSG | | |
| CD19 scFv; CD8α | SGSGTDYSLTISNLEQEDIATYFCQQGNTLPYTFGGGTKLEIT | | |
| Hinge-TM; 4-1BB | GGGGSGGGGSGGGGSEVKLQESGPGLVAPSQSLSVTCTVSG | | |
| signaling domain; | VSLPDYGVSWIRQPPRKGLEWLGVIWGSETTYYNSALKSRLT | | |
| CD3 zeta signaling | IIKDNSKSQVFLKMNSLQTDDTAIYYCAKHYYYGGSYAMDY | | |
| domain; Linker | WGQGTSVTVSSTTTPAPRPPTPAPTIASQPLSLRPEACRPAAG | | |
| (GS); ER (aa | GAVHTRGLDFACDIYIWAPLAGTCGVLLLSLVITLYCKRGRK | | |
| 305-549 of WT, | KLLYIFKQPFMRPVQTTQEEDGCSCRFPEEEEGGCELRVKFSR | | |
| R335G, L384M, | SADAPAYKQGQNQLYNELNLGRREEYDVLDKRRGRDPEMG | | |
| M421G, G521R, | GKPRRKNPQEGLYNELQKDKMAEAYSEIGMKGERRRGKGH | | |
| E523G, Y537S, | DGLYQGLSTATKDTYDALHMQALPPRGSSLALSLTADQMVS | | |
| A546T); stop) | ALLDAEPPILYSEYDPTGPFSEASMMGLLTNLADRELVHMIN | | |
| | WAKRVPGFVDLTLHDQVHLLECAWMEILMIGLVWRSMEHP | | |
| | GKLLFAPNLLLDRNQGKCVEGGVEIHMLLATSSRFRMMNL | | |
| | QGEEFVCLKSIILLNSGVYTFLSSTLKSLEEKDHIHRVLDKITD | | |
| | TLIHLMAKAGLTLQQQHQRLAQLLLILSHIRHMSNKRMGHL | | |
| | YSMKCKNVVPLSDLLLEMLDTHRL* | | |
| OT-001789 (CD8α | MALPVTALLLPLALLLHAARPDIQMTQTTSSLSASLGDRVTIS | 390 | 391 |
| leader sequence; | CRASQDISKYLNWYQQKPDGTVKLLIYHTSRLHSGVPSRFSG | | |
| CD19 scFv; CD8α | SGSGTDYSLTISNLEQEDIATYFCQQGNTLPYTFGGGTKLEIT | | |
| Hinge-TM; 4-1BB | GGGGSGGGGSGGGGSEVKLQESGPGLVAPSQSLSVTCTVSG | | |
| signaling domain; | VSLPDYGVSWIRQPPRKGLEWLGVIWGSETTYYNSALKSRLT | | |
| CD3 zeta signaling | IIKDNSKSQVFLKMNSLQTDDTAIYYCAKHYYYGGSYAMDY | | |
| domain; Linker | WGQGTSVTVSSTTTPAPRPPTPAPTIASQPLSLRPEACRPAAG | | |
| (GS); ER (aa | GAVHTRGLDFACDIYIWAPLAGTCGVLLLSLVITLYCKRGRK | | |
| 305-549 of WT, | KLLYIFKQPFMRPVQTTQEEDGCSCRFPEEEEGGCELRVKFSR | | |
| L384M; M421G; | SADAPAYKQGQNQLYNELNLGRREEYDVLDKRRGRDPEMG | | |
| T431I; G521R, | GKPRRKNPQEGLYNELQKDKMAEAYSEIGMKGERRRGKGH | | |
| Y537S) ; stop) | DGLYQGLSTATKDTYDALHMQALPPRGSSLALSLTADQMVS | | |
| | ALLDAEPPILYSEYDPTRPFSEASMMGLLTNLADRELVHMIN | | |
| | WAKRVPGFVDLTLHDQVHLLECAWMEILMIGLVWRSMEHP | | |
| | GKLLFAPNLLLDRNQGKCVEGGVEIHMLLAISSRFRMMNL | | |
| | QGEEFVCLKSIILLNSGVYTFLSSTLKSLEEKDHIHRVLDKITD | | |
| | TLIHLMAKAGLTLQQQHQRLAQLLLILSHIRHMSNKRMEHL | | |
| | YSMKCKNVVPLSDLLLEMLDAHRL* | | |
| OT-001788 (CD8α | MALPVTALLLPLALLLHAARPDIQMTQTTSSLSASLGDRVTIS | 392 | 393 |
| leader sequence; | CRASQDISKYLNWYQQKPDGTVKLLIYHTSRLHSGVPSRFSG | | |
| CD19 scFv; CD8α | SGSGTDYSLTISNLEQEDIATYFCQQGNTLPYTFGGGTKLEIT | | |
| Hinge-TM; 4-1BB | GGGGSGGGGSGGGGSEVKLQESGPGLVAPSQSLSVTCTVSG | | |
| signaling domain; | VSLPDYGVSWIRQPPRKGLEWLGVIWGSETTYYNSALKSRLT | | |
| CD3 zeta signaling | IIKDNSKSQVFLKMNSLQTDDTAIYYCAKHYYYGGSYAMDY | | |
| domain; Linker | WGQGTSVTVSSTTTPAPRPPTPAPTIASQPLSLRPEACRPAAG | | |
| (GS); ER (aa | GAVHTRGLDFACDIYIWAPLAGTCGVLLLSLVITLYCKRGRK | | |
| 305-549 of WT, | KLLYIFKQPFMRPVQTTQEEDGCSCRFPEEEEGGCELRVKFSR | | |
| L384M, N413D, | SADAPAYKQGQNQLYNELNLGRREEYDVLDKRRGRDPEMG | | |
| M421G, G521R, | GKPRRKNPQEGLYNELQKDKMAEAYSEIGMKGERRRGKGH | | |
| Y537S); stop) | DGLYQGLSTATKDTYDALHMQALPPRGSSLALSLTADQMVS | | |
| | ALLDAEPPILYSEYDPTRPFSEASMMGLLTNLADRELVHMIN | | |
| | WAKRVPGFVDLTLHDQVHLLECAWMEILMIGLVWRSMEHP | | |
| | GKLLFAPNLLLDRDQGKCVEGGVEIFDMLLATSSRFRMMNL | | |
| | QGEEFVCLKSIILLNSGVYTFLSSTLKSLEEKDHIHRVLDKITD | | |
| | TLIHLMAKAGLTLQQQHQRLAQLLLILSHIRHMSNKRMEHL | | |
| | YSMKCKNVVPLSDLLLEMLDAHRL* | | |
| OT-001787 (CD8α | MALPVTALLLPLALLLHAARPDIQMTQTTSSLSASLGDRVTIS | 394 | 395 |
| leader sequence; | CRASQDISKYLNWYQQKPDGTVKLLIYHTSRLHSGVPSRFSG | | |
| CD19 scFv; CD8α | SGSGTDYSLTISNLEQEDIATYFCQQGNTLPYTFGGGTKLEIT | | |
| Hinge-TM; 4-1BB | GGGGSGGGGSGGGGSEVKLQESGPGLVAPSQSLSVTCTVSG | | |
| signaling domain; | VSLPDYGVSWIRQPPRKGLEWLGVIWGSETTYYNSALKSRLT | | |
| CD3 zeta signaling | IIKDNSKSQVFLKMNSLQTDDTAIYYCAKHYYYGGSYAMDY | | |
| domain; Linker | WGQGTSVTVSSTTTPAPRPPTPAPTIASQPLSLRPEACRPAAG | | |
| (GS); ER (aa | GAVHTRGLDFACDIYIWAPLAGTCGVLLLSLVITLYCKRGRK | | |
| 305-549 of WT, | KLLYIFKQPFMRPVQTTQEEDGCSCRFPEEEEGGCELRVKFSR | | |
| L384M, M421G, | SADAPAYKQGQNQLYNELNLGRREEYDVLDKRRGRDPEMG | | |
| N519S, G521R, | GKPRRKNPQEGLYNELQKDKMAEAYSEIGMKGERRRGKGH | | |
| Y537S); stop) | DGLYQGLSTATKDTYDALHMQALPPRGSSLALSLTADQMVS | | |
| | ALLDAEPPILYSEYDPTRPFSEASMMGLLTNLADRELVHMIN | | |
| | WAKRVPGFVDLTLHDQVHLLECAWMEILMIGLVWRSMEHP | | |
| | GKLLFAPNLLLDRNQGKCVEGGVEIFDMLLATSSRFRMMNL | | |
| | QGEEFVCLKSIILLNSGVYTFLSSTLKSLEEKDHIHRVLDKITD | | |
| | TLIHLMAKAGLTLQQQHQRLAQLLLILSHIRHMSSKRMEHLY | | |
| | SMKCKNVVPLSDLLLEMLDAHRL* | | |
| OT-001786 (CD8α | MALPVTALLLPLALLLHAARPDIQMTQTTSSLSASLGDRVTIS | 396 | 397 |
| leader sequence; | CRASQDISKYLNWYQQKPDGTVKLLIYHTSRLHSGVPSRFSG | | |
| CD19 scFv; CD8α | SGSGTDYSLTISNLEQEDIATYFCQQGNTLPYTFGGGTKLEIT | | |
| Hinge-TM; 4-1BB | GGGGSGGGGSGGGGSEVKLQESGPGLVAPSQSLSVTCTVSG | | |
| signaling domain; | VSLPDYGVSWIRQPPRKGLEWLGVIWGSETTYYNSALKSRLT | | |
| CD3 zeta signaling | IIKDNSKSQVFLKMNSLQTDDTAIYYCAKHYYYGGSYAMDY | | |
| domain; Linker | WGQGTSVTVSSTTTPAPRPPTPAPTIASQPLSLRPEACRPAAG | | |
| (GS); ER (aa | GAVHTRGLDFACDIYIWAPLAGTCGVLLLSLVITLYCKRGRK | | |
| 305-549 of WT, | KLLYIFKQPFMRPVQTTQEEDGCSCRFPEEEEGGCELRVKFSR | | |
| L384M, M421G, | SADAPAYKQGQNQLYNELNLGRREEYDVLDKRRGRDPEMG | | |
| Q502R, G521R, | GKPRRKNPQEGLYNELQKDKMAEAYSEIGMKGERRRGKGH | | |
| Y537S); stop) | DGLYQGLSTATKDTYDALHMQALPPRGSSLALSLTADQMVS | | |
| | ALLDAEPPILYSEYDPTRPFSEASMMGLLTNLADRELVHMIN | | |
| | WAKRVPGFVDLTLHDQVHLLECAWMEILMIGLVWRSMEHP | | |
| | GKLLFAPNLLLDRNQGKCVEGGVEIFDMLLATSSRFRMMNL | | |
| | QGEEFVCLKSIILLNSGVYTFLSSTLKSLEEKDHIHRVLDKITD | | |
| | TLIHLMAKAGLTLQQQHRRLAQLLLILSHIRHMSNKRMEHL | | |
| | YSMKCKNVVPLSDLLLEMLDAHRL* | | |
| OT-001785 (CD8α | MALPVTALLLPLALLLHAARPDIQMTQTTSSLSASLGDRVTIS | 398 | 399 |
| leader sequence; | CRASQDISKYLNWYQQKPDGTVKLLIYHTSRLHSGVPSRFSG | | |
| CD19 scFv; CD8α | SGSGTDYSLTISNLEQEDIATYFCQQGNTLPYTFGGGTKLEIT | | |
| Hinge-TM; 4-1BB | GGGGSGGGGSGGGGSEVKLQESGPGLVAPSQSLSVTCTVSG | | |
| signaling domain; | VSLPDYGVSWIRQPPRKGLEWLGVIWGSETTYYNSALKSRLT | | |
| CD3 zeta signaling | IIKDNSKSQVFLKMNSLQTDDTAIYYCAKHYYYGGSYAMDY | | |
| domain; Linker | WGQGTSVTVSSTTTPAPRPPTPAPTIASQPLSLRPEACRPAAG | | |
| (GS); ER (aa | GAVHTRGLDFACDIYIWAPLAGTCGVLLLSLVITLYCKRGRK | | |
| 305-549 of WT, | KLLYIFKQPFMRPVQTTQEEDGCSCRFPEEEEGGCELRVKFSR | | |
| T371A, L384M, | SADAPAYKQGQNQLYNELNLGRREEYDVLDKRRGRDPEMG | | |
| M421G, N519S, | GKPRRKNPQEGLYNELQKDKMAEAYSEIGMKGERRRGKGH | | |
| G521R, Y537S); | DGLYQGLSTATKDTYDALHMQALPPRGSSLALSLTADQMVS | | |
| stop) | ALLDAEPPILYSEYDPTRPFSEASMMGLLTNLADRELVHMIN | | |
| | WAKRVPGFVDLALHDQVHLLECAWMEILMIGLVWRSMEHP | | |
| | GKLLFAPNLLLDRNQGKCVEGGVEIFDMLLATSSRFRMMNL | | |
| | QGEEFVCLKSIILLNSGVYTFLSSTLKSLEEKDHIHRVLDKITD | | |
| | TLIHLMAKAGLTLQQQHQRLAQLLLILSHIRHMSSKRMEHLY | | |
| | SMKCKNVVPLSDLLLEMLDAHRL* | | |
| OT-001784 (CD8α | MALPVTALLLPLALLLHAARPDIQMTQTTSSLSASLGDRVTIS | 400 | 401 |
| leader sequence; | CRASQDISKYLNWYQQKPDGTVKLLIYHTSRLHSGVPSRFSG | | |
| CD19 scFv; CD8α | SGSGTDYSLTISNLEQEDIATYFCQQGNTLPYTFGGGTKLEIT | | |
| Hinge-TM; 4-1BB | GGGGSGGGGSGGGGSEVKLQESGPGLVAPSQSLSVTCTVSG | | |
| signaling domain; | VSLPDYGVSWIRQPPRKGLEWLGVIWGSETTYYNSALKSRLT | | |
| CD3 zeta signaling | IIKDNSKSQVFLKMNSLQTDDTAIYYCAKHYYYGGSYAMDY | | |
| domain; Linker | WGQGTSVTVSSTTTPAPRPPTPAPTIASQPLSLRPEACRPAAG | | |
| (GS); ER (aa | GAVHTRGLDFACDIYIWAPLAGTCGVLLLSLVITLYCKRGRK | | |
| 305-549 of WT, | KLLYIFKQPFMRPVQTTQEEDGCSCRFPEEEEGGCELRVKFSR | | |
| S305N, L384M, | SADAPAYKQGQNQLYNELNLGRREEYDVLDKRRGRDPEMG | | |
| M421G, G442V, | GKPRRKNPQEGLYNELQKDKMAEAYSEIGMKGERRRGKGH | | |
| G521R, Y537S); | DGLYQGLSTATKDTYDALHMQALPPRGSNLALSLTADQMV | | |
| stop) | SALLDAEPPILYSEYDPTRPFSEASMMGLLTNLADRELVHMI | | |
| | NWAKRVPGFVDLTLHDQVHLLECAWMEILMIGLVWRSMEH | | |
| | PGKLLFAPNLLLDRNQGKCVEGGVEIFDMLLATSSRFRMMN | | |
| | LQVEEFVCLKSIILLNSGVYTFLSSTLKSLEEKDHIHRVLDKIT | | |
| | DTLIHLMAKAGLTLQQQHQRLAQLLLILSHIRHMSNKRMEH | | |
| | LYSMKCKNVVPLSDLLLEMLDAHRL* | | |
| OT-001793 (CD8α | MALPVTALLLPLALLLHAARPSLALSLTADQMVSALLDAEPP | 402 | 403 |
| leader sequence; | ILYSEYDPTRPFSEASMMGLLTNLADRELVHMINWAKRVPG | | |
| ER (aa 305-549 of | FVDLALHDQVHLLECAWMEILMIGLVWRSMEHPGKLLFAPN | | |
| WT, T371A, | LLLDRNQGKCVEGGVEIFDMLLATSSRFRMMNLQGEEFVCL | | |
| L384M, M421G, | KSIILLNSGVYTFLSSTLKSLEEKDHIHRVLDKITDTLIHLMAK | | |
| N519S, G521R, | AGLTLQQQHQRLAQLLLILSHIRHMSSKRMEHLYSMKCKNV | | |
| Y537S); Linker | VPLSDLLLEMLDAHRLGSDIQMTQTTSSLSASLGDRVTISCRA | | |
| (GS); CD19 scFv; | SQDISKYLNWYQQKPDGTVKLLIYHTSRLHSGVPSRFSGSGS | | |
| CD8α Hinge-TM; | GTDYSLTISNLEQEDIATYFCQQGNTLPYTFGGGTKLEITGGG | | |
| 4-1BB signaling | GSGGGGSGGGGSEVKLQESGPGLVAPSQSLSVTCTVSGVSLP | | |
| domain; CD3 zeta | DYGVSWIRQPPRKGLEWLGVIWGSETTYYNSALKSRLTIIKD | | |
| signaling domain; | NSKSQVFLKMNSLQTDDTAIYYCAKHYYYGGSYAMDYWG | | |
| stop) | QGTSVTVSSTTTPAPRPPTPAPTIASQPLSLRPEACRPAAGGA | | |
| | VHTRGLDFACDIYIWAPLAGTCGVLLLSLVITLYCKRGRKKL | | |
| | LYIFKQPFMRPVQTTQEEDGCSCRFPEEEEGGCELRVKFSRSA | | |
| | DAPAYKQGQNQLYNELNLGRREEYDVLDKRRGRDPEMGGK | | |
| | PRRKNPQEGLYNELQKDKMAEAYSEIGMKGERRRGKGHDG | | |
| | LYQGLSTATKDTYDALHMQALPPR* | | |
| OT-001791 (CD8α | MALPVTALLLPLALLLHAARPRSSLALSLTADQMVSALLDAE | 404 | 405 |
| leader sequence; | PPILYSEYDPTRPFSEASMMGLLTNLADRELVHMINWAKRVP | | |
| Linker (RS); ER | GFVDLALHDQVHLLECAWMEILMIGLVWRSMEHPGKLLFAP | | |
| (aa 305-549 of | NLLLDRNQGKCVEGGVEIFDMLLATSSRFRMMNLQGEEFVC | | |
| WT, T371A, | LKSIILLNSGVYTFLSSTLKSLEEKDHIHRVLDKITDTLIHLMA | | |
| L384M, M421G, | KAGLTLQQQHQRLAQLLLILSHIRHMSSKRMEHLYSMKCKN | | |
| N519S, G521R, | VVPLSDLLLEMLDAHRLGTGAEDPRPSRKRRSLGDVGEIVM | | |
| Y537S); Furin | TQSPATLSLSPGERATLSCRASQDISKYLNWYQQKPGQAPRL | | |
| cleavage site; | LIYHTSRLHSGIPARFSGSGSGTDYTLTISSLQPEDFAVYFCQQ | | |
| CD19 scFv; CD8α | GNTLPYTFGQGTKLEIKGGGGSGGGGSGGGGSQVQLQESGP | | |
| Hinge-TM; 4-1BB | GLVKPSETLSLTCTVSGVSLPDYGVSWIRQPPGKGLEWIGVI | | |
| signaling domain; | WGSETTYYSSSLKSRVTISKDNSKNQVSLKLSSVTAADTAVY | | |
| CD3 zeta signaling | YCAKHYYYGGSYAMDYWGQGTLVTVSSTTTPAPRPPTPAPT | | |
| domain; stop) | IASQPLSLRPEACRPAAGGAVHTRGLDFACDIYIWAPLAGTC | | |
| | GVLLLSLVITLYCKRGRKKLLYIFKQPFMRPVQTTQEEDGCS | | |
| | CRFPEEEEGGCELRVKFSRSADAPAYKQGQNQLYNELNLGR | | |
| | REEYDVLDKRRGRDPEMGGKPRRKNPQEGLYNELQKDKMA | | |
| | EAYSEIGMKGERRRGKGHDGLYQGLSTATKDTYDALHMQA | | |
| | LPPR* | | |
| OT-001792 (CD8α | MALPVTALLLPLALLLHAARPRSSLALSLTADQMVSALLDAE | 406 | 407 |
| leader sequence; | PPILYSEYDPTRPFSEASMMGLLTNLADRELVHMINWAKRVP | | |
| Linker (RS); ER | GFVDLTLHDQVHLLECAWMEILMIGLVWRSMEHPGKLLFAP | | |
| (aa 305-549 of | NLLLDRNQGKCVEGGVEIFDMLLATSSRFRMMNLQGEEFVC | | |
| WT, L384M, | LKSIILLNSGVYTFLSSTLKSLEEKDHIHRVLDKITDTLIHLMA | | |
| M421G, G521R, | KAGLTLQQQHQRLAQLLLILSHIRHMSNKRMEHLYSMKCKN | | |
| Y537S); Furin | VVPLSDLLLEMLDAHRLGTGAEDPRPSRKRRSLGDVGEIVM | | |
| cleavage site; | TQSPATLSLSPGERATLSCRASQDISKYLNWYQQKPGQAPRL | | |
| CD19 scFv; CD8α | LIYHTSRLHSGIPARFSGSGSGTDYTLTISSLQPEDFAVYFCQQ | | |
| Hinge-TM; 4-1BB | GNTLPYTFGQGTKLEIKGGGGSGGGGSGGGGSQVQLQESGP | | |
| signaling domain; | GLVKPSETLSLTCTVSGVSLPDYGVSWIRQPPGKGLEWIGVI | | |
| CD3 zeta signaling | WGSETTYYSSSLKSRVTISKDNSKNQVSLKLSSVTAADTAVY | | |
| domain; stop) | YCAKHYYYGGSYAMDYWGQGTLVTVSSTTTPAPRPPTPAPT | | |
| | IASQPLSLRPEACRPAAGGAVHTRGLDFACDIYIWAPLAGTC | | |
| | GVLLLSLVITLYCKRGRKKLLYIFKQPFMRPVQTTQEEDGCS | | |
| | CRFPEEEEGGCELRVKFSRSADAPAYKQGQNQLYNELNLGR | | |
| | REEYDVLDKRRGRDPEMGGKPRRKNPQEGLYNELQKDKMA | | |
| | EAYSEIGMKGERRRGKGHDGLYQGLSTATKDTYDALHMQA | | |
| | LPPR* | | |
| OT-002157 (Met; | MALPVTALLLPLALLLHAARPGSSLALSLTADQMVSALLDA | 410 | 411 |
| CD8α leader; | EPPILYSEYDPTRPFSEASMMGLLTNLADRELVHMINWAKRV | | |
| Linker (GS); ER | PGFVDLTLHDQVHLLECAWMEILMIGLVWRSMEHPGKLLFA | | |
| (aa 305-549 of | PNLLLDRNQGKCVEGGVEIFDMLLATSSRFRMMNLQGEEFV | | |
| WT, L384M, | CLKSIILLNSGVYTFLSSTLKSLEEKDHIHRVLDKITDTLIHLM | | |
| M421G, N519S, | AKAGLTLQQQHQRLAQLLLILSHIRHMSSKRMEHLYSMKCK | | |
| G521R, Y537S); | NVVPLSDLLLEMLDAHRLGSESRRVRRNKRSKDIQMTQTTSS | | |
| Linker (GS); | LSASLGDRVTISCRASQDISKYLNWYQQKPDGTVKLLIYHTS | | |
| Modified Furin; | RLHSGVPSRFSGSGSGTDYSLTISNLEQEDIATYFCQQGNTLP | | |
| CD19 scFv; CD8a | YTFGGGTKLEITGGGGSGGGGSGGGGSEVKLQESGPGLVAPS | | |
| Hinge and | QSLSVTCTVSGVSLPDYGVSWIRQPPRKGLEWLGVIWGSETT | | |
| Transmembrane | YYNSALKSRLTIIKDNSKSQVFLKMNSLQTDDTAIYYCAKHY | | |
| Domain; CD28 co- | YYGGSYAMDYWGQGTSVTVSSTTTPAPRPPTPAPTIASQPLS | | |
| stimulatory | LRPEACRPAAGGAVHTRGLDFACDIYIWAPLAGTCGVLLLSL | | |
| domain/4-1BB | VITLYCKRGRKKLLYIFKQPFMRPVQTTQEEDGCSCRFPEEEE | | |
| intracellular | GGCELRVKFSRSADAPAYKQGQNQLYNELNLGRREEYDVL | | |
| domain; CD3 zeta | DKRRGRDPEMGGKPRRKNPQEGLYNELQKDKMAEAYSEIG | | |
| intracellular | MKGERRRGKGHDGLYQGLSTATKDTYDALHMQALPPR* | | |
| domain; stop) | | | |
| OT-002158 (Met; | MALPVTALLLPLALLLHAARPGSSLALSLTADQMVSALLDA | 412 | 413 |
| CD8α leader; | EPPILYSEYDPTRPFSEASMMGLLTNLADRELVHMINWAKRV | | |
| Linker (GS); ER | PGFVDLTLHDQVHLLECAWMEILMIGLVWRSMEHPGKLLFA | | |
| (aa 305-549 of | PNLLLDRNQGKCVEGGVEIFDMLLATSSRFRMMNLQGEEFV | | |
| WT, L384M, | CLKSIILLNSGVYTFLSSTLKSLEEKDHIHRVLDKITDTLIHLM | | |
| M421G, Q502R, | AKAGLTLQQQHRRLAQLLLILSHIRHMSNKRMEHLYSMKCK | | |
| G521R, Y537S); | NVVPLSDLLLEMLDAHRLGSESRRVRRNKRSKDIQMTQTTSS | | |
| Linker (GS); | LSASLGDRVTISCRASQDISKYLNWYQQKPDGTVKLLIYHTS | | |
| Modified Furin; | RLHSGVPSRFSGSGSGTDYSLTISNLEQEDIATYFCQQGNTLP | | |
| CD19 scFv; CD8a | YTFGGGTKLEITGGGGSGGGGSGGGGSEVKLQESGPGLVAPS | | |
| Hinge and | QSLSVTCTVSGVSLPDYGVSWIRQPPRKGLEWLGVIWGSETT | | |
| Transmembrane | YYNSALKSRLTIIKDNSKSQVFLKMNSLQTDDTAIYYCAKHY | | |
| Domain; CD28 co- | YYGGSYAMDYWGQGTSVTVSSTTTPAPRPPTPAPTIASQPLS | | |
| stimulatory | LRPEACRPAAGGAVHTRGLDFACDIYIWAPLAGTCGVLLLSL | | |
| domain/4-1BB | VITLYCKRGRKKLLYIFKQPFMRPVQTTQEEDGCSCRFPEEEE | | |
| intracellular | GGCELRVKFSRSADAPAYKQGQNQLYNELNLGRREEYDVL | | |
| domain; CD3 zeta | DKRRGRDPEMGGKPRRKNPQEGLYNELQKDKMAEAYSEIG | | |
| intracellular | MKGERRRGKGHDGLYQGLSTATKDTYDALHMQALPPR* | | |
| domain; stop) | | | |
| OT-002159 (Met; | MALPVTALLLPLALLLHAARPGSSLALSLTADQMVSALLDA | 414 | 415 |
| CD8α leader; | EPPILYSEYDPTGPFSEASMMGLLTNLADRELVHMINWAKRV | | |
| Linker (GS); | ERPGFVDLTLHDQVHLLECAWMEILMIGLVWRSMEHPGKLLFA | | |
| (aa 305-549 of | PNLLLDRNQGKCVEGGVEIFDMLLATSSRFRMMNLQGEEFV | | |
| WT, R335G, | CLKSIILLNSGVYTFLSSTLKSLEEKDHIHRVLDKITDTLIHLM | | |
| L384M, M421G, | AKAGLTLQQQHQRLAQLLLILSHIRHMSNKRMGHLYSMKCK | | |
| G521R, E523G, | NVVPLSDLLLEMLDTHRLGSESRRVRRNKRSKDIQMTQTTSS | | |
| Y537S, A546T); | LSASLGDRVTISCRASQDISKYLNWYQQKPDGTVKLLIYHTS | | |
| Linker (GS); | RLHSGVPSRFSGSGSGTDYSLTISNLEQEDIATYFCQQGNTLP | | |
| Modified Furin; | YTFGGGTKLEITGGGGSGGGGSGGGGSEVKLQESGPGLVAPS | | |
| CD19 scFv; CD8a | QSLSVTCTVSGVSLPDYGVSWIRQPPRKGLEWLGVIWGSETT | | |
| Hinge and | YYNSALKSRLTIIKDNSKSQVFLKMNSLQTDDTAIYYCAKHY | | |
| Transmembrane | YYGGSYAMDYWGQGTSVTVSSTTTPAPRPPTPAPTIASQPLS | | |
| Domain; CD28 co- | LRPEACRPAAGGAVHTRGLDFACDIYIWAPLAGTCGVLLLSL | | |
| stimulatory | VITLYCKRGRKKLLYIFKQPFMRPVQTTQEEDGCSCRFPEEEE | | |
| domain/4-1BB | GGCELRVKFSRSADAPAYKQGQNQLYNELNLGRREEYDVL | | |
| intracellular | DKRRGRDPEMGGKPRRKNPQEGLYNELQKDKMAEAYSEIG | | |
| domain; CD3 zeta | MKGERRRGKGHDGLYQGLSTATKDTYDALHMQALPPR* | | |
| intracellular | | | |
| domain; stop) | | | |
| OT-002160 (Met; | MALPVTALLLPLALLLHAARPGSSLALSLTADQMVSALLDA | 416 | 417 |
| CD8α leader; | EPPILYSEYDPTRPFSEASMMGLLTNLADRELVHMINWAKRV | | |
| Linker (GS); ER | PGFVDLTLHDQVHLLECAWMEILMIGLVWRSMEHPGKLLFA | | |
| (aa 305-549 of | PNLLLDRNQGKCVEGGVEIFDMLLAISSRFRMMNLQGEEFV | | |
| WT, L384M; | CLKSIILLNSGVYTFLSSTLKSLEEKDHIHRVLDKITDTLIHLM | | |
| M421G; T431I; | AKAGLTLQQQHQRLAQLLLILSHIRHMSNKRMEHLYSMKCK | | |
| G521R, Y537S); | NVVPLSDLLLEMLDAHRLGSESRRVRRNKRSKDIQMTQTTSS | | |
| Linker (GS); | LSASLGDRVTISCRASQDISKYLNWYQQKPDGTVKLLIYHTS | | |
| Modified Furin; | RLHSGVPSRFSGSGSGTDYSLTISNLEQEDIATYFCQQGNTLP | | |
| CD19 scFv; CD8a | YTFGGGTKLEITGGGGSGGGGSGGGGSEVKLQESGPGLVAPS | | |
| Hinge and | QSLSVTCTVSGVSLPDYGVSWIRQPPRKGLEWLGVIWGSETT | | |
| Transmembrane | YYNSALKSRLTIIKDNSKSQVFLKMNSLQTDDTAIYYCAKHY | | |
| Domain; CD28 co- | YYGGSYAMDYWGQGTSVTVSSTTTPAPRPPTPAPTIASQPLS | | |
| stimulatory | LRPEACRPAAGGAVHTRGLDFACDIYIWAPLAGTCGVLLLSL | | |
| domain/4-1BB | VITLYCKRGRKKLLYIFKQPFMRPVQTTQEEDGCSCRFPEEEE | | |
| intracellular | GGCELRVKFSRSADAPAYKQGQNQLYNELNLGRREEYDVL | | |
| domain; CD3 zeta | DKRRGRDPEMGGKPRRKNPQEGLYNELQKDKMAEAYSEIG | | |
| intracellular | MKGERRRGKGHDGLYQGLSTATKDTYDALHMQALPPR* | | |
| domain; stop) | | | |
| OT-002161 (Met; | MALPVTALLLPLALLLHAARPGSSLALSLTADQMVSALLDA | 418 | 419 |
| CD8α leader; | EPPILYSEYDPTRPFSEASMMGLLTNLADRELVHMINWAKRV | | |
| Linker (GS); ER | PGFVDLTLHDQVHLLECAWMEILMIGLVWRSMEHPGKLLFA | | |
| (aa 305-549 of | PNLLLDRDQGKCVEGGVEIFDMLLATSSRFRMMNLQGEEFV | | |
| WT, L384M, | CLKSIILLNSGVYTFLSSTLKSLEEKDHIHRVLDKITDTLIHLM | | |
| N413D, M421G, | AKAGLTLQQQHQRLAQLLLILSHIRHMSNKRMEHLYSMKCK | | |
| G521R, Y537S); | NVVPLSDLLLEMLDAHRLGSESRRVRRNKRSKDIQMTQTTSS | | |
| Linker (GS); | LSASLGDRVTISCRASQDISKYLNWYQQKPDGTVKLLIYHTS | | |
| Modified Furin; | RLHSGVPSRFSGSGSGTDYSLTISNLEQEDIATYFCQQGNTLP | | |
| CD19 scFv; CD8a | YTFGGGTKLEITGGGGSGGGGSGGGGSEVKLQESGPGLVAPS | | |
| Hinge and | QSLSVTCTVSGVSLPDYGVSWIRQPPRKGLEWLGVIWGSETT | | |
| Transmembrane | YYNSALKSRLTIIKDNSKSQVFLKMNSLQTDDTAIYYCAKHY | | |
| Domain; CD28 co- | YYGGSYAMDYWGQGTSVTVSSTTTPAPRPPTPAPTIASQPLS | | |
| stimulatory | LRPEACRPAAGGAVHTRGLDFACDIYIWAPLAGTCGVLLLSL | | |
| domain/4-1BB | VITLYCKRGRKKLLYIFKQPFMRPVQTTQEEDGCSCRFPEEEE | | |
| intracellular | GGCELRVKFSRSADAPAYKQGQNQLYNELNLGRREEYDVL | | |
| domain; CD3 zeta | DKRRGRDPEMGGKPRRKNPQEGLYNELQKDKMAEAYSEIG | | |
| intracellular | MKGERRRGKGHDGLYQGLSTATKDTYDALHMQALPPR* | | |
| domain; stop) | | | |
| OT-002162 (Met; | MALPVTALLLPLALLLHAARPGSSLALSLTADQMVSALLDA | 420 | 421 |
| CD8α leader; | EPPILYSEYDPTRPFSEASMMGLLTNLADRELVHMINWAKRV | | |
| Linker (GS); ER | PGFVDLALHDQVHLLECAWMEILMIGLVWRSMEHPGKLLFA | | |
| (aa 305-549 of | PNLLLDRNQGKCVEGGVEIFDMLLATSSRFRMMNLQGEEFV | | |
| WT, T371A, | CLKSIILLNSGVYTFLSSTLKSLEEKDHIHRVLDKITDTLIHLM | | |
| L384M, M421G, | AKAGLTLQQQHQRLAQLLLILSHIRHMSSKRMEHLYSMKCK | | |
| N519S, G521R, | NVVPLSDLLLEMLDAHRLGSESRRVRRNKRSKDIQMTQTTSS | | |
| Y537S); Linker | LSASLGDRVTISCRASQDISKYLNWYQQKPDGTVKLLIYHTS | | |
| (GS); Modified | RLHSGVPSRFSGSGSGTDYSLTISNLEQEDIATYFCQQGNTLP | | |
| Furin; CD19 scFv; | YTFGGGTKLEITGGGGSGGGGSGGGGSEVKLQESGPGLVAPS | | |
| CD8a Hinge and | QSLSVTCTVSGVSLPDYGVSWIRQPPRKGLEWLGVIWGSETT | | |
| Transmembrane | YYNSALKSRLTIIKDNSKSQVFLKMNSLQTDDTAIYYCAKHY | | |
| Domain; CD28 co- | YYGGSYAMDYWGQGTSVTVSSTTTPAPRPPTPAPTIASQPLS | | |
| stimulatory | LRPEACRPAAGGAVHTRGLDFACDIYIWAPLAGTCGVLLLSL | | |
| domain/4-1BB | VITLYCKRGRKKLLYIFKQPFMRPVQTTQEEDGCSCRFPEEEE | | |
| intracellular | GGCELRVKFSRSADAPAYKQGQNQLYNELNLGRREEYDVL | | |
| domain; CD3 zeta | DKRRGRDPEMGGKPRRKNPQEGLYNELQKDKMAEAYSEIG | | |
| intracellular | MKGERRRGKGHDGLYQGLSTATKDTYDALHMQALPPR* | | |
| domain; stop) | | | |
| OT-002163 (Met; | MALPVTALLLPLALLLHAARPGSNLALSLTADQMVSALLDA | 422 | 423 |
| CD8α leader; | EPPILYSEYDPTRPFSEASMMGLLTNLADRELVHMINWAKRV | | |
| Linker (GS); ER | PGFVDLTLHDQVHLLECAWMEILMIGLVWRSMEHPGKLLFA | | |
| (aa 305-549 of | PNLLLDRNQGKCVEGGVEIFDMLLATSSRFRMMNLQVEEFV | | |
| WT, S305N, | CLKSIILLNSGVYTFLSSTLKSLEEKDHIHRVLDKITDTLIHLM | | |
| L384M, M421G, | AKAGLTLQQQHQRLAQLLLILSHIRHMSNKRMEHLYSMKCK | | |
| G442V, G521R, | NVVPLSDLLLEMLDAHRLGSESRRVRRNKRSKDIQMTQTTSS | | |
| Y537S); Linker | LSASLGDRVTISCRASQDISKYLNWYQQKPDGTVKLLIYHTS | | |
| (GS); Modified | RLHSGVPSRFSGSGSGTDYSLTISNLEQEDIATYFCQQGNTLP | | |
| Furin; CD19 scFv; | YTFGGGTKLEITGGGGSGGGGSGGGGSEVKLQESGPGLVAPS | | |
| CD8a Hinge and | QSLSVTCTVSGVSLPDYGVSWIRQPPRKGLEWLGVIWGSETT | | |
| Transmembrane | YYNSALKSRLTIIKDNSKSQVFLKMNSLQTDDTAIYYCAKHY | | |
| Domain; CD28 co- | YYGGSYAMDYWGQGTSVTVSSTTTPAPRPPTPAPTIASQPLS | | |
| stimulatory | LRPEACRPAAGGAVHTRGLDFACDIYIWAPLAGTCGVLLLSL | | |
| domain/4-1BB | VITLYCKRGRKKLLYIFKQPFMRPVQTTQEEDGCSCRFPEEEE | | |
| intracellular | GGCELRVKFSRSADAPAYKQGQNQLYNELNLGRREEYDVL | | |
| domain; CD3 zeta | DKRRGRDPEMGGKPRRKNPQEGLYNELQKDKMAEAYSEIG | | |
| intracellular | MKGERRRGKGHDGLYQGLSTATKDTYDALHMQALPPR* | | |
| domain; stop) | | | |
| OT-002164 (Met; | MALPVTALLLPLALLLHAARPGSSLALSLTADQMVSALLDA | 424 | 425 |
| CD8α leader; | EPPILYSEYDPTGPFSEASMMGLLTNLADRELVHMINWAKRV | | |
| Linker (GS); ER | PGFVDLTLHDQVHLLECAWMEILMIGLVWRSMEHPGKLLFA | | |
| (aa 305-549 of | PNLLLDRNQGKCVEGGVEIFDMLLATSSRFRMMNLQGEEFV | | |
| WT, R335G, | CLKSIILLNSGVYTFLSSTLKSLEEKDHIHRVLDKITDTLIHLM | | |
| L384M, M421G, | AKAGLTLQQQHQRLAQLLLILSHIRHMSSKRMEHLYSMKCK | | |
| N519S, G521R, | NVVPLSDLLLEMLDAHRLGSESRRVRRNKRSKDIQMTQTTSS | | |
| Y537S); Linker | LSASLGDRVTISCRASQDISKYLNWYQQKPDGTVKLLIYHTS | | |
| (GS); Modified | RLHSGVPSRFSGSGSGTDYSLTISNLEQEDIATYFCQQGNTLP | | |
| Furin; CD19 scFv; | YTFGGGTKLEITGGGGSGGGGSGGGGSEVKLQESGPGLVAPS | | |
| CD8a Hinge and | QSLSVTCTVSGVSLPDYGVSWIRQPPRKGLEWLGVIWGSETT | | |
| Transmembrane | YYNSALKSRLTIIKDNSKSQVFLKMNSLQTDDTAIYYCAKHY | | |
| Domain; CD28 co- | YYGGSYAMDYWGQGTSVTVSSTTTPAPRPPTPAPTIASQPLS | | |
| stimulatory | LRPEACRPAAGGAVHTRGLDFACDIYIWAPLAGTCGVLLLSL | | |
| domain/4-1BB | VITLYCKRGRKKLLYIFKQPFMRPVQTTQEEDGCSCRFPEEEE | | |
| intracellular | GGCELRVKFSRSADAPAYKQGQNQLYNELNLGRREEYDVL | | |
| domain; CD3 zeta | DKRRGRDPEMGGKPRRKNPQEGLYNELQKDKMAEAYSEIG | | |
| intracellular | MKGERRRGKGHDGLYQGLSTATKDTYDALHMQALPPR* | | |
| domain; stop) | | | |

In some embodiments, payloads of the disclosure may be cytokines, and fragments, variants, analogs and derivatives thereof, including but not limited to interleukins, tumor necrosis factors (TNFs), interferons (IFNs), TGF beta and chemokines.

In some embodiments, a cytokine may be an interleukin (IL) selected from IL′, IL1alpha (also called hematopoietin-1), IL1beta (catabolin), IL1 delta, IL1epsilon, IL1eta, IL1 zeta, interleukin-1 family member 1 to 11 (IL1F1 to IL1F11), interleukin-1 homolog 1 to 4 (IL1H1 to IL1H4), IL1 related protein 1 to 3 (IL1RP1 to IL1RP3), IL2, IL3, IL4, IL5, IL6, IL7, IL8, IL9, IL10, IL10C, IL10D, IL11, IL11a, IL11b, IL12, IL13, IL14, IL15, IL16, IL17, IL17A, Il17B, IL17C, IL17E, IL17F, 1118, IL19, IL20, IL20 like (IL20L), 1121, IL22, IL23, IL23A, IL23-p19, IL23-p40, IL24, 1125, IL26, IL27, IL28A, IL28B, IL29, IL30, IL31, IL32, IL33, IL34, IL35, IL36 alpha, IL36 beta, IL36 gamma, IL36RN, IL37, IL37a, IL37b, IL37c, IL37d, IL37e and IL38.

In certain embodiments, a cytokine may be a type I interferons (IFN) including IFN-alpha subtypes (IFN-α1, IFN-α1b, IFN-α1c), IFN-beta, IFN-delta subtypes (IFN-delta 1, IFN-delta 2, IFN-delta 8), IFN-gamma, IFN-kappa, and IFN-epsilon, IFN-lambda, IFN-omega, IFN-tau and IFN-zeta. In certain embodiments, a cytokine may be a member of tumor necrosis factor (TNF) superfamily, including TNF-alpha, TNF-beta (also known as lymphotoxin-alpha (LT-α)), lymphotoxin-beta (LT-β), CD40L(CD154), CD27L (CD70), CD30L(CD153), FASL(CD178), 4-1BBL (CD137L), OX40L, TRAIL (TNF-related apoptosis inducing ligand), APRIL (a proliferation-inducing ligand), TWEAK, TRANCE, TALL-1, GITRL, LIGHT and TNFSF1 to TNFSF20 (TNF ligand superfamily member 1 to 20).

It is understood in the art that certain gene and/or protein nomenclature for the same gene or protein may be inclusive or exclusive of punctuation such as a dash “-” or symbolic such as Greek letters. Whether these are included or excluded herein, the meaning is not meant to be changed as would be understood by one of skill in the art. For example, IL2, IL2 and IL 2 refer to the same interleukin. Likewise, TN alpha, TNFα, TNF-alpha, TNF-α, TNF alpha and TNF α all refer to the same protein.

In certain embodiments, a cytokine may be a chemokine selected from SCYA1-28 (CCL1-28), SCYB1-16 (CXCL1-16), SCYC1-2 (XCL1-2), SCYD-1, SCYE-1, XCL1, XCL2, CCL1, CCL2, CCL3, CCL4, CCL5, CCL6, CCL7, CCL8, CCL9, CCL10, CCL11, CCL12, CCL13, CCL14, CCL15, CCL16, CCL17, CCL18, CCL19, CCL20, CCL21, CCL22, CCL23, CCL24, CCL25, CCL26, CCL27, CCL28, CXCL1, CXCL2, CXCL3, CXCL4, CXCL5, CXCL6, CXCL7, CXCL8, CXCL9, CXCL10, CXCL11, CXCL12, CXCL13, CXCL14, CXCL15, CXCL16, CXCL17 and CX3CL1; or a chemokine receptor selected from XCR1, CCR1, CCR2, CCR3, CCR4, CCR5, CCR6, CCR7, CCR8, CCR9, CCR10, CXCR1, CXCR2, CXCR3, CXCR4, CXCR5 and CX3CR1.

In one aspect, the payload of the disclosure may be IL12 fusion. This regulatable DD-IL12 fusion polypeptide may be directly used as an immunotherapeutic agent or be transduced into an immune effector cell (T cells and TIL cells) to generate modified T cells with greater in vivo expansion and survival capabilities for adoptive cell transfer. In some embodiments, the IL12 may be referred to as “Flexi IL12,” wherein both p35 and p40 subunits are encoded by a single cDNA that produces a single chain polypeptide. In some aspects, the human ER-IL12 comprises the amino acid sequences described in Table 7. Nucleic acid sequences encoding the amino acid sequences are also described in Table 7. In Table 7, the amino acid sequences may comprise a stop codon at the end which is denoted in the table with a “*”. The components of the ER-IL12 constructs are described in Table 4.

TABLE 7
IL12 Constructs

| | | | AA | NA |
|---|---|---|---|---|
| | | | SEQ | SEQ |
| Construct | | | ID | ID |
| ID | Description | AA sequence | NO. | NO. |
| OT-001563 | IL12B (p40) | MCHQQLVISWFSLVFLASPLVAIWELKKDVYVVELD | 158 | 159 |
| | signal | WYPDAPGEMVVLTCDTPEEDGITWTLDQSSEVLGSGK | | |
| | sequence; | TLTIQVKEFGDAGQYTCHKGGEVLSHSLLLLHKKEDG | | |
| | IL12B (p40) | IWSTDILKDQKEPKNKTFLRCEAKNYSGRFTCWWLTT | | |
| | (23-328 of | ISTDLTFSVKSSRGSSDPQGVTCGAATLSAERVRGDNK | | |
| | WT); Linker | EYEYSVECQEDSACPAAEESLPIEVMVDAVHKLKYEN | | |
| | ((G4S)3; IL12A | YTSSFFIRDIIKPDPPKNLQLKPLKNSRQVEVSWEYPDT | | |
| | (p35) (57-253 | WSTPHSYFSLTFCVQVQGKSKREKKDRVFTDKTSATV | | |
| | of WT); Linker | ICRKNASISVRAQDRYYSSSWSEWASVPCSGGGGSGG | | |
| | (GS); stop | GGSGGGGSRNLPVATPDPGMFPCLHHSQNLLRAVSN | | |
| | | MLQKARQTLEFYPCTSEEIDHEDITKDKTSTVEACLPL | | |
| | | ELTKNESCLNSRETSFITNGSCLASRKTSFMMALCLSSI | | |
| | | YEDLKMYQVEFKTMNAKLLMDPKRQIFLDQNMLAVI | | |
| | | DELMQALNFNSETVPQKSSLEEPDFYKTKIKLCILLHA | | |
| | | FRIRAVTIDRVMSYLNASGS* | | |
| OT-001569 | IL12B (p40) | MCHQQLVISWFSLVFLASPLVAGSSLALSLTADQMVS | 160 | 161 |
| | signal | ALLDAEPPILYSEYDPTRPFSEASMMGLLTNLADRELV | | |
| | sequence; | HMINWAKRVPGFVDLALHDQVHLLECAWMEILMIGL | | |
| | Linker (GS); | VWRSMEHPGKLLFAPNLLLDRNQGKCVEGGVEIFDM | | |
| | ER (aa 305-549 | LLATSSRFRMMNLQGEEFVCLKSIILLNSGVYTFLSSTL | | |
| | of WT, T371A, | KSLEEKDHIHRVLDKITDTLIHLMAKAGLTLQQQHQR | | |
| | L384M, | LAQLLLILSHIRHMSSKRMEHLYSMKCKNVVPLSDLL | | |
| | M421G, | LEMLDAHRLGSIWELKKDVYVVELDWYPDAPGEMV | | |
| | N519S, G521R, | VLTCDTPEEDGITWTLDQSSEVLGSGKTLTIQVKEFGD | | |
| | Y537S); Linker | AGQYTCHKGGEVLSHSLLLLHKKEDGIWSTDILKDQK | | |
| | (GS); IL12B | EPKNKTFLRCEAKNYSGRFTCWWLTTISTDLTFSVKSS | | |
| | (p40) (23-328 | RGSSDPQGVTCGAATLSAERVRGDNKEYEYSVECQED | | |
| | of WT); Linker | SACPAAEESLPIEVMVDAVHKLKYENYTSSHIRDIIKP | | |
| | ((G4S)3; IL12A | DPPKNLQLKPLKNSRQVEVSWEYPDTWSTPHSYFSLT | | |
| | (p35) (57-253 | FCVQVQGKSKREKKDRVFTDKTSATVICRKNASISVR | | |
| | of WT); stop | AQDRYYSSSWSEWASVPCSGGGGSGGGGSGGGGSRN | | |
| | | LPVATPDPGMFPCLHHSQNLLRAVSNMLQKARQTLEF | | |
| | | YPCTSEEIDHEDITKDKTSTVEACLPLELTKNESCLNSR | | |
| | | ETSFITNGSCLASRKTSFMMALCLSSIYEDLKMYQVEF | | |
| | | KTMNAKLLMDPKRQIFLDQNMLAVIDELMQALNFNS | | |
| | | ETVPQKSSLEEPDFYKTKIKLCILLHAFRIRAVTIDRVM | | |
| | | SYLNAS* | | |
| OT-001707 | IL12B (p40) | MCHQQLVISWFSLVFLASPLVAIWELKKDVYVVELD | 162 | 163 |
| | signal | WYPDAPGEMVVLTCDTPEEDGITWTLDQSSEVLGSGK | | |
| | sequence; | TLTIQVKEFGDAGQYTCHKGGEVLSHSLLLLHKKEDG | | |
| | IL12B (p40) | IWSTDILKDQKEPKNKTFLRCEAKNYSGRFTCWWLTT | | |
| | (23-328 of | ISTDLTFSVKSSRGSSDPQGVTCGAATLSAERVRGDNK | | |
| | WT); Linker | EYEYSVECQEDSACPAAEESLPIEVMVDAVHKLKYEN | | |
| | ((G4S)3; IL12A | YTSSFFIRDIIKPDPPKNLQLKPLKNSRQVEVSWEYPDT | | |
| | (p35) (57-253 | WSTPHSYFSLTFCVQVQGKSKREKKDRVFTDKTSATV | | |
| | of WT); Linker | ICRKNASISVRAQDRYYSSSWSEWASVPCSGGGGSGG | | |
| | (GS); ER (aa | GGSGGGGSRNLPVATPDPGMFPCLHHSQNLLRAVSN | | |
| | 305-549 of WT, | MLQKARQTLEFYPCTSEEIDHEDITKDKTSTVEACLPL | | |
| | L384M, | ELTKNESCLNSRETSFITNGSCLASRKTSFMMALCLSSI | | |
| | N413D, | YEDLKMYQVEFKTMNAKLLMDPKRQIFLDQNMLAVI | | |
| | M421G, | DELMQALNFNSETVPQKSSLEEPDFYKTKIKLCILLHA | | |
| | G521R, | FRIRAVTIDRVMSYLNASGSSLALSLTADQMVSALLD | | |
| | Y537S); stop | AEPPILYSEYDPTRPFSEASMMGLLTNLADRELVHMIN | | |
| | | WAKRVPGFVDLTLHDQVHLLECAWMEILMIGLVWRS | | |
| | | MEHPGKLLFAPNLLLDRDQGKCVEGGVEIFDMLLATS | | |
| | | SRFRMMNLQGEEFVCLKSIILLNSGVYTFLSSTLKSLEE | | |
| | | KDHIHRVLDKITDTLIHLMAKAGLTLQQQHQRLAQLL | | |
| | | LILSHIRHMSNKRMEHLYSMKCKNVVPLSDLLLEMLD | | |
| | | AHRL* | | |
| OT-001706 | IL12B (p40) | MCHQQLVISWFSLVFLASPLVAIWELKKDVYVVELD | 164 | 165 |
| | signal | WYPDAPGEMVVLTCDTPEEDGITWTLDQSSEVLGSGK | | |
| | sequence; | TLTIQVKEFGDAGQYTCHKGGEVLSHSLLLLHKKEDG | | |
| | IL12B (p40) | IWSTDILKDQKEPKNKTFLRCEAKNYSGRFTCWWLTT | | |
| | (23-328 of | ISTDLTFSVKSSRGSSDPQGVTCGAATLSAERVRGDNK | | |
| | WT); Linker | EYEYSVECQEDSACPAAEESLPIEVMVDAVHKLKYEN | | |
| | ((G4S)3; IL12A | YTSSFFIRDIIKPDPPKNLQLKPLKNSRQVEVSWEYPDT | | |
| | (p35) (57-253 | WSTPHSYFSLTFCVQVQGKSKREKKDRVFTDKTSATV | | |
| | of WT); Linker | ICRKNASISVRAQDRYYSSSWSEWASVPCSGGGGSGG | | |
| | (GS); ER (aa | GGSGGGGSRNLPVATPDPGMFPCLHHSQNLLRAVSN | | |
| | 305-549 of WT, | MLQKARQTLEFYPCTSEEIDHEDITKDKTSTVEACLPL | | |
| | L384M, | ELTKNESCLNSRETSFITNGSCLASRKTSFMMALCLSSI | | |
| | N413F, | YEDLKMYQVEFKTMNAKLLMDPKRQIFLDQNMLAVI | | |
| | M421G, | DELMQALNFNSETVPQKSSLEEPDFYKTKIKLCILLHA | | |
| | G521R, | FRIRAVTIDRVMSYLNASGSSLALSLTADQMVSALLD | | |
| | Y537S; stop | AEPPILYSEYDPTRPFSEASMMGLLTNLADRELVHMIN | | |
| | | WAKRVPGFVDLTLHDQVHLLECAWMEILMIGLVWRS | | |
| | | MEHPGKLLFAPNLLLDRFQGKCVEGGVEIFDMLLATS | | |
| | | SRFRMMNLQGEEFVCLKSIILLNSGVYTFLSSTLKSLEE | | |
| | | KDHIHRVLDKITDTLIHLMAKAGLTLQQQHQRLAQLL | | |
| | | LILSHIRHMSNKRMEHLYSMKCKNVVPLSDLLLEMLD | | |
| | | AHRL* | | |
| OT-001705 | IL12B (p40) | MCHQQLVISWFSLVFLASPLVAIWELKKDVYVVELD | 166 | 167 |
| | signal | WYPDAPGEMVVLTCDTPEEDGITWTLDQSSEVLGSGK | | |
| | sequence; | TLTIQVKEFGDAGQYTCHKGGEVLSHSLLLLHKKEDG | | |
| | IL12B (p40) | IWSTDILKDQKEPKNKTFLRCEAKNYSGRFTCWWLTT | | |
| | (23-328 of | ISTDLTFSVKSSRGSSDPQGVTCGAATLSAERVRGDNK | | |
| | WT); Linker | EYEYSVECQEDSACPAAEESLPIEVMVDAVHKLKYEN | | |
| | ((G4S)3; IL12A | YTSSFFIRDIIKPDPPKNLQLKPLKNSRQVEVSWEYPDT | | |
| | (p35) (57-253 | WSTPHSYFSLTFCVQVQGKSKREKKDRVFTDKTSATV | | |
| | of WT); Linker | ICRKNASISVRAQDRYYSSSWSEWASVPCSGGGGSGG | | |
| | (GS); ER (aa | GGSGGGGSRNLPVATPDPGMFPCLHHSQNLLRAVSN | | |
| | 305-549 of WT, | MLQKARQTLEFYPCTSEEIDHEDITKDKTSTVEACLPL | | |
| | L384M, | ELTKNESCLNSRETSFITNGSCLASRKTSFMMALCLSSI | | |
| | N413L, | YEDLKMYQVEFKTMNAKLLMDPKRQIFLDQNMLAVI | | |
| | M421G, | DELMQALNFNSETVPQKSSLEEPDFYKTKIKLCILLHA | | |
| | G521R, | FRIRAVTIDRVMSYLNASGSSLALSLTADQMVSALLD | | |
| | Y537S); stop | AEPPILYSEYDPTRPFSEASMMGLLTNLADRELVHMIN | | |
| | | WAKRVPGFVDLTLHDQVHLLECAWMEILMIGLVWRS | | |
| | | MEHPGKLLFAPNLLLDRLQGKCVEGGVEIFDMLLATS | | |
| | | SRFRMMNLQGEEFVCLKSIILLNSGVYTFLSSTLKSLEE | | |
| | | KDHIHRVLDKITDTLIHLMAKAGLTLQQQHQRLAQLL | | |
| | | LILSHIRHMSNKRMEHLYSMKCKNVVPLSDLLLEMLD | | |
| | | AHRL* | | |
| OT-001704 | IL12B (p40) | MCHQQLVISWFSLVFLASPLVAIWELKKDVYVVELD | 168 | 169 |
| | signal | WYPDAPGEMVVLTCDTPEEDGITWTLDQSSEVLGSGK | | |
| | sequence; | TLTIQVKEFGDAGQYTCHKGGEVLSHSLLLLHKKEDG | | |
| | IL12B (p40) | IWSTDILKDQKEPKNKTFLRCEAKNYSGRFTCWWLTT | | |
| | (23-328 of | ISTDLTFSVKSSRGSSDPQGVTCGAATLSAERVRGDNK | | |
| | WT); Linker | EYEYSVECQEDSACPAAEESLPIEVMVDAVHKLKYEN | | |
| | ((G4S)3; IL12A | YTSSFFIRDIIKPDPPKNLQLKPLKNSRQVEVSWEYPDT | | |
| | (p35) (57-253 | WSTPHSYFSLTFCVQVQGKSKREKKDRVFTDKTSATV | | |
| | of WT); Linker | ICRKNASISVRAQDRYYSSSWSEWASVPCSGGGGSGG | | |
| | (GS); ER (aa | GGSGGGGSRNLPVATPDPGMFPCLHHSQNLLRAVSN | | |
| | 305-549 of WT, | MLQKARQTLEFYPCTSEEIDHEDITKDKTSTVEACLPL | | |
| | L384M, | ELTKNESCLNSRETSFITNGSCLASRKTSFMMALCLSSI | | |
| | N413Y, | YEDLKMYQVEFKTMNAKLLMDPKRQIFLDQNMLAVI | | |
| | M421G, | DELMQALNFNSETVPQKSSLEEPDFYKTKIKLCILLHA | | |
| | G521R, | FRIRAVTIDRVMSYLNASGSSLALSLTADQMVSALLD | | |
| | Y537S); stop | AEPPILYSEYDPTRPFSEASMMGLLTNLADRELVHMIN | | |
| | | WAKRVPGFVDLTLHDQVHLLECAWMEILMIGLVWRS | | |
| | | MEHPGKLLFAPNLLLDRYQGKCVEGGVEIFDMLLATS | | |
| | | SRFRMMNLQGEEFVCLKSIILLNSGVYTFLSSTLKSLEE | | |
| | | KDHIHRVLDKITDTLIHLMAKAGLTLQQQHQRLAQLL | | |
| | | LILSHIRHMSNKRMEHLYSMKCKNVVPLSDLLLEMLD | | |
| | | AHRL* | | |
| OT-001703 | IL12B (p40) | MCHQQLVISWFSLVFLASPLVAIWELKKDVYVVELD | 170 | 171 |
| | signal | WYPDAPGEMVVLTCDTPEEDGITWTLDQSSEVLGSGK | | |
| | sequence; | TLTIQVKEFGDAGQYTCHKGGEVLSHSLLLLHKKEDG | | |
| | IL12B (p40) | IWSTDILKDQKEPKNKTFLRCEAKNYSGRFTCWWLTT | | |
| | (23-328 of | ISTDLTFSVKSSRGSSDPQGVTCGAATLSAERVRGDNK | | |
| | WT); Linker | EYEYSVECQEDSACPAAEESLPIEVMVDAVHKLKYEN | | |
| | ((G4S)3; IL12A | YTSSFFIRDIIKPDPPKNLQLKPLKNSRQVEVSWEYPDT | | |
| | (p35) (57-253 | WSTPHSYFSLTFCVQVQGKSKREKKDRVFTDKTSATV | | |
| | of WT); Linker | ICRKNASISVRAQDRYYSSSWSEWASVPCSGGGGSGG | | |
| | (GS); ER (aa | GGSGGGGSRNLPVATPDPGMFPCLHHSQNLLRAVSN | | |
| | 305-549 of WT, | MLQKARQTLEFYPCTSEEIDHEDITKDKTSTVEACLPL | | |
| | L384M, | ELTKNESCLNSRETSFITNGSCLASRKTSFMMALCLSSI | | |
| | N413H, | YEDLKMYQVEFKTMNAKLLMDPKRQIFLDQNMLAVI | | |
| | M421G, | DELMQALNFNSETVPQKSSLEEPDFYKTKIKLCILLHA | | |
| | G521R, | FRIRAVTIDRVMSYLNASGSSLALSLTADQMVSALLD | | |
| | Y537S); stop | AEPPILYSEYDPTRPFSEASMMGLLTNLADRELVHMIN | | |
| | | WAKRVPGFVDLTLHDQVHLLECAWMEILMIGLVWRS | | |
| | | MEHPGKLLFAPNLLLDRHQGKCVEGGVEIFDMLLATS | | |
| | | SRFRMMNLQGEEFVCLKSIILLNSGVYTFLSSTLKSLEE | | |
| | | KDHIHRVLDKITDTLIHLMAKAGLTLQQQHQRLAQLL | | |
| | | LILSHIRHMSNKRMEHLYSMKCKNVVPLSDLLLEMLD | | |
| | | AHRL* | | |
| OT-001702 | IL12B (p40) | MCHQQLVISWFSLVFLASPLVAIWELKKDVYVVELD | 172 | 173 |
| | signal | WYPDAPGEMVVLTCDTPEEDGITWTLDQSSEVLGSGK | | |
| | sequence; | TLTIQVKEFGDAGQYTCHKGGEVLSHSLLLLHKKEDG | | |
| | IL12B (p40) | IWSTDILKDQKEPKNKTFLRCEAKNYSGRFTCWWLTT | | |
| | (23-328 of | ISTDLTFSVKSSRGSSDPQGVTCGAATLSAERVRGDNK | | |
| | WT); Linker | EYEYSVECQEDSACPAAEESLPIEVMVDAVHKLKYEN | | |
| | ((G4S)3; IL12A | YTSSFFIRDIIKPDPPKNLQLKPLKNSRQVEVSWEYPDT | | |
| | (p35) (57-253 | WSTPHSYFSLTFCVQVQGKSKREKKDRVFTDKTSATV | | |
| | of WT); Linker | ICRKNASISVRAQDRYYSSSWSEWASVPCSGGGGSGG | | |
| | (GS); ER (aa | GGSGGGGSRNLPVATPDPGMFPCLHHSQNLLRAVSN | | |
| | 305-549 of WT, | MLQKARQTLEFYPCTSEEIDHEDITKDKTSTVEACLPL | | |
| | L384M, | ELTKNESCLNSRETSFITNGSCLASRKTSFMMALCLSSI | | |
| | N413Q, | YEDLKMYQVEFKTMNAKLLMDPKRQIFLDQNMLAVI | | |
| | M421G, | DELMQALNFNSETVPQKSSLEEPDFYKTKIKLCILLHA | | |
| | G521R, | FRIRAVTIDRVMSYLNASGSSLALSLTADQMVSALLD | | |
| | Y537S); stop | AEPPILYSEYDPTRPFSEASMMGLLTNLADRELVHMIN | | |
| | | WAKRVPGFVDLTLHDQVHLLECAWMEILMIGLVWRS | | |
| | | MEHPGKLLFAPNLLLDRQQGKCVEGGVEIFDMLLATS | | |
| | | SRFRMMNLQGEEFVCLKSIILLNSGVYTFLSSTLKSLEE | | |
| | | KDHIHRVLDKITDTLIHLMAKAGLTLQQQHQRLAQLL | | |
| | | LILSHIRHMSNKRMEHLYSMKCKNVVPLSDLLLEMLD | | |
| | | AHRL* | | |
| OT-001701 | IL12B (p40) | MCHQQLVISWFSLVFLASPLVAIWELKKDVYVVELD | 174 | 175 |
| | signal | WYPDAPGEMVVLTCDTPEEDGITWTLDQSSEVLGSGK | | |
| | sequence; | TLTIQVKEFGDAGQYTCHKGGEVLSHSLLLLHKKEDG | | |
| | IL12B (p40) | IWSTDILKDQKEPKNKTFLRCEAKNYSGRFTCWWLTT | | |
| | (23-328 of | ISTDLTFSVKSSRGSSDPQGVTCGAATLSAERVRGDNK | | |
| | WT); Linker | EYEYSVECQEDSACPAAEESLPIEVMVDAVHKLKYEN | | |
| | ((G4S)3; IL12A | YTSSFFIRDIIKPDPPKNLQLKPLKNSRQVEVSWEYPDT | | |
| | (p35) (57-253 | WSTPHSYFSLTFCVQVQGKSKREKKDRVFTDKTSATV | | |
| | of WT); Linker | ICRKNASISVRAQDRYYSSSWSEWASVPCSGGGGSGG | | |
| | (GS); ER (aa | GGSGGGGSRNLPVATPDPGMFPCLHHSQNLLRAVSN | | |
| | 305-549 of WT, | MLQKARQTLEFYPCTSEEIDHEDITKDKTSTVEACLPL | | |
| | L384M, N413I, | ELTKNESCLNSRETSFITNGSCLASRKTSFMMALCLSSI | | |
| | M421G, | YEDLKMYQVEFKTMNAKLLMDPKRQIFLDQNMLAVI | | |
| | G521R, | DELMQALNFNSETVPQKSSLEEPDFYKTKIKLCILLHA | | |
| | Y537S); stop | FRIRAVTIDRVMSYLNASGSSLALSLTADQMVSALLD | | |
| | | AEPPILYSEYDPTRPFSEASMMGLLTNLADRELVHMIN | | |
| | | WAKRVPGFVDLTLHDQVHLLECAWMEILMIGLVWRS | | |
| | | MEHPGKLLFAPNLLLDRIQGKCVEGGVEIFDMLLATSS | | |
| | | RFRMMNLQGEEFVCLKSIILLNSGVYTFLSSTLKSLEE | | |
| | | KDHIHRVLDKITDTLIHLMAKAGLTLQQQHQRLAQLL | | |
| | | LILSHIRHMSNKRMEHLYSMKCKNVVPLSDLLLEMLD | | |
| | | AHRL* | | |
| OT-001700 | IL12B (p40) | MCHQQLVISWFSLVFLASPLVAIWELKKDVYVVELD | 176 | 177 |
| | signal | WYPDAPGEMVVLTCDTPEEDGITWTLDQSSEVLGSGK | | |
| | sequence; | TLTIQVKEFGDAGQYTCHKGGEVLSHSLLLLHKKEDG | | |
| | IL12B (p40) | IWSTDILKDQKEPKNKTFLRCEAKNYSGRFTCWWLTT | | |
| | (23-328 of | ISTDLTFSVKSSRGSSDPQGVTCGAATLSAERVRGDNK | | |
| | WT); Linker | EYEYSVECQEDSACPAAEESLPIEVMVDAVHKLKYEN | | |
| | ((G4S)3; IL12A | YTSSFFIRDIIKPDPPKNLQLKPLKNSRQVEVSWEYPDT | | |
| | (p35) (57-253 | WSTPHSYFSLTFCVQVQGKSKREKKDRVFTDKTSATV | | |
| | of WT); Linker | ICRKNASISVRAQDRYYSSSWSEWASVPCSGGGGSGG | | |
| | (GS); ER (aa | GGSGGGGSRNLPVATPDPGMFPCLHHSQNLLRAVSN | | |
| | 305-549 of WT, | MLQKARQTLEFYPCTSEEIDHEDITKDKTSTVEACLPL | | |
| | L384M, | ELTKNESCLNSRETSFITNGSCLASRKTSFMMALCLSSI | | |
| | N413M, | YEDLKMYQVEFKTMNAKLLMDPKRQIFLDQNMLAVI | | |
| | M421G, | DELMQALNFNSETVPQKSSLEEPDFYKTKIKLCILLHA | | |
| | G521R, | FRIRAVTIDRVMSYLNASGSSLALSLTADQMVSALLD | | |
| | Y537S); stop | AEPPILYSEYDPTRPFSEASMMGLLTNLADRELVHMIN | | |
| | | WAKRVPGFVDLTLHDQVHLLECAWMEILMIGLVWRS | | |
| | | MEHPGKLLFAPNLLLDRMQGKCVEGGVEIFDMLLATS | | |
| | | SRFRMMNLQGEEFVCLKSIILLNSGVYTFLSSTLKSLEE | | |
| | | KDHIHRVLDKITDTLIHLMAKAGLTLQQQHQRLAQLL | | |
| | | LILSHIRHMSNKRMEHLYSMKCKNVVPLSDLLLEMLD | | |
| | | AHRL* | | |
| OT-001699 | IL12B (p40) | MCHQQLVISWFSLVFLASPLVAIWELKKDVYVVELD | 178 | 179 |
| | signal | WYPDAPGEMVVLTCDTPEEDGITWTLDQSSEVLGSGK | | |
| | sequence; | TLTIQVKEFGDAGQYTCHKGGEVLSHSLLLLHKKEDG | | |
| | IL12B (p40) | IWSTDILKDQKEPKNKTFLRCEAKNYSGRFTCWWLTT | | |
| | (23-328 of | ISTDLTFSVKSSRGSSDPQGVTCGAATLSAERVRGDNK | | |
| | WT); Linker | EYEYSVECQEDSACPAAEESLPIEVMVDAVHKLKYEN | | |
| | ((G4S)3; IL12A | YTSSFFIRDIIKPDPPKNLQLKPLKNSRQVEVSWEYPDT | | |
| | (p35) (57-253 | WSTPHSYFSLTFCVQVQGKSKREKKDRVFTDKTSATV | | |
| | of WT); Linker | ICRKNASISVRAQDRYYSSSWSEWASVPCSGGGGSGG | | |
| | (GS); ER (aa | GGSGGGGSRNLPVATPDPGMFPCLHHSQNLLRAVSN | | |
| | 305-549 of WT, | MLQKARQTLEFYPCTSEEIDHEDITKDKTSTVEACLPL | | |
| | L384M, | ELTKNESCLNSRETSFITNGSCLASRKTSFMMALCLSSI | | |
| | M421G, | YEDLKMYQVEFKTMNAKLLMDPKRQIFLDQNMLAVI | | |
| | G521R, | DELMQALNFNSETVPQKSSLEEPDFYKTKIKLCILLHA | | |
| | Y537S); stop | FRIRAVTIDRVMSYLNASGSSLALSLTADQMVSALLD | | |
| | | AEPPILYSEYDPTRPFSEASMMGLLTNLADRELVHMIN | | |
| | | WAKRVPGFVDLTLHDQVHLLECAWMEILMIGLVWRS | | |
| | | MEHPGKLLFAPNLLLDRNQGKCVEGGVEIFDMLLATS | | |
| | | SRFRMMNLQGEEFVCLKSIILLNSGVYTFLSSTLKSLEE | | |
| | | KDHIHRVLDKITDTLIHLMAKAGLTLQQQHQRLAQLL | | |
| | | LILSHIRHMSNKRMEHLYSMKCKNVVPLSDLLLEMLD | | |
| | | AHRL* | | |
| OT-001698 | IL12B (p40) | MCHQQLVISWFSLVFLASPLVAIWELKKDVYVVELD | 180 | 181 |
| | signal | WYPDAPGEMVVLTCDTPEEDGITWTLDQSSEVLGSGK | | |
| | sequence; | TLTIQVKEFGDAGQYTCHKGGEVLSHSLLLLHKKEDG | | |
| | IL12B (p40) | IWSTDILKDQKEPKNKTFLRCEAKNYSGRFTCWWLTT | | |
| | (23-328 of | ISTDLTFSVKSSRGSSDPQGVTCGAATLSAERVRGDNK | | |
| | WT); Linker | EYEYSVECQEDSACPAAEESLPIEVMVDAVHKLKYEN | | |
| | ((G4S)3; IL12A | YTSSFFIRDIIKPDPPKNLQLKPLKNSRQVEVSWEYPDT | | |
| | (p35) (57-253 | WSTPHSYFSLTFCVQVQGKSKREKKDRVFTDKTSATV | | |
| | of WT); Linker | ICRKNASISVRAQDRYYSSSWSEWASVPCSGGGGSGG | | |
| | (GS); ER (aa | GGSGGGGSRNLPVATPDPGMFPCLHHSQNLLRAVSN | | |
| | 305-549 of WT, | MLQKARQTLEFYPCTSEEIDHEDITKDKTSTVEACLPL | | |
| | L384M, | ELTKNESCLNSRETSFITNGSCLASRKTSFMMALCLSSI | | |
| | N413K, | YEDLKMYQVEFKTMNAKLLMDPKRQIFLDQNMLAVI | | |
| | M421G, | DELMQALNFNSETVPQKSSLEEPDFYKTKIKLCILLHA | | |
| | G521R, | FRIRAVTIDRVMSYLNASGSSLALSLTADQMVSALLD | | |
| | Y537S); stop | AEPPILYSEYDPTRPFSEASMMGLLTNLADRELVHMIN | | |
| | | WAKRVPGFVDLTLHDQVHLLECAWMEILMIGLVWRS | | |
| | | MEHPGKLLFAPNLLLDRKQGKCVEGGVEIFDMLLATS | | |
| | | SRFRMMNLQGEEFVCLKSIILLNSGVYTFLSSTLKSLEE | | |
| | | KDHIHRVLDKITDTLIHLMAKAGLTLQQQHQRLAQLL | | |
| | | LILSHIRHMSNKRMEHLYSMKCKNVVPLSDLLLEMLD | | |
| | | AHRL* | | |
| OT-001697 | IL12B (p40) | MCHQQLVISWFSLVFLASPLVAIWELKKDVYVVELD | 182 | 183 |
| | signal | WYPDAPGEMVVLTCDTPEEDGITWTLDQSSEVLGSGK | | |
| | sequence; | TLTIQVKEFGDAGQYTCHKGGEVLSHSLLLLHKKEDG | | |
| | IL12B (p40) | IWSTDILKDQKEPKNKTFLRCEAKNYSGRFTCWWLTT | | |
| | (23-328 of | ISTDLTFSVKSSRGSSDPQGVTCGAATLSAERVRGDNK | | |
| | WT); Linker | EYEYSVECQEDSACPAAEESLPIEVMVDAVHKLKYEN | | |
| | ((G4S)3; IL12A | YTSSFFIRDIIKPDPPKNLQLKPLKNSRQVEVSWEYPDT | | |
| | (p35) (57-253 | WSTPHSYFSLTFCVQVQGKSKREKKDRVFTDKTSATV | | |
| | of WT); Linker | ICRKNASISVRAQDRYYSSSWSEWASVPCSGGGGSGG | | |
| | (GS); ER (aa | GGSGGGGSRNLPVATPDPGMFPCLHHSQNLLRAVSN | | |
| | 305-549 of WT, | MLQKARQTLEFYPCTSEEIDHEDITKDKTSTVEACLPL | | |
| | L384M, | ELTKNESCLNSRETSFITNGSCLASRKTSFMMALCLSSI | | |
| | N413V, | YEDLKMYQVEFKTMNAKLLMDPKRQIFLDQNMLAVI | | |
| | M421G, | DELMQALNFNSETVPQKSSLEEPDFYKTKIKLCILLHA | | |
| | G521R, | FRIRAVTIDRVMSYLNASGSSLALSLTADQMVSALLD | | |
| | Y537S); stop | AEPPILYSEYDPTRPFSEASMMGLLTNLADRELVHMIN | | |
| | | WAKRVPGFVDLTLHDQVHLLECAWMEILMIGLVWRS | | |
| | | MEHPGKLLFAPNLLLDRVQGKCVEGGVEIFDMLLATS | | |
| | | SRFRMMNLQGEEFVCLKSIILLNSGVYTFLSSTLKSLEE | | |
| | | KDHIHRVLDKITDTLIHLMAKAGLTLQQQHQRLAQLL | | |
| | | LILSHIRHMSNKRMEHLYSMKCKNVVPLSDLLLEMLD | | |
| | | AHRL* | | |
| OT-001696 | IL12B (p40) | MCHQQLVISWFSLVFLASPLVAIWELKKDVYVVELD | 184 | 185 |
| | signal | WYPDAPGEMVVLTCDTPEEDGITWTLDQSSEVLGSGK | | |
| | sequence; | TLTIQVKEFGDAGQYTCHKGGEVLSHSLLLLHKKEDG | | |
| | IL12B (p40) | IWSTDILKDQKEPKNKTFLRCEAKNYSGRFTCWWLTT | | |
| | (23-328 of | ISTDLTFSVKSSRGSSDPQGVTCGAATLSAERVRGDNK | | |
| | WT); Linker | EYEYSVECQEDSACPAAEESLPIEVMVDAVHKLKYEN | | |
| | ((G4S)3; IL12A | YTSSFFIRDIIKPDPPKNLQLKPLKNSRQVEVSWEYPDT | | |
| | (p35) (57-253 | WSTPHSYFSLTFCVQVQGKSKREKKDRVFTDKTSATV | | |
| | of WT); Linker | ICRKNASISVRAQDRYYSSSWSEWASVPCSGGGGSGG | | |
| | (GS); ER (aa | GGSGGGGSRNLPVATPDPGMFPCLHHSQNLLRAVSN | | |
| | 305-549 of WT, | MLQKARQTLEFYPCTSEEIDHEDITKDKTSTVEACLPL | | |
| | L384M, | ELTKNESCLNSRETSFITNGSCLASRKTSFMMALCLSSI | | |
| | N413S, | YEDLKMYQVEFKTMNAKLLMDPKRQIFLDQNMLAVI | | |
| | M421G, | DELMQALNFNSETVPQKSSLEEPDFYKTKIKLCILLHA | | |
| | G521R, | FRIRAVTIDRVMSYLNASGSSLALSLTADQMVSALLD | | |
| | Y537S); stop | AEPPILYSEYDPTRPFSEASMMGLLTNLADRELVHMIN | | |
| | | WAKRVPGFVDLTLHDQVHLLECAWMEILMIGLVWRS | | |
| | | MEHPGKLLFAPNLLLDRSQGKCVEGGVEIFDMLLATS | | |
| | | SRFRMMNLQGEEFVCLKSIILLNSGVYTFLSSTLKSLEE | | |
| | | KDHIHRVLDKITDTLIHLMAKAGLTLQQQHQRLAQLL | | |
| | | LILSHIRHMSNKRMEHLYSMKCKNVVPLSDLLLEMLD | | |
| | | AHRL* | | |
| OT-001695 | IL12B (p40) | MCHQQLVISWFSLVFLASPLVAIWELKKDVYVVELD | 186 | 187 |
| | signal | WYPDAPGEMVVLTCDTPEEDGITWTLDQSSEVLGSGK | | |
| | sequence; | TLTIQVKEFGDAGQYTCHKGGEVLSHSLLLLHKKEDG | | |
| | IL12B (p40) | IWSTDILKDQKEPKNKTFLRCEAKNYSGRFTCWWLTT | | |
| | (23-328 of | ISTDLTFSVKSSRGSSDPQGVTCGAATLSAERVRGDNK | | |
| | WT); Linker | EYEYSVECQEDSACPAAEESLPIEVMVDAVHKLKYEN | | |
| | ((G4S)3; IL12A | YTSSFFIRDIIKPDPPKNLQLKPLKNSRQVEVSWEYPDT | | |
| | (p35) (57-253 | WSTPHSYFSLTFCVQVQGKSKREKKDRVFTDKTSATV | | |
| | of WT); Linker | ICRKNASISVRAQDRYYSSSWSEWASVPCSGGGGSGG | | |
| | (GS); ER (aa | GGSGGGGSRNLPVATPDPGMFPCLHHSQNLLRAVSN | | |
| | 305-549 of WT, | MLQKARQTLEFYPCTSEEIDHEDITKDKTSTVEACLPL | | |
| | L384M, | ELTKNESCLNSRETSFITNGSCLASRKTSFMMALCLSSI | | |
| | N413C, | YEDLKMYQVEFKTMNAKLLMDPKRQIFLDQNMLAVI | | |
| | M421G, | DELMQALNFNSETVPQKSSLEEPDFYKTKIKLCILLHA | | |
| | G521R, | FRIRAVTIDRVMSYLNASGSSLALSLTADQMVSALLD | | |
| | Y537S); stop | AEPPILYSEYDPTRPFSEASMMGLLTNLADRELVHMIN | | |
| | | WAKRVPGFVDLTLHDQVHLLECAWMEILMIGLVWRS | | |
| | | MEHPGKLLFAPNLLLDRCQGKCVEGGVEIFDMLLATS | | |
| | | SRFRMMNLQGEEFVCLKSIILLNSGVYTFLSSTLKSLEE | | |
| | | KDHIHRVLDKITDTLIHLMAKAGLTLQQQHQRLAQLL | | |
| | | LILSHIRHMSNKRMEHLYSMKCKNVVPLSDLLLEMLD | | |
| | | AHRL* | | |
| OT-001694 | IL12B (p40) | MCHQQLVISWFSLVFLASPLVAIWELKKDVYVVELD | 188 | 189 |
| | signal | WYPDAPGEMVVLTCDTPEEDGITWTLDQSSEVLGSGK | | |
| | sequence; | TLTIQVKEFGDAGQYTCHKGGEVLSHSLLLLHKKEDG | | |
| | IL12B (p40) | IWSTDILKDQKEPKNKTFLRCEAKNYSGRFTCWWLTT | | |
| | (23-328 of | ISTDLTFSVKSSRGSSDPQGVTCGAATLSAERVRGDNK | | |
| | WT); Linker | EYEYSVECQEDSACPAAEESLPIEVMVDAVHKLKYEN | | |
| | ((G4S)3; IL12A | YTSSFFIRDIIKPDPPKNLQLKPLKNSRQVEVSWEYPDT | | |
| | (p35) (57-253 | WSTPHSYFSLTFCVQVQGKSKREKKDRVFTDKTSATV | | |
| | of WT); Linker | ICRKNASISVRAQDRYYSSSWSEWASVPCSGGGGSGG | | |
| | (GS); ER (aa | GGSGGGGSRNLPVATPDPGMFPCLHHSQNLLRAVSN | | |
| | 305-549 of WT, | MLQKARQTLEFYPCTSEEIDHEDITKDKTSTVEACLPL | | |
| | L384M, | ELTKNESCLNSRETSFITNGSCLASRKTSFMMALCLSSI | | |
| | N413W, | YEDLKMYQVEFKTMNAKLLMDPKRQIFLDQNMLAVI | | |
| | M421G, | DELMQALNFNSETVPQKSSLEEPDFYKTKIKLCILLHA | | |
| | G521R, | FRIRAVTIDRVMSYLNASGSSLALSLTADQMVSALLD | | |
| | Y537S); stop | AEPPILYSEYDPTRPFSEASMMGLLTNLADRELVHMIN | | |
| | | WAKRVPGFVDLTLHDQVHLLECAWMEILMIGLVWRS | | |
| | | MEHPGKLLFAPNLLLDRWQGKCVEGGVEIHMLLATS | | |
| | | SRFRMMNLQGEEFVCLKSIILLNSGVYTFLSSTLKSLEE | | |
| | | KDHIHRVLDKITDTLIHLMAKAGLTLQQQHQRLAQLL | | |
| | | LILSHIRHMSNKRMEHLYSMKCKNVVPLSDLLLEMLD | | |
| | | AHRL* | | |
| OT-001693 | IL12B (p40) | MCHQQLVISWFSLVFLASPLVAIWELKKDVYVVELD | 190 | 191 |
| | signal | WYPDAPGEMVVLTCDTPEEDGITWTLDQSSEVLGSGK | | |
| | sequence; | TLTIQVKEFGDAGQYTCHKGGEVLSHSLLLLHKKEDG | | |
| | IL12B (p40) | IWSTDILKDQKEPKNKTFLRCEAKNYSGRFTCWWLTT | | |
| | (23-328 of | ISTDLTFSVKSSRGSSDPQGVTCGAATLSAERVRGDNK | | |
| | WT); Linker | EYEYSVECQEDSACPAAEESLPIEVMVDAVHKLKYEN | | |
| | ((G4S)3; IL12A | YTSSFFIRDIIKPDPPKNLQLKPLKNSRQVEVSWEYPDT | | |
| | (p35) (57-253 | WSTPHSYFSLTFCVQVQGKSKREKKDRVFTDKTSATV | | |
| | of WT); Linker | ICRKNASISVRAQDRYYSSSWSEWASVPCSGGGGSGG | | |
| | (GS); ER (aa | GGSGGGGSRNLPVATPDPGMFPCLHHSQNLLRAVSN | | |
| | 305-549 of WT, | MLQKARQTLEFYPCTSEEIDHEDITKDKTSTVEACLPL | | |
| | L384M, | ELTKNESCLNSRETSFITNGSCLASRKTSFMMALCLSSI | | |
| | N413P, | YEDLKMYQVEFKTMNAKLLMDPKRQIFLDQNMLAVI | | |
| | M421G, | DELMQALNFNSETVPQKSSLEEPDFYKTKIKLCILLHA | | |
| | G521R, | FRIRAVTIDRVMSYLNASGSSLALSLTADQMVSALLD | | |
| | Y537S); stop | AEPPILYSEYDPTRPFSEASMMGLLTNLADRELVHMIN | | |
| | | WAKRVPGFVDLTLHDQVHLLECAWMEILMIGLVWRS | | |
| | | MEHPGKLLFAPNLLLDRPQGKCVEGGVEIHMLLATS | | |
| | | SRFRMMNLQGEEFVCLKSIILLNSGVYTFLSSTLKSLEE | | |
| | | KDHIHRVLDKITDTLIHLMAKAGLTLQQQHQRLAQLL | | |
| | | LILSHIRHMSNKRMEHLYSMKCKNVVPLSDLLLEMLD | | |
| | | AHRL* | | |
| OT-001692 | IL12B (p40) | MCHQQLVISWFSLVFLASPLVAIWELKKDVYVVELD | 192 | 193 |
| | signal | WYPDAPGEMVVLTCDTPEEDGITWTLDQSSEVLGSGK | | |
| | sequence; | TLTIQVKEFGDAGQYTCHKGGEVLSHSLLLLHKKEDG | | |
| | IL12B (p40) | IWSTDILKDQKEPKNKTFLRCEAKNYSGRFTCWWLTT | | |
| | (23-328 of | ISTDLTFSVKSSRGSSDPQGVTCGAATLSAERVRGDNK | | |
| | WT); Linker | EYEYSVECQEDSACPAAEESLPIEVMVDAVHKLKYEN | | |
| | ((G4S)3; IL12A | YTSSFFIRDIIKPDPPKNLQLKPLKNSRQVEVSWEYPDT | | |
| | (p35) (57-253 | WSTPHSYFSLTFCVQVQGKSKREKKDRVFTDKTSATV | | |
| | of WT); Linker | ICRKNASISVRAQDRYYSSSWSEWASVPCSGGGGSGG | | |
| | (GS); ER (aa | GGSGGGGSRNLPVATPDPGMFPCLHHSQNLLRAVSN | | |
| | 305-549 of WT, | MLQKARQTLEFYPCTSEEIDHEDITKDKTSTVEACLPL | | |
| | L384M, | ELTKNESCLNSRETSFITNGSCLASRKTSFMMALCLSSI | | |
| | N413R, | YEDLKMYQVEFKTMNAKLLMDPKRQIFLDQNMLAVI | | |
| | M421G, | DELMQALNFNSETVPQKSSLEEPDFYKTKIKLCILLHA | | |
| | G521R, | FRIRAVTIDRVMSYLNASGSSLALSLTADQMVSALLD | | |
| | Y537S); stop | AEPPILYSEYDPTRPFSEASMMGLLTNLADRELVHMIN | | |
| | | WAKRVPGFVDLTLHDQVHLLECAWMEILMIGLVWRS | | |
| | | MEHPGKLLFAPNLLLDRRQGKCVEGGVEIFDMLLATS | | |
| | | SRFRMMNLQGEEFVCLKSIILLNSGVYTFLSSTLKSLEE | | |
| | | KDHIHRVLDKITDTLIHLMAKAGLTLQQQHQRLAQLL | | |
| | | LILSHIRHMSNKRMEHLYSMKCKNVVPLSDLLLEMLD | | |
| | | AHRL* | | |
| OT-001691 | IL12B (p40) | MCHQQLVISWFSLVFLASPLVAIWELKKDVYVVELD | 194 | 195 |
| | signal | WYPDAPGEMVVLTCDTPEEDGITWTLDQSSEVLGSGK | | |
| | sequence; | TLTIQVKEFGDAGQYTCHKGGEVLSHSLLLLHKKEDG | | |
| | IL12B (p40) | IWSTDILKDQKEPKNKTFLRCEAKNYSGRFTCWWLTT | | |
| | (23-328 of | ISTDLTFSVKSSRGSSDPQGVTCGAATLSAERVRGDNK | | |
| | WT); Linker | EYEYSVECQEDSACPAAEESLPIEVMVDAVHKLKYEN | | |
| | ((G4S)3; IL12A | YTSSFFIRDIIKPDPPKNLQLKPLKNSRQVEVSWEYPDT | | |
| | (p35) (57-253 | WSTPHSYFSLTFCVQVQGKSKREKKDRVFTDKTSATV | | |
| | of WT); Linker | ICRKNASISVRAQDRYYSSSWSEWASVPCSGGGGSGG | | |
| | (GS); ER (aa | GGSGGGGSRNLPVATPDPGMFPCLHHSQNLLRAVSN | | |
| | 305-549 of WT, | MLQKARQTLEFYPCTSEEIDHEDITKDKTSTVEACLPL | | |
| | L384M, | ELTKNESCLNSRETSFITNGSCLASRKTSFMMALCLSSI | | |
| | N413T, | YEDLKMYQVEFKTMNAKLLMDPKRQIFLDQNMLAVI | | |
| | M421G, | DELMQALNFNSETVPQKSSLEEPDFYKTKIKLCILLHA | | |
| | G521R, | FRIRAVTIDRVMSYLNASGSSLALSLTADQMVSALLD | | |
| | Y537S); stop | AEPPILYSEYDPTRPFSEASMMGLLTNLADRELVHMIN | | |
| | | WAKRVPGFVDLTLHDQVHLLECAWMEILMIGLVWRS | | |
| | | MEHPGKLLFAPNLLLDRTQGKCVEGGVEIFDMLLATS | | |
| | | SRFRMMNLQGEEFVCLKSIILLNSGVYTFLSSTLKSLEE | | |
| | | KDHIHRVLDKITDTLIHLMAKAGLTLQQQHQRLAQLL | | |
| | | LILSHIRHMSNKRMEHLYSMKCKNVVPLSDLLLEMLD | | |
| | | AHRL* | | |
| OT-001690 | IL12B (p40) | MCHQQLVISWFSLVFLASPLVAIWELKKDVYVVELD | 196 | 197 |
| | signal | WYPDAPGEMVVLTCDTPEEDGITWTLDQSSEVLGSGK | | |
| | sequence; | TLTIQVKEFGDAGQYTCHKGGEVLSHSLLLLHKKEDG | | |
| | IL12B (p40) | IWSTDILKDQKEPKNKTFLRCEAKNYSGRFTCWWLTT | | |
| | (23-328 of | ISTDLTFSVKSSRGSSDPQGVTCGAATLSAERVRGDNK | | |
| | WT); Linker | EYEYSVECQEDSACPAAEESLPIEVMVDAVHKLKYEN | | |
| | ((G4S)3; IL12A | YTSSFFIRDIIKPDPPKNLQLKPLKNSRQVEVSWEYPDT | | |
| | (p35) (57-253 | WSTPHSYFSLTFCVQVQGKSKREKKDRVFTDKTSATV | | |
| | of WT); Linker | ICRKNASISVRAQDRYYSSSWSEWASVPCSGGGGSGG | | |
| | (GS); ER (aa | GGSGGGGSRNLPVATPDPGMFPCLHHSQNLLRAVSN | | |
| | 305-549 of WT, | MLQKARQTLEFYPCTSEEIDHEDITKDKTSTVEACLPL | | |
| | L384M, | ELTKNESCLNSRETSFITNGSCLASRKTSFMMALCLSSI | | |
| | N413A, | YEDLKMYQVEFKTMNAKLLMDPKRQIFLDQNMLAVI | | |
| | M421G, | DELMQALNFNSETVPQKSSLEEPDFYKTKIKLCILLHA | | |
| | G521R, | FRIRAVTIDRVMSYLNASGSSLALSLTADQMVSALLD | | |
| | Y537S); stop | AEPPILYSEYDPTRPFSEASMMGLLTNLADRELVHMIN | | |
| | | WAKRVPGFVDLTLHDQVHLLECAWMEILMIGLVWRS | | |
| | | MEHPGKLLFAPNLLLDRAQGKCVEGGVEIFDMLLATS | | |
| | | SRFRMMNLQGEEFVCLKSIILLNSGVYTFLSSTLKSLEE | | |
| | | KDHIHRVLDKITDTLIHLMAKAGLTLQQQHQRLAQLL | | |
| | | LILSHIRHMSNKRMEHLYSMKCKNVVPLSDLLLEMLD | | |
| | | AHRL* | | |
| OT-001689 | IL12B (p40) | MCHQQLVISWFSLVFLASPLVAIWELKKDVYVVELD | 198 | 199 |
| | signal | WYPDAPGEMVVLTCDTPEEDGITWTLDQSSEVLGSGK | | |
| | sequence; | TLTIQVKEFGDAGQYTCHKGGEVLSHSLLLLHKKEDG | | |
| | IL12B (p40) | IWSTDILKDQKEPKNKTFLRCEAKNYSGRFTCWWLTT | | |
| | (23-328 of | ISTDLTFSVKSSRGSSDPQGVTCGAATLSAERVRGDNK | | |
| | WT); Linker | EYEYSVECQEDSACPAAEESLPIEVMVDAVHKLKYEN | | |
| | ((G4S)3; IL12A | YTSSFFIRDIIKPDPPKNLQLKPLKNSRQVEVSWEYPDT | | |
| | (p35) (57-253 | WSTPHSYFSLTFCVQVQGKSKREKKDRVFTDKTSATV | | |
| | of WT); Linker | ICRKNASISVRAQDRYYSSSWSEWASVPCSGGGGSGG | | |
| | (GS); ER (aa | GGSGGGGSRNLPVATPDPGMFPCLHHSQNLLRAVSN | | |
| | 305-549 of WT, | MLQKARQTLEFYPCTSEEIDHEDITKDKTSTVEACLPL | | |
| | L384M, | ELTKNESCLNSRETSFITNGSCLASRKTSFMMALCLSSI | | |
| | N413E, | YEDLKMYQVEFKTMNAKLLMDPKRQIFLDQNMLAVI | | |
| | M421G, | DELMQALNFNSETVPQKSSLEEPDFYKTKIKLCILLHA | | |
| | G521R, | FRIRAVTIDRVMSYLNASGSSLALSLTADQMVSALLD | | |
| | Y537S); stop | AEPPILYSEYDPTRPFSEASMMGLLTNLADRELVHMIN | | |
| | | WAKRVPGFVDLTLHDQVHLLECAWMEILMIGLVWRS | | |
| | | MEHPGKLLFAPNLLLDREQGKCVEGGVEIFDMLLATS | | |
| | | SRFRMMNLQGEEFVCLKSIILLNSGVYTFLSSTLKSLEE | | |
| | | KDHIHRVLDKITDTLIHLMAKAGLTLQQQHQRLAQLL | | |
| | | LILSHIRHMSNKRMEHLYSMKCKNVVPLSDLLLEMLD | | |
| | | AHRL* | | |
| OT-001688 | IL12B (p40) | MCHQQLVISWFSLVFLASPLVAIWELKKDVYVVELD | 200 | 201 |
| | signal | WYPDAPGEMVVLTCDTPEEDGITWTLDQSSEVLGSGK | | |
| | sequence; | TLTIQVKEFGDAGQYTCHKGGEVLSHSLLLLHKKEDG | | |
| | IL12B (p40) | IWSTDILKDQKEPKNKTFLRCEAKNYSGRFTCWWLTT | | |
| | (23-328 of | ISTDLTFSVKSSRGSSDPQGVTCGAATLSAERVRGDNK | | |
| | WT); Linker | EYEYSVECQEDSACPAAEESLPIEVMVDAVHKLKYEN | | |
| | ((G4S)3; IL12A | YTSSFFIRDIIKPDPPKNLQLKPLKNSRQVEVSWEYPDT | | |
| | (p35) (57-253 | WSTPHSYFSLTFCVQVQGKSKREKKDRVFTDKTSATV | | |
| | of WT); Linker | ICRKNASISVRAQDRYYSSSWSEWASVPCSGGGGSGG | | |
| | (GS); ER (aa | GGSGGGGSRNLPVATPDPGMFPCLHHSQNLLRAVSN | | |
| | 305-549 of WT, | MLQKARQTLEFYPCTSEEIDHEDITKDKTSTVEACLPL | | |
| | L384M, | ELTKNESCLNSRETSFITNGSCLASRKTSFMMALCLSSI | | |
| | N413G, | YEDLKMYQVEFKTMNAKLLMDPKRQIFLDQNMLAVI | | |
| | M421G, | DELMQALNFNSETVPQKSSLEEPDFYKTKIKLCILLHA | | |
| | G521R, | FRIRAVTIDRVMSYLNASGSSLALSLTADQMVSALLD | | |
| | Y537S); stop | AEPPILYSEYDPTRPFSEASMMGLLTNLADRELVHMIN | | |
| | | WAKRVPGFVDLTLHDQVHLLECAWMEILMIGLVWRS | | |
| | | MEHPGKLLFAPNLLLDRGQGKCVEGGVEIFDMLLATS | | |
| | | SRFRMMNLQGEEFVCLKSIILLNSGVYTFLSSTLKSLEE | | |
| | | KDHIHRVLDKITDTLIHLMAKAGLTLQQQHQRLAQLL | | |
| | | LILSHIRHMSNKRMEHLYSMKCKNVVPLSDLLLEMLD | | |
| | | AHRL* | | |
| OT-001726 | IL12B (p40) | MCHQQLVISWFSLVFLASPLVAIWELKKDVYVVELD | 202 | 203 |
| | signal | WYPDAPGEMVVLTCDTPEEDGITWTLDQSSEVLGSGK | | |
| | sequence; | TLTIQVKEFGDAGQYTCHKGGEVLSHSLLLLHKKEDG | | |
| | IL12B (p40) | IWSTDILKDQKEPKNKTFLRCEAKNYSGRFTCWWLTT | | |
| | (23-328 of | ISTDLTFSVKSSRGSSDPQGVTCGAATLSAERVRGDNK | | |
| | WT); Linker | EYEYSVECQEDSACPAAEESLPIEVMVDAVHKLKYEN | | |
| | ((G4S)3; IL12A | YTSSFFIRDIIKPDPPKNLQLKPLKNSRQVEVSWEYPDT | | |
| | (p35) (57-253 | WSTPHSYFSLTFCVQVQGKSKREKKDRVFTDKTSATV | | |
| | of WT); Linker | ICRKNASISVRAQDRYYSSSWSEWASVPCSGGGGSGG | | |
| | (GS); ER (aa | GGSGGGGSRNLPVATPDPGMFPCLHHSQNLLRAVSN | | |
| | 305-549 of WT, | MLQKARQTLEFYPCTSEEIDHEDITKDKTSTVEACLPL | | |
| | L384M, | ELTKNESCLNSRETSFITNGSCLASRKTSFMMALCLSSI | | |
| | M421G, | YEDLKMYQVEFKTMNAKLLMDPKRQIFLDQNMLAVI | | |
| | Q502F, G521R, | DELMQALNFNSETVPQKSSLEEPDFYKTKIKLCILLHA | | |
| | Y537S); stop | FRIRAVTIDRVMSYLNASGSSLALSLTADQMVSALLD | | |
| | | AEPPILYSEYDPTRPFSEASMMGLLTNLADRELVHMIN | | |
| | | WAKRVPGFVDLTLHDQVHLLECAWMEILMIGLVWRS | | |
| | | MEHPGKLLFAPNLLLDRNQGKCVEGGVEIFDMLLATS | | |
| | | SRFRMMNLQGEEFVCLKSIILLNSGVYTFLSSTLKSLEE | | |
| | | KDHIHRVLDKITDTLIHLMAKAGLTLQQQHFRLAQLL | | |
| | | LILSHIRHMSNKRMEHLYSMKCKNVVPLSDLLLEMLD | | |
| | | AHRL* | | |
| OT-001725 | IL12B (p40) | MCHQQLVISWFSLVFLASPLVAIWELKKDVYVVELD | 204 | 205 |
| | signal | WYPDAPGEMVVLTCDTPEEDGITWTLDQSSEVLGSGK | | |
| | sequence; | TLTIQVKEFGDAGQYTCHKGGEVLSHSLLLLHKKEDG | | |
| | IL12B (p40) | IWSTDILKDQKEPKNKTFLRCEAKNYSGRFTCWWLTT | | |
| | (23-328 of | ISTDLTFSVKSSRGSSDPQGVTCGAATLSAERVRGDNK | | |
| | WT); Linker | EYEYSVECQEDSACPAAEESLPIEVMVDAVHKLKYEN | | |
| | ((G4S)3; IL12A | YTSSFFIRDIIKPDPPKNLQLKPLKNSRQVEVSWEYPDT | | |
| | (p35) (57-253 | WSTPHSYFSLTFCVQVQGKSKREKKDRVFTDKTSATV | | |
| | of WT); Linker | ICRKNASISVRAQDRYYSSSWSEWASVPCSGGGGSGG | | |
| | (GS); ER (aa | GGSGGGGSRNLPVATPDPGMFPCLHHSQNLLRAVSN | | |
| | 305-549 of WT, | MLQKARQTLEFYPCTSEEIDHEDITKDKTSTVEACLPL | | |
| | L384M, | ELTKNESCLNSRETSFITNGSCLASRKTSFMMALCLSSI | | |
| | M421G, | YEDLKMYQVEFKTMNAKLLMDPKRQIFLDQNMLAVI | | |
| | Q502L, G521R, | DELMQALNFNSETVPQKSSLEEPDFYKTKIKLCILLHA | | |
| | Y537S); stop | FRIRAVTIDRVMSYLNASGSSLALSLTADQMVSALLD | | |
| | | AEPPILYSEYDPTRPFSEASMMGLLTNLADRELVHMIN | | |
| | | WAKRVPGFVDLTLHDQVHLLECAWMEILMIGLVWRS | | |
| | | MEHPGKLLFAPNLLLDRNQGKCVEGGVEIHMLLATS | | |
| | | SRFRMMNLQGEEFVCLKSIILLNSGVYTFLSSTLKSLEE | | |
| | | KDHIHRVLDKITDTLIHLMAKAGLTLQQQHLRLAQLL | | |
| | | LILSHIRHMSNKRMEHLYSMKCKNVVPLSDLLLEMLD | | |
| | | AHRL* | | |
| OT-001724 | IL12B (p40) | MCHQQLVISWFSLVFLASPLVAIWELKKDVYVVELD | 206 | 207 |
| | signal | WYPDAPGEMVVLTCDTPEEDGITWTLDQSSEVLGSGK | | |
| | sequence; | TLTIQVKEFGDAGQYTCHKGGEVLSHSLLLLHKKEDG | | |
| | IL12B (p40) | IWSTDILKDQKEPKNKTFLRCEAKNYSGRFTCWWLTT | | |
| | (23-328 of | ISTDLTFSVKSSRGSSDPQGVTCGAATLSAERVRGDNK | | |
| | WT); Linker | EYEYSVECQEDSACPAAEESLPIEVMVDAVHKLKYEN | | |
| | ((G4S)3; IL12A | YTSSFFIRDIIKPDPPKNLQLKPLKNSRQVEVSWEYPDT | | |
| | (p35) (57-253 | WSTPHSYFSLTFCVQVQGKSKREKKDRVFTDKTSATV | | |
| | of WT); Linker | ICRKNASISVRAQDRYYSSSWSEWASVPCSGGGGSGG | | |
| | (GS); ER (aa | GGSGGGGSRNLPVATPDPGMFPCLHHSQNLLRAVSN | | |
| | 305-549 of WT, | MLQKARQTLEFYPCTSEEIDHEDITKDKTSTVEACLPL | | |
| | L384M, | ELTKNESCLNSRETSFITNGSCLASRKTSFMMALCLSSI | | |
| | M421G, | YEDLKMYQVEFKTMNAKLLMDPKRQIFLDQNMLAVI | | |
| | Q502Y, | DELMQALNFNSETVPQKSSLEEPDFYKTKIKLCILLHA | | |
| | G521R, | FRIRAVTIDRVMSYLNASGSSLALSLTADQMVSALLD | | |
| | Y537S); stop | AEPPILYSEYDPTRPFSEASMMGLLTNLADRELVHMIN | | |
| | | WAKRVPGFVDLTLHDQVHLLECAWMEILMIGLVWRS | | |
| | | MEHPGKLLFAPNLLLDRNQGKCVEGGVEIHMLLATS | | |
| | | SRFRMMNLQGEEFVCLKSIILLNSGVYTFLSSTLKSLEE | | |
| | | KDHIHRVLDKITDTLIHLMAKAGLTLQQQHYRLAQLL | | |
| | | LILSHIRHMSNKRMEHLYSMKCKNVVPLSDLLLEMLD | | |
| | | AHRL* | | |
| OT-001723 | IL12B (p40) | MCHQQLVISWFSLVFLASPLVAIWELKKDVYVVELD | 208 | 209 |
| | signal | WYPDAPGEMVVLTCDTPEEDGITWTLDQSSEVLGSGK | | |
| | sequence; | TLTIQVKEFGDAGQYTCHKGGEVLSHSLLLLHKKEDG | | |
| | IL12B (p40) | IWSTDILKDQKEPKNKTFLRCEAKNYSGRFTCWWLTT | | |
| | (23-328 of | ISTDLTFSVKSSRGSSDPQGVTCGAATLSAERVRGDNK | | |
| | WT); Linker | EYEYSVECQEDSACPAAEESLPIEVMVDAVHKLKYEN | | |
| | ((G4S)3; IL12A | YTSSFFIRDIIKPDPPKNLQLKPLKNSRQVEVSWEYPDT | | |
| | (p35) (57-253 | WSTPHSYFSLTFCVQVQGKSKREKKDRVFTDKTSATV | | |
| | of WT); Linker | ICRKNASISVRAQDRYYSSSWSEWASVPCSGGGGSGG | | |
| | (GS); ER (aa | GGSGGGGSRNLPVATPDPGMFPCLHHSQNLLRAVSN | | |
| | 305-549 of WT, | MLQKARQTLEFYPCTSEEIDHEDITKDKTSTVEACLPL | | |
| | L384M, | ELTKNESCLNSRETSFITNGSCLASRKTSFMMALCLSSI | | |
| | M421G, | YEDLKMYQVEFKTMNAKLLMDPKRQIFLDQNMLAVI | | |
| | Q502H, | DELMQALNFNSETVPQKSSLEEPDFYKTKIKLCILLHA | | |
| | G521R, | FRIRAVTIDRVMSYLNASGSSLALSLTADQMVSALLD | | |
| | Y537S); stop | AEPPILYSEYDPTRPFSEASMMGLLTNLADRELVHMIN | | |
| | | WAKRVPGFVDLTLHDQVHLLECAWMEILMIGLVWRS | | |
| | | MEHPGKLLFAPNLLLDRNQGKCVEGGVEIFDMLLATS | | |
| | | SRFRMMNLQGEEFVCLKSIILLNSGVYTFLSSTLKSLEE | | |
| | | KDHIHRVLDKITDTLIHLMAKAGLTLQQQHHRLAQLL | | |
| | | LILSHIRHMSNKRMEHLYSMKCKNVVPLSDLLLEMLD | | |
| | | AHRL* | | |
| OT-001722 | IL12B (p40) | MCHQQLVISWFSLVFLASPLVAIWELKKDVYVVELD | 210 | 211 |
| | signal | WYPDAPGEMVVLTCDTPEEDGITWTLDQSSEVLGSGK | | |
| | sequence; | TLTIQVKEFGDAGQYTCHKGGEVLSHSLLLLHKKEDG | | |
| | IL12B (p40) | IWSTDILKDQKEPKNKTFLRCEAKNYSGRFTCWWLTT | | |
| | (23-328 of | ISTDLTFSVKSSRGSSDPQGVTCGAATLSAERVRGDNK | | |
| | WT); Linker | EYEYSVECQEDSACPAAEESLPIEVMVDAVHKLKYEN | | |
| | ((G4S)3; IL12A | YTSSFFIRDIIKPDPPKNLQLKPLKNSRQVEVSWEYPDT | | |
| | (p35) (57-253 | WSTPHSYFSLTFCVQVQGKSKREKKDRVFTDKTSATV | | |
| | of WT); Linker | ICRKNASISVRAQDRYYSSSWSEWASVPCSGGGGSGG | | |
| | (GS); ER (aa | GGSGGGGSRNLPVATPDPGMFPCLHHSQNLLRAVSN | | |
| | 305-549 of WT, | MLQKARQTLEFYPCTSEEIDHEDITKDKTSTVEACLPL | | |
| | L384M, | ELTKNESCLNSRETSFITNGSCLASRKTSFMMALCLSSI | | |
| | M421G, Q5021, | YEDLKMYQVEFKTMNAKLLMDPKRQIFLDQNMLAVI | | |
| | G521R, | DELMQALNFNSETVPQKSSLEEPDFYKTKIKLCILLHA | | |
| | Y537S); stop | FRIRAVTIDRVMSYLNASGSSLALSLTADQMVSALLD | | |
| | | AEPPILYSEYDPTRPFSEASMMGLLTNLADRELVHMIN | | |
| | | WAKRVPGFVDLTLHDQVHLLECAWMEILMIGLVWRS | | |
| | | MEHPGKLLFAPNLLLDRNQGKCVEGGVEIFDMLLATS | | |
| | | SRFRMMNLQGEEFVCLKSIILLNSGVYTFLSSTLKSLEE | | |
| | | KDHIHRVLDKITDTLIHLMAKAGLTLQQQHIRLAQLLL | | |
| | | ILSHIRHMSNKRMEHLYSMKCKNVVPLSDLLLEMLDA | | |
| | | HRL* | | |
| OT-001721 | IL12B (p40) | MCHQQLVISWFSLVFLASPLVAIWELKKDVYVVELD | 212 | 213 |
| | signal | WYPDAPGEMVVLTCDTPEEDGITWTLDQSSEVLGSGK | | |
| | sequence; | TLTIQVKEFGDAGQYTCHKGGEVLSHSLLLLHKKEDG | | |
| | IL12B (p40) | IWSTDILKDQKEPKNKTFLRCEAKNYSGRFTCWWLTT | | |
| | (23-328 of | ISTDLTFSVKSSRGSSDPQGVTCGAATLSAERVRGDNK | | |
| | WT); Linker | EYEYSVECQEDSACPAAEESLPIEVMVDAVHKLKYEN | | |
| | ((G4S)3; IL12A | YTSSFFIRDIIKPDPPKNLQLKPLKNSRQVEVSWEYPDT | | |
| | (p35) (57-253 | WSTPHSYFSLTFCVQVQGKSKREKKDRVFTDKTSATV | | |
| | of WT); Linker | ICRKNASISVRAQDRYYSSSWSEWASVPCSGGGGSGG | | |
| | (GS); ER (aa | GGSGGGGSRNLPVATPDPGMFPCLHHSQNLLRAVSN | | |
| | 305-549 of WT, | MLQKARQTLEFYPCTSEEIDHEDITKDKTSTVEACLPL | | |
| | L384M, | ELTKNESCLNSRETSFITNGSCLASRKTSFMMALCLSSI | | |
| | M421G, | YEDLKMYQVEFKTMNAKLLMDPKRQIFLDQNMLAVI | | |
| | Q502M, | DELMQALNFNSETVPQKSSLEEPDFYKTKIKLCILLHA | | |
| | G521R, | FRIRAVTIDRVMSYLNASGSSLALSLTADQMVSALLD | | |
| | Y537S); stop | AEPPILYSEYDPTRPFSEASMMGLLTNLADRELVHMIN | | |
| | | WAKRVPGFVDLTLHDQVHLLECAWMEILMIGLVWRS | | |
| | | MEHPGKLLFAPNLLLDRNQGKCVEGGVEIFDMLLATS | | |
| | | SRFRMMNLQGEEFVCLKSIILLNSGVYTFLSSTLKSLEE | | |
| | | KDHIHRVLDKITDTLIHLMAKAGLTLQQQHMRLAQLL | | |
| | | LILSHIRHMSNKRMEHLYSMKCKNVVPLSDLLLEMLD | | |
| | | AHRL* | | |
| OT-001720 | IL12B (p40) | MCHQQLVISWFSLVFLASPLVAIWELKKDVYVVELD | 214 | 215 |
| | signal | WYPDAPGEMVVLTCDTPEEDGITWTLDQSSEVLGSGK | | |
| | sequence; | TLTIQVKEFGDAGQYTCHKGGEVLSHSLLLLHKKEDG | | |
| | IL12B (p40) | IWSTDILKDQKEPKNKTFLRCEAKNYSGRFTCWWLTT | | |
| | (23-328 of | ISTDLTFSVKSSRGSSDPQGVTCGAATLSAERVRGDNK | | |
| | WT); Linker | EYEYSVECQEDSACPAAEESLPIEVMVDAVHKLKYEN | | |
| | ((G4S)3 ; IL12A | YTSSFFIRDIIKPDPPKNLQLKPLKNSRQVEVSWEYPDT | | |
| | (p35) (57-253 | WSTPHSYFSLTFCVQVQGKSKREKKDRVFTDKTSATV | | |
| | of WT); Linker | ICRKNASISVRAQDRYYSSSWSEWASVPCSGGGGSGG | | |
| | (GS); ER (aa | GGSGGGGSRNLPVATPDPGMFPCLHHSQNLLRAVSN | | |
| | 305-549 of WT, | MLQKARQTLEFYPCTSEEIDHEDITKDKTSTVEACLPL | | |
| | L384M, | ELTKNESCLNSRETSFITNGSCLASRKTSFMMALCLSSI | | |
| | M421G, | YEDLKMYQVEFKTMNAKLLMDPKRQIFLDQNMLAVI | | |
| | Q502N, | DELMQALNFNSETVPQKSSLEEPDFYKTKIKLCILLHA | | |
| | G521R, | FRIRAVTIDRVMSYLNASGSSLALSLTADQMVSALLD | | |
| | Y537S); stop | AEPPILYSEYDPTRPFSEASMMGLLTNLADRELVHMIN | | |
| | | WAKRVPGFVDLTLHDQVHLLECAWMEILMIGLVWRS | | |
| | | MEHPGKLLFAPNLLLDRNQGKCVEGGVEIFDMLLATS | | |
| | | SRFRMMNLQGEEFVCLKSIILLNSGVYTFLSSTLKSLEE | | |
| | | KDHIHRVLDKITDTLIHLMAKAGLTLQQQHNRLAQLL | | |
| | | LILSHIRHMSNKRMEHLYSMKCKNVVPLSDLLLEMLD | | |
| | | AHRL* | | |
| OT-001719 | IL12B (p40) | MCHQQLVISWFSLVFLASPLVAIWELKKDVYVVELD | 216 | 217 |
| | signal | WYPDAPGEMVVLTCDTPEEDGITWTLDQSSEVLGSGK | | |
| | sequence; | TLTIQVKEFGDAGQYTCHKGGEVLSHSLLLLHKKEDG | | |
| | IL12B (p40) | IWSTDILKDQKEPKNKTFLRCEAKNYSGRFTCWWLTT | | |
| | (23-328 of | ISTDLTFSVKSSRGSSDPQGVTCGAATLSAERVRGDNK | | |
| | WT); Linker | EYEYSVECQEDSACPAAEESLPIEVMVDAVHKLKYEN | | |
| | ((G4S)3; IL12A | YTSSFFIRDIIKPDPPKNLQLKPLKNSRQVEVSWEYPDT | | |
| | (p35) (57-253 | WSTPHSYFSLTFCVQVQGKSKREKKDRVFTDKTSATV | | |
| | of WT); Linker | ICRKNASISVRAQDRYYSSSWSEWASVPCSGGGGSGG | | |
| | (GS); ER (aa | GGSGGGGSRNLPVATPDPGMFPCLHHSQNLLRAVSN | | |
| | 305-549 of WT, | MLQKARQTLEFYPCTSEEIDHEDITKDKTSTVEACLPL | | |
| | L384M, | ELTKNESCLNSRETSFITNGSCLASRKTSFMMALCLSSI | | |
| | M421G, | YEDLKMYQVEFKTMNAKLLMDPKRQIFLDQNMLAVI | | |
| | Q502K, | DELMQALNFNSETVPQKSSLEEPDFYKTKIKLCILLHA | | |
| | G521R, | FRIRAVTIDRVMSYLNASGSSLALSLTADQMVSALLD | | |
| | Y537S); stop | AEPPILYSEYDPTRPFSEASMMGLLTNLADRELVHMIN | | |
| | | WAKRVPGFVDLTLHDQVHLLECAWMEILMIGLVWRS | | |
| | | MEHPGKLLFAPNLLLDRNQGKCVEGGVEIFDMLLATS | | |
| | | SRFRMMNLQGEEFVCLKSIILLNSGVYTFLSSTLKSLEE | | |
| | | KDHIHRVLDKITDTLIHLMAKAGLTLQQQHKRLAQLL | | |
| | | LILSHIRHMSNKRMEHLYSMKCKNVVPLSDLLLEMLD | | |
| | | AHRL* | | |
| OT-001718 | IL12B (p40) | MCHQQLVISWFSLVFLASPLVAIWELKKDVYVVELD | 218 | 219 |
| | signal | WYPDAPGEMVVLTCDTPEEDGITWTLDQSSEVLGSGK | | |
| | sequence; | TLTIQVKEFGDAGQYTCHKGGEVLSHSLLLLHKKEDG | | |
| | IL12B (p40) | IWSTDILKDQKEPKNKTFLRCEAKNYSGRFTCWWLTT | | |
| | (23-328 of | ISTDLTFSVKSSRGSSDPQGVTCGAATLSAERVRGDNK | | |
| | WT); Linker | EYEYSVECQEDSACPAAEESLPIEVMVDAVHKLKYEN | | |
| | ((G4S)3 ; IL12A | YTSSFFIRDIIKPDPPKNLQLKPLKNSRQVEVSWEYPDT | | |
| | (p35) (57-253 | WSTPHSYFSLTFCVQVQGKSKREKKDRVFTDKTSATV | | |
| | of WT); Linker | ICRKNASISVRAQDRYYSSSWSEWASVPCSGGGGSGG | | |
| | (GS); ER (aa | GGSGGGGSRNLPVATPDPGMFPCLHHSQNLLRAVSN | | |
| | 305-549 of WT, | MLQKARQTLEFYPCTSEEIDHEDITKDKTSTVEACLPL | | |
| | L384M, | ELTKNESCLNSRETSFITNGSCLASRKTSFMMALCLSSI | | |
| | M421G, | YEDLKMYQVEFKTMNAKLLMDPKRQIFLDQNMLAVI | | |
| | Q502V, | DELMQALNFNSETVPQKSSLEEPDFYKTKIKLCILLHA | | |
| | G521R, | FRIRAVTIDRVMSYLNASGSSLALSLTADQMVSALLD | | |
| | Y537S); stop | AEPPILYSEYDPTRPFSEASMMGLLTNLADRELVHMIN | | |
| | | WAKRVPGFVDLTLHDQVHLLECAWMEILMIGLVWRS | | |
| | | MEHPGKLLFAPNLLLDRNQGKCVEGGVEIFDMLLATS | | |
| | | SRFRMMNLQGEEFVCLKSIILLNSGVYTFLSSTLKSLEE | | |
| | | KDHIHRVLDKITDTLIHLMAKAGLTLQQQHVRLAQLL | | |
| | | LILSHIRHMSNKRMEHLYSMKCKNVVPLSDLLLEMLD | | |
| | | AHRL* | | |
| OT-001717 | IL12B (p40) | MCHQQLVISWFSLVFLASPLVAIWELKKDVYVVELD | 220 | 221 |
| | signal | WYPDAPGEMVVLTCDTPEEDGITWTLDQSSEVLGSGK | | |
| | sequence; | TLTIQVKEFGDAGQYTCHKGGEVLSHSLLLLHKKEDG | | |
| | IL12B (p40) | IWSTDILKDQKEPKNKTFLRCEAKNYSGRFTCWWLTT | | |
| | (23-328 of | ISTDLTFSVKSSRGSSDPQGVTCGAATLSAERVRGDNK | | |
| | WT); Linker | EYEYSVECQEDSACPAAEESLPIEVMVDAVHKLKYEN | | |
| | ((G4S)3; IL12A | YTSSFFIRDIIKPDPPKNLQLKPLKNSRQVEVSWEYPDT | | |
| | (p35) (57-253 | WSTPHSYFSLTFCVQVQGKSKREKKDRVFTDKTSATV | | |
| | of WT); Linker | ICRKNASISVRAQDRYYSSSWSEWASVPCSGGGGSGG | | |
| | (GS); ER (aa | GGSGGGGSRNLPVATPDPGMFPCLHHSQNLLRAVSN | | |
| | 305-549 of WT, | MLQKARQTLEFYPCTSEEIDHEDITKDKTSTVEACLPL | | |
| | L384M, | ELTKNESCLNSRETSFITNGSCLASRKTSFMMALCLSSI | | |
| | M421G, | YEDLKMYQVEFKTMNAKLLMDPKRQIFLDQNMLAVI | | |
| | Q502S , G521R, | DELMQALNFNSETVPQKSSLEEPDFYKTKIKLCILLHA | | |
| | Y537S); stop | FRIRAVTIDRVMSYLNASGSSLALSLTADQMVSALLD | | |
| | | AEPPILYSEYDPTRPFSEASMMGLLTNLADRELVHMIN | | |
| | | WAKRVPGFVDLTLHDQVHLLECAWMEILMIGLVWRS | | |
| | | MEHPGKLLFAPNLLLDRNQGKCVEGGVEIHMLLATS | | |
| | | SRFRMMNLQGEEFVCLKSIILLNSGVYTFLSSTLKSLEE | | |
| | | KDHIHRVLDKITDTLIHLMAKAGLTLQQQHSRLAQLL | | |
| | | LILSHIRHMSNKRMEHLYSMKCKNVVPLSDLLLEMLD | | |
| | | AHRL* | | |
| OT-001716 | IL12B (p40) | MCHQQLVISWFSLVFLASPLVAIWELKKDVYVVELD | 222 | 223 |
| | signal | WYPDAPGEMVVLTCDTPEEDGITWTLDQSSEVLGSGK | | |
| | sequence; | TLTIQVKEFGDAGQYTCHKGGEVLSHSLLLLHKKEDG | | |
| | IL12B (p40) | IWSTDILKDQKEPKNKTFLRCEAKNYSGRFTCWWLTT | | |
| | (23-328 of | ISTDLTFSVKSSRGSSDPQGVTCGAATLSAERVRGDNK | | |
| | WT); Linker | EYEYSVECQEDSACPAAEESLPIEVMVDAVHKLKYEN | | |
| | ((G4S)3; IL12A | YTSSFFIRDIIKPDPPKNLQLKPLKNSRQVEVSWEYPDT | | |
| | (p35) (57-253 | WSTPHSYFSLTFCVQVQGKSKREKKDRVFTDKTSATV | | |
| | of WT); Linker | ICRKNASISVRAQDRYYSSSWSEWASVPCSGGGGSGG | | |
| | (GS); ER (aa | GGSGGGGSRNLPVATPDPGMFPCLHHSQNLLRAVSN | | |
| | 305-549 of WT, | MLQKARQTLEFYPCTSEEIDHEDITKDKTSTVEACLPL | | |
| | L384M, | ELTKNESCLNSRETSFITNGSCLASRKTSFMMALCLSSI | | |
| | M421G, | YEDLKMYQVEFKTMNAKLLMDPKRQIFLDQNMLAVI | | |
| | Q502C, | DELMQALNFNSETVPQKSSLEEPDFYKTKIKLCILLHA | | |
| | G521R, | FRIRAVTIDRVMSYLNASGSSLALSLTADQMVSALLD | | |
| | Y537S); stop | AEPPILYSEYDPTRPFSEASMMGLLTNLADRELVHMIN | | |
| | | WAKRVPGFVDLTLHDQVHLLECAWMEILMIGLVWRS | | |
| | | MEHPGKLLFAPNLLLDRNQGKCVEGGVEIHMLLATS | | |
| | | SRFRMMNLQGEEFVCLKSIILLNSGVYTFLSSTLKSLEE | | |
| | | KDHIHRVLDKITDTLIHLMAKAGLTLQQQHCRLAQLL | | |
| | | LILSHIRHMSNKRMEHLYSMKCKNVVPLSDLLLEMLD | | |
| | | AHRL* | | |
| OT-001715 | IL12B (p40) | MCHQQLVISWFSLVFLASPLVAIWELKKDVYVVELD | 224 | 225 |
| | signal | WYPDAPGEMVVLTCDTPEEDGITWTLDQSSEVLGSGK | | |
| | sequence; | TLTIQVKEFGDAGQYTCHKGGEVLSHSLLLLHKKEDG | | |
| | IL12B (p40) | IWSTDILKDQKEPKNKTFLRCEAKNYSGRFTCWWLTT | | |
| | (23-328 of | ISTDLTFSVKSSRGSSDPQGVTCGAATLSAERVRGDNK | | |
| | WT); Linker | EYEYSVECQEDSACPAAEESLPIEVMVDAVHKLKYEN | | |
| | ((G4S)3; IL12A | YTSSFFIRDIIKPDPPKNLQLKPLKNSRQVEVSWEYPDT | | |
| | (p35) (57-253 | WSTPHSYFSLTFCVQVQGKSKREKKDRVFTDKTSATV | | |
| | of WT); Linker | ICRKNASISVRAQDRYYSSSWSEWASVPCSGGGGSGG | | |
| | (GS); ER (aa | GGSGGGGSRNLPVATPDPGMFPCLHHSQNLLRAVSN | | |
| | 305-549 of WT, | MLQKARQTLEFYPCTSEEIDHEDITKDKTSTVEACLPL | | |
| | L384M, | ELTKNESCLNSRETSFITNGSCLASRKTSFMMALCLSSI | | |
| | M421G, | YEDLKMYQVEFKTMNAKLLMDPKRQIFLDQNMLAVI | | |
| | Q502W, | DELMQALNFNSETVPQKSSLEEPDFYKTKIKLCILLHA | | |
| | G521R, | FRIRAVTIDRVMSYLNASGSSLALSLTADQMVSALLD | | |
| | Y537S); stop | AEPPILYSEYDPTRPFSEASMMGLLTNLADRELVHMIN | | |
| | | WAKRVPGFVDLTLHDQVHLLECAWMEILMIGLVWRS | | |
| | | MEHPGKLLFAPNLLLDRNQGKCVEGGVEIFDMLLATS | | |
| | | SRFRMMNLQGEEFVCLKSIILLNSGVYTFLSSTLKSLEE | | |
| | | KDHIHRVLDKITDTLIHLMAKAGLTLQQQHWRLAQLL | | |
| | | LILSHIRHMSNKRMEHLYSMKCKNVVPLSDLLLEMLD | | |
| | | AHRL* | | |
| OT-001714 | IL12B (p40) | MCHQQLVISWFSLVFLASPLVAIWELKKDVYVVELD | 226 | 227 |
| | signal | WYPDAPGEMVVLTCDTPEEDGITWTLDQSSEVLGSGK | | |
| | sequence; | TLTIQVKEFGDAGQYTCHKGGEVLSHSLLLLHKKEDG | | |
| | IL12B (p40) | IWSTDILKDQKEPKNKTFLRCEAKNYSGRFTCWWLTT | | |
| | (23-328 of | ISTDLTFSVKSSRGSSDPQGVTCGAATLSAERVRGDNK | | |
| | WT); Linker | EYEYSVECQEDSACPAAEESLPIEVMVDAVHKLKYEN | | |
| | ((G4S)3; IL12A | YTSSFFIRDIIKPDPPKNLQLKPLKNSRQVEVSWEYPDT | | |
| | (p35) (57-253 | WSTPHSYFSLTFCVQVQGKSKREKKDRVFTDKTSATV | | |
| | of WT); Linker | ICRKNASISVRAQDRYYSSSWSEWASVPCSGGGGSGG | | |
| | (GS); ER (aa | GGSGGGGSRNLPVATPDPGMFPCLHHSQNLLRAVSN | | |
| | 305-549 of WT, | MLQKARQTLEFYPCTSEEIDHEDITKDKTSTVEACLPL | | |
| | L384M, | ELTKNESCLNSRETSFITNGSCLASRKTSFMMALCLSSI | | |
| | M421G, | YEDLKMYQVEFKTMNAKLLMDPKRQIFLDQNMLAVI | | |
| | Q502P, G521R, | DELMQALNFNSETVPQKSSLEEPDFYKTKIKLCILLHA | | |
| | Y537S); stop | FRIRAVTIDRVMSYLNASGSSLALSLTADQMVSALLD | | |
| | | AEPPILYSEYDPTRPFSEASMMGLLTNLADRELVHMIN | | |
| | | WAKRVPGFVDLTLHDQVHLLECAWMEILMIGLVWRS | | |
| | | MEHPGKLLFAPNLLLDRNQGKCVEGGVEIFDMLLATS | | |
| | | SRFRMMNLQGEEFVCLKSIILLNSGVYTFLSSTLKSLEE | | |
| | | KDHIHRVLDKITDTLIHLMAKAGLTLQQQHPRLAQLL | | |
| | | LILSHIRHMSNKRMEHLYSMKCKNVVPLSDLLLEMLD | | |
| | | AHRL* | | |
| OT-001713 | IL12B (p40) | MCHQQLVISWFSLVFLASPLVAIWELKKDVYVVELD | 228 | 229 |
| | signal | WYPDAPGEMVVLTCDTPEEDGITWTLDQSSEVLGSGK | | |
| | sequence; | TLTIQVKEFGDAGQYTCHKGGEVLSHSLLLLHKKEDG | | |
| | IL12B (p40) | IWSTDILKDQKEPKNKTFLRCEAKNYSGRFTCWWLTT | | |
| | (23-328 of | ISTDLTFSVKSSRGSSDPQGVTCGAATLSAERVRGDNK | | |
| | WT); Linker | EYEYSVECQEDSACPAAEESLPIEVMVDAVHKLKYEN | | |
| | ((G4S)3; IL12A | YTSSFFIRDIIKPDPPKNLQLKPLKNSRQVEVSWEYPDT | | |
| | (p35) (57-253 | WSTPHSYFSLTFCVQVQGKSKREKKDRVFTDKTSATV | | |
| | of WT); Linker | ICRKNASISVRAQDRYYSSSWSEWASVPCSGGGGSGG | | |
| | (GS); ER (aa | GGSGGGGSRNLPVATPDPGMFPCLHHSQNLLRAVSN | | |
| | 305-549 of WT, | MLQKARQTLEFYPCTSEEIDHEDITKDKTSTVEACLPL | | |
| | L384M, | ELTKNESCLNSRETSFITNGSCLASRKTSFMMALCLSSI | | |
| | M421G, | YEDLKMYQVEFKTMNAKLLMDPKRQIFLDQNMLAVI | | |
| | Q502T, G521R, | DELMQALNFNSETVPQKSSLEEPDFYKTKIKLCILLHA | | |
| | Y537S); stop | FRIRAVTIDRVMSYLNASGSSLALSLTADQMVSALLD | | |
| | | AEPPILYSEYDPTRPFSEASMMGLLTNLADRELVHMIN | | |
| | | WAKRVPGFVDLTLHDQVHLLECAWMEILMIGLVWRS | | |
| | | MEHPGKLLFAPNLLLDRNQGKCVEGGVEIFDMLLATS | | |
| | | SRFRMMNLQGEEFVCLKSIILLNSGVYTFLSSTLKSLEE | | |
| | | KDHIHRVLDKITDTLIHLMAKAGLTLQQQHTRLAQLL | | |
| | | LILSHIRHMSNKRMEHLYSMKCKNVVPLSDLLLEMLD | | |
| | | AHRL* | | |
| OT-001712 | IL12B (p40) | MCHQQLVISWFSLVFLASPLVAIWELKKDVYVVELD | 230 | 231 |
| | signal | WYPDAPGEMVVLTCDTPEEDGITWTLDQSSEVLGSGK | | |
| | sequence; | TLTIQVKEFGDAGQYTCHKGGEVLSHSLLLLHKKEDG | | |
| | IL12B (p40) | IWSTDILKDQKEPKNKTFLRCEAKNYSGRFTCWWLTT | | |
| | (23-328 of | ISTDLTFSVKSSRGSSDPQGVTCGAATLSAERVRGDNK | | |
| | WT); Linker | EYEYSVECQEDSACPAAEESLPIEVMVDAVHKLKYEN | | |
| | ((G4S)3; IL12A | YTSSFFIRDIIKPDPPKNLQLKPLKNSRQVEVSWEYPDT | | |
| | (p35) (57-253 | WSTPHSYFSLTFCVQVQGKSKREKKDRVFTDKTSATV | | |
| | of WT); Linker | ICRKNASISVRAQDRYYSSSWSEWASVPCSGGGGSGG | | |
| | (GS); ER (aa | GGSGGGGSRNLPVATPDPGMFPCLHHSQNLLRAVSN | | |
| | 305-549 of WT, | MLQKARQTLEFYPCTSEEIDHEDITKDKTSTVEACLPL | | |
| | L384M, | ELTKNESCLNSRETSFITNGSCLASRKTSFMMALCLSSI | | |
| | M421G, | YEDLKMYQVEFKTMNAKLLMDPKRQIFLDQNMLAVI | | |
| | Q502A, | DELMQALNFNSETVPQKSSLEEPDFYKTKIKLCILLHA | | |
| | G521R, | FRIRAVTIDRVMSYLNASGSSLALSLTADQMVSALLD | | |
| | Y537S); stop | AEPPILYSEYDPTRPFSEASMMGLLTNLADRELVHMIN | | |
| | | WAKRVPGFVDLTLHDQVHLLECAWMEILMIGLVWRS | | |
| | | MEHPGKLLFAPNLLLDRNQGKCVEGGVEIFDMLLATS | | |
| | | SRFRMMNLQGEEFVCLKSIILLNSGVYTFLSSTLKSLEE | | |
| | | KDHIHRVLDKITDTLIHLMAKAGLTLQQQHARLAQLL | | |
| | | LILSHIRHMSNKRMEHLYSMKCKNVVPLSDLLLEMLD | | |
| | | AHRL* | | |
| OT-001711 | IL12B (p40) | MCHQQLVISWFSLVFLASPLVAIWELKKDVYVVELD | 232 | 233 |
| | signal | WYPDAPGEMVVLTCDTPEEDGITWTLDQSSEVLGSGK | | |
| | sequence; | TLTIQVKEFGDAGQYTCHKGGEVLSHSLLLLHKKEDG | | |
| | IL12B (p40) | IWSTDILKDQKEPKNKTFLRCEAKNYSGRFTCWWLTT | | |
| | (23-328 of | ISTDLTFSVKSSRGSSDPQGVTCGAATLSAERVRGDNK | | |
| | WT); Linker | EYEYSVECQEDSACPAAEESLPIEVMVDAVHKLKYEN | | |
| | ((G4S)3; IL12A | YTSSFFIRDIIKPDPPKNLQLKPLKNSRQVEVSWEYPDT | | |
| | (p35) (57-253 | WSTPHSYFSLTFCVQVQGKSKREKKDRVFTDKTSATV | | |
| | of WT); Linker | ICRKNASISVRAQDRYYSSSWSEWASVPCSGGGGSGG | | |
| | (GS); ER (aa | GGSGGGGSRNLPVATPDPGMFPCLHHSQNLLRAVSN | | |
| | 305-549 of WT, | MLQKARQTLEFYPCTSEEIDHEDITKDKTSTVEACLPL | | |
| | L384M, | ELTKNESCLNSRETSFITNGSCLASRKTSFMMALCLSSI | | |
| | M421G, | YEDLKMYQVEFKTMNAKLLMDPKRQIFLDQNMLAVI | | |
| | Q502D, | DELMQALNFNSETVPQKSSLEEPDFYKTKIKLCILLHA | | |
| | G521R, | FRIRAVTIDRVMSYLNASGSSLALSLTADQMVSALLD | | |
| | Y537S); stop | AEPPILYSEYDPTRPFSEASMMGLLTNLADRELVHMIN | | |
| | | WAKRVPGFVDLTLHDQVHLLECAWMEILMIGLVWRS | | |
| | | MEHPGKLLFAPNLLLDRNQGKCVEGGVEIFDMLLATS | | |
| | | SRFRMMNLQGEEFVCLKSIILLNSGVYTFLSSTLKSLEE | | |
| | | KDHIHRVLDKITDTLIHLMAKAGLTLQQQHDRLAQLL | | |
| | | LILSHIRHMSNKRMEHLYSMKCKNVVPLSDLLLEMLD | | |
| | | AHRL* | | |
| OT-001710 | IL12B (p40) | MCHQQLVISWFSLVFLASPLVAIWELKKDVYVVELD | 234 | 235 |
| | signal | WYPDAPGEMVVLTCDTPEEDGITWTLDQSSEVLGSGK | | |
| | sequence; | TLTIQVKEFGDAGQYTCHKGGEVLSHSLLLLHKKEDG | | |
| | IL12B (p40) | IWSTDILKDQKEPKNKTFLRCEAKNYSGRFTCWWLTT | | |
| | (23-328 of | ISTDLTFSVKSSRGSSDPQGVTCGAATLSAERVRGDNK | | |
| | WT); Linker | EYEYSVECQEDSACPAAEESLPIEVMVDAVHKLKYEN | | |
| | ((G4S)3; IL12A | YTSSFFIRDIIKPDPPKNLQLKPLKNSRQVEVSWEYPDT | | |
| | (p35) (57-253 | WSTPHSYFSLTFCVQVQGKSKREKKDRVFTDKTSATV | | |
| | of WT); Linker | ICRKNASISVRAQDRYYSSSWSEWASVPCSGGGGSGG | | |
| | (GS); ER (aa | GGSGGGGSRNLPVATPDPGMFPCLHHSQNLLRAVSN | | |
| | 305-549 of WT, | MLQKARQTLEFYPCTSEEIDHEDITKDKTSTVEACLPL | | |
| | L384M, | ELTKNESCLNSRETSFITNGSCLASRKTSFMMALCLSSI | | |
| | M421G, | YEDLKMYQVEFKTMNAKLLMDPKRQIFLDQNMLAVI | | |
| | Q502E, G521R, | DELMQALNFNSETVPQKSSLEEPDFYKTKIKLCILLHA | | |
| | Y537S); stop | FRIRAVTIDRVMSYLNASGSSLALSLTADQMVSALLD | | |
| | | AEPPILYSEYDPTRPFSEASMMGLLTNLADRELVHMIN | | |
| | | WAKRVPGFVDLTLHDQVHLLECAWMEILMIGLVWRS | | |
| | | MEHPGKLLFAPNLLLDRNQGKCVEGGVEIFDMLLATS | | |
| | | SRFRMMNLQGEEFVCLKSIILLNSGVYTFLSSTLKSLEE | | |
| | | KDHIHRVLDKITDTLIHLMAKAGLTLQQQHERLAQLL | | |
| | | LILSHIRHMSNKRMEHLYSMKCKNVVPLSDLLLEMLD | | |
| | | AHRL* | | |
| OT-001709 | IL12B (p40) | MCHQQLVISWFSLVFLASPLVAIWELKKDVYVVELD | 236 | 237 |
| | signal | WYPDAPGEMVVLTCDTPEEDGITWTLDQSSEVLGSGK | | |
| | sequence; | TLTIQVKEFGDAGQYTCHKGGEVLSHSLLLLHKKEDG | | |
| | IL12B (p40) | IWSTDILKDQKEPKNKTFLRCEAKNYSGRFTCWWLTT | | |
| | (23-328 of | ISTDLTFSVKSSRGSSDPQGVTCGAATLSAERVRGDNK | | |
| | WT); Linker | EYEYSVECQEDSACPAAEESLPIEVMVDAVHKLKYEN | | |
| | ((G4S)3; IL12A | YTSSFFIRDIIKPDPPKNLQLKPLKNSRQVEVSWEYPDT | | |
| | (p35) (57-253 | WSTPHSYFSLTFCVQVQGKSKREKKDRVFTDKTSATV | | |
| | of WT); Linker | ICRKNASISVRAQDRYYSSSWSEWASVPCSGGGGSGG | | |
| | (GS); ER (aa | GGSGGGGSRNLPVATPDPGMFPCLHHSQNLLRAVSN | | |
| | 305-549 of WT, | MLQKARQTLEFYPCTSEEIDHEDITKDKTSTVEACLPL | | |
| | L384M, | ELTKNESCLNSRETSFITNGSCLASRKTSFMMALCLSSI | | |
| | M421G, | YEDLKMYQVEFKTMNAKLLMDPKRQIFLDQNMLAVI | | |
| | Q502G, | DELMQALNFNSETVPQKSSLEEPDFYKTKIKLCILLHA | | |
| | G521R, | FRIRAVTIDRVMSYLNASGSSLALSLTADQMVSALLD | | |
| | Y537S); stop | AEPPILYSEYDPTRPFSEASMMGLLTNLADRELVHMIN | | |
| | | WAKRVPGFVDLTLHDQVHLLECAWMEILMIGLVWRS | | |
| | | MEHPGKLLFAPNLLLDRNQGKCVEGGVEIHMLLATS | | |
| | | SRFRMMNLQGEEFVCLKSIILLNSGVYTFLSSTLKSLEE | | |
| | | KDHIHRVLDKITDTLIHLMAKAGLTLQQQHGRLAQLL | | |
| | | LILSHIRHMSNKRMEHLYSMKCKNVVPLSDLLLEMLD | | |
| | | AHRL* | | |
| OT-001708 | IL12B (p40) | MCHQQLVISWFSLVFLASPLVAIWELKKDVYVVELD | 238 | 239 |
| | signal | WYPDAPGEMVVLTCDTPEEDGITWTLDQSSEVLGSGK | | |
| | sequence; | TLTIQVKEFGDAGQYTCHKGGEVLSHSLLLLHKKEDG | | |
| | IL12B (p40) | IWSTDILKDQKEPKNKTFLRCEAKNYSGRFTCWWLTT | | |
| | (23-328 of | ISTDLTFSVKSSRGSSDPQGVTCGAATLSAERVRGDNK | | |
| | WT); Linker | EYEYSVECQEDSACPAAEESLPIEVMVDAVHKLKYEN | | |
| | ((G4S)3; IL12A | YTSSFFIRDIIKPDPPKNLQLKPLKNSRQVEVSWEYPDT | | |
| | (p35) (57-253 | WSTPHSYFSLTFCVQVQGKSKREKKDRVFTDKTSATV | | |
| | of WT); Linker | ICRKNASISVRAQDRYYSSSWSEWASVPCSGGGGSGG | | |
| | (GS); ER (305- | GGSGGGGSRNLPVATPDPGMFPCLHHSQNLLRAVSN | | |
| | 549 of WT, | MLQKARQTLEFYPCTSEEIDHEDITKDKTSTVEACLPL | | |
| | L384M, | ELTKNESCLNSRETSFITNGSCLASRKTSFMMALCLSSI | | |
| | M421G, | YEDLKMYQVEFKTMNAKLLMDPKRQIFLDQNMLAVI | | |
| | Q502R, | DELMQALNFNSETVPQKSSLEEPDFYKTKIKLCILLHA | | |
| | G521R, | FRIRAVTIDRVMSYLNASGSSLALSLTADQMVSALLD | | |
| | Y537S); stop | AEPPILYSEYDPTRPFSEASMMGLLTNLADRELVHMIN | | |
| | | WAKRVPGFVDLTLHDQVHLLECAWMEILMIGLVWRS | | |
| | | MEHPGKLLFAPNLLLDRNQGKCVEGGVEIHMLLATS | | |
| | | SRFRMMNLQGEEFVCLKSIILLNSGVYTFLSSTLKSLEE | | |
| | | KDHIHRVLDKITDTLIHLMAKAGLTLQQQHRRLAQLL | | |
| | | LILSHIRHMSNKRMEHLYSMKCKNVVPLSDLLLEMLD | | |
| | | AHRL* | | |
| OT-001737 | IL12B (p40) | MCHQQLVISWFSLVFLASPLVAGSSLALSLTADQMVS | 240 | 241 |
| | signal | ALLDAEPPILYSEYDPTRPFSEASMMGLLTNLADRELV | | |
| | sequence; | HMINWAKRVPGFVDLALHDQVHLLECAWMEILMIGL | | |
| | Linker (GS); | VWRSMEHPGKLLFAPNLLLDRNQGKCVEGGVEIFDM | | |
| | ER (aa 305-549 | LLATSSRFRMMNLQGEEFVCLKSIILLNSGVYTFLSSTL | | |
| | of WT, T371A, | KSLEEKDHIHRVLDKITDTLIHLMAKAGLTLQQQHQR | | |
| | L384M, | LAQLLLILSHIRHMSSKRMEHLYSMKCKNVVPLSDLL | | |
| | M421G, | LEMLDAHRLRTKRIWELKKDVYVVELDWYPDAPGE | | |
| | N519S, G521R, | MVVLTCDTPEEDGITWTLDQSSEVLGSGKTLTIQVKEF | | |
| | Y537S); Furin | GDAGQYTCHKGGEVLSHSLLLLHKKEDGIWSTDILKD | | |
| | cleavage site | QKEPKNKTFLRCEAKNYSGRFTCWWLTTISTDLTFSV | | |
| | (RTKR); IL12B | KSSRGSSDPQGVTCGAATLSAERVRGDNKEYEYSVEC | | |
| | (p40) (23-328 | QED SACPAAEESLPIEVMVDAVHKLKYENYTSSFFIRD | | |
| | of WT); Linker | IIKPDPPKNLQLKPLKNSRQVEVSWEYPDTWSTPHSYF | | |
| | ((G4S)3; IL12A | SLTFCVQVQGKSKREKKDRVFTDKTSATVICRKNASIS | | |
| | (p35) (57-253 | VRAQDRYYSSSWSEWASVPCSGGGGSGGGGSGGGGS | | |
| | of WT); stop | RNLPVATPDPGMFPCLHHSQNLLRAVSNMLQKARQT | | |
| | | LEFYPCTSEEIDHEDITKDKTSTVEACLPLELTKNESCL | | |
| | | NSRETSFITNGSCLASRKTSFMMALCLSSIYEDLKMYQ | | |
| | | VEFKTMNAKLLMDPKRQIFLDQNMLAVIDELMQALN | | |
| | | FNSETVPQKSSLEEPDFYKTKIKLCILLHAFRIRAVTIDR | | |
| | | VMSYLNAS* | | |
| OT-001738 | IL12B (p40) | MCHQQLVISWFSLVFLASPLVAGSSLALSLTADQMVS | 242 | 243 |
| | signal | ALLDAEPPILYSEYDPTRPFSEASMMGLLTNLADRELV | | |
| | sequence; | HMINWAKRVPGFVDLALHDQVHLLECAWMEILMIGL | | |
| | Linker (GS); | VWRSMEHPGKLLFAPNLLLDRNQGKCVEGGVEIFDM | | |
| | ER (aa 305-549 | LLATSSRFRMMNLQGEEFVCLKSIILLNSGVYTFLSSTL | | |
| | of WT, T371A, | KSLEEKDHIHRVLDKITDTLIHLMAKAGLTLQQQHQR | | |
| | L384M, | LAQLLLILSHIRHMSSKRMEHLYSMKCKNVVPLSDLL | | |
| | M421G, | LEMLDAHRLESRRVRRNKRSKIWELKKDVYVVELDW | | |
| | N519S, G521R, | YPDAPGEMVVLTCDTPEEDGITWTLDQSSEVLGSGKT | | |
| | Y537S); | LTIQVKEFGDAGQYTCHKGGEVLSHSLLLLHKKEDGI | | |
| | Modified Furin | WSTDILKDQKEPKNKTFLRCEAKNYSGRFTCWWLTTI | | |
| | (ESRRVRRNK | STDLTFSVKSSRGSSDPQGVTCGAATLSAERVRGDNK | | |
| | RSK); IL12B | EYEYSVECQEDSACPAAEESLPIEVMVDAVHKLKYEN | | |
| | (p40) (23-328 | YTSSFFIRDIIKPDPPKNLQLKPLKNSRQVEVSWEYPDT | | |
| | of WT); Linker | WSTPHSYFSLTFCVQVQGKSKREKKDRVFTDKTSATV | | |
| | ((G4S)3; IL12A | ICRKNASISVRAQDRYYSSSWSEWASVPCSGGGGSGG | | |
| | (p35) (57-253 | GGSGGGGSRNLPVATPDPGMFPCLHHSQNLLRAVSN | | |
| | of WT; stop | MLQKARQTLEFYPCTSEEIDHEDITKDKTSTVEACLPL | | |
| | | ELTKNESCLNSRETSFITNGSCLASRKTSFMMALCLSSI | | |
| | | YEDLKMYQVEFKTMNAKLLMDPKRQIFLDQNMLAVI | | |
| | | DELMQALNFNSETVPQKSSLEEPDFYKTKIKLCILLHA | | |
| | | FRIRAVTIDRVMSYLNAS* | | |

In some embodiments, the payload of the present disclosure may be a cytokine fused to a cytokine receptor. In one embodiment, the payload may be IL15 fused to IL15 Receptor alpha subunit. A unique feature of IL15 mediated activation is the mechanism of trans-presentation in which IL15 is presented as a complex with the alpha subunit of IL15 receptor (IL15Ra) that binds to and activates membrane bound IL15 beta/gamma receptor, either on the same cell or a different cell. The IL15/IL15Ra complex is much more effective in activating IL15 signaling, than IL15 by itself. In one embodiment, the may be a IL15/IL15Ra fusion polypeptide described in US patent publication NO. US20160158285A1 (the contents of which are incorporated herein by reference in their entirety). The IL15 receptor alpha comprises an extracellular domain called the sushi domain that is considered to contain most of the structural elements necessary for binding to IL15. Thus, in some embodiments, the payload may be the IL15/IL15Ra sushi domain fusion polypeptide described in US Patent Publication NO. US20090238791A1 (the contents of which are incorporated herein by reference in their entirety). The effector modules containing IL15/IL15Ra, and/or DD-IL15/IL15Ra sushi domain may be designed to be secreted (using e.g. IL2 signal sequence) or membrane bound (using e.g. IgE or CD8a signal sequence).

In some embodiments, the payloads may proteins that induce the expression of IL15 such as proteins in the STING pathway and/or Type I interferons. In one aspect, the payload may be STING agonist. In one aspect, such expressing IL15 directly as a payload or using payloads that induce IL15 expression may use to regulate the tumor-infiltrating lymphocyte numbers in the tumor microenvironment. Any of the payloads described by Carrero et al. to induce IL15 expression may be used herein (Carrero et al. 2019 PNAS January 2019, 116 (2) 599-608; DOI: 10.1073/pnas.1814642116; the contents of which are incorporated herein by reference in their entirety).

IL15/IL15Ra construct components are described in Table 8. IL15/IL15Ra constructs are described in Table 9. In Table 9, the amino acid sequences may comprise a stop codon at the end which is denoted in Table 9 with a “*”.

TABLE 8
IL15/IL15Ra construct components

| | | AA SEQ | NA SEQ |
|---|---|---|---|
| Description | AA Sequence | ID NO. | ID NO. |
| IgE Leader | MDWTWILFLVAAATRVHS | 244 | 245 |
| IL15 (aa 49-162 of | NWVNVISDLKKIEDLIQSMHIDATLYIESDVHPSC | 246 | 247 |
| WT) | KVTAMKCFLLELQVISLESGDASIHDTVENLIILA | | |
| | NNSLSSNGNVTESGCKECEELEEKNIKEFLQSFVH | | |
| | IVQMFINTS | | |
| IL15Ra (31-267 of | ITCPPPMSVEHADIWVKSYSLYSRERYICNSGFKR | 248 | 249 |
| WT) | KAGTSSLTECVLNKATNVAHWTTPSLKCIRDPAL | | |
| | VHQRPAPPSTVTTAGVTPQPESLSPSGKEPAASSP | | |
| | SSNNTAATTAAIVPGSQLMPSKSPSTGTTEISSHES | | |
| | SHGTPSQTTAKNWELTASASHQPPGVYPQGHSDT | | |
| | TVAISTSTVLLCGLSAVSLLACYLKSRQTPPLASV | | |
| | EMEAMEALPVTWGTSSRDEDLENCSHHL | | |
| SG3-(SG4)3-SG3- | SGGGSGGGGSGGGGSGGGGSGGGSLQ | 250 | 251 |
| SLQ | | | |
| CD8α leader | MALPVTALLLPLALLLHAARP | 99 | 100 |
| Linker (G4S)3 | GGGGSGGGGSGGGGS | 125 | 126 |

TABLE 9
IL15/IL15Ra constructs

| | | | AA | NA |
|---|---|---|---|---|
| | | | SEQ | SEQ |
| Construct | | | ID | ID |
| ID | Description | AA Sequence | NO. | NO. |
| OT-001734 | IgE Leader; | MDWTWILFLVAAATRVHSNWVNVISDLKKIEDLI | 252 | 253 |
| | IL15 (aa 49-162 | QSMHIDATLYTESDVHPSCKVTAMKCFLLELQVI | | |
| | of WT); | SLESGDASIHDTVENLIILANNSLSSNGNVTESGC | | |
| | Artificial linker | KECEELEEKNIKEFLQSFVHIVQMFINTSSGGGSG | | |
| | (SG3-(SG4)3- | GGGSGGGGSGGGGSGGGSLQITCPPPMSVEHADI | | |
| | SG3-SLQ); | WVKSYSLYSRERYICNSGFKRKAGTSSLTECVLN | | |
| | IL15Ra (31-267 | KATNVAHWTTPSLKCIRDPALVHQRPAPPSTVIT | | |
| | of WT); Linker | AGVTPQPESLSPSGKEPAASSPSSNNTAATTAAIV | | |
| | (GS); ER (aa | PGSQLMPSKSPSTGTTEISSHESSHGTPSQTTAKN | | |
| | 305-549 of WT, | WELTASASHQPPGVYPQGHSDTTVAISTSTVLLC | | |
| | R335G, | GLSAVSLLACYLKSRQTPPLASVEMEAMEALPVT | | |
| | L384M, | WGTSSRDEDLENCSHHLGSSLALSLTADQMVSAL | | |
| | M421G, | LDAEPPILYSEYDPTGPFSEASMMGLLTNLADREL | | |
| | G521R, E523 G, | VHMINWAKRVPGFVDLTLHDQVHLLECAWMEIL | | |
| | Y537S, | MIGLVWRSMEHPGKLLFAPNLLLDRNQGKCVEG | | |
| | A546T); stop | GVEIFDMLLATSSRFRMMNLQGEEFVCLKSIILLN | | |
| | | SGVYTFLSSTLKSLEEKDHIHRVLDKITDTLIHLM | | |
| | | AKAGLTLQQQHQRLAQLLLILSHIRHMSNKRMG | | |
| | | HLYSMKCKNVVPLSDLLLEMLDTHRL* | | |
| OT-001733 | IgE Leader; | MDWTWILFLVAAATRVHSNWVNVISDLKKIEDLI | 254 | 255 |
| | IL15 (aa 49-162 | QSMHIDATLYTESDVHPSCKVTAMKCFLLELQVI | | |
| | of WT); | SLESGDASIHDTVENLIILANNSLSSNGNVTESGC | | |
| | Artificial linker | KECEELEEKNIKEFLQSFVHIVQMFINTSSGGGSG | | |
| | (SG3-(SG4)3- | GGGSGGGGSGGGGSGGGSLQITCPPPMSVEHADI | | |
| | SG3-SLQ); | WVKSYSLYSRERYICNSGFKRKAGTSSLTECVLN | | |
| | IL15Ra (31-267 | KATNVAHWTTPSLKCIRDPALVHQRPAPPSTVIT | | |
| | of WT); Linker | AGVTPQPESLSPSGKEPAASSPSSNNTAATTAAIV | | |
| | (GS); ER (aa | PGSQLMPSKSPSTGTTEISSHESSHGTPSQTTAKN | | |
| | 305-549 of WT, | WELTASASHQPPGVYPQGHSDTTVAISTSTVLLC | | |
| | L384M; | GLSAVSLLACYLKSRQTPPLASVEMEAMEALPVT | | |
| | M421G; T431I; | WGTSSRDEDLENCSHHLGSSLALSLTADQMVSAL | | |
| | G521R,Y537S); | LDAEPPILYSEYDPTRPFSEASMMGLLTNLADREL | | |
| | stop | VHMINWAKRVPGFVDLTLHDQVHLLECAWMEIL | | |
| | | MIGLVWRSMEHPGKLLFAPNLLLDRNQGKCVEG | | |
| | | GVEIFDMLLAISSRFRMMNLQGEEFVCLKSIILLN | | |
| | | SGVYTFLSSTLKSLEEKDHIHRVLDKITDTLIHLM | | |
| | | AKAGLTLQQQHQRLAQLLLILSHIRHMSNKRMEH | | |
| | | LYSMKCKNVVPLSDLLLEMLDAHRL* | | |
| OT-001732 | IgE Leader; | MDWTWILFLVAAATRVHSNWVNVISDLKKIEDLI | 256 | 257 |
| | IL15 (aa 49-162 | QSMHIDATLYTESDVHPSCKVTAMKCFLLELQVI | | |
| | of WT); | SLESGDASIHDTVENLIILANNSLSSNGNVTESGC | | |
| | Artificial linker | KECEELEEKNIKEFLQSFVHIVQMFINTSSGGGSG | | |
| | (SG3-(SG4)3- | GGGSGGGGSGGGGSGGGSLQITCPPPMSVEHADI | | |
| | SG3-SLQ); | WVKSYSLYSRERYICNSGFKRKAGTSSLTECVLN | | |
| | IL15Ra (31-267 | KATNVAHWTTPSLKCIRDPALVHQRPAPPSTVIT | | |
| | of WT); Linker | AGVTPQPESLSPSGKEPAASSPSSNNTAATTAAIV | | |
| | (GS); ER(aa | PGSQLMPSKSPSTGTTEISSHESSHGTPSQTTAKN | | |
| | 305-549 of WT, | WELTASASHQPPGVYPQGHSDTTVAISTSTVLLC | | |
| | L384M, | GLSAVSLLACYLKSRQTPPLASVEMEAMEALPVT | | |
| | N413D, | WGTSSRDEDLENCSHHLGSSLALSLTADQMVSAL | | |
| | M421G, | LDAEPPILYSEYDPTRPFSEASMMGLLTNLADREL | | |
| | G521R, | VHMINWAKRVPGFVDLTLHDQVHLLECAWMEIL | | |
| | Y537S); stop | MIGLVWRSMEHPGKLLFAPNLLLDRDQGKCVEG | | |
| | | GVEIFDMLLATSSRFRMMNLQGEEFVCLKSIILLN | | |
| | | SGVYTFLSSTLKSLEEKDHIHRVLDKITDTLIHLM | | |
| | | AKAGLTLQQQHQRLAQLLLILSHIRHMSNKRMEH | | |
| | | LYSMKCKNVVPLSDLLLEMLDAHRL* | | |
| OT-001735 | IgE Leader; | MDWTWILFLVAAATRVHSNWVNVISDLKKIEDLI | 258 | 259 |
| | IL15 (aa 49-162 | QSMHIDATLYTESDVHPSCKVTAMKCFLLELQVI | | |
| | of WT); | SLESGDASIHDTVENLIILANNSLSSNGNVTESGC | | |
| | Artificial linker | KECEELEEKNIKEFLQSFVHIVQMFINTSSGGGSG | | |
| | (SG3-(SG4)3- | GGGSGGGGSGGGGSGGGSLQITCPPPMSVEHADI | | |
| | SG3-SLQ); | WVKSYSLYSRERYICNSGFKRKAGTSSLTECVLN | | |
| | IL15Ra (31-267 | KATNVAHWTTPSLKCIRDPALVHQRPAPPSTVIT | | |
| | of WT); Linker | AGVTPQPESLSPSGKEPAASSPSSNNTAATTAAIV | | |
| | (GS); ER (aa | PGSQLMPSKSPSTGTTEISSHESSHGTPSQTTAKN | | |
| | 305-549 of WT, | WELTASASHQPPGVYPQGHSDTTVAISTSTVLLC | | |
| | L384M, | GLSAVSLLACYLKSRQTPPLASVEMEAMEALPVT | | |
| | M421G, | WGTSSRDEDLENCSHHLGSSLALSLTADQMVSAL | | |
| | N519S, G521R, | LDAEPPILYSEYDPTRPFSEASMMGLLTNLADREL | | |
| | Y537S); stop | VHMINWAKRVPGFVDLTLHDQVHLLECAWMEIL | | |
| | | MIGLVWRSMEHPGKLLFAPNLLLDRNQGKCVEG | | |
| | | GVEIFDMLLATSSRFRMMNLQGEEFVCLKSIILLN | | |
| | | SGVYTFLSSTLKSLEEKDHIHRVLDKITDTLIHLM | | |
| | | AKAGLTLQQQHQRLAQLLLILSHIRHMSSKRMEH | | |
| | | LYSMKCKNVVPLSDLLLEMLDAHRL* | | |
| OT-001731 | IgE Leader; | MDWTWILFLVAAATRVHSNWVNVISDLKKIEDLI | 260 | 261 |
| | IL 15 (aa 49-162 | QSMHIDATLYTESDVHPSCKVTAMKCFLLELQVI | | |
| | of WT); | SLESGDASIHDTVENLIILANNSLSSNGNVTESGC | | |
| | Artificial linker | KECEELEEKNIKEFLQSFVHIVQMFINTSSGGGSG | | |
| | (SG3-(SG4)3- | GGGSGGGGSGGGGSGGGSLQITCPPPMSVEHADI | | |
| | SG3-SLQ); | WVKSYSLYSRERYICNSGFKRKAGTSSLTECVLN | | |
| | IL15Ra (31-267 | KATNVAHWTTPSLKCIRDPALVHQRPAPPSTVIT | | |
| | of WT); Linker | AGVTPQPESLSPSGKEPAASSPSSNNTAATTAAIV | | |
| | (GS); ER (305- | PGSQLMPSKSPSTGTTEISSHESSHGTPSQTTAKN | | |
| | 549 of WT, | WELTASASHQPPGVYPQGHSDTTVAISTSTVLLC | | |
| | L384M, | GLSAVSLLACYLKSRQTPPLASVEMEAMEALPVT | | |
| | M421G, | WGTSSRDEDLENCSHHLGSSLALSLTADQMVSAL | | |
| | Q502R, | LDAEPPILYSEYDPTRPFSEASMMGLLTNLADREL | | |
| | G521R, | VHMINWAKRVPGFVDLTLHDQVHLLECAWMEIL | | |
| | Y537S); stop | MIGLVWRSMEHPGKLLFAPNLLLDRNQGKCVEG | | |
| | | GVEIFDMLLATSSRFRMMNLQGEEFVCLKSIILLN | | |
| | | SGVYTFLSSTLKSLEEKDHIHRVLDKITDTLIHLM | | |
| | | AKAGLTLQQQHRRLAQLLLILSHIRHMSNKRMEH | | |
| | | LYSMKCKNVVPLSDLLLEMLDAHRL* | | |
| OT-001730 | IgE Leader; | MDWTWILFLVAAATRVHSNWVNVISDLKKIEDLI | 262 | 263 |
| | IL15 (aa 49-162 | QSMHIDATLYTESDVHPSCKVTAMKCFLLELQVI | | |
| | of WT); | SLESGDASIHDTVENLIILANNSLSSNGNVTESGC | | |
| | Artificial linker | KECEELEEKNIKEFLQSFVHIVQMFINTSSGGGSG | | |
| | (SG3-(SG4)3- | GGGSGGGGSGGGGSGGGSLQITCPPPMSVEHADI | | |
| | SG3-SLQ); | WVKSYSLYSRERYICNSGFKRKAGTSSLTECVLN | | |
| | IL15Ra (31-267 | KATNVAHWTTPSLKCIRDPALVHQRPAPPSTVIT | | |
| | of WT); Linker | AGVTPQPESLSPSGKEPAASSPSSNNTAATTAAIV | | |
| | (GS); ER (aa | PGSQLMPSKSPSTGTTEISSHESSHGTPSQTTAKN | | |
| | 305-549 of WT, | WELTASASHQPPGVYPQGHSDTTVAISTSTVLLC | | |
| | T371A, | GLSAVSLLACYLKSRQTPPLASVEMEAMEALPVT | | |
| | L384M, | WGTSSRDEDLENCSHHLGSSLALSLTADQMVSAL | | |
| | M421G, | LDAEPPILYSEYDPTRPFSEASMMGLLTNLADREL | | |
| | N519S, G521R, | VHMINWAKRVPGFVDLALHDQVHLLECAWMEIL | | |
| | Y537S); stop | MIGLVWRSMEHPGKLLFAPNLLLDRNQGKCVEG | | |
| | | GVEIFDMLLATSSRFRMMNLQGEEFVCLKSIILLN | | |
| | | SGVYTFLSSTLKSLEEKDHIHRVLDKITDTLIHLM | | |
| | | AKAGLTLQQQHQRLAQLLLILSHIRHMSSKRMEH | | |
| | | LYSMKCKNVVPLSDLLLEMLDAHRL* | | |
| OT-001729 | IgE Leader; | MDWTWILFLVAAATRVHSNWVNVISDLKKIEDLI | 264 | 265 |
| | IL15 (aa 49-162 | QSMHIDATLYTESDVHPSCKVTAMKCFLLELQVI | | |
| | of WT); | SLESGDASIHDTVENLIILANNSLSSNGNVTESGC | | |
| | Artificial linker | KECEELEEKNIKEFLQSFVHIVQMFINTSSGGGSG | | |
| | (SG3-(SG4)3- | GGGSGGGGSGGGGSGGGSLQITCPPPMSVEHADI | | |
| | SG3-SLQ); | WVKSYSLYSRERYICNSGFKRKAGTSSLTECVLN | | |
| | IL15Ra (31-267 | KATNVAHWTTPSLKCIRDPALVHQRPAPPSTVIT | | |
| | of WT); Linker | AGVTPQPESLSPSGKEPAASSPSSNNTAATTAAIV | | |
| | (GS); ER (aa | PGSQLMPSKSPSTGTTEISSHESSHGTPSQTTAKN | | |
| | 305-549 of WT, | WELTASASHQPPGVYPQGHSDTTVAISTSTVLLC | | |
| | S305N, | GLSAVSLLACYLKSRQTPPLASVEMEAMEALPVT | | |
| | L384M, | WGTSSRDEDLENCSHHLGSNLALSLTADQMVSA | | |
| | MV421G, | LLDAEPPILYSEYDPTRPFSEASMMGLLTNLADRE | | |
| | G442V, | LVHMINWAKRVPGFVDLTLHDQVHLLECAWMEI | | |
| | G521R, | LMIGLVWRSMEHPGKLLFAPNLLLDRNQGKCVE | | |
| | Y537S); stop | GGVEIFDMLLATSSRFRMMNLQVEEFVCLKSTILL | | |
| | | NSGVYTFLSSTLKSLEEKDHIHRVLDKITDTLIHL | | |
| | | MAKAGLTLQQQHQRLAQLLLILSHIRHMSNKRM | | |
| | | EHLYSMKCKNVVPLSDLLLEMLDAHRL* | | |
| OT-001728 | IgE Leader; | MDWTWILFLVAAATRVHSNWVNVISDLKKIEDLI | 266 | 267 |
| | IL 15 (aa 49-162 | QSMHIDATLYTESDVHPSCKVTAMKCFLLELQVI | | |
| | of WT); | SLESGDASIHDTVENLIILANNSLSSNGNVTESGC | | |
| | Artificial linker | KECEELEEKNIKEFLQSFVHIVQMFINTSSGGGSG | | |
| | (SG3-(SG4)3- | GGGSGGGGSGGGGSGGGSLQITCPPPMSVEHADI | | |
| | SG3-SLQ); | WVKSYSLYSRERYICNSGFKRKAGTSSLTECVLN | | |
| | IL15Ra (31-267 | KATNVAHWTTPSLKCIRDPALVHQRPAPPSTVIT | | |
| | of WT); Linker | AGVTPQPESLSPSGKEPAASSPSSNNTAATTAAIV | | |
| | (GS); ER (aa | PGSQLMPSKSPSTGTTEISSHESSHGTPSQTTAKN | | |
| | 305-549 of WT, | WELTASASHQPPGVYPQGHSDTTVAISTSTVLLC | | |
| | R335G, | GLSAVSLLACYLKSRQTPPLASVEMEAMEALPVT | | |
| | L384M, | WGTSSRDEDLENCSHHLGSSLALSLTADQMVSAL | | |
| | M421G, | LDAEPPILYSEYDPTGPFSEASMMGLLTNLADREL | | |
| | N519S, G521R, | VHMINWAKRVPGFVDLTLHDQVHLLECAWMEIL | | |
| | Y537S); stop | MIGLVWRSMEHPGKLLFAPNLLLDRNQGKCVEG | | |
| | | GVEIFDMLLATSSRFRMMNLQGEEFVCLKSIILLN | | |
| | | SGVYTFLSSTLKSLEEKDHIHRVLDKITDTLIHLM | | |
| | | AKAGLTLQQQHQRLAQLLLILSHIRHMSSKRMEH | | |
| | | LYSMKCKNVVPLSDLLLEMLDAHRL* | | |
| OT-001727 | CD8a leader | MALPVTALLLPLALLLHAARPGSSLALSLTADQM | 268 | 269 |
| | sequence; | VSALLDAEPPILYSEYDPTRPFSEASMMGLLTNLA | | |
| | Linker (GS); | DRELVHMINWAKRVPGFVDLALHDQVHLLECA | | |
| | ER (aa 305-549 | WMEILMIGLVWRSMEHPGKLLFAPNLLLDRNQG | | |
| | of WT, T371A, | KCVEGGVEIFDMLLATSSRFRMMNLQGEEFVCL | | |
| | L384M, | KSIILLNSGVYTFLSSTLKSLEEKDHIHRVLDKITD | | |
| | M421G, | TLIHLMAKAGLTLQQQHQRLAQLLLILSHIRHMSS | | |
| | N519S, G521R, | KRMEHLYSMKCKNVVPLSDLLLEMLDAHRLGSN | | |
| | Y537S); Linker | WVNVISDLKKIEDLIQSMHIDATLYTESDVHPSCK | | |
| | (GS); IL15 (aa | VTAMKCFLLELQVISLESGDASIHDTVENLIILAN | | |
| | 49-162 of WT); | NSLSSNGNVTESGCKECEELEEKNIKEFLQSFVHI | | |
| | Artificial linker | VQMFINTSSGGGSGGGGSGGGGSGGGGSGGGSL | | |
| | (SG3-(SG4)3- | QITCPPPMSVEHADIWVKSYSLYSRERYICNSGFK | | |
| | SG3-SLQ); | RKAGTSSLTECVLNKATNVAHWTTPSLKCIRDPA | | |
| | IL15Ra (31-267 | LVHQRPAPPSTVTTAGVTPQPESLSPSGKEPAASS | | |
| | of WT); stop | PSSNNTAATTAAIVPGSQLMPSKSPSTGTTEISSHE | | |
| | | SSHGTPSQTTAKNWELTASASHQPPGVYPQGHSD | | |
| | | TTVAISTSTVLLCGLSAVSLLACYLKSRQTPPLAS | | |
| | | VEMEAMEALPVTWGTSSRDEDLENCSHHL* | | |

In some embodiments, payloads fused to the DDs of the disclosure may be an inhibitor of an immunosuppressive molecule such as TGF-beta and IDO.

In some embodiments, payloads of the present disclosure may comprise SRE regulated safety switches that can eliminate adoptively transferred cells in the case of severe toxicity, thereby mitigating the adverse effects of T cell therapy. Adoptively transferred T cells in immunotherapy may attack normal cells in response to normal tissue expression of TAA. Even on-tumor target activity of adoptively transferred T cells can result in toxicities such as tumor lysis syndrome, cytokine release syndrome and the related macrophage activation syndrome. Safety switches may be utilized to eliminate inappropriately activated adoptively transferred cells by induction of apoptosis or by immunosurveillance.

In some embodiments, payloads of the present disclosure may comprise inducible killer/suicide genes that acts as a safety switch. The killer/suicide gene when introduced into adoptively transferred immune cells, could control their alloreactivity. The killer/suicide gene may be an apoptotic gene (e.g., any Caspase gene) which allows conditional apoptosis of the transduced cells by administration of a non-therapeutic ligand of the SRE (e.g., DD).

In some embodiments, the payloads of the present disclosure may be Caspase 9. In some instances, Caspase 9 may be modified to have low basal expression and lacking the Caspase recruitment domain (CARD) (SEQ ID NO. 26 and SEQ ID NO. 28 of U.S. Pat. No. 9,434,935B2; the contents of which are incorporated by reference in their entirety).

In one embodiment, the payload of the present disclosure is a suicide gene system, iCasp9/Chemical induced dimerization (CID) system which consists of a polypeptide derived from the Caspase9 gene fused to a drug binding domain derived from the human FK506 protein. Administration of bioinert, small molecule AP1903 (rimiducid), induces cross linking of the drug binding domains and dimerization of the fusion protein and in turn the dimerization of Caspase 9. This results in the activation of downstream effector Caspase 3 and subsequent induction of cellular apoptosis (Straathof et al., Blood, 2005, 105: 4247-4254; incorporated herein by reference in its entirety). Preclinical trials using CART including an iCasp9 gene have shown effective elimination of CAR T cells in vivo in mouse models and demonstrate the potential efficacy of this approach. (Budde et al, Plos One, 2013, 8: e82742.10.1371; Hoyos et al., Leukemia, 2010; 24(6):1160-1170).

In some instances, the iCasp9/CID system has been shown to have a basal rate of dimerization even in the absence of rimiducid, resulting in unintended cell death. Regulating the expression levels of iCasp9/CID is critical for maximizing the efficacy of iCasp9/CID system. Biocircuits of the present disclosure and/or any of their components may be utilized in regulating or tuning the iCasp9/CID system to optimize its utility. Other examples of proteins used in dimerization-induced apoptosis paradigm may include, but are not limited to Fas receptor, the death effector domain of Fas-associated protein, FADD, Caspase 1, Caspase 3, Caspase 7 and Caspase 8. (Belshaw P. J. et al, Chem Biol., 996, 3:731-738; MacCorkle R. A. et al, Proc Natl Acad Sci, 1998, 95:3655-3660; Spencer, D. M. et al., Curr Biol. 1996; 6:839-847; the contents of each of which are incorporated herein by reference in their entirety).

In some embodiments, the safety switch of the present disclosure may comprise a metabolic enzyme, such as herpes simplex virus thymidine kinase (HSV-TK) and cytosine deaminase (CD). HSV-TK phosphorylates nucleoside analogs, including acyclovir and ganciclovir (GCV) to generate triphosphate form of nucleosides. When incorporated into DNA, it leads to chain termination and cell death. Unlike the mammalian thymidine kinase, HSV-TK is characterized by 1000-fold higher affinity to nucleoside analogs such as GCV, making it suitable for use as a suicide gene in mammalian cells. Cytosine deaminase (CD) can converts 5-fluorocytosine (5-FC) into the cytotoxic 5-fluorouracil (5-FU) (Tiraby et al., FEMS Lett., 1998, 167: 41-49).

In some embodiments, the safety switch of the present disclosure may comprise a CYP4B1 mutant (as suicide gene), which may be co-expressed in a CAR engineered T cells (Roellecker et al., Gen Ther., 2016, May 19, doi: 10.1038/gt.2016.38.).

In some embodiments, the payload of the present disclosure may comprise a fusion construct that can induce cell death, for example, a polypeptide with the formula of St-R1-S1-Q-52-R2, wherein the St is a stalk sequence, R1/2 and Q are different epitopes; and S1/2 are optional spacer sequences (See International Patent Publication NO. WO2013153391; the content of which are incorporated herein by reference in their entirety).

In some embodiments, safety switch may be mediated by therapeutic antibodies which specifically bind to an antigen that is expressed in the plasma membrane of adoptively transferred cells. The antigen-antibody interaction allows cell removal after administration of a specific monoclonal antibody against the antigen. As non-limiting examples, payloads of the present disclosure may comprise the antigen and antibody pair used to mediate safety switch such as CD20 and anti-CD20 antibody (Griffioen et al., Haematologica, 2009, 94:1316-1320), a protein tag and anti-tag antibody (Kieback et al., Natl. Acad. Sci. USA., 2008, 105: 623-628), a compact suicide gene (RQR8) combining epitopes from CD34 (as a marker moiety) and CD20 (as a suicide moiety) which enables CD34 selection, cell tracking, as well as cell deletion after anti-CD20 monoclonal antibody administration (Philip et al., Blood, 2014, 124: 1277-1287); truncated human EGFR polypeptide and anti-EGFR monoclonal antibody (Wang et al., Blood, 2011, 118:1255-1263); and a compact polypeptide safety switch having a structural formula as discussed in U.S. Patent Application Publication NO. US20150093401; the contents of each of which are incorporated herein by reference in their entirety.

In one embodiment, the payload may be a regulated CD28 superagonist ScFv, which is capable of exogenously controlling the co-stimulation of TCR or 4-1BB CAR engineered T-cells

In one embodiment, the payload is c-myc. Regulated expression of c-myc may be utilized to confer activated metabolic state in T-cells

In some embodiments, the promoter of the disclosure may be a Tet-ON promoter. Combination of the transcription regulation Tet system with the DDs permits simultaneous control of gene expression and protein stability. Any of the dual-Tet ON-DD systems described by Pedone et al. (2018) doi: https://doi.org/10.1101/404699 may be useful in the present disclosure (the contents of which are herein incorporated by reference in their entirety).

In some embodiments, the payload may be CD28 co-receptors and/or 4-1BBL, which may be used as a costimulatory signal for T-cells.

In some embodiments, the payload of the disclosure may be a homing signal molecule which includes a D3 exodomain, an IgG1 hinge, and a CD6 transmembrane and signaling domains. Biocircuits comprising such payloads may be successful in guiding T cells to activated leukocyte cell adhesion molecule (ALCAM) positive tumors such as but not limited to glioblastomas. Any of the homing signal molecules taught by Samaha et al. (2018) Nature 561, 331-337 may be useful in the present disclosure (the contents of which are incorporated by reference in their entirety).

In one embodiment, regulated cytokines may enable CAR-T in solid tumors to overcome stromal barriers by improving tumor homing, reducing immunosuppression, and reducing tumor promoting conditions. In one embodiment, regulated cytokines may enable CAR-T in solid tumors to overcome Antigen negative escape by promoting epitope spreading, antigen presenting cell trafficking, activation and licensing. In one embodiment, regulated cytokines may enable CAR-T in solid tumors to overcome Antigen positive escape by improving expansion, increasing persistence, reducing exhaustion of T cells. In one embodiment, regulated cytokines enable local, on demand production of cytokines can safely improve efficacy. In one embodiment, regulated cytokines enable pulsatile production that can reduce feedback inhibition of cytokine signaling. In one embodiment, regulated cytokines can reduce senescence or exhaustion. In one embodiment, regulated cytokines enable on demand expression in patient which may reduce any effect of cytokine on cell phenotype during product manufacturing.

In some embodiments, the payload may be a cytokine based CAR, wherein the antigen binding domain of the CAR is substituted by a cytokine which may bind to its cognate receptor. In one embodiment, the payload is humanized interleukin-13 receptor α2 (IL-13Rα2) chimeric antigen receptors (CARs), Hu07BBz and Hu08BBz, that recognized human IL-13Rα2, but not IL-13Rα1. The efficacy of such CAR may further be improved by PD-1 and TIM-3 blockade. Any of the CARs described in Yin et al. Mol Ther Oncolytics. 2018 Aug. 28; 11:20-38, may be useful in the present invention (the contents of which are incorporated by reference in their entirety).

### 4. Genomic Editing Systems

In some embodiments, payloads of the present disclosure may be components of gene editing systems including a CRISPR (Clustered Regularly Interspaced Short Palindromic Repeats), CRISPR enzyme (Cas9), CRISPR-Cas9 or CRISPR system and CRISPR-CAS9 complex. It may also be other genomic editing systems, such as Zinc finger nucleases, TALEN (Transcription activator-like effector-based nucleases) and meganucleases.

### Additional Features

The effector module of the present disclosure may further comprise a signal sequence which regulates the distribution of the payload, a cleavage and/or processing feature which facilitate cleavage of the payload from the effector module construct, a targeting and/or penetrating signal which can regulate the cellular localization of the effector module, and/or one or more linker sequences which link different components (e.g. a DD and a payload) of the effector module.

### 1. Signal Sequences

In addition to the SRE (e.g., DD) and payload region, effector modules of the disclosure may further comprise one or more signal sequences. Signal sequences (sometimes referred to as signal peptides, targeting signals, target peptides, localization sequences, transit peptides, leader sequences or leader peptides) direct proteins (e.g., the effector module of the present disclosure) to their designated cellular and/or extracellular locations. Protein signal sequences play a central role in the targeting and translocation of nearly all secreted proteins and many integral membrane proteins.

A signal sequence is a short (5-30 amino acids long) peptide present at the N-terminus of the majority of newly synthesized proteins that are destined towards a particular location. Signal sequences can be recognized by signal recognition particles (SRPs) and cleaved using type I and type II signal peptide peptidases. Signal sequences derived from human proteins can be incorporated as a regulatory module of the effector module to direct the effector module to a particular cellular and/or extracellular location. These signal sequences are experimentally verified and can be cleaved (Zhang et al., Protein Sci. 2004, 13:2819-2824).

In some embodiments, a signal sequence may be, although not necessarily, located at the N-terminus or C-terminus of the effector module, and may be, although not necessarily, cleaved off the desired effector module to yield a “mature” payload, i.e., an immunotherapeutic agent as discussed herein.

In some examples, a signal sequence may be a secreted signal sequence derived from a naturally secreted protein, and its variant thereof. In some instances, the secreted signal sequences may be cytokine signal sequences such as, but not limited to, IL2 signal sequence comprising amino acid of SEQ ID NO. 270, encoded by the nucleotide of SEQ ID NOs. 271-274 and/or p40 signal sequence comprising the amino acid sequence of SEQ ID NO. 101, encoded by the nucleotide of SEQ ID NOs. 102, 275-282 or a GMCSF leader sequence comprising the amino acid sequence of SEQ ID NOs. 283-285.

In some instances, signal sequences directing the payload to the surface membrane of the target cell may be used. Expression of the payload on the surface of the target cell may be useful to limit the diffusion of the payload to non-target in vivo environments, thereby potentially improving the safety profile of the payloads. Additionally, the membrane presentation of the payload may allow for physiologically and qualitative signaling as well as stabilization and recycling of the payload for a longer half-life. Membrane sequences may be the endogenous signal sequence of the N terminal component of the payload. Optionally, it may be desirable to exchange this sequence for a different signal sequence. Signal sequences may be selected based on their compatibility with the secretory pathway of the cell type of interest so that the payload is presented on the surface of the T cell. In some embodiments, the signal sequence may be IgE signal sequence comprising amino acid SEQ ID NO. 244 and nucleotide sequence of SEQ ID NO. 245, a CD8a signal sequence comprising amino acid SEQ ID NO. 99 and nucleotide sequence of SEQ ID NOs. 100, 286-292 or an IL15Ra signal sequence, comprising amino acid SEQ ID NO. 293, encoded by SEQ ID NO. 294.

Other examples of signal sequences include, a variant may be a modified signal sequence discussed in U.S. Pat. Nos. 8,148,494, 8,258,102, 9,133,265, 9,279,007, and U.S. Patent Application Publication No. 2007/0141666; and International Patent Publication No. WO1993/018181; the contents of each of which are incorporated herein by reference in their entirety. In other examples, a signal sequence may be a heterogeneous signal sequence from other organisms such as virus, yeast and bacteria, which can direct an effector module to a particular cellular site, such as a nucleus (e.g., EP 1209450). Other examples may include Aspartic Protease (NSP24) signal sequences from Trichoderma that can increase secretion of fused protein such as enzymes (e.g., U.S. Pat. No. 8,093,016 to Cervin and Kim), bacterial lipoprotein signal sequences (e.g., International Patent Publication No. WO1991/09952 to Lau and Rioux), E. coli enterotoxin II signal peptides (e.g., U.S. Pat. No. 6,605,697 to Kwon et al.), E. coli secretion signal sequence (e.g., U.S. Patent Publication No. 2016/090404 to Malley et al.), a lipase signal sequence from a methylotrophic yeast (e.g., U.S. Pat. No. 8,975,041), and signal peptides for DNases derived from Coryneform bacteria (e.g., U.S. Pat. No. 4,965,197); the contents of each of which are incorporated herein by reference in their entirety.

Signal sequences may also include nuclear localization signals (NLSs), nuclear export signals (NESs), polarized cell tubulo-vesicular structure localization signals (See, e.g., U.S. Pat. No. 8,993,742; Cour et al., Nucleic Acids Res. 2003, 31(1): 393-396; the contents of each of which are incorporated herein by reference in their entirety), extracellular localization signals, signals to subcellular locations (e.g. lysosome, endoplasmic reticulum, golgi, mitochondria, plasma membrane and peroxisomes, etc.) (See, e.g., U.S. Pat. No. 7,396,811; and Negi et al., Database, 2015, 1-7; the contents of each of which are incorporated herein by reference in their entirety).

In some embodiments, signal sequences of the present disclosure, include without limitation, any of those taught in Table 6 of co-pending commonly owned U.S. Provisional Patent Application No. 62/320,864 filed on Apr. 11, 2016, or in U.S. Provisional Application No. 62/466,596 filed Mar. 3, 2017, and the International Publication WO2017/180587, the contents of each of which are incorporated herein by reference in their entirety.

Any of the signal sequences described in the present disclosure that include a Methionine residue at position 1 of the sequence may also be utilized without the methionine at position 1 of the amino acid sequence. For example, a CD8a signal sequence, MALPVTALLLPLALLLHAARP (SEQ ID NO. 99; encoded by SEQ ID NO. 100 or SEQ ID NO. 367) comprising a methionine sequence may also be utilized without the methionine at position one i.e. as ALPVTALLLPLALLLHAARP (SEQ ID NO. 408; encoded by 409).

### 2. Cleavage Sites

In some embodiments, the effector module comprises a cleavage and/or processing feature. The effector module of the present disclosure may include at least one protein cleavage signal/site. The protein cleavage signal/site may be located at the N-terminus, the C-terminus, at any space between the N- and the C-termini such as, but not limited to, half-way between the N- and C-termini, between the N-terminus and the half-way point, between the half-way point and the C-terminus, and combinations thereof.

The effector module may include one or more cleavage signal(s)/site(s) of any proteinases. The proteinases may be a serine proteinase, a cysteine proteinase, an endopeptidase, a dipeptidase, a metalloproteinase, a glutamic proteinase, a threonine proteinase and an aspartic proteinase. In some aspects, the cleavage site may be a signal sequence of furin, actinidain, calpain-1, carboxypeptidase A, carboxypeptidase P, carboxypeptidase Y, caspase-1, caspase-2, caspase-3, caspase-4, caspase-5, caspase-6, caspase-7, caspase-8, caspase-9, caspase-10, cathepsin B, cathepsin C, cathepsin G, cathepsin H, cathepsin K, cathepsin L, cathepsin S, cathepsin V, clostripain, chymase, chymotrypsin, elastase, endoproteinase, enterokinase, factor Xa, formic acid, granzyme B, Matrix metallopeptidase-2, Matrix metallopeptidase-3, pepsin, proteinase K, SUMO protease, subtilisin, TEV protease, thermolysin, thrombin, trypsin and TAGZyme.

In one embodiment, the cleavage site is a furin cleavage site comprising the amino acid sequence SARNRQKRS (SEQ ID NO. 295, encoded by nucleotide sequence of SEQ ID NO. 296), or a revised furin cleavage site comprising the amino acid sequence ARNRQKRS (SEQ ID NO. 297, encoded by nucleotide sequence of SEQ ID NO. 298); or a modified furin site comprising the amino acid sequence ESRRVRRNKRSK (SEQ ID NO. 299, encoded by nucleotide sequence of SEQ ID NO. 300-302); or a SGESRRVRRNKRSK (SEQ ID NO. 303, encoded by the nucleotide sequence of SEQ ID NO. 304). In some instances, the cleavage site is a P2A cleavage site, ATNFSLLKQAGDVEENPGP (SEQ ID NO. 120, encoded by SEQ ID NO. 121), wherein NPGP (SEQ ID NO. 305) is the P2A site.

In some embodiments, cleavage sites of the present disclosure, include without limitation, any of those taught in Table 7 of co-pending commonly owned U.S. Provisional Patent Application No. 62/320,864 filed on Apr. 11, 2016, or in U.S. Provisional Application No. 62/466,596 filed Mar. 3, 2017, and the International Publication WO2017/180587, the contents of each of which are incorporated herein by reference in their entirety.

### 3. Protein Tags

In some embodiments, the effector module of the disclosure may comprise a protein tag. The protein tag may be used for detecting and monitoring the process of the effector module. The effector module may include one or more tags such as an epitope tag (e.g., a FLAG or hemagglutinin (HA) tag). A large number of protein tags may be used for the present effector modules. They include, but are not limited to, self-labeling polypeptide tags (e.g., haloalkane dehalogenase (halotag2 or halotag7), ACP tag, clip tag, MCP tag, snap tag), epitope tags (e.g., FLAG, HA, His, and Myc), fluorescent tags (e.g., green fluorescent protein (GFP), red fluorescent protein (RFP), yellow fluorescent protein (YFP), and its variants), bioluminescent tags (e.g. luciferase and its variants), affinity tags (e.g., maltose-binding protein (MBP) tag, glutathione-S-transferase (GST) tag), immunogenic affinity tags (e.g., protein A/G, IRS, AU1, AU5, glu-glu, KT3, S-tag, HSV, VSV-G, Xpress and V5), and other tags (e.g., biotin (small molecule), StrepTag (StrepII), SBP, biotin carboxyl carrier protein (BCCP), eXact, CBP, CYD, HPC, CBD intein-chitin binding domain, Trx, NorpA, and NusA.

In other embodiments, a tag may also be selected from those disclosed in U.S. Pat. Nos. 8,999,897, 8,357,511, 7,094,568, 5,011,912, 4,851,341, and 4,703,004; U.S Patent Application Publication Nos. 2013/115635 and 2013/012687; and International Patent Publication No. WO2013/091661; the contents of each of which are incorporated herein by reference in their entirety.

In some aspects, a multiplicity of protein tags, either the same or different tags, may be used; each of the tags may be located at the same N- or C-terminus, whereas in other cases these tags may be located at each terminus.

In some embodiments, protein tags of the present disclosure, include without limitation, any of those taught in Table 8 of co-pending commonly owned U.S. Provisional Patent Application No. 62/320,864 filed on Apr. 11, 2016, or in U.S. Provisional Application No. 62/466,596 filed Mar. 3, 2017, and the International Publication WO2017/180587, the contents of which are incorporated herein by reference in their entirety.

### 4. Targeting Peptides

In some embodiments, the effector module of the disclosure may further comprise a targeting and/or penetrating peptide. Small targeting and/or penetrating peptides that selectively recognize cell surface markers (e.g. receptors, trans-membrane proteins, and extra-cellular matrix molecules) can be employed to target the effector module to the desired organs, tissues or cells. Short peptides (5-50 amino acid residues) synthesized in vitro and naturally occurring peptides, or analogs, variants, derivatives thereof, may be incorporated into the effector module for homing the effector module to the desired organs, tissues and cells, and/or subcellular locations inside the cells.

In some embodiments, a targeting sequence and/or penetrating peptide may be included in the effector module to drive the effector module to a target organ, or a tissue, or a cell (e.g., a cancer cell). In other embodiments, a targeting and/or penetrating peptide may direct the effector module to a specific subcellular location inside a cell.

A targeting peptide has any number of amino acids from about 6 to about 30 inclusive. The peptide may have 6, 7, 8, 9, 10, 11, 12, 13, 14, 15, 16, 17, 18, 19, 20, 21, 22, 23, 24, 25, 26, 27, 28, 29 or 30 amino acids. Generally, a targeting peptide may have 25 or fewer amino acids, for example, 20 or fewer, for example 15 or fewer.

Exemplary targeting peptides may include, but are not limited to, those disclosed in the art, e.g., U.S. Pat. Nos. 9,206,231, 9,110,059, 8,706,219, and 8,772,449; and U.S. Patent Application Publication Nos. 2016/089447, 2016/060296, 2016/060314, 2016/060312, 2016/060311, 2016/009772, 2016/002613, 2015/314011, and 2015/166621; and International Patent Publication Nos. WO2015/179691 and WO2015/183044; the contents of each of which are incorporated herein by reference in their entirety. In some embodiments, targeting peptides of the present disclosure, include without limitation, any of those taught in Table 9 of co-pending commonly owned U.S. Provisional Patent Application No. 62/320,864 filed on Apr. 11, 2016, or in U.S. Provisional Application No. 62/466,596 filed Mar. 3, 2017, and the International Publication WO2017/180587, the contents of each of which are incorporated herein by reference in their entirety.

### 5. Linkers

In some embodiments, the effector module of the disclosure may further comprise a linker sequence. The linker region serves primarily as a spacer between two or more polypeptides within the effector module. The “linker” or “spacer”, as used herein, refers to a molecule or group of molecules that connects two molecules, or two parts of a molecule such as two domains of a recombinant protein.

In some embodiments, “Linker” (L) or “linker domain” or “linker region” or “linker module” or “peptide linker” as used herein refers to an oligo- or polypeptide region of from about 1 to 100 amino acids in length, which links together any of the domains/regions of the effector module (also called peptide linker). The peptide linker may be 1-40 amino acids in length, or 2-30 amino acids in length, or 20-80 amino acids in length, or 50-100 amino acids in length. Linker length may also be optimized depending on the type of payload utilized and based on the crystal structure of the payload. In some instances, a shorter linker length may be preferably selected. In some aspects, the peptide linker is made up of amino acids linked together by peptide bonds, preferably from 1 to 20 amino acids linked by peptide bonds, wherein the amino acids are selected from the 20 naturally occurring amino acids: Glycine (G), Alanine (A), Valine (V), Leucine (L), Isoleucine (I), Serine (S), Cysteine (C), Threonine (T), Methionine (M), Proline (P), Phenylalanine (F), Tyrosine (Y), Tryptophan (W), Histidine (H), Lysine (K), Arginine (R), Aspartate (D), Glutamic acid (E), Asparagine (N), and Glutamine (Q). One or more of these amino acids may be glycosylated, as is understood by those in the art. In some aspects, amino acids of a peptide linker may be selected from Alanine (A), Glycine (G), Proline (P), Asparagine (R), Serine (S), Glutamine (Q) and Lysine (K).

In some embodiments, an artificially designed peptide linker may be composed of a polymer of flexible residues such as Glycine (G) and Serine (S) so that the adjacent protein domains are free to move relative to one another. Longer linkers may be used when it is desirable to ensure that two adjacent domains do not interfere with one another. The choice of a particular linker sequence may be of concern if it affects biological activity, stability, folding, targeting and/or pharmacokinetic features of the fusion construct. Examples of peptide linkers include but are not limited to are provided in Table 10.

TABLE 10
Linkers

| | AA | NA |
|---|---|---|
| | SEQ ID | SEQ ID |
| Linker Sequence | NO. | NO. |
| GGSG | 306 | 307 |
| GGSGG | 308 | 309-313 |
| GGSGGG | 314 | 315-316 |
| SGGGS | 317 | 318 |
| GGSGGGSGG | 319 | 320 |
| GGGGG | 321 | — |
| GGGGS | 322 | — |
| GGGGSGGGGS | 323 | — |
| GGGGSGGGGSGGGGSGGGGS | 324 | — |
| GGGGSGGGGSGGGGSGGGGSG | 325 | — |
| GGGS | | |
| GGGGSGGGGSGGGGSGGGGSG | 326 | — |
| GGGSGGGGS | | |
| SSSSG | 327 | — |
| SSSSGSSSSG | 328 | — |
| SSSSGSSSSGSSSSG | 329 | — |
| SSSSGSSSSGSSSSGSSSSG | 330 | — |
| SSSSGSSSSGSSSSGSSSSGSSSSG | 331 | — |
| SSSSGSSSSGSSSSGSSSSGSSSSG | 332 | — |
| SSSSG | | |
| SGGGSGGGGSGGGGSGGGGSG | 250 | 251 |
| GGSLQ | | |
| EFSTEF | 333 | 334-335 |
| SGGGS | 336 | 337 |
| GKSSGSGSESKS | 338 | — |
| GGSTSGSGKSSEGKG | 339 | — |
| GSTSGSGKSSSEGSGSTKG | 340 | — |
| GSTSGSGKPGSGEGSTKG | 341 | — |
| VDYPYDVPDYALD | 342 | 343 |
| EGKSSGSGSESKEF | 344 | 125; |
| | | 345-349 |
| SGGGSGGGGSGGGGSGGGGSG | 350 | 351 |
| GGSLQYPYDVPDYA | | |
| DYKDDDDK | 352 | 353 |
| SGGGSGGGGSGGGGSGGGGSG | 354 | 355 |
| GGGSGGGGSGGGS | | |
| SGGGSGGGGSGGGGSGGGGSYP | 356 | 357 |
| YDVPDYASGGGS | | |
| GSGATNFSLLKQAGDVEENPGP | 358 | 359 |
| SGGGGSGGGGSGGGGSGGGSLQ | 360 | — |
| GGGG | 361 | — |
| GGGGGGGG | 362 | — |

The linkers described herein are exemplary, and linkers that are much longer and which include other residues are contemplated by the present disclosure. In one embodiment, the linker may be MH or EF.

A linker sequence may be a natural linker derived from a multi-domain protein. A natural linker is a short peptide sequence that separates two different domains or motifs within a protein.

In some aspects, linkers may be flexible or rigid. In other aspects, linkers may be cleavable or non-cleavable. As used herein, the terms “cleavable linker domain or region” or “cleavable peptide linker” are used interchangeably. In some embodiments, the linker sequence may be cleaved enzymatically and/or chemically. Examples of enzymes (e.g., proteinase/peptidase) useful for cleaving the peptide linker include, but are not limited, to Arg-C proteinase, Asp-N endopeptidase, chymotrypsin, clostripain, enterokinase, Factor Xa, glutamyl endopeptidase, Granzyme B, Achromobacter proteinase I, pepsin, proline endopeptidase, proteinase K, Staphylococcal peptidase I, thermolysin, thrombin, trypsin, and members of the Caspase family of proteolytic enzymes (e.g. Caspases 1-10). Chemical sensitive cleavage sites may also be included in a linker sequence. Examples of chemical cleavage reagents include, but are not limited to, cyanogen bromide, which cleaves methionine residues; N-chloro succinimide, iodobenzoic acid or BNPS-skatole [2-(2-nitrophenylsulfenyl)-3-methylindole], which cleaves tryptophan residues; dilute acids, which cleave at aspartyl-prolyl bonds; and e aspartic acid-proline acid cleavable recognition sites (i.e., a cleavable peptide linker comprising one or more D-P dipeptide moieties). The fusion module may include multiple regions encoding peptides of interest separated by one or more cleavable peptide linkers.

In other embodiments, a cleavable linker may be a “self-cleaving” linker peptide, such as 2A linker (for example T2A), 2A-like linkers or functional equivalents thereof and combinations thereof. In some embodiments, the linkers include the picornaviral 2A-like linker, CHYSEL sequences of porcine teschovirus (P2A), Thosea asigna virus (T2A) or combinations, variants and functional equivalents thereof. In some embodiments, the biocircuits of the present disclosure may include 2A peptides. The 2A peptide is a sequence of about 20 amino acid residues from a virus that is recognized by a protease (2A peptidases) endogenous to the cell. The 2A peptide was identified among picornaviruses, a typical example of which is the Foot-and Mouth disease virus (Robertson B H, et. al., J Virol 1985, 54:651-660). 2A-like sequences have also been found in Picornaviridae like equine rhinitis A virus, as well as unrelated viruses such as porcine teschovirus-1 and the insect Thosea asigna virus (TaV). In such viruses, multiple proteins are derived from a large polyprotein encoded by an open reading frame. The 2A peptide mediates the co-translational cleavage of this polyprotein at a single site that forms the junction between the virus capsid and replication polyprotein domains. These sequences are thought to act co-translationally, preventing the formation of a normal peptide bond between the glycine and last proline, resulting in the ribosome skipping of the next codon (Donnelly M L et al. (2001). J Gen Virol, 82:1013-1025). After cleavage, the short peptide remains fused to the C-terminus of the protein upstream of the cleavage site, while the proline is added to the N-terminus of the protein downstream of the cleavage site. Of the 2A peptides identified to date, four have been widely used namely FMDV 2A (abbreviated herein as F2A); equine rhinitis A virus (ERAV) 2A (E2A); porcine teschovirus-1 2A (P2A) and Thosea asigna virus 2A (T2A). In some embodiments, the 2A peptide sequences useful in the present disclosure are selected from SEQ ID NO. 8-11 of International Patent Publication WO2010042490, the contents of which are incorporated by reference in its entirety.

Other linkers will be apparent to those skilled in the art and may be used in connection with alternate embodiments of the disclosure.

The linkers of the present disclosure may also be non-peptide linkers. For example, alkyl linkers such as —NH—(CH₂)a-C(O)—, wherein a=2-20 can be used. These alkyl linkers may further be substituted by any non-sterically hindering group such as lower alkyl (e.g., C₁-C₆) lower acyl, halogen (e.g., Cl, Br), CN, NH₂, phenyl, etc.

In some aspects, the linker may be an artificial linker from U.S. Pat. Nos. 4,946,778, 5,525,491, 5,856,456; and International Patent Publication No. WO2012/083424; the contents of each of which are incorporated herein by reference in their entirety.

In some embodiments, linkers of the present disclosure, include without limitation, any of those taught in Table 11 of co-pending commonly owned U.S. Provisional Patent Application No. 62/320,864 filed on Apr. 11, 2016, or in U.S. Provisional Application No. 62/466,596 filed Mar. 3, 2017, and the International Publication WO2017/180587, the contents of each of which are incorporated herein by reference in their entirety.

### 6. Embedded Stimulus, Signals and Other Regulatory Features

microRNAs (or miRNA) are 19-25 nucleotide long noncoding RNAs that bind to the 3′UTR of nucleic acid molecules and down-regulate gene expression either by reducing nucleic acid molecule stability or by inhibiting translation. The polynucleotides of the disclosure may comprise one or more microRNA target sequences, microRNA sequences, or microRNA seeds. Such sequences may correspond to any known microRNA such as those taught in U.S. Publication Nos. US2005/0261218 and US2005/0059005, the contents of each of which are incorporated herein by reference in their entirety. As a non-limiting embodiment, known microRNAs, their sequences and their binding site sequences in the human genome are in Table 14 of the co-pending commonly owned U.S. Ser. No. 62/320,864 filed on Apr. 11, 2016, or in U.S. Provisional Application No. 62/466,596 filed Mar. 3, 2017, and the International Publication WO2017/180587, the contents of each of which are incorporated herein by reference in their entirety.

A microRNA sequence comprises a “seed” region, i.e., a sequence in the region of positions 2-8 of the mature microRNA, which sequence has perfect Watson-Crick complementarity to the miRNA target sequence. A microRNA seed may comprise positions 2-8 or 2-7 of the mature microRNA. In some embodiments, a microRNA seed may comprise 7 nucleotides (e.g., nucleotides 2-8 of the mature microRNA), wherein the seed-complementary site in the corresponding miRNA target is flanked by an adenine (A) opposed to microRNA position 1. In some embodiments, a microRNA seed may comprise 6 nucleotides (e.g., nucleotides 2-7 of the mature microRNA), wherein the seed-complementary site in the corresponding miRNA target is flanked by an adenine (A) opposed to microRNA position 1. See for example, Grimson A, Farh K K, Johnston W K, Garrett-Engele P, Lim L P, Bartel D P; Mol Cell. 2007 Jul. 6; 27(1):91-105. The bases of the microRNA seed have complete complementarity with the target sequence. By engineering microRNA target sequences into the polynucleotides encoding the biocircuit components, effector modules, SREs or payloads of the disclosure one can target the molecule for degradation or reduced translation, provided the microRNA in question is available. This process will reduce the hazard of off target effects upon nucleic acid molecule delivery.

Identification of microRNA, microRNA target regions, and their expression patterns and role in biology have been reported (Bonauer et al., Curr Drug Targets 2010 11:943-949; Anand and Cheresh Curr Opin Hematol 2011 18:171-176; Contreras and Rao Leukemia 2012 26:404-413 (2011 Dec. 20. doi: 10.1038/leu.2011.356); Bartel Cell 2009 136:215-233; Landgraf et al, Cell, 2007 129:1401-1414; Gentner and Naldini, Tissue Antigens. 2012 80:393-403 and all references therein; each of which is herein incorporated by reference in its entirety).

For example, if the polynucleotide is not intended to be delivered to the liver but ends up there, then miR-122, a microRNA abundant in liver, can inhibit the expression of the polynucleotide if one or multiple target sites of miR-122 are engineered into the polynucleotide. Introduction of one or multiple binding sites for different microRNA can be engineered to further decrease the longevity, stability, and protein translation of a polynucleotide hence providing an additional layer of tenability beyond the stimulus selection, SRE design and payload variation.

As used herein, the term “microRNA site” refers to a microRNA target site or a microRNA recognition site, or any nucleotide sequence to which a microRNA binds or associates. It should be understood that “binding” may follow traditional Watson-Crick hybridization rules or may reflect any stable association of the microRNA with the target sequence at or adjacent to the microRNA site.

Conversely, for the purposes of the polynucleotides of the present disclosure, microRNA binding sites can be engineered out of (i.e. removed from) sequences in which they naturally occur in order to increase protein expression in specific tissues. For example, miR-122 binding sites may be removed to improve protein expression in the liver.

Regulation of expression in multiple tissues can be accomplished through introduction or removal or one or several microRNA binding sites.

Specifically, microRNAs are known to be differentially expressed in immune cells (also called hematopoietic cells), such as antigen presenting cells (APCs) (e.g. dendritic cells and macrophages), macrophages, monocytes, B lymphocytes, T lymphocytes, granulocytes, natural killer cells, etc. Immune cell specific microRNAs are involved in immunogenicity, autoimmunity, the immune-response to infection, inflammation, as well as unwanted immune response after gene therapy and tissue/organ transplantation. Immune cells specific microRNAs also regulate many aspects of development, proliferation, differentiation and apoptosis of hematopoietic cells (immune cells). For example, miR-142 and miR-146 are exclusively expressed in the immune cells, particularly abundant in myeloid dendritic cells. Introducing the miR-142 binding site into the 3′-UTR of a polypeptide of the present disclosure can selectively suppress the gene expression in the antigen presenting cells through miR-142 mediated mRNA degradation, limiting antigen presentation in professional APCs (e.g. dendritic cells) and thereby preventing antigen-mediated immune response after gene delivery (see, Annoni A et al., blood, 2009, 114, 5152-5161, the content of which is herein incorporated by reference in its entirety.)

In one embodiment, microRNAs binding sites that are known to be expressed in immune cells, in particular, the antigen presenting cells, can be engineered into the polynucleotides to suppress the expression of the polynucleotide in APCs through microRNA mediated RNA degradation, subduing the antigen-mediated immune response, while the expression of the polynucleotide is maintained in non-immune cells where the immune cell specific microRNAs are not expressed.

Many microRNA expression studies have been conducted, and are described in the art, to profile the differential expression of microRNAs in various cancer cells/tissues and other diseases. Some microRNAs are abnormally over-expressed in certain cancer cells and others are under-expressed. For example, microRNAs are differentially expressed in cancer cells (WO2008/154098, US2013/0059015, US2013/0042333, WO2011/157294); cancer stem cells (US2012/0053224); pancreatic cancers and diseases (US2009/0131348, US2011/0171646, US2010/0286232, U.S. Pat. No. 8,389,210); asthma and inflammation (U.S. Pat. No. 8,415,096); prostate cancer (US2013/0053264); hepatocellular carcinoma (WO2012/151212, US2012/0329672, WO2008/054828, U.S. Pat. No. 8,252,538); lung cancer cells (WO2011/076143, WO2013/033640, WO2009/070653, US2010/0323357); cutaneous T cell lymphoma (WO2013/011378); colorectal cancer cells (WO2011/0281756, WO2011/076142); cancer positive lymph nodes (WO2009/100430, US2009/0263803); nasopharyngeal carcinoma (EP2112235); chronic obstructive pulmonary disease (US2012/0264626, US2013/0053263); thyroid cancer (WO2013/066678); ovarian cancer cells (US2012/0309645, WO2011/095623); breast cancer cells (WO2008/154098, WO2007/081740, US2012/0214699), leukemia and lymphoma (WO2008/073915, US2009/0092974, US2012/0316081, US2012/0283310, WO2010/018563, the content of each of which is incorporated herein by reference in their entirety).

In one embodiment, microRNA may be used as described herein in support of the creation of tunable biocircuits.

In some embodiments, effector modules may be designed to encode (as a DNA or RNA or mRNA) one or more payloads, SREs and/or regulatory sequence such as a microRNA or microRNA binding site. In some embodiments, any of the encoded payloads or SREs may be stabilized or de-stabilized by mutation and then combined with one or more regulatory sequences to generate a dual or multi-tuned effector module or biocircuit system.

Each aspect or tuned modality may bring to the effector module or biocircuit a differentially tuned feature. For example, an SRE may represent a destabilizing domain, while mutations in the protein payload may alter its cleavage sites or dimerization properties or half-life and the inclusion of one or more microRNA or microRNA binding site may impart cellular detargeting or trafficking features. Consequently, the present disclosure embraces biocircuits which are multifactorial in their tenability.

In some embodiments, compositions of the disclosure may include optional proteasome adaptors. As used herein, the term “proteasome adaptor” refers to any nucleotide/amino acid sequence that targets the appended payload for degradation. In some aspects, the adaptors target the payload for degradation directly thereby circumventing the need for ubiquitination reactions. Proteasome adaptors may be used in conjunction with destabilizing domains to reduce the basal expression of the payload. Exemplary proteasome adaptors include the UbL domain of Rad23 or hHR23b, HPV E7 which binds to both the target protein Rb and the S4 subunit of the proteasome with high affinity, which allows direct proteasome targeting, bypassing the ubiquitination machinery; the protein gankyrin which binds to Rb and the proteasome subunit S6.

Such biocircuits may be engineered to contain one, two, three, four or more tuned features.

In some embodiments, microRNA sequences of the present disclosure, include without limitation, any of those taught in Table 13 of co-pending commonly owned U.S. Provisional Patent Application No. 62/320,864 filed on Apr. 11, 2016, or in U.S. Provisional Application No. 62/466,596 filed Mar. 3, 2017, and the International Publication WO2017/180587, the contents of each of which are incorporated herein by reference in their entirety.

### Degrons

In some embodiments, the effector modules of the present disclosure may include degrons at their C termini. The degrons may comprise -GG, -RG, -KG, -QG, -WG, -PG, and -AG as the penultimate and the ultimate amino acids of the SREs. Furthermore, certain −2 amino acids (D, E, V, I and L) may be more enriched in the C terminus of the of the effector modules. Other degrons include, but are not limited, to RxxG motif, wherein x is any amino acid, C-terminal twin glutamic acid (EE) motif, and motifs that comprise an arginine at the −3 positions. Degrons may also be selected from the R-3 motif, G-end, R at −3, A-end, A at −2, V at −2 positions. Any of the degrons described in Koren et al., 2018, Cell 173, 1-14, may be useful in the present disclosure (the contents of which are incorporated by reference in their entirety). In some aspects, the expression of the effector module may be tuned by altering its overall amino acid composition. In some aspects, the amino acid composition of the effector module may be tuned to reduce basal expression. In some embodiments, basal expression may be tuned by increasing the number of bulky aromatic residues such as tryptophan (W), phenylalanine (F), and tyrosine (Y) in the effector module. Such bulky amino acids are known to reduce protein stability. In some embodiments, the amino acid composition of the SREs may be enriched with acidic residues such as, but not limited to, aspartic acid (D) and glutamic acid (E), and positively charged lysine (K), if an increase in the basal expression of the SRE is desired.

### Polynucleotides

The present disclosure provides polynucleotides encoding novel ER DDs, effector modules comprising payloads and associated DDs, biocircuit systems comprising DDs and effector modules, and other components of the present disclosure.

The term “polynucleotide” or “nucleic acid molecule” in its broadest sense, includes any compound and/or substance that comprise a polymer of nucleotides, e.g., linked nucleosides. These polymers are often referred to as polynucleotides. Exemplary nucleic acids or polynucleotides of the disclosure include, but are not limited to, ribonucleic acids (RNAs), deoxyribonucleic acids (DNAs), threose nucleic acids (TNAs), glycol nucleic acids (GNAs), peptide nucleic acids (PNAs), locked nucleic acids (LNAs, including LNA having a β-D-ribo configuration, α-LNA having an α-L-ribo configuration (a diastereomer of LNA), 2′-amino-LNA having a 2′-amino functionalization, and 2′-amino-α-LNA having a 2′-amino functionalization) or hybrids thereof.

In some embodiments, polynucleotides of the disclosure may be a messenger RNA (mRNA) or any nucleic acid molecule and may or may not be chemically modified. In one aspect, the nucleic acid molecule is a mRNA. As used herein, the term “messenger RNA (mRNA)” refers to any polynucleotide which encodes a polypeptide of interest and which is capable of being translated to produce the encoded polypeptide of interest in vitro, in vivo, in situ or ex vivo.

Traditionally, the basic components of an mRNA molecule include at least a coding region, a 5′UTR, a 3′UTR, a 5′ cap and a poly-A tail. Building on this wild type modular structure, the present disclosure expands the scope of functionality of traditional mRNA molecules by providing payload constructs which maintain a modular organization, but which comprise one or more structural and/or chemical modifications or alterations which impart useful properties to the polynucleotide, for example tenability of function. As used herein, a “structural” feature or modification is one in which two or more linked nucleosides are inserted, deleted, duplicated, inverted or randomized in a polynucleotide without significant chemical modification to the nucleosides themselves. Because chemical bonds will necessarily be broken and reformed to effect a structural modification, structural modifications are of a chemical nature and hence are chemical modifications. However, structural modifications will result in a different sequence of nucleotides. For example, the polynucleotide “ATCG” may be chemically modified to “AT-5meC-G”. The same polynucleotide may be structurally modified from “ATCG” to “ATCCCG”. Here, the dinucleotide “CC” has been inserted, resulting in a structural modification to the polynucleotide.

In some embodiments, polynucleotides of the present disclosure may harbor 5′UTR sequences which play a role in translation initiation. 5′UTR sequences may include features such as Kozak sequences which are commonly known to be involved in the process by which the ribosome initiates translation of genes, Kozak sequences have the consensus XCCR(A/G)CC-start codon (AUG), where R is a purine (adenine or guanine) three bases upstream of the start codon (AUG) and X is any nucleotide. In one embodiment, the Kozak sequence is ACCGCC. By engineering the features that are typically found in abundantly expressed genes of target cells or tissues, the stability and protein production of the polynucleotides of the disclosure can be enhanced.

Further provided are polynucleotides, which may contain an internal ribosome entry site (IRES) which play an important role in initiating protein synthesis in the absence of 5′ cap structure in the polynucleotide. An IRES may act as the sole ribosome binding site or may serve as one of the multiple binding sites. Polynucleotides of the disclosure containing more than one functional ribosome binding site may encode several peptides or polypeptides that are translated independently by the ribosomes giving rise to bicistronic and/or multicistronic nucleic acid molecules.

In some embodiments, polynucleotides encoding biocircuits, effector modules, DDs and payloads may include from about 30 to about 100,000 nucleotides (e.g., from 30 to 50, from 30 to 100, from 30 to 250, from 30 to 500, from 30 to 1,000, from 30 to 1,500, from 30 to 3,000, from 30 to 5,000, from 30 to 7,000, from 30 to 10,000, from 30 to 25,000, from 30 to 50,000, from 30 to 70,000, from 100 to 250, from 100 to 500, from 100 to 1,000, from 100 to 1,500, from 100 to 3,000, from 100 to 5,000, from 100 to 7,000, from 100 to 10,000, from 100 to 25,000, from 100 to 50,000, from 100 to 70,000, from 100 to 100,000, from 500 to 1,000, from 500 to 1,500, from 500 to 2,000, from 500 to 3,000, from 500 to 5,000, from 500 to 7,000, from 500 to 10,000, from 500 to 25,000, from 500 to 50,000, from 500 to 70,000, from 500 to 100,000, from 1,000 to 1,500, from 1,000 to 2,000, from 1,000 to 3,000, from 1,000 to 5,000, from 1,000 to 7,000, from 1,000 to 10,000, from 1,000 to 25,000, from 1,000 to 50,000, from 1,000 to 70,000, from 1,000 to 100,000, from 1,500 to 3,000, from 1,500 to 5,000, from 1,500 to 7,000, from 1,500 to 10,000, from 1,500 to 25,000, from 1,500 to 50,000, from 1,500 to 70,000, from 1,500 to 100,000, from 2,000 to 3,000, from 2,000 to 5,000, from 2,000 to 7,000, from 2,000 to 10,000, from 2,000 to 25,000, from 2,000 to 50,000, from 2,000 to 70,000, and from 2,000 to 100,000 nucleotides). In some aspects, polynucleotides of the disclosure may include more than 10,000 nucleotides.

Regions of the polynucleotides which encode certain features such as cleavage sites, linkers, trafficking signals, tags or other features may range independently from 10-1,000 nucleotides in length (e.g., greater than 20, 30, 40, 45, 50, 55, 60, 70, 80, 90, 100, 120, 140, 160, 180, 200, 250, 300, 350, 400, 450, 500, 600, 700, 800, and 900 nucleotides or at least 10, 15, 20, 25, 30, 35, 40, 45, 50, 55, 60, 70, 80, 90, 100, 120, 140, 160, 180, 200, 250, 300, 350, 400, 450, 500, 600, 700, 800, 900, and 1,000 nucleotides).

In some embodiments, polynucleotides of the present disclosure may further comprise embedded regulatory moieties such as microRNA binding sites within the 3′UTR of nucleic acid molecules which when bind to microRNA molecules, down-regulate gene expression either by reducing nucleic acid molecule stability or by inhibiting translation. Conversely, for the purposes of the polynucleotides of the present disclosure, microRNA binding sites can be engineered out of (i.e. removed from) sequences in which they naturally occur in order to increase protein expression in specific tissues. For example, miR-142 and miR-146 binding sites may be removed to improve protein expression in the immune cells. In some embodiments, any of the encoded payloads may be regulated by an SRE and then combined with one or more regulatory sequences to generate a dual or multi-tuned effector module or biocircuit system.

In some embodiments, polynucleotides of the present disclosure may encode fragments, variants, derivatives of polypeptides of the disclosures. In some aspects, the variant sequence may keep the same or a similar activity. Alternatively, the variant may have an altered activity (e.g., increased or decreased) relative to the start sequence. Generally, variants of a particular polynucleotide or polypeptide of the disclosure will have at least about 40%, 45%, 50%, 55%, 60%, 65%, 70%, 75%, 80%, 85%, 90%, 91%, 92%, 93%, 94%, 95%, 96%, 97%, 98%, 99% but less than 100% sequence identity to that particular reference polynucleotide or polypeptide as determined by sequence alignment programs and parameters described herein and known to those skilled in the art. Such tools for alignment include those of the BLAST suite (Stephen et al., Gapped BLAST and PSI-BLAST: a new generation of protein database search programs, Nucleic Acids Res., 1997, 25:3389-3402.)

In some embodiments, polynucleotides of the present disclosure may be modified. As used herein, the terms “modified”, or as appropriate, “modification” refers to chemical modification with respect to A, G, U (T in DNA) or C nucleotides. Modifications may be on the nucleoside base and/or sugar portion of the nucleosides which comprise the polynucleotide. In some embodiments, multiple modifications are included in the modified nucleic acid or in one or more individual nucleoside or nucleotide. For example, modifications to a nucleoside may include one or more modifications to the nucleobase and the sugar. Modifications to the polynucleotides of the present disclosure may include any of those taught in, for example, International Publication No. WO2013/052523, the contents of which are incorporated herein by reference in its entirety.

As described herein “nucleoside” is defined as a compound containing a sugar molecule (e.g., a pentose or ribose) or a derivative thereof in combination with an organic base (e.g., a purine or pyrimidine) or a derivative thereof (also referred to herein as “nucleobase”). As described herein, “nucleotide” is defined as a nucleoside including a phosphate group.

In some embodiments, the modification may be on the internucleoside linkage (e.g., phosphate backbone). Herein, in the context of the polynucleotide backbone, the phrases “phosphate” and “phosphodiester” are used interchangeably. Backbone phosphate groups can be modified by replacing one or more of the oxygen atoms with a different substituent. Further, the modified nucleosides and nucleotides can include the wholesale replacement of an unmodified phosphate moiety with another internucleoside linkage. Examples of modified phosphate groups include, but are not limited to, phosphorothioate, phosphoroselenates, boranophosphates, boranophosphate esters, hydrogen phosphonates, phosphoramidates, phosphorodiamidates, alkyl or aryl phosphonates, and phosphotriesters. Phosphorodithioates have both non-linking oxygens replaced by sulfur. The phosphate linker can also be modified by the replacement of a linking oxygen with nitrogen (bridged phosphoramidates), sulfur (bridged phosphorothioates), and carbon (bridged methylene-phosphonates). Other modifications which may be used are taught in, for example, International Application No. WO2013/052523, the contents of which are incorporated herein by reference in their entirety.

Chemical modifications and/or substitution of the nucleotides or nucleobases of the polynucleotides of the disclosure which are useful in the present disclosure include any modified substitutes known in the art, for example, (±)1-(2-Hydroxypropyl)pseudouridine TP, (2R)-1-(2-Hydroxypropyl)pseudouridine TP, 1-(4-Methoxy-phenyl)pseudo-UTP, 2′-O-dimethyladenosine, 1,2′-O-dimethylguanosine, 1,2′-O-dimethylinosine, 1-Hexyl-pseudo-UTP, 1-Homoallylpseudouridine TP, 1-Hydroxymethylpseudouridine TP, 1-iso-propyl-pseudo-UTP, 1-Me-2-thio-pseudo-UTP, 1-Me-4-thio-pseudo-UTP, 1-Me-alpha-thio-pseudo-UTP, 1-Me-GTP, 2′-Amino-2′-deoxy-ATP, 2′-Amino-2′-deoxy-CTP, 2′-Amino-2′-deoxy-GTP, 2′-Amino-2′-deoxy-UTP, 2′-Azido-2′-deoxy-ATP, tubercidine, undermodified hydroxywybutosine, uridine 5-oxyacetic acid, uridine 5-oxyacetic acid methyl ester, wybutosine, wyosine, xanthine, Xanthosine-5′-TP, xylo-adenosine, zebularine, α-thio-adenosine, α-thio-cytidine, α-thio-guanosine, and/or α-thio-uridine.

Polynucleotides of the present disclosure may comprise one or more of the modifications taught herein. Different sugar modifications, base modifications, nucleotide modifications, and/or internucleoside linkages (e.g., backbone structures) may exist at various positions in the polynucleotide of the disclosure. One of ordinary skill in the art will appreciate that the nucleotide analogs or other modification(s) may be located at any position(s) of a polynucleotide such that the function of the polynucleotide is not substantially decreased. A modification may also be a 5′ or 3′ terminal modification. The polynucleotide may contain from about 1% to about 100% modified nucleotides (either in relation to overall nucleotide content, or in relation to one or more types of nucleotide, i.e. any one or more of A, G, U or C) or any intervening percentage (e.g., from 1% to 20%, from 1% to 25%, from 1% to 50%, from 1% to 60%, from 1% to 70%, from 1% to 80%, from 1% to 90%, from 1% to 95%, from 10% to 20%, from 10% to 25%, from 10% to 50%, from 10% to 60%, from 10% to 70%, from 10% to 80%, from 10% to 90%, from 10% to 95%, from 10% to 100%, from 20% to 25%, from 20% to 50%, from 20% to 60%, from 20% to 70%, from 20% to 80%, from 20% to 90%, from 20% to 95%, from 20% to 100%, from 50% to 60%, from 50% to 70%, from 50% to 80%, from 50% to 90%, from 50% to 95%, from 50% to 100%, from 70% to 80%, from 70% to 90%, from 70% to 95%, from 70% to 100%, from 80% to 90%, from 80% to 95%, from 80% to 100%, from 90% to 95%, from 90% to 100%, and from 95% to 100%).

In some embodiments, one or more codons of the polynucleotides of the present disclosure may be replaced with other codons encoding the native amino acid sequence to tune the expression of the SREs, through a process referred to as codon selection. Since mRNA codon, and tRNA anticodon pools tend to vary among organisms, cell types, sub cellular locations and over time, the codon selection described herein is a spatiotemporal (ST) codon selection.

In some embodiments of the disclosure, certain polynucleotide features may be codon optimized. Codon optimization refers to a process of modifying a nucleic acid sequence for enhanced expression in the host cell by replacing at least 1, 2, 3, 4, 5, 10, 15, 20, 25, 50 or more codons of the native sequence with codons that are most frequently used in the genes of that host cell while maintaining the native amino acid sequence. Codon usage may be measured using the Codon Adaptation Index (CAI) which measures the deviation of a coding polynucleotide sequence from a reference gene set. Codon usage tables are available at the Codon Usage Database (http://www.kazusa.or.jp/codon/) and the CAI can be calculated by EMBOSS CAI program (http://emboss.sourceforge.net/). Codon optimization methods are known in the art and may be useful in efforts to achieve one or more of several goals. These goals include to match codon frequencies in target and host organisms to ensure proper folding, bias nucleotide content to alter stability or reduce secondary structures, minimize tandem repeat codons or base runs that may impair gene construction or expression, customize transcriptional and translational control regions, insert or remove protein signaling sequences, remove/add post translation modification sites in encoded protein (e.g. glycosylation sites), add, remove or shuffle protein domains, insert or delete restriction sites, modify ribosome binding sites and degradation sites, to adjust translational rates to allow the various domains of the protein to fold properly, or to reduce or eliminate problem secondary structures within the polynucleotide. Codon optimization tools, algorithms and services are known in the art, and non-limiting examples include services from GeneArt (Life Technologies), DNA2.0 (Menlo Park Calif.), OptimumGene (GenScript, Piscataway, N.J.), algorithms such as but not limited to, DNAWorks v3.2.3, and/or proprietary methods. In one embodiment, a polynucleotide sequence or portion thereof is codon optimized using optimization algorithms. Codon options for each amino acid are well-known in the art as are various species table for optimizing for expression in that particular species.

In some embodiments of the disclosure, certain polynucleotide features may be codon optimized. For example, a preferred region for codon optimization may be upstream (5′) or downstream (3′) to a region which encodes a polypeptide. These regions may be incorporated into the polynucleotide before and/or after codon optimization of the payload encoding region or open reading frame (ORF).

After optimization (if desired), the polynucleotide components are reconstituted and transformed into a vector such as, but not limited to, plasmids, viruses, cosmids, and artificial chromosomes.

Spatiotemporal codon selection may impact the expression of the polynucleotides of the disclosure, since codon composition determines the rate of translation of the mRNA species and its stability. For example, tRNA anticodons to optimized codons are abundant, and thus translation may be enhanced. In contrast, tRNA anticodons to less common codons are fewer and thus translation may proceed at a slower rate. Presnyak et al. have shown that the stability of an mRNA species is dependent on the codon content, and higher stability and thus higher protein expression may be achieved by utilizing optimized codons (Presnyak et al. (2015) Cell 160, 1111-1124; the contents of which are incorporated herein by reference in their entirety). Thus, in some embodiments, ST codon selection may include the selection of optimized codons to enhance the expression of the SRES, effector modules and biocircuits of the disclosure. In other embodiments, spatiotemporal codon selection may involve the selection of codons that are less commonly used in the genes of the host cell to decrease the expression of the compositions of the disclosure. The ratio of optimized codons to codons less commonly used in the genes of the host cell may also be varied to tune expression.

In some embodiments, certain regions of the polynucleotide may be preferred for codon selection. For example, a preferred region for codon selection may be upstream (5′) or downstream (3′) to a region which encodes a polypeptide. These regions may be incorporated into the polynucleotide before and/or after codon selection of the payload encoding region or open reading frame (ORF).

The stop codon of the polynucleotides of the present disclosure may be modified to include sequences and motifs to alter the expression levels of the SREs, payloads and effector modules of the present disclosure. Such sequences may be incorporated to induce stop codon readthrough, wherein the stop codon may specify amino acids e.g. selenocysteine or pyrrolysine. In other instances, stop codons may be skipped altogether to resume translation through an alternate open reading frame. Stop codon read through may be utilized to tune the expression of components of the effector modules at a specific ratio (e.g. as dictated by the stop codon context). Examples of preferred stop codon motifs include UGAN, UAAN, and UAGN, where N is either C or U.

Polynucleotide modifications and manipulations can be accomplished by methods known in the art such as, but not limited to, site directed mutagenesis and recombinant technology. The resulting modified molecules may then be tested for activity using in vitro or in vivo assays such as those described herein, or any other suitable screening assay known in the art.

In some embodiments, polynucleotides of the disclosure may comprise two or more effector module sequences, or two or more payload sequences, which are in a pattern such as ABABAB or AABBAABBAABB or ABCABCABC or variants thereof repeated once, twice, or more than three times. In these patterns, each letter, A, B, or C represent a different effector module component.

In yet another embodiment, polynucleotides of the disclosure may comprise two or more effector module component sequences with each component having one or more SRE sequences (DD sequences), or two or more payload sequences. As a non-limiting example, the sequences may be in a pattern such as ABABAB or AABBAABBAABB or ABCABCABC or variants thereof repeated once, twice, or more than three times in each of the regions. As another non-limiting example, the sequences may be in a pattern such as ABABAB or AABBAABBAABB or ABCABCABC or variants thereof repeated once, twice, or more than three times across the entire polynucleotide. In these patterns, each letter, A, B, or C represent a different sequence or component.

According to the present disclosure, polynucleotides encoding distinct biocircuits, effector modules, SREs and payload constructs may be linked together through the 3′-end using nucleotides which are modified at the 3′-terminus. Chemical conjugation may be used to control the stoichiometry of delivery into cells. Polynucleotides can be designed to be conjugated to other polynucleotides, dyes, intercalating agents (e.g. acridines), cross-linkers (e.g. psoralene, mitomycin C), porphyrins (TPPC4, texaphyrin, Sapphyrin), polycyclic aromatic hydrocarbons (e.g., phenazine, dihydrophenazine), artificial endonucleases (e.g. EDTA), alkylating agents, phosphate, amino, mercapto, PEG (e.g., PEG-40K), MPEG, [MPEG]₂, polyamino, alkyl, substituted alkyl, radiolabeled markers, enzymes, haptens (e.g. biotin), transport/absorption facilitators (e.g., aspirin, vitamin E, folic acid), synthetic ribonucleases, proteins, e.g., glycoproteins, or peptides, e.g., molecules having a specific affinity for a co-ligand, or antibodies e.g., an antibody, that binds to a specified cell type such as a cancer cell, endothelial cell, or bone cell, hormones and hormone receptors, non-peptidic species, such as lipids, lectins, carbohydrates, vitamins, cofactors, or a drug. As non-limiting examples, they may be conjugates with other immune conjugates.

In some embodiments, the compositions of the polynucleotides of the disclosure may be generated by combining the various components of the effector modules using the Gibson assembly method. The Gibson assembly reaction consists of three isothermal reactions, each relying on a different enzymatic activity including a 5′ exonuclease which generates long overhangs, a polymerase which fills in the gaps of the annealed single strand regions and a DNA ligase which seals the nicks of the annealed and filled-in gaps. Polymerase chain reactions are performed prior to Gibson assembly which may be used to generate PCR products with overlapping sequence. These methods can be repeated sequentially, to assemble larger and larger molecules. For example, the method can comprise repeating a method as above to join a second set of two or more DNA molecules of interest to one another, and then repeating the method again to join the first and second set DNA molecules of interest, and so on. At any stage during these multiple rounds of assembly, the assembled DNA can be amplified by transforming it into a suitable microorganism, or it can be amplified in vitro (e.g., with PCR).

### Molecular Switches in Biocircuits, Genetic Circuits

### Molecular Switches

In some embodiments, the DDs described herein can include aspects of molecular switches. Alternatively or additionally, molecular switches can be constructed using DDs as described. The term “molecular switch” as used herein refers to any molecule that can be reversibly shifted between two or more stable states in response to a stimulus (e.g., a ligand). Molecular switches can be employed in biocircuits or genetic circuits, i.e., engineered input-responsive biological circuits.

In some embodiments, molecular switches can be RNA-based switches. As a non-limiting example, a DD-regulated effector module can be provided in an RNA molecule that comprises a coding sequence for a destabilization domain fused to a translational repressor such as, for example, L7Ae, a kink-turn (K-turn) motif in the 5′ UTR of a payload mRNA, and the coding sequence for the payload (e.g., peptide or protein) (see, e.g., International Patent Publication NO: WO2016040395, the contents of which are incorporated herein by reference in their entirety). L7Ae is an archaeal protein that binds K-turn and K-loop motifs with high affinity. In the absence of a ligand that stabilizes the destabilization domain, fusion L7Ae protein is degraded and the payload peptide or protein is expressed (ON state), whereas in the presence of the ligand, the fusion L7Ae protein is not destabilized and binds to the K-turn motif and represses expression of the payload peptide or protein (OFF state). Other translational regulation systems can also be used, such as, but not limited to, MS2-tethered repressors, Tet repressors, and microRNAs. Other uses of tunable expression repressors are contemplated; for example, effector modules comprising DD-regulated repressor payloads can be used to control inducible expression of transgenes, including to block background or “leaky” expression of transgenes (see U.S. Pat. No. 9,388,425, the contents of which are incorporated herein by reference in their entirety).

DD-regulated effector modules as described herein can be utilized to establish tunable bistable genetic toggles, wherein ligand-mediated stabilization and/or destabilization of a SRE appended, for example, to a payload of interest stably shifts expression of a gene of interest between on and off states (see U.S. Pat. No. 6,841,376, the contents of which are incorporated herein by reference in their entirety). Trans-acting SREs can also be utilized to tunably transition cis-acting self-repressive expression cassettes (see U.S. Pat. No. 10,208,312, the contents of which are incorporated herein by reference in their entirety).

RNA-based molecular switches can be synthetic RNA. In some cases, such RNA molecules can comprise modified bases (modRNA) or self-replicating RNAs (replicons). RNA-based molecular switches can be responsive to small molecules, for example, small molecule-responsive RNA binding proteins (RBPs) (e.g., Wagner et al., Nat. Chem. Bio. 2018 14:1043-50). In this way, effector modules as described herein can be RBPs useful in regulating RNA circuits.

In some embodiments, molecular switches can be switched on/off via dimerization. For example, a first effector module can comprise a DD-regulated SRE which is a first member of a dimerization pair and optionally a first payload, and a second effector module can comprise a DD-regulated SRE which is second member of a dimerization pair and optionally a second payload, wherein the two members of the dimerization pair dimerize upon addition of a dimerization ligand. In some embodiments, dimerization can restore stability of the members of the dimerization pair and/or the attached payloads. Dimerization induces interaction, whether direct or indirect, of the two payloads to create a desired effect. In some embodiments, dimerization can induce degradation of the dimerization pair and/or the attached payloads. For example, bivalent small molecules can dimerize two molecules of an E3 ubiquitin ligase to induce self-degradation (see, e.g., Maniaci et al., Nat Commun. 2017 Oct. 10; 8(1):830, the contents of which are incorporated herein by reference in their entirety). A dimerization pair can comprise, or be derived from, for example, an antibody and its antigen, two fragments of an antibody, a ligand-binding domain and a cognate receptor (e.g., any of those described in International Patent Publication No: WO2017120546, the contents of which are incorporated herein by reference in their entirety), and an E3 ubiquitin ligase and a substrate (e.g., as described in Maniaci et al.).

In some embodiments, molecular switches can be conditionally active in specific cell types or under specific cellular conditions. For example, the stimulus required to stimulate the DD-regulated SREs of the effector modules may only be present in a particular cell type or under a particular cellular condition. This allows various applications, such as cell-type specific delivery of a payload, or detection of a particular cellular target. Based on this property, biocircuits or effector modules described herein can be developed as biosensors. For example, a DD can be fused to a reporter protein (e.g., GFP). Such DD, in the context of an SRE, can contain mutations such that the SRE is conditionally stable only in the presence of a stabilizing ligand which is also the target to be detected. When the target is present in the cell, the fusion protein is stabilized and the reporter activity can be detected (see, e.g., International Patent Publication Nos: WO2009137136, WO2017048316, and WO2017156238; and U.S. Pat. Nos. 8,329,889 and 9,766,255, the contents of each of which are incorporated herein by reference in their entireties). In some embodiments, genetic circuits may include a conditional NF-kB (nuclear factor kappa-B) responsive promoter driving expression of the effector modules described herein. Such circuits are described in the International Patent Publication WO2018170390, the contents of which are incorporated by reference in their entirety.

In some embodiments, engineered cell circuits that enable multifactorial modulation within and/or near a tumor (a tumor microenvironment (TME)) may be utilized. Such circuits are described in the International Patent Publication WO2018191619, the contents of which are incorporated by reference in their entirety.

In some embodiments, the biocircuits described herein may include SynNotch switches that include a synthetic notch (SynNotch) receptors. SynNotch receptors contain the core regulatory domain of the juxtacrine signaling receptor Notch, linked to a chimeric extracellular recognition domain (such as but not limited to an scFv and a chimeric intracellular transcriptional domain. Upon interacting with the cognate ligand which may be present on a neighboring cell, the SynNotch receptor undergoes cleavage of the transmembrane region, releasing the intracellular transcriptional domain to enter the nucleus and drive the expression of user-specified target genes. SynNotch circuits may be generated by linking one or more transcriptional outputs to SynNotch receptors. Any of the SynNotch circuits described by Toda et al. may be used in the biocircuits described herein (Programming self-organizing multicellular structures with synthetic cell-cell signaling Science 13 Jul. 2018: Vol. 361, Issue 6398, pp. 156-162; the contents of which are herein incorporated by reference in their entirety).

In some embodiments, the biocircuits described herein may include a proteolytic switch. Such switches may include a set of split proteases with highly specific orthogonal cleavage motifs which may be combined with cleavage sites and orthogonal coiled coils dimerizing domains strategically positioned in payloads of interest. Proteolytic cleavage induces conformational rearrangements within and between the CC modules, thus leading to the reconstitution of downstream functional protein domains resulting in a cellular outcome. Split-protease-cleavable orthogonal-CC-based (SPOC) logic circuits described by Fink et al. may be utilized in the biocircuits described herein (Design of fast proteolysis-based signaling and logic circuits in mammalian cells, Nat. Chem. Bio 15, 115-122 (2019); the contents of which are herein incorporated by reference in their entirety).

In one embodiment, the molecular switches may be based on chemically induced proximity (CIP) systems, wherein two proteins are colocalized upon addition of a bridging small molecule. In some aspects, the molecular switches may be based on chemically disrupted proximity (CDP) systems, which may include systems that allow the interaction of two basally colocalized proteins to be rapidly disrupted with a small. As non-limiting example, the interaction between the antiapoptotic protein BCL-xL and a BH3 peptide may be used as a chemically disruptable proximity system for intramolecularly controlling the activities of various enzymes. Intermolecular CDP systems that allow a basally localized activity to be chemically disrupted may be used as off switches. In one embodiment, the CDP system may be based on the hepatitis C virus protease (HCVp) NS3a and its interaction with a peptide inhibitor. Clinically approved protease inhibitors that efficiently disrupt the NS3a/peptide interaction may be utilized as bio-orthogonal inputs for this system.

In some embodiments, Zapalog, a small molecule dimerizer that undergoes photolysis when exposed to blue light. Zapalog dimerizes any two proteins tagged with the FKBP and DHFR domains until exposure to light causes its photolysis. Dimerization can be repeatedly restored with uncleaved Zapalog. In some embodiments, molecular switches described herein may be heterodimerization switches. Heterodimerization may include chemical induced dimerization domains (CIDs) with two orthogonal ligand moieties that exhibit high specificity and affinity for their binding domains and lack alternative endogenous binding partners in mammalian cells at useful concentrations. The CIDs may be membrane permeable. In some aspects, the linker may be long enough to allow both binding domains to be simultaneously engaged without steric interference. The linker may in some instances be a photocleavable linker. Linkers that respond to a wavelength that is neither cytotoxic nor interferes with imaging common fluorophores may be preferentially selected. In some embodiments, the heterodimerization switches may include FKBP and DHFR domains and Zapalog, a small molecule dimerizer that undergoes photolysis when exposed to blue light. Zapalog may dimerize any two proteins tagged with the FKBP and DHFR domains until exposure to light causes its photolysis. Dimerization may be restored with uncleaved Zapalog. In some embodiments, Zapalog may include the heterodimerizer trimethoprim-synthetic ligand of FK506-binding protein (TMP-SLF), wherein the SLF portion binds the FK506-binding protein (FKBP) domain of one chimeric partner; the TMP portion binds the Escherichia coli dihydrofolate reductase (DHFR) domain of another. To render dimerization reversible with light, the alkyl linker between TMP and SLF may be replaced with a dialkoxynitrobenzyl (DANB) moiety. Two payloads described herein are tagged with DHFR and FKBP domains. Addition of Zapalog may induce dimerization of the tagged proteins. Exposure to 405 nm light may photocleave Zapalog, causing rapid dissociation of the dimer. Dimerization may be restored by the addition of uncleaved Zapalog which may outcompete the photolyzed Zapalog moieties. Any of the ligands described herein may be utilized to build Zapalog-like ligands and any of the SREs described herein may be used to generate CIDs.

### Boolean Switches

DD-regulated effector modules described herein can also be incorporated into the design of cellular Boolean switches. As used herein, a Boolean switch refers to a circuit that is designed to perform a logical operation based on one or more inputs and which produces an output. Logical operations performed by Boolean switches include but are not limited to, AND, OR, NOR, NAND, NOT, IMPLY, NIMPLY, XOR, and XNOR. AND and OR gates represent the most fundament logical operations, where OR represents a scenario where any of the one or more inputs is required to produce an output, and AND represents a scenario where all of the inputs are required to generate an output. Compound Boolean switches that consist of multiple logical operations can also be generated using effector modules as described herein. In some embodiments, biocircuits and/or any of their components can represent one or more inputs in a Boolean switch. In other embodiments, DDs described herein can be combined with switches known in the art to generate Boolean switches. The output of a Boolean switch can depend on the payload of the effector module utilized. In one embodiment, the DDs and/or the biocircuits can be utilized in NOT gates. NOT gates are inverters whose function is to invert the input i.e. stimulus. In some embodiments, the NOT gates described herein can be used to convert an immunosuppressive stimulus into an immune potentiating output such as the expression and/or function of an immune potentiating (promoting) payload. In another non-limiting example, an AND based Boolean switch may be generated where a first input comprises a biocircuit with gene editing nuclease, Cas9, as the payload and a second input comprises a biocircuit with transcriptional activator, VPR, as the payload. In the presence of the target gene guide RNA, addition of the stimuli to both inputs is required for the transcriptional activation of the target gene (see Gao Y et al. (2016) Nat Methods, 13(12):1043-1049; the contents of which are incorporated herein by reference in their entirety).

### Safety Switches

In some embodiments, effector module payloads can comprise DD-regulated safety switches that can eliminate adoptively transferred cells in the case of severe toxicity, thereby mitigating the adverse effects of T cell therapy. Adoptively transferred T cells in immunotherapy can attack normal cells in response to normal tissue expression of TAA. Even on-tumor target activity of adoptively transferred T cells can result in toxicities such as tumor lysis syndrome, cytokine release syndrome and the related macrophage activation syndrome. Safety switches can be utilized to eliminate inappropriately activated adoptively transferred cells by induction of apoptosis or by immunosurveillance.

In some embodiments, DD-regulated payloads can comprise inducible killer/suicide genes that act as a safety switch. The killer/suicide gene when introduced into adoptively transferred immune cells, could control their alloreactivity. The killer/suicide gene can be an apoptotic gene (e.g., any Caspase gene) which allows conditional apoptosis of the transduced cells by administration of a non-therapeutic ligand of the SRE (e.g., DD).

In some embodiments, the payload can be Caspase 9. In some instances, Caspase 9 can be modified to have low basal expression and lacking the caspase recruitment domain (CARD) (SEQ ID NO. 26 and SEQ ID NO. 28 of U.S. Pat. No. 9,434,935B2; the contents of which are incorporated by reference in their entirety). In some embodiments, the safety switches may also be referred to as genetic erasers. In one aspect, the genetic eraser may include a selection marker such as those described in International Patent Publication, WO2018022747, the contents of which are incorporated by reference in their entirety.

In some embodiments, DDs, SREs and/or payloads of effector modules as described can include molecular safeguards for T-cell immunotherapies. Molecular safeguards can allow, in the event of side-effects, selective depletion of engineered T-cells (e.g., CAR T-cells). Molecular safeguards can respond to activating agents which trigger on-demand depletion of the engineered T-cells. Non-limiting examples of molecular safeguards to be used in combination with CAR expression include CD20, which respond to the antibody Rituximab; RQR8, which responds to the antibody Rituximab; huEGFRtr, which responds to the antibody Erbitux; HSV-TK, which responds to the small molecule Ganciclovir; ICasp9, which responds to the small molecule Rimudicid; and CubiCAR, which is a CAR architecture with built-in safeguard CD20 that responds to the antibody Rituximab (Valton et al., Scientific Reports volume 8, Article number: 8972 (2018), the contents of which are incorporated herein by reference in their entirety).

In one embodiment, the payload is a suicide gene system, iCasp9/Chemical induced dimerization (CID) system which consists of a polypeptide derived from the Caspase9 gene fused to a drug binding domain derived from the human FK506 protein. Administration of bioinert, small molecule AP1903 (rimiducid), induces cross linking of the drug binding domains and dimerization of the fusion protein and in turn the dimerization of Caspase 9. This results in the activation of downstream effector Caspase 3 and subsequent induction of cellular apoptosis (Straathof et al., Blood, 2005, 105: 4247-4254; incorporated herein by reference in its entirety). Preclinical trials using CART including an iCasp9 gene have shown effective elimination of CAR T cells in vivo in mouse models and demonstrate the potential efficacy of this approach. (Budde et al, Plos One, 2013, 8: e82742.10.1371; Hoyos et al., Leukemia, 2010; 24(6):1160-1170). In one embodiment, the payload can comprise Caspase 9. In one aspect, the effector module can be a DD-Caspase9 fusion polypeptide.

### Regulatory Switches

The utility of adoptive cell therapy (ACT) has been limited by the high incidence of graft versus host disease (GVHD). GVHD occurs when adoptively transferred T cells elicit an immune response resulting in host tissue damage. Recognition of host antigens by the graft cells triggers a proinflammatory cytokine storm cascade that signifies acute GVHD. GVHD is characterized as an imbalance between the effector and the regulatory arms of the immune system. In some embodiments, the payloads described herein can be used as regulatory switches. As used herein “regulatory switch” refers proteins, which when expressed in target cells increase tolerance to the graft by enhancing the regulatory arm of the immune system.

In one embodiment, regulatory switches can include DD-regulated payloads that preferentially promote the expansion of regulatory T (Treg cells). Tregs are a distinct population of cells that are positively selected on high affinity ligands in the thymus and play an important role in the tolerance to self-antigens. In addition, Tregs have also been shown to play a role in peripheral tolerance to foreign antigens. Since Tregs promote immune tolerance, expansion of Tregs with compositions as described herein can be desirable to limit GVHD.

In some embodiments, the regulatory switch can include, but is not limited to T regs activation factors such NF-kappa B (NFKB or NFκB) FOXO, nuclear receptor Nr4a, Retinoic acid receptor alpha, NFAT, AP-1 and SMAD. Such factors can result in the expression of Fork headbox P3 (FOXP3) in T cells resulting in the activation of the regulatory T cell program and the expansion of T cells.

In some embodiments, the regulatory switch can be FOXP3, a transcriptional regulator in T cells. A function of FOXP3 is to suppress the function of NFAT, which leads to the suppression of expression of many genes including IL2 and effector T-cell cytokines. FOXP3 acts also as a transcription activator for genes such as CD2S, Cytotoxic T-Lymphocyte Antigen Cytotoxic T-Lymphocyte Antigen 4 (CTLA4), glucocorticoid-induced TNF receptor family gene (GITR) and folate receptor 4. FOXP3 also inhibits the differentiation of IL17 producing helper T-cells (Th17) by antagonizing RORC (RAR related orphan receptor C). Isoforms of FOXP3 lacking exon2 (FOXP3 delta 2) or exon 7 (FOXP3 delta 7) can also be used as regulatory switches. In one aspect, an effector module as described herein can be a DD-FOXP3 fusion polypeptide.

Regulatory switches can be designed using Boolean switches, e.g. NOT gates. For example, an immunosuppressive signal can be converted into an immune potentiating output such as expression and/or function of an immune potentiating (promoting) payload. The stimulus of a NOT gate can be, for example, phosphorylated SHP2, a signaling molecule produced by the activation of the immunosuppressive PD1 signaling pathway. Another non-limiting example for a NOT gate can be metabolite kynurenine, which is produced by the indoleamine 2,3-dioxygenase pathway.

### Epigenetic Switches

In some embodiments, the DDs described herein can include epigenetic switches. Alternatively or additionally, epigenetic switches can be constructed using DDs described herein. Epigenetic switches utilize chemical modification of nucleic acids, the propagation, and the actuation of the chemical modifications. In some embodiments, the epigenetic switches may be built by utilizing chemical modifications of nucleic acids that are naturally found in bacterial such as 6-methyl-adenosine (m6A). m6A switches are described in Park et al. 2019 (Engineering Epigenetic Regulation Using Synthetic Read-Write Modules. Cell. January 10; 176(1-2):227-238.; the contents of which are incorporated by reference in their entirety). The epigenetic circuits described herein may include a “writer” protein that adds m6A at specific DNA sequences. The writer module may be based on a methylase such as but not limited to dam methylase, which adds m6A to GATC sequences in Escherichia coli and is active when expressed in human cells. By fusing dam to a zinc finger proteins (ZF), allows targeting m6A methylation to specific loci. Specificity of the dam methylases to ZF targeted sequences may be achieved using by engineering mutations that reduce the catalytic activity of dam. In some embodiments, the epigenetic switch may include a “reader” protein that can recognize the m6A methylation marks. In one aspect, the reader domain may be the binding domain of the restriction enzyme DpnI. This reader domain (RD) may be fused to DAM to propagate m6A marks at nearby and complementary GATC sites in DNA, thereby spreading methylation across a locus and cell divisions. The RD may also be fused to proteins that activate or repress transcription (EDs), thus creating m6A-dependent transcriptional regulation.

### III. Pharmaceutical Compostions and Formulations

The present disclosure further provides pharmaceutical compositions comprising the DDs of the disclosure, one or more stimuli, effector modules and biocircuit systems comprising the same, and optionally at least one pharmaceutically acceptable excipient or inert ingredient.

As used herein the term “pharmaceutical composition” refers to a preparation of activate agents (e.g., DDs, ligands of the DDs, effector modules and biocircuits), other components, vectors, cells and described herein, or pharmaceutically acceptable salts thereof, optionally with other chemical components such as physiologically suitable carriers and excipients. The pharmaceutical compositions of the disclosure comprise an effective amount of one or more active compositions of the disclosure. The preparation of a pharmaceutical composition that contains at least one composition of the present disclosure and/or an additional active ingredient will be known to those skilled in the art in light of the present disclosure, as exemplified by Remington's Pharmaceutical Sciences, 18th Ed. Mack Printing Company, 1990, incorporated herein by reference.

The term “excipient” or “inert ingredient” refers to an inactive substance added to a pharmaceutical composition and formulation to further facilitate administration of an active ingredient. For the purposes of the present disclosure, the phrase “active ingredient” generally refers to any one or more biocircuits, effector modules, DDs, stimuli and payloads (i.e., immunotherapeutic agents), other components, vectors, and cells to be delivered as described herein. The phrases “pharmaceutically acceptable” refers to molecular entities and compositions that do not produce an adverse, allergic or other untoward reaction when administered to an animal, such as, for example, a human, as appropriate.

In some embodiments, pharmaceutical compositions and formulations are administered to humans, human patients or subjects. Although the descriptions of pharmaceutical compositions provided herein are principally directed to pharmaceutical compositions which are suitable for administration to humans, it will be understood by the skilled artisan that such compositions are generally suitable for administration to any other animal, e.g., to non-human animals, e.g. non-human mammals. Subjects to which administration of the pharmaceutical compositions is contemplated include, but are not limited to, non-human mammals, including agricultural animals such as cattle, horses, chickens and pigs, domestic animals such as cats, dogs, or research animals such as mice, rats, rabbits, dogs and non-human primates. It will be understood that, for human administration, preparations should meet sterility, pyrogenicity, general safety and purity standards as required by FDA Office of Biological Standards.

A pharmaceutical composition and formulation in accordance with the disclosure may be prepared, packaged, and/or sold in bulk, as a single unit dose, and/or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

The compositions of the present disclosure may be formulated in any manner suitable for delivery. The formulation may be, but is not limited to, nanoparticles, poly (lactic-co-glycolic acid) (PLGA) microspheres, lipidoids, lipoplex, liposome, polymers, carbohydrates (including simple sugars), cationic lipids and combinations thereof.

In one embodiment, the formulation is a nanoparticle which may comprise at least one lipid. The lipid may be selected from, but is not limited to, DLin-DMA, DLin-K-DMA, 98N12-5, C12-200, DLin-MC3-DMA, DLin-KC2-DMA, DODMA, PLGA, PEG, PEG-DMG and PEGylated lipids. In another aspect, the lipid may be a cationic lipid such as, but not limited to, DLin-DMA, DLin-D-DMA, DLin-MC3-DMA, DLin-KC2-DMA and DODMA.

For polynucleotides of the disclosure, the formulation may be selected from any of those taught, for example, in International Application PCT/US2012/069610, the contents of which are incorporated herein by reference in its entirety.

Relative amounts of the active ingredient, the pharmaceutically acceptable excipient or inert ingredient, and/or any additional ingredients in a pharmaceutical composition in accordance with the disclosure will vary, depending upon the identity, size, and/or condition of the subject treated and further depending upon the route by which the composition is to be administered. By way of example, the composition may comprise between 0.1% and 100%, e.g., between 0.5 and 50%, between 1-30%, between 5-80%, at least 80% (w/w) active ingredient.

Efficacy of treatment or amelioration of disease can be assessed, for example by measuring disease progression, disease remission, symptom severity, reduction in pain, quality of life, dose of a medication required to sustain a treatment effect, level of a disease marker or any other measurable parameter appropriate for a given disease being treated or targeted for prevention. It is well within the ability of one skilled in the art to monitor efficacy of treatment or prevention by measuring any one of such parameters, or any combination of parameters. In connection with the administration of compositions of the present disclosure, “effective against” for example a cancer, indicates that administration in a clinically appropriate manner results in a beneficial effect for at least a statistically significant fraction of patients, such as an improvement of symptoms, a cure, a reduction in disease load, reduction in tumor mass or cell numbers, extension of life, improvement in quality of life, or other effect generally recognized as positive by medical doctors familiar with treating the particular type of cancer.

A treatment or preventive effect is evident when there is a statistically significant improvement in one or more parameters of disease status, or by a failure to worsen or to develop symptoms where they would otherwise be anticipated. As an example, a favorable change of at least 10% in a measurable parameter of disease, and preferably at least 20%, 30%, 40%, 50% or more can be indicative of effective treatment. Efficacy for a given composition or formulation of the present disclosure can also be judged using an experimental animal model for the given disease as known in the art. When using an experimental animal model, efficacy of treatment is evidenced when a statistically significant change is observed.

In some embodiments, the polypeptides of the disclosure may be delivered to the cell directly. In one embodiment, the polypeptides of the disclosure may be delivered using synthetic peptides comprising an endosomal leakage domain (ELD) fused to a cell penetration domain (CLD). The polypeptides of the disclosure are co introduced into the cell with the ELD-CLD-synthetic peptide. ELDs facilitate the escape of proteins that are trapped in the endosome, into the cytosol. Such domains are derived proteins of microbial and viral origin and have been described in the art. CPDs allow the transport of proteins across the plasma membrane and have also been described in the art. The ELD-CLD fusion proteins synergistically increase the transduction efficiency when compared to the co-transduction with either domain alone. In some embodiments, a histidine rich domain may optionally be added to the shuttle construct as an additional method of allowing the escape of the cargo from the endosome into the cytosol. The shuttle may also include a cysteine residue at the N or C terminus to generate multimers of the fusion peptide. Multimers of the ELD-CLD fusion peptides generated by the addition of cysteine residue to the terminus of the peptide show even greater transduction efficiency when compared to the single fusion peptide constructs. The polypeptides of the disclosure may also be appended to appropriate localization signals to direct the cargo to the appropriate sub-cellular location e.g. nucleus. In some embodiments any of the ELDs, CLDs or the fusion ELD-CLD synthetic peptides taught in the International Patent Publication, WO2016161516 and WO2017175072 may be useful in the present disclosure (the contents of each of which are herein incorporated by reference in their entirety).

### Therapeutic Uses

### 1. Cancer

Various cancers may be treated with pharmaceutical compositions, biocircuits, biocircuit components, effector modules including their SREs or payloads of the present disclosure. As used herein, the term “cancer” refers to any of various malignant neoplasms characterized by the proliferation of anaplastic cells that tend to invade surrounding tissue and metastasize to new body sites and also refers to the pathological condition characterized by such malignant neoplastic growths. Cancers may be tumors or hematological malignancies, and include but are not limited to, all types of lymphomas/leukemias, carcinomas and sarcomas, such as those cancers or tumors found in the anus, bladder, bile duct, bone, brain, breast, cervix, colon/rectum, endometrium, esophagus, eye, gallbladder, head and neck, liver, kidney, larynx, lung, mediastinum (chest), mouth, ovaries, pancreas, penis, prostate, skin, small intestine, stomach, spinal marrow, tailbone, testicles, thyroid and uterus.

Types of carcinomas which may be treated with the compositions of the present disclosure include, but are not limited to, papilloma/carcinoma, choriocarcinoma, endodermal sinus tumor, teratoma, adenoma/adenocarcinoma, melanoma, fibroma, lipoma, leiomyoma, rhabdomyoma, mesothelioma, angioma, osteoma, chondroma, glioma, lymphoma/leukemia, squamous cell carcinoma, small cell carcinoma, large cell undifferentiated carcinomas, basal cell carcinoma and sinonasal undifferentiated carcinoma.

Types of carcinomas which may be treated with the compositions of the present disclosure include, but are not limited to, soft tissue sarcoma such as alveolar soft part sarcoma, angiosarcoma, dermatofibrosarcoma, desmoid tumor, desmoplastic small round cell tumor, extraskeletal chondrosarcoma, extraskeletal osteosarcoma, fibrosarcoma, hemangiopericytoma, hemangiosarcoma, Kaposi's sarcoma, leiomyosarcoma, liposarcoma, lymphangiosarcoma, lymphosarcoma, malignant fibrous histiocytoma, neurofibrosarcoma, rhabdomyosarcoma, synovial sarcoma, and Askin's tumor, Ewing's sarcoma (primitive neuroectodermal tumor), malignant hemangioendothelioma, malignant schwannoma, osteosarcoma, and chondrosarcoma.

As a non-limiting example, the carcinoma which may be treated may be Acute granulocytic leukemia, Acute lymphocytic leukemia, Acute myelogenous leukemia, Adenocarcinoma, Adenosarcoma, Adrenal cancer, Adrenocortical carcinoma, Anal cancer, Anaplastic astrocytoma, Angiosarcoma, Appendix cancer, Astrocytoma, Basal cell carcinoma, B-Cell lymphoma), Bile duct cancer, Bladder cancer, Bone cancer, Bowel cancer, Brain cancer, Brain stem glioma, Brain tumor, Breast cancer, Carcinoid tumors, Cervical cancer, Cholangiocarcinoma, Chondrosarcoma, Chronic lymphocytic leukemia, Chronic myelogenous leukemia, Colon cancer, Colorectal cancer, Craniopharyngioma, Cutaneous lymphoma, Cutaneous melanoma, Diffuse astrocytoma, Ductal carcinoma in situ, Endometrial cancer, Ependymoma, Epithelioid sarcoma, Esophageal cancer, Ewing sarcoma, Extrahepatic bile duct cancer, Eye cancer, Fallopian tube cancer, Fibrosarcoma, Gallbladder cancer, Gastric cancer, Gastrointestinal cancer, Gastrointestinal carcinoid cancer, Gastrointestinal stromal tumors, General, Germ cell tumor, Glioblastoma multiforme, Glioma, Hairy cell leukemia, Head and neck cancer, Hemangioendothelioma, Hodgkin lymphoma, Hodgkin's disease, Hodgkin's lymphoma, Hypopharyngeal cancer, Infiltrating ductal carcinoma, Infiltrating lobular carcinoma, Inflammatory breast cancer, Intestinal Cancer, Intrahepatic bile duct cancer, Invasive/infiltrating breast cancer, Islet cell cancer, Jaw cancer, Kaposi sarcoma, Kidney cancer, Laryngeal cancer, Leiomyosarcoma, Leptomeningeal metastases, Leukemia, Lip cancer, Liposarcoma, Liver cancer, Lobular carcinoma in situ, Low-grade astrocytoma, Lung cancer, Lymph node cancer, Lymphoma, Male breast cancer, Medullary carcinoma, Medulloblastoma, Melanoma, Meningioma, Merkel cell carcinoma, Mesenchymal chondrosarcoma, Mesenchymous, Mesothelioma, Metastatic breast cancer, Metastatic melanoma, Metastatic squamous neck cancer, Mixed gliomas, Mouth cancer, Mucinous carcinoma, Mucosal melanoma, Multiple myeloma, Nasal cavity cancer, Nasopharyngeal cancer, Neck cancer, Neuroblastoma, Neuroendocrine tumors, Non-Hodgkin lymphoma, Non-Hodgkin's lymphoma, Non-small cell lung cancer, Oat cell cancer, Ocular cancer, Ocular melanoma, Oligodendroglioma, Oral cancer, Oral cavity cancer, Oropharyngeal cancer, Osteogenic sarcoma, Osteosarcoma, Ovarian cancer, Ovarian epithelial cancer, Ovarian germ cell tumor, Ovarian primary peritoneal carcinoma, Ovarian sex cord stromal tumor, Paget's disease, Pancreatic cancer, Papillary carcinoma, Paranasal sinus cancer, Parathyroid cancer, Pelvic cancer, Penile cancer, Peripheral nerve cancer, Peritoneal cancer, Pharyngeal cancer, Pheochromocytoma, Pilocytic astrocytoma, Pineal region tumor, Pineoblastoma, Pituitary gland cancer, Primary central nervous system lymphoma, Prostate cancer, Rectal cancer, Renal cell cancer, Renal pelvis cancer, Rhabdomyosarcoma, Salivary gland cancer, Sarcoma, Sarcoma, bone, Sarcoma, soft tissue, Sarcoma, uterine, Sinus cancer, Skin cancer, Small cell lung cancer, Small intestine cancer, Soft tissue sarcoma, Spinal cancer, Spinal column cancer, Spinal cord cancer, Spinal tumor, Squamous cell carcinoma, Stomach cancer, Synovial sarcoma, T-cell lymphoma), Testicular cancer, Throat cancer, Thymoma/thymic carcinoma, Thyroid cancer, Tongue cancer, Tonsil cancer, Transitional cell cancer, Transitional cell cancer, Transitional cell cancer, Triple-negative breast cancer, Tubal cancer, Tubular carcinoma, Ureteral cancer, Ureteral cancer, Urethral cancer, Uterine adenocarcinoma, Uterine cancer, Uterine sarcoma, Vaginal cancer, and Vulvar cancer.

### 2. Combination Treatments

The disclosure further relates to the use of pharmaceutical compositions, biocircuits, biocircuit components, effector modules including their SREs or payloads of the present disclosure for treating one or more forms of cancer, in combination with other pharmaceuticals and/or other therapeutic methods, e.g., with known pharmaceuticals and/or known therapeutic methods, such as, for example, those which are currently employed for treating these disorders. For example, the pharmaceutical compositions, biocircuits, biocircuit components, effector modules including their SREs or payloads of the present disclosure can also be administered in conjunction with one or more additional anti-cancer treatments, such as biological, chemotherapy and radiotherapy. Accordingly, a treatment can include, for example, imatinib (Gleevac), all-trans-retinoic acid, a monoclonal antibody treatment (gemtuzumab, ozogamicin), chemotherapy (for example, chlorambucil, prednisone, prednisolone, vincristine, cytarabine, clofarabine, farnesyl transferase inhibitors, decitabine, inhibitors of MDR1), rituximab, interferon-α, anthracycline drugs (such as daunorubicin or idarubicin), L-asparaginase, doxorubicin, cyclophosphamide, doxorubicin, bleomycin, fludarabine, etoposide, pentostatin, or cladribine), bone marrow transplant, stem cell transplant, radiation therapy, anti-metabolite drugs (methotrexate and 6-mercaptopurine), or any of the antibodies taught herein such as those in Table 6 of the co-pending commonly owned U.S. Ser. No. 62/320,864 filed on Apr. 11, 2016, or in U.S. Provisional Application No. 62/466,596 filed Mar. 3, 2017 and the International Publication WO2017/180587, the contents of each of which are incorporated herein by reference in their entirety or combinations thereof.

3. Combinations with Radiation

Radiation therapy (also called radiotherapy, X-ray therapy, or irradiation) is the use of ionizing radiation to kill cancer cells and shrink tumors. Radiation therapy can be administered externally via external beam radiotherapy (EBRT) or internally via brachytherapy. The effects of radiation therapy are localized and confined to the region being treated. Radiation therapy may be used to treat almost every type of solid tumor, including cancers of the brain, breast, cervix, larynx, lung, pancreas, prostate, skin, stomach, uterus, or soft tissue sarcomas. Radiation is also used to treat leukemia and lymphoma.

4. Combination with Chemotherapy

Chemotherapy is the treatment of cancer with drugs that can destroy cancer cells. In current usage, the term “chemotherapy” usually refers to cytotoxic drugs which affect rapidly dividing cells in general, in contrast with targeted therapy. Chemotherapy drugs interfere with cell division in various possible ways, e.g. with the duplication of DNA or the separation of newly formed chromosomes. Most forms of chemotherapy target all rapidly dividing cells and are not specific to cancer cells, although some degree of specificity may come from the inability of many cancer cells to repair DNA damage, while normal cells generally can.

Most chemotherapy regimens are given in combination. Exemplary chemotherapeutic agents include, but are not limited to, 5-FU Enhancer, 9-AC, AG2037, AG3340, Aggrecanase Inhibitor, Aminoglutethimide, Amsacrine (m-AMSA), Asparaginase, Azacitidine, Batimastat (BB94), BAY 12-9566, BCH-4556, Bis-Naphtalimide, Busulfan, Capecitabine, Carboplatin, Carmustaine+Polifepr Osan, cdk4/cdk2 inhibitors, Chlorambucil, CI-994, Cisplatin, Cladribine, CS-682, Cytarabine HCl, D2163, Dactinomycin, Daunorubicin HCl, DepoCyt, Dexifosamide, Docetaxel, Dolastain, Doxifluridine, Doxorubicin, DX8951f, E 7070, EGFR, Epirubicin, Erythropoietin, Estramustine phosphate sodium, Etoposide (VP16-213), Farnesyl Transferase Inhibitor, FK 317, Flavopiridol, Floxuridine, Fludarabine, Fluorouracil (5-FU), Flutamide, Fragyline, Gemcitabine, Hexamethylmelamine (HMM), Hydroxyurea (hydroxycarbamide), Ifosfamide, Interferon Alfa-2a, Interferon Alfa-2b, Interleukin-2, Irinotecan, ISI 641, Krestin, Lemonal DP 2202, Leuprolide acetate (LHRH-releasing factor analogue), Levamisole, LiGLA (lithium-gamma linolenate), Lodine Seeds, Lometexol, Lomustine (CCNU), Marimistat, Mechlorethamine HCl (nitrogen mustard), Megestrol acetate, Meglamine GLA, Mercaptopurine, Mesna, Mitoguazone (methyl-GAG; methyl glyoxal bis-guanylhydrazone; MGBG), Mitotane (o.p′-DDD), Mitoxantrone, Mitoxantrone HCl, MMI 270, MMP, MTA/LY 231514, Octreotide, ODN 698, OK-432, Oral Platinum, Oral Taxoid, Paclitaxel (TAXOL®), PARP Inhibitors, PD 183805, Pentostatin (2′ deoxycoformycin), PKC 412, Plicamycin, Procarbazine HCl, PSC 833, Ralitrexed, RAS Farnesyl Transferase Inhibitor, RAS Oncogene Inhibitor, Semustine (methyl-CCNU), Streptozocin, Suramin, Tamoxifen citrate, Taxane Analog, Temozolomide, Teniposide (VM-26), Thioguanine, Thiotepa, Topotecan, Tyrosine Kinase, UFT (Tegafur/Uracil), Valrubicin, Vinblastine sulfate, Vindesine sulfate, VX-710, VX-853, YM 116, ZD 0101, ZD 0473/Anormed, ZD 1839, ZD 9331.

### 5. Immuno-Oncology and Cell Therapies

Recent progress in the field of cancer immunology has allowed the development of several approaches to help the immune system keep the cancer at bay. Such immunotherapy approaches include the targeting of cancer antigens through monoclonal antibodies or through adoptive transfer of ex vivo engineered T cells (e.g., which contain chimeric antigen receptors or engineered T cell receptors).

In some embodiments, pharmaceutical compositions, biocircuits, biocircuit components, effector modules including their SREs or payloads of the present disclosure may be used in the modulation or alteration or exploitation of the immune system to target one or more cancers. This approach may also be considered with other such biological approaches, e.g., immune response modifying therapies such as the administration of interferons, interleukins, colony-stimulating factors, other monoclonal antibodies, vaccines, gene therapy, and nonspecific immunomodulating agents are also envisioned as anti-cancer therapies to be combined with the pharmaceutical compositions, biocircuits, biocircuit components, effector modules including their SREs or payloads of the present disclosure.

Cancer immunotherapy refers to a diverse set of therapeutic strategies designed to induce the patient's own immune system to fight the cancer. In some embodiments, pharmaceutical compositions, biocircuits, biocircuit components, effector modules including their SREs or payloads of the present disclosure are designed as immune-oncology therapeutics.

Also provided herein are methods of increasing the survival in a subject with cancer. The methods may comprise introducing into the subject, any of the compositions described herein. The subject may be administered at least one stimulus described herein. In one embodiment the stimulus may be a ligand. In one embodiment, administration of the ligand may result in the modulation of the payload. Payloads such as but not limited to CARs when expressed in response to the ligand, may trigger an anti-cancer immune response. Such anti-cancer immune responses may result in reduction of tumor burden which in turn may increase the survival of the subject with cancer. Ligand may be administered prior to, during or after the introduction of the compositions into the subject. Survival may be measured and quantified using any of the methods known in the art. In some embodiment, survival may be quantified using Kaplan Meier analysis and/or the log rank test. In some embodiments, survival may be quantified using the survival ratio, which may be defined as the ratio of the median survival observed in a group to the median survival of the untransduced-vehicle treatment group. In some embodiments, the survival ratio is at least 1. In some embodiments, the survival ratio is at least 1.1, 1.2, 1.3, 1.4, 1.5, 1.6, 1.7 1.8, 1.9, 2, 3, 4, 5, 6, 7, 8, 9, or 10.

The present invention also provides methods of reducing tumor burden in a subject. In some embodiments. As used herein, “tumor burden” refers to the number of cancer cells, or the amount of cancer in a subject. In some aspects tumor burden also refers to tumor load. In some embodiments, the tumor may be disseminated throughout the body of the subject. In one aspect, the tumor may be a liquid tumor such as leukemia or a lymphoma. The methods of reducing tumor burden may involve administering to the subject, a therapeutically effective amount of the immune cells. Immune cells may be engineered to express the compositions described herein. In some embodiments, the immune cells expressing the compositions of the invention may be administered to the subject via any of the routes of delivery described herein. Also provided herein are dosing regimens for administering the immune cells. In some embodiments, the subject may also be administered a therapeutically effective amount of the stimulus to tune the expression of the immunotherapeutic agent. In some aspects, the immunotherapeutic agents may be capable of reducing the tumor burden. Regimens for ligand/stimulus dosing are also provided. Reduction in tumor burden may be measured by any of the methods known in the art including tumor imaging, and measurement of marker proteins. In some aspects, bioluminescent imaging may be used to measure tumor burden. Bioluminescence imaging utilizes native light emission from bioluminescent proteins such as luciferase. Such bioluminescent proteins can participate in chemical reactions that release photons by the addition of suitable substrates. The release of photons can be captured by sensitive detection methods and quantified. Tumor cells may be engineered to express luciferase and the efficacy of the compositions described herein to reduce tumor burden may quantified by imaging. In some aspects, the tumor burden may be measured by the flux of photons (photons per sec). In some embodiments, photon flux positively correlates with tumor burden.

Parameters for improving CAR-T therapy outcome are described in Finney et al. JCI. 2019; 129(5):2123-2132 (the contents of which are herein incorporated by reference in their entirety). The levels of biomarker LAG3 (high)/TNF-α (low) in peripheral blood CD8+ T cells at the time of apheresis may also predict a subsequent dysfunctional response in subjects with high antigen load who do not achieve complete response that is durable for more than a few weeks. T cell-intrinsic features that are a consequence of the starting T cell repertoire and the effects of the manufacturing process converge with CD19 antigen-induced activation following adoptive transfer may also play a role in the outcome of CAR-T therapy. The starting T cell repertoire may in part be affected by the timing of the apheresis. In one embodiment, the apheresis may be performed prior to chemotherapy. Cumulative burden of CD19 expressing leukemic and normal B cells, as evaluated in the bone marrow prior to lymphodepleting chemotherapy may be important for determining CAR-T therapy outcome. According to Finney et al., increase antigen burden improves CAR-T therapy outcome. To increase CD19 antigen burden in vivo, subjects may also be infused with expanded subject derived T cells genetically modified to express CD19 (also referred to as T-APCs).

### 6. Cell Therapies

There are several types of cellular immunotherapies, including tumor infiltrating lymphocyte (TIL) therapy, genetically engineered T cells bearing chimeric antigen receptors (CARs), and recombinant TCR technology.

According to the present disclosure, the biocircuits and systems may be used in the development and implementation of cell therapies such as adoptive cell therapy. The biocircuits, their components, effector modules and their SREs and payloads may be used in cell therapies to effect TCR removal-TCR gene disruption, TCR engineering, to regulate epitope tagged receptors, in APC platforms for stimulating T cells, as a tool to enhance ex vivo APC stimulation, to improve methods of T cell expansion, in ex vivo stimulation with antigen, in TCR/CAR combinations, in the manipulation or regulation of TILs, in allogeneic cell therapy, in combination T cell therapy with other treatment lines (e.g. radiation, cytokines), to encode engineered TCRs, or modified TCRs, or to enhance T cells other than TCRs (e.g. by introducing cytokine genes, genes for the checkpoint inhibitors PD1, CTLA4).

In some embodiments, improved response rates are obtained in support of cell therapies.

Expansion and persistence of cell populations may be achieved through regulation or fine tuning of the payloads, e.g., the receptors or pathway components in T cells, NK cells or other immune-related cells. In some embodiments, biocircuits, their components, SREs or effector modules are designed to spatially and/or temporally control the expression of proteins which enhance T-cell or NK cell response. In some embodiments, biocircuits, their components, SREs or effector modules are designed to spatially and/or temporally control the expression of proteins which inhibit T-cell or NK cell response.

In some embodiments, biocircuits, their components, SREs or effector modules are designed to reshape the tumor microenvironment in order to extend utility of the biocircuit or a pharmaceutical composition beyond direct cell killing.

In some embodiments, biocircuits, their components, SREs or effector modules are designed to reduce, mitigate or eliminate the CAR cytokine storm. In some embodiments such reduction, mitigation and/or elimination occurs in solid tumors or tumor microenvironments.

In some embodiments the effector modules may encode one or more cytokines. In some embodiments, the cytokine is IL15. Effector modules encoding IL15 may be designed to induce proliferation in cytotoxic populations and avoid stimulation of Tregs. In other cases, the effector modules which induce proliferation in cytotoxic populations may also stimulate NK and NKT cells.

In some embodiments, effector modules may encode, or be tuned or induced to produce, one or more cytokines for expansion of cells in the biocircuits of the disclosure. In such cases the cells may be tested for actual expansion. Expansion may be at least 5%, 10%, 20%, 30%, 40%, 50%, 60%, 70%, 80%, 90% or more.

In some embodiments, the tumor microenvironment may be remodeled using a biocircuit containing an effector module encoding IL17.

In some embodiments, biocircuits, their components, SREs or effector modules are designed to modulate Tregs to attenuate autoimmune disorders. In such a case, IL2 may be regulated using a singly tuned module or one having multiple tuned features as described herein.

In some embodiments, biocircuits, their components, SREs or effector modules are designed to be significantly less immunogenic than other biocircuits or switches in the art.

As used herein, “significantly less immunogenic” refers to a detectable decrease in immunogenicity. In another embodiment, the term refers to a fold decrease in immunogenicity. In another embodiment, the term refers to a decrease such that an effective amount of the biocircuits, their components, SREs or effector modules which can be administered without triggering a detectable immune response. In another embodiment, the term refers to a decrease such that the biocircuits, their components, SREs or effector modules can be repeatedly administered without eliciting an immune response. In another embodiment, the decrease is such that the biocircuits, their components, SREs or effector modules can be repeatedly administered without eliciting an immune response.

In another embodiment, the biocircuits, their components, SREs or effector modules is 2-fold less immunogenic than its unmodified counterpart or reference compound. In another embodiment, immunogenicity is reduced by a 3-fold factor. In another embodiment, immunogenicity is reduced by a 5-fold factor. In another embodiment, immunogenicity is reduced by a 7-fold factor. In another embodiment, immunogenicity is reduced by a 10-fold factor. In another embodiment, immunogenicity is reduced by a 15-fold factor. In another embodiment, immunogenicity is reduced by a fold factor. In another embodiment, immunogenicity is reduced by a 50-fold factor. In another embodiment, immunogenicity is reduced by a 100-fold factor. In another embodiment, immunogenicity is reduced by a 200-fold factor. In another embodiment, immunogenicity is reduced by a 500-fold factor. In another embodiment, immunogenicity is reduced by a 1000-fold factor. In another embodiment, immunogenicity is reduced by a 2000-fold factor. In another embodiment, immunogenicity is reduced by another fold difference.

Methods of determining immunogenicity are well known in the art, and include, e.g. measuring secretion of cytokines (e.g. IL12, IFN alpha, TNF-alpha, RANTES, MIP-1alpha or beta, IL6, IFN-beta, or IL8), measuring expression of DC activation markers (e.g. CD83, HLA-DR, CD80 and CD86), or measuring ability to act as an adjuvant for an adaptive immune response.

In one embodiment, the chimeric antigen receptor (CAR) of the present disclosure may be a conditionally active CAR. A wild type protein or domain thereof, such as those described herein may be used to generate a conditionally active biologic protein which are reversibly or irreversibly inactivated at the wild type normal physiological conditions as well as to such conditionally active biologic proteins and domains and uses of such conditional active biologic proteins and domains are provided. Such methods and conditionally active proteins are taught in, for example, International Publication No. WO2016033331, the contents of which are incorporated herein by reference in their entirety. As a non-limiting example, the CAR comprises at least one antigen specific targeting region evolved from a wild type protein or a domain thereof and one or more of a decrease in activity in the assay at the normal physiological condition compared to the antigen specific targeting region of the wild-type protein or a domain thereof, and an increase in activity in the assay under the aberrant condition compared to the antigen specific targeting region of the wild-type protein or a domain thereof.

### 7. Diseases and Toxins

Various infectious diseases may be treated with pharmaceutical compositions, biocircuits, biocircuit components, effector modules including their SREs or payloads of the present disclosure. As used herein, the term “infectious disease” refers to any disorders caused by organisms such as bacteria, viruses, fungi or parasites. As a non-limiting example, the infectious disease may be Acute bacterial rhinosinusitis, 14-day measles, Acne, Acrodermatitis chronica atrophicans (ACA)-(late skin manifestation of latent Lyme disease), Acute hemorrhagic conjunctivitis, Acute hemorrhagic cystitis, Acute rhinosinusitis, Adult T-cell Leukemia-Lymphoma (ATLL), African Sleeping Sickness, AIDS (Acquired Immunodeficiency Syndrome), Alveolar hydatid, Amebiasis, Amebic meningoencephalitis, Anaplasmosis, Anthrax, Arboviral or parainfectious, Ascariasis—(Roundworm infections), Aseptic meningitis, Athlete's foot (Tinea pedis), Australian tick typhus, Avian Influenza, Babesiosis, Bacillary angiomatosis, Bacterial meningitis, Bacterial vaginosis, Balanitis, Balantidiasis, Bang's disease, Barmah Forest virus infection, Bartonellosis (Verruga peruana; Carrion's disease; Oroya fever), Bat Lyssavirus Infection, Bay sore (Chiclero's ulcer), Baylisascaris infection (Racoon roundworm infection), Beaver fever, Beef tapeworm, Bejel (endemic syphilis), Biphasic meningoencephalitis, Black Bane, Black death, Black piedra, Blackwater Fever, Blastomycosis, Blennorrhea of the newborn, Blepharitis, Boils, Bornholm disease (pleurodynia), Borrelia miyamotoi Disease, Botulism, Boutonneuse fever, Brazilian purpuric fever, Break Bone fever, Brill, Bronchiolitis, Bronchitis, Brucellosis (Bang's disease), Bubonic plague, Bullous impetigo, Burkholderia mallei (Glanders), Burkholderia pseudomallei (Melioidosis), Buruli ulcers (also Mycoburuli ulcers), Busse, Busse-Buschke disease (Cryptococcosis), California group encephalitis, Campylobacteriosis, Candidiasis, Canfield fever (Canicola fever; 7-day fever; Weil's disease; leptospirosis; canefield fever), Canicola fever, Capillariasis, Carate, Carbapenem-resistant Enterobacteriaceae (CRE), Carbuncle, Carrion's disease, Cat Scratch fever, Cave disease, Central Asian hemorrhagic fever, Central European tick, Cervical cancer, Chagas disease, Chancroid (Soft chancre), Chicago disease, Chickenpox (Varicella), Chiclero's ulcer, Chikungunya fever, Chlamydial infection, Cholera, Chromoblastomycosis, Ciguatera, Clap, Clonorchiasis (Liver fluke infection), Clostridium difficile Infection, ClostriDium perfringens (Epsilon Toxin), Coccidioidomycosis fungal infection (Valley fever; desert rheumatism), Coenurosis, Colorado tick fever, Condyloma accuminata, Condyloma accuminata (Warts), Condyloma lata, Congo fever, Congo hemorrhagic fever virus, Conjunctivitis, cowpox, Crabs, Crimean, Croup, Cryptococcosis, Cryptosporidiosis (Crypto), Cutaneous Larval Migrans, Cyclosporiasis, Cystic hydatid, Cysticercosis, Cystitis, Czechoslovak tick, D68 (EV-D68), Dacryocytitis, Dandy fever, Darling's Disease, Deer fly fever, Dengue fever (1, 2, 3 and 4), Desert rheumatism, Devil's grip, Diphasic milk fever, Diphtheria, Disseminated Intravascular Coagulation, Dog tapeworm, Donovanosis, Donovanosis (Granuloma inguinale), Dracontiasis, Dracunculosis, Duke's disease, Dum Dum Disease, Durand-Nicholas-Favre disease, Dwarf tapeworm, E. Coli infection (E. coli), Eastern equine encephalitis, Ebola Hemorrhagic Fever (Ebola virus disease EVD), Ectothrix, Ehrlichiosis (Sennetsu fever), Encephalitis, Endemic Relapsing fever, Endemic syphilis, Endophthalmitis, Endothrix, Enterobiasis (Pinworm infection), Enterotoxin—B Poisoning (Staph Food Poisoning), Enterovirus Infection, Epidemic Keratoconjunctivitis, Epidemic Relapsing fever, Epidemic typhus, Epiglottitis, Erysipelis, Erysipeloid (Erysipelothricosis), Erythema chronicum migrans, Erythema infectiosum, Erythema marginatum, Erythema multiforme, Erythema nodosum, Erythema nodosum leprosum, Erythrasma, Espundia, Eumycotic mycetoma, European blastomycosis, Exanthem subitum (Sixth disease), Eyeworm, Far Eastern tick, Fascioliasis, Fievre boutonneuse (Tick typhus), Fifth Disease (erythema infectiosum), Filatow-Dukes' Disease (Scalded Skin Syndrome; Ritter's Disease), Fish tapeworm, Fitz-Hugh-Curtis syndrome—Perihepatitis, Flinders Island Spotted Fever, Flu (Influenza), Folliculitis, Four Corners Disease, Four Corners Disease (Human Pulmonary Syndrome (HPS)), Frambesia, Francis disease, Furunculosis, Gas gangrene, Gastroenteritis, Genital Herpes, Genital Warts, German measles, Gerstmann-Straussler-Scheinker (GSS), Giardiasis, Gilchrist's disease, Gingivitis, Gingivostomatitis, Glanders, Glandular fever (infectious mononucleosis), Gnathostomiasis, Gonococcal Infection (Gonorrhea), Gonorrhea, Granuloma inguinale (Donovanosis), Guinea Worm, Haemophilus Influenza disease, Hamburger disease, Hansen's disease—leprosy, Hantaan disease, Hantaan-Korean hemorrhagic fever, Hantavirus Pulmonary Syndrome, Hantavirus Pulmonary Syndrome (HPS), Hard chancre, Hard measles, Haverhill fever—Rat bite fever, Head and Body Lice, Heartland fever, Helicobacterosis, Hemolytic Uremic Syndrome (HUS), Hepatitis A, Hepatitis B, Hepatitis C, Hepatitis D, Hepatitis E, Herpangina, Herpes—genital, Herpes labialis, Herpes—neonatal, Hidradenitis, Histoplasmosis, Histoplasmosis infection (Histoplasmosis), His-Werner disease, HIV infection, Hookworm infections, Hordeola, Hordeola (Stye), HTLV, HTLV-associated myelopathy (HAM), Human granulocytic ehrlichiosis, Human monocytic ehrlichiosis, Human Papilloma virus (HPV), Human Pulmonary Syndrome, Hydatid cyst, Hydrophobia, Impetigo, Including congenital (German Measles), Inclusion conjunctivitis, Inclusion conjunctivitis—Swimming Pool conjunctivitis—Pannus, Infantile diarrhea, Infectious Mononucleosis, Infectious myocarditis, Infectious pericarditis, Influenza, Isosporiasis, Israeli spotted fever, Japanese Encephalitis, Jock itch, Jorge Lobo disease—lobomycosis, Jungle yellow fever, Junin Argentinian hemorrhagic fever, Kala Azar, Kaposi's sarcoma, Keloidal blastomycosis, Keratoconjunctivitis, Kuru, Kyasanur forest disease, LaCrosse encephalitis, Lassa hemorrhagic fever, Legionellosis (Legionnaires Disease), Legionnaire's pneumonia, Lemierre's Syndrome (Postanginal septicemia), Lemming fever, Leprosy, Leptospirosis (Nanukayami fever; Weil's disease), Listeriosis (Listeria), Liver fluke infection, Lobo's mycosis, Lockjaw, Loiasis, Louping Ill, Ludwig's angina, Lung fluke infection, Lung fluke infection (Paragonimiasis), Lyme disease, Lymphogranuloma venereum infection (LGV), Machupo Bolivian hemorrhagic fever, Madura foot, Mal del pinto, Malaria, Malignant pustule, Malta fever, Marburg hemorrhagic fever, Masters disease, Maternal Sepsis (Puerperal fever), Measles, Mediterranean spotted fever, Melioidosis (Whitmore's disease), Meningitis, Meningococcal Disease, MERS, Milker's nodule, Molluscum contagiosum, Moniliasis, monkeypox, Mononucleosis, Mononucleosis-like syndrome, Montezuma's Revenge, Morbilli, MRSA (methicillin-resistant Staphylococcus aureus) infection, Mucormycosis—Zygomycosis, Multiple Organ Dysfunction Syndrome or MODS, Multiple-system atrophy (MSA), Mumps, Murine typhus, Murray Valley Encephalitis (MVE), Mycoburuli ulcers, Mycoburuli ulcers—Buruli ulcers, Mycotic vulvovaginitis, Myositis, Nanukayami fever, Necrotizing fasciitis, Necrotizing fasciitis—Type 1, Necrotizing fasciitis-Type 2, Negishi, New world spotted fever, Nocardiosis, Nongonococcal urethritis, Non-Polio (Non-Polio Enterovirus), Norovirus infection, North American blastomycosis, North Asian tick typhus, Norwalk virus infection, Norwegian itch, O'Hara disease, Omsk hemorrhagic fever, Onchoceriasis, Onychomycosis, Opisthorchiasis, Opthalmia neonatorium, Oral hairy leukoplakia, Orf, Oriental Sore, Oriental Spotted Fever, Ornithosis (Parrot fever; Psittacosis), Oroya fever, Otitis externa, Otitis media, Pannus, Paracoccidioidomycosis, Paragonimiasis, Paralytic Shellfish Poisoning (Paralytic Shellfish Poisoning), Paronychia (Whitlow), Parotitis, PCP pneumonia, Pediculosis, Peliosis hepatica, Pelvic Inflammatory Disease, Pertussis (also called Whooping cough), Phaeohyphomycosis, Pharyngoconjunctival fever, Piedra (White Piedra), Piedra (Black Piedra), Pigbel, Pink eye conjunctivitis, Pinta, Pinworm infection, Pitted Keratolysis, Pityriasis versicolor (Tinea versicolor), Plague; Bubonic, Pleurodynia, Pneumococcal Disease, Pneumocystosis, Pneumonia, Pneumonic (Plague), Polio or Poliomyelitis, Polycystic hydatid, Pontiac fever, Pork tapeworm, Posada-Wernicke disease, Postanginal septicemia, Powassan, Progressive multifocal leukencephalopathy, Progressive Rubella Panencephalitis, Prostatitis, Pseudomembranous colitis, Psittacosis, Puerperal fever, Pustular Rash diseases (Small pox), Pyelonephritis, Pylephlebitis, Q-Fever, Quinsy, Quintana fever (5-day fever), Rabbit fever, Rabies, Racoon roundworm infection, Rat bite fever, Rat tapeworm, Reiter Syndrome, Relapsing fever, Respiratory syncytial virus (RSV) infection, Rheumatic fever, Rhodotorulosis, Ricin Poisoning, Rickettsialpox, Rickettsiosis, Rift Valley Fever, Ringworm, Ritter's Disease, River Blindness, Rocky Mountain spotted fever, Rose Handler's disease (Sporotrichosis), Rose rash of infants, Roseola, Ross River fever, Rotavirus infection, Roundworm infections, Rubella, Rubeola, Russian spring, Salmonellosis gastroenteritis, San Joaquin Valley fever, Sao Paulo Encephalitis, Sao Paulo fever, SARS, Scabies Infestation (Scabies) (Norwegian itch), Scalded Skin Syndrome, Scarlet fever (Scarlatina), Schistosomiasis, Scombroid, Scrub typhus, Sennetsu fever, Sepsis (Septic shock), Severe Acute Respiratory Syndrome, Severe Acute Respiratory Syndrome (SARS), Shiga Toxigenic Escherichia coli (STEC/VTEC), Shigellosis gastroenteritis (Shigella), Shinbone fever, Shingles, Shipping fever, Siberian tick typhus, Sinusitis, Sixth disease, Slapped cheek disease, Sleeping sickness, Smallpox (Variola), Snail Fever, Soft chancre, Southern tick associated rash illness, Sparganosis, Spelunker's disease, Sporadic typhus, Sporotrichosis, Spotted fever, Spring, St. Louis encephalitis, Staphylococcal Food Poisoning, Staphylococcal Infection, Strep. throat, Streptococcal Disease, Streptococcal Toxic-Shock Syndrome, Strongyloiciasis, Stye, Subacute Sclerosing Panencephalitis, Subacute Sclerosing Panencephalitis (SSPE), Sudden Acute Respiratory Syndrome, Sudden Rash, Swimmer's ear, Swimmer's Itch, Swimming Pool conjunctivitis, Sylvatic yellow fever, Syphilis, Systemic Inflammatory Response Syndrome (SIRS), Tabes dorsalis (tertiary syphilis), Taeniasis, Taiga encephalitis, Tanner's disease, Tapeworm infections, Temporal lobe encephalitis, Temporal lobe encephalitis, tetani (Lock Jaw), Tetanus Infection, Threadworm infections, Thrush, Tick, Tick typhus, Tinea barbae, Tinea capitis, Tinea corporis, Tinea cruris, Tinea manuum, Tinea nigra, Tinea pedis, Tinea unguium, Tinea versicolor, Torulopsosis, Torulosis, Toxic Shock Syndrome, Toxoplasmosis, transmissible spongioform (CJD), Traveler's diarrhea, Trench fever 5, Trichinellosis, Trichomoniasis, Trichomycosis axillaris, Trichuriasis, Tropical Spastic Paraparesis (TSP), Trypanosomiasis, Tuberculosis (TB), Tuberculosis, Tularemia, Typhoid Fever, Typhus fever, Ulcus molle, Undulant fever, Urban yellow fever, Urethritis, Vaginitis, Vaginosis, Vancomycin Intermediate (VISA), Vancomycin Resistant (VRSA), Varicella, Venezuelan Equine encephalitis, Verruga peruana, Vibrio cholerae (Cholera), Vibriosis (Vibrio), Vincent's disease or Trench mouth, Viral conjunctivitis, Viral Meningitis, Viral meningoencephalitis, Viral rash, Visceral Larval Migrans, Vomito negro, Vulvovaginitis, Warts, Waterhouse, Weil's disease, West Nile Fever, Western equine encephalitis, Whipple's disease, Whipworm infection, White Piedra, Whitlow, Whitmore's disease, Winter diarrhea, Wolhynia fever, Wool sorters' disease, Yaws, Yellow Fever, Yersinosis, Yersinosis (Yersinia), Zahorsky's disease, Zika virus disease, Zoster, Zygomycosis, John Cunningham Virus (JCV), Human immunodeficiency virus (HIV), Influenza virus, Hepatitis B, Hepatitis C, Hepatitis D, Respiratory syncytial virus (RSV), Herpes simplex virus 1 and 2, Human Cytomegalovirus, Epstein-Barr virus, Varicella zoster virus, Coronaviruses, Poxviruses, Enterovirus 71, Rubella virus, Human papilloma virus, Streptococcus pneumoniae, Streptococcus viridans., Staphylococcus aureus (S. aureus), Methicillin-resistant Staphylococcus aureus (MRSA), Vancomycin-intermediate Staphylococcus aureus (VISA), Vancomycin-resistant Staphylococcus aureus (VRSA), Staphylococcus epidermidis (S. epidermidis), Clostridium tetani, Bordetella pertussis, Bordetella paratussis, Mycobacterium, Francisella Tularensis, Toxoplasma gondii, Candida (C. albicans, C. glabrata, C. parapsilosis, C. tropicalis, C. krusei and C. lusitaniae) and/or any other infectious diseases, disorders or syndromes.

Various toxins may be treated with pharmaceutical compositions, biocircuits, biocircuit components, effector modules including their SREs or payloads of the present disclosure. Non-limited examples of toxins include Ricin, Bacillus anthracis, Shiga toxin and Shiga-like toxin, Botulinum toxins.

Various tropical diseases may be treated with pharmaceutical compositions, biocircuits, biocircuit components, effector modules including their SREs or payloads of the present disclosure. Non-limited examples of tropical diseases include Chikungunya fever, Dengue fever, Chagas disease, Rabies, Malaria, Ebola virus, Marburg virus, West Nile Virus, Yellow Fever, Japanese encephalitis virus, St. Louis encephalitis virus.

Various foodborne illnesses and gastroenteritis may be treated with pharmaceutical compositions, biocircuits, biocircuit components, effector modules including their SREs or payloads of the present disclosure. Non-limited examples of foodborne illnesses and gastroenteritis include Rotavirus, Norwalk virus (Norovirus), Campylobacter jejuni, Clostridium difficile, Entamoeba histolytica, Helicobacter pylori, Enterotoxin B of Staphylococcus aureus, Hepatitis A virus (HAV), Hepatitis E, Listeria monocytogenes, Salmonella, Clostridium perfringens, and Salmonella.

Various infectious agents may be treated with pharmaceutical compositions, biocircuits, biocircuit components, effector modules including their SREs or payloads of the present disclosure. Non-limited examples of infectious agents include adenoviruses, Anaplasma phagocytophilium, Ascaris lumbricoides, Bacillus anthracis, Bacillus cereus, Bacteroides sp, Barmah Forest virus, Bartonella bacilliformis, Bartonella henselae, Bartonella quintana, beta-toxin of Clostridium perfringens, Bordetella pertussis, Bordetella parapertussis, Borrelia burgdorferi, Borrelia miyamotoi, Borrelia recurrentis, Borrelia sp., Botulinum toxin, Brucella sp., Burkholderia pseudomallei, California encephalitis virus, Campylobacter, Candida albicans, chikungunya virus, Chlamydia psittaci, Chlamydia trachomatis, Clonorchis sinensis, Clostridium difficile bacteria, Clostridium tetani, Colorado tick fever virus, Corynebacterium diphtheriae, Corynebacterium minutissimum, Coxiella burnetii, coxsackie A, coxsackie B, Crimean-Congo hemorrhagic fever virus, cytomegalovirus, dengue virus, Eastern Equine encephalitis virus, Ebola viruses, echovirus, Ehrlichia chaffeensis., Ehrlichia equi., Ehrlichia sp., Entamoeba histolytica, Enterobacter sp., Enterococcus faecalis, Enterovirus 71, Epstein-Barr virus (EBV), Erysipelothrix rhusiopathiae, Escherichia coli, Flavivirus, Fusobacterium necrophorum, Gardnerella vaginalis, Group B streptococcus, Haemophilus aegyptius, Haemophilus ducreyi, Haemophilus influenzae, hantavirus, Helicobacter pylori, Hepatitis A, Hepatitis B, Hepatitis C, Hepatitis D, Hepatitis E, herpes simplex virus 1 and 2, human herpes virus 6, human herpes Virus 8, human immunodeficiency virus 1 and 2, human T-cell leukemia viruses I and II, influenza viruses (A, B, C), Jamestown Canyon virus, Japanese encephalitis antigenic, Japanese encephalitis virus, John Cunningham virus, juninvirus, Kaposi's Sarcoma-associated Herpes Virus (KSHV), Klebsiella granulomatis, Klebsiella sp., Kyasanur Forest Disease virus, La Crosse virus, Lassavirus, Legionella pneumophila, Leptospira interrogans, Listeria monocytogenes, lymphocytic choriomeningitis virus, lyssavirus, Machupovirus, Marburg virus, measles virus, MERS coronavirus (MERS-CoV), Micrococcus sedentarius, Mobiluncus sp., Molluscipoxvirus, Moraxella catarrhalis, Morbilli-Rubeola virus, Mumpsvirus, Mycobacterium leprae, Mycobacterium tuberculosis, Mycobacterium ulcerans, Mycoplasma genitalium, Mycoplasma sp, Nairovirus, Neisseria gonorrhoeae, Neisseria meningitidis, Nocardia, Norwalk virus, norovirus, Omsk hemorrhagic fever virus, papilloma virus, parainfluenza viruses 1-3, parapoxvirus, parvovirus B19, Peptostreptococccus sp., Plasmodium sp., polioviruses types I, II, and III, Proteus sp., Pseudomonas aeruginosa, Pseudomonas pseudomallei, Pseudomonas sp., rabies virus, respiratory syncytial virus, ricin toxin, Rickettsia australis, Rickettsia conori, Rickettsia honei, Rickettsia prowazekii, Ross River Virus, rotavirus, rubellavirus, Saint Louis encephalitis, Salmonella Typhi, Sarcoptes scabiei, SARS-associated coronavirus (SARS-CoV), Serratia sp., Shiga toxin and Shiga-like toxin, Shigella sp., Sin Nombre Virus, Snowshoe hare virus, Staphylococcus aureus, Staphylococcus epidermidis, Streptobacillus moniliformis, Streptococcus pneumoniae, Streptococcus agalactiae, Streptococcus agalactiae, Streptococcus group A-H, Streptococcus pneumoniae, Streptococcus pyogenes, Treponema pallidum subsp. Pallidum, Treponema pallidum var. carateum, Treponema pallidum var. endemicum, Tropheryma whippelii, Ureaplasma urealyticum, Varicella-Zoster virus, variola virus, Vibrio cholerae, West Nile virus, yellow fever virus, Yersinia enterocolitica, Yersinia pestis, and Zika virus.

Various rare diseases may be treated with pharmaceutical compositions, biocircuits, biocircuit components, effector modules including their SREs or payloads of the present disclosure. As used herein, the term “rare disease” refers to any disease that affects a small percentage of the population. As a non-limiting example, the rare disease may be Acrocephalosyndactylia, Acrodermatitis, Addison Disease, Adie Syndrome, Alagille Syndrome, Amylose, Amyotrophic Lateral Sclerosis, Angelman Syndrome, Angiolymphoid Hyperplasia with Eosinophilia, Arnold-Chiari Malformation, Arthritis, Juvenile Rheumatoid, Asperger Syndrome, Bardet-Biedl Syndrome, Barrett Esophagus, Beckwith-Wiedemann Syndrome, Behcet Syndrome, Bloom Syndrome, Bowen's Disease, Brachial Plexus Neuropathies, Brown-Sequard Syndrome, Budd-Chiari Syndrome, Burkitt Lymphoma, Carcinoma 256, Walker, Caroli Disease, Charcot-Marie-Tooth Disease, Chediak-Higashi Syndrome, Chiari-Frommel Syndrome, Chondrodysplasia Punctata, Colonic Pseudo-Obstruction, Colorectal Neoplasms, Hereditary Nonpolyposis, Craniofacial Dysostosis, Creutzfeldt-Jakob Syndrome, Crohn Disease, Cushing Syndrome, Cystic Fibrosis, Dandy-Walker Syndrome, De Lange Syndrome, Dementia, Vascular, Dermatitis Herpetiformis, DiGeorge Syndrome, Diffuse Cerebral Sclerosis of Schilder, Duane Retraction Syndrome, Dupuytren Contracture, Ebstein Anomaly, Eisenmenger Complex, Ellis-Van Creveld Syndrome, Encephalitis, Enchondromatosis, Epidermal Necrolysis, Toxic, Facial Hemiatrophy, Factor XII Deficiency, Fanconi Anemia, Felty's Syndrome, Fibrous Dysplasia, Polyostotic, Fox-Fordyce Disease, Friedreich Ataxia, Fusobacterium, Gardner Syndrome, Gaucher Disease, Gerstmann Syndrome, Giant Lymph Node Hyperplasia, Glycogen Storage Disease Type I, Glycogen Storage Disease Type II, Glycogen Storage Disease Type IV, Glycogen Storage Disease Type V, Glycogen Storage Disease Type VII, Goldenhar Syndrome, Guillain-Barre Syndrome, Hallermann's Syndrome, Hamartoma Syndrome, Multiple, Hartnup Disease, Hepatolenticular Degeneration, Hepatolenticular Degeneration, Hereditary Sensory and Motor Neuropathy, Hirschsprung Disease, Histiocytic Necrotizing Lymphadenitis, Histiocytosis, Langerhans-Cell, Hodgkin Disease, Horner Syndrome, Huntington Disease, Hyperaldosteronism, Hyperhidrosis, Hyperostosis, Diffuse Idiopathic Skeletal, Hypopituitarism, Inappropriate ADH Syndrome, Intestinal Polyps, Isaacs Syndrome, Kartagener Syndrome, Kearns-Sayre Syndrome, Klippel-Feil Syndrome, Klippel-Trenaunay-Weber Syndrome, Kluver-Bucy Syndrome, Korsakoff Syndrome, Lafora Disease, Lambert-Eaton Myasthenic Syndrome, Landau-Kleffner Syndrome, Langer-Giedion Syndrome, Leigh Disease, Lesch-Nyhan Syndrome, Leukodystrophy, Globoid Cell, Li-Fraumeni Syndrome, Long QT Syndrome, Machado-Joseph Disease, Mallory-Weiss Syndrome, Marek Disease, Marfan Syndrome, Meckel Diverticulum, Meige Syndrome, Melkersson-Rosenthal Syndrome, Meniere Disease, Mikulicz' Disease, Miller Fisher Syndrome, Mobius Syndrome, Moyamoya Disease, Mucocutaneous Lymph Node Syndrome, Mucopolysaccharidosis I, Mucopolysaccharidosis II, Mucopolysaccharidosis III, Mucopolysaccharidosis IV, Mucopolysaccharidosis VI, Multiple Endocrine Neoplasia Type 1, Munchausen Syndrome by Proxy, Muscular Atrophy, Spinal, Narcolepsy, Neuroaxonal Dystrophies, Neuromyelitis Optica, Neuronal Ceroid-Lipofuscinoses, Niemann-Pick Diseases, Noonan Syndrome, Optic Atrophies, Hereditary, Osteitis Deformans, Osteochondritis, Osteochondrodysplasias, Osteolysis, Essential, Paget Disease Extramammary, Paget's Disease, Mammary, Panniculitis, Nodular Nonsuppurative, Papillon-Lefevre Disease, Paralysis, Pelizaeus-Merzbacher Disease, Pemphigus, Benign Familial, Penile Induration, Pericarditis, Constrictive, Peroxisomal Disorders, Peutz-Jeghers Syndrome, Pick Disease of the Brain, Pierre Robin Syndrome, Pigmentation Disorders, Pityriasis Lichenoides, Polycystic Ovary Syndrome, Polyendocrinopathies, Autoimmune, Prader-Willi Syndrome, Pupil Disorders, Rett Syndrome, Reye Syndrome, Rubinstein-Taybi Syndrome, Sandhoff Disease, Sarcoma, Ewing's, Schnitzler Syndrome, Sjogren's Syndrome, Sjogren-Larsson Syndrome, Smith-Lemli-Opitz Syndrome, Spinal Muscular Atrophies of Childhood, Sturge-Weber Syndrome, Sweating, Gustatory, Takayasu Arteritis, Tangier Disease, Tay-Sachs Disease, Thromboangiitis Obliterans, Thyroiditis, Autoimmune, Tietze's Syndrome, Togaviridae Infections, Tolosa-Hunt Syndrome, Tourette Syndrome, Uveomeningoencephalitic Syndrome, Waardenburg's Syndrome, Wegener Granulomatosis, Weil Disease, Werner Syndrome, Williams Syndrome, Wilms Tumor, Wolff-Parkinson-White Syndrome, Wolfram Syndrome, Wolman Disease, Zellweger Syndrome, Zollinger-Ellison Syndrome, and von Willebrand Diseases.

Various autoimmune diseases and autoimmune-related diseases may be treated with pharmaceutical compositions, biocircuits, biocircuit components, effector modules including their SREs or payloads of the present disclosure. As used herein, the term “autoimmune disease” refers to a disease in which the body produces antibodies that attack its own tissues. As a non-limiting example, the autoimmune disease may be Acute Disseminated Encephalomyelitis (ADEM), Acute necrotizing hemorrhagic leukoencephalitis, Addison's disease, Agammaglobulinemia, Alopecia areata, Amyloidosis, Ankylosing spondylitis, Anti-GBM/Anti-TBM nephritis, Antiphospholipid syndrome (APS), Autoimmune angioedema, Autoimmune aplastic anemia, Autoimmune dysautonomia, Autoimmune hepatitis, Autoimmune hyperlipidemia, Autoimmune immunodeficiency, Autoimmune inner ear disease (AIED), Autoimmune myocarditis, Autoimmune oophoritis, Autoimmune pancreatitis, Autoimmune retinopathy, Autoimmune thrombocytopenic purpura (ATP), Autoimmune thyroid disease, Autoimmune urticaria, Axonal & neuronal neuropathies, Balo disease, Behcet's disease, Bullous pemphigoid, Cardiomyopathy, Castleman disease, Celiac disease, Chagas disease, Chronic fatigue syndrome, Chronic inflammatory demyelinating polyneuropathy (CIDP), Chronic recurrent multifocal ostomyelitis (CRMO), Churg-Strauss syndrome, Cicatricial pemphigoid/benign mucosal pemphigoid, Crohn's disease, Cogans syndrome, Cold agglutinin disease, Congenital heart block, Coxsackie myocarditis, CREST disease, Essential mixed cryoglobulinemia, Demyelinating neuropathies, Dermatitis herpetiformis, Dermatomyositis, Devic's disease (neuromyelitis optica), Discoid lupus, Dressler's syndrome, Endometriosis, Eosinophilic esophagitis, Eosinophilic fasciitis, Erythema nodosum, Experimental allergic encephalomyelitis, Evans syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis (GPA) (formerly called Wegener's Granulomatosis), Graves' disease, Guillain-Barre syndrome, Hashimoto's encephalitis, Hashimoto's thyroiditis, Hemolytic anemia, Henoch-Schonlein purpura, Herpes gestationis, Hypogammaglobulinemia, Idiopathic thrombocytopenic purpura (ITP), IgA nephropathy, IgG4-related sclerosing disease, Immunoregulatory lipoproteins, Inclusion body myositis, Interstitial cystitis, Juvenile arthritis, Juvenile diabetes (Type 1 diabetes), Juvenile myositis, Kawasaki syndrome, Lambert-Eaton syndrome, Leukocytoclastic vasculitis, Lichen planus, Lichen sclerosis, Ligneous conjunctivitis, Linear IgA disease (LAD), Lupus (SLE), Lyme disease, chronic, Meniere's disease, Microscopic polyangiitis, Mixed connective tissue disease (MCTD), Mooren's ulcer, Mucha-Habermann disease, Multiple sclerosis, Myasthenia gravis, Myositis, Narcolepsy, Neuromyelitis optica (Devic's), Neutropenia, Ocular cicatricial pemphigoid, Optic neuritis, Palindromic rheumatism, PANDAS (Pediatric Autoimmune Neuropsychiatric Disorders Associated with Streptococcus), Paraneoplastic cerebellar degeneration, Paroxysmal nocturnal hemoglobinuria (PNH), Parry Romberg syndrome, Parsonnage-Turner syndrome, Pars planitis (peripheral uveitis), Pemphigus, Peripheral neuropathy, Perivenous encephalomyelitis, Pernicious anemia, POEMS syndrome, Polyarteritis nodosa, Type I, II, & III autoimmune polyglandular syndromes, Polymyalgia rheumatica, Polymyositis, Postmyocardial infarction syndrome, Postpericardiotomy syndrome, Progesterone dermatitis, Primary biliary cirrhosis, Primary sclerosing cholangitis, Psoriasis, Psoriatic arthritis, Idiopathic pulmonary fibrosis, Pyoderma gangrenosum, Pure red cell aplasia, Raynauds phenomenon, Reactive Arthritis, Reflex sympathetic dystrophy, Reiter's syndrome, Relapsing polychondritis, Restless legs syndrome, Retroperitoneal fibrosis, Rheumatic fever, Rheumatoid arthritis, Sarcoidosis, Schmidt syndrome, Scleritis, Scleroderma, Sjogren's syndrome, Sperm & testicular autoimmunity, Stiff person syndrome, Subacute bacterial endocarditis (SBE), Susac's syndrome, Sympathetic ophthalmia, Takayasu's arteritis, Temporal arteritis/Giant cell arteritis, Thrombocytopenic purpura (TTP), Tolosa-Hunt syndrome, Transverse myelitis, Ulcerative colitis, Undifferentiated connective tissue disease (UCTD), Uveitis, Vasculitis, Vesiculobullous dermatosis, Vitiligo, and Wegener's granulomatosis (now termed Granulomatosis with Polyangiitis (GPA).

Various kidney diseases may be treated with pharmaceutical compositions, biocircuits, biocircuit components, effector modules including their SREs or payloads of the present disclosure. As a non-limiting example, the kidney disease Abderhalden-Kaufmann-Lignac syndrome (Nephropathic Cystinosis), Abdominal Compartment Syndrome, Acute Kidney Failure/Acute Kidney Injury, Acute Lobar Nephronia, Acute Phosphate Nephropathy, Acute Tubular Necrosis, Adenine Phosphoribosyltransferase Deficiency, Adenovirus Nephritis, Alport Syndrome, Amyloidosis, ANCA Vasculitis Related to Endocarditis and Other Infections, Angiomyolipoma, Analgesic Nephropathy, Anorexia Nervosa and Kidney Disease, Angiotensin Antibodies and Focal Segmental Glomerulosclerosis, Antiphospholipid Syndrome, Anti-TNF-α Therapy-related Glomerulonephritis, APOL1 Mutations, Apparent Mineralocorticoid Excess Syndrome, Aristolochic Acid Nephropathy, Chinese Herbal Nephropathy, Balkan Endemic Nephropathy, Bartter Syndrome, Beeturia, β-Thalassemia Renal Disease, Bile Cast Nephropathy, BK Polyoma Virus Nephropathy in the Native Kidney, Bladder Rupture, Bladder Sphincter Dyssynergia, Bladder Tamponade, Border-Crossers' Nephropathy, Bourbon Virus and Acute Kidney Injury, Burnt Sugarcane Harvesting and Acute Renal Dysfunction, Byetta and Renal Failure, Clq Nephropathy, Cannabinoid Hyperemesis Acute Renal Failure, Cardiorenal syndrome, Carfilzomib-Indiced Renal Injury, CFHR5 nephropathy, Charcot-Marie-Tooth Disease with Glomerulopathy, Cherry Concentrate and Acute Kidney Injury, Cholesterol Emboli, Churg-Strauss syndrome, Chyluria, Colistin Nephrotoxicity, Collagenofibrotic Glomerulopathy, Collapsing Glomerulopathy, Collapsing Glomerulopathy Related to CMV, Congenital Nephrotic Syndrome, Conorenal syndrome (Mainzer-Saldino Syndrome or Saldino-Mainzer Disease), Contrast Nephropathy, Copper Sulpfate Intoxication, Cortical Necrosis, Crizotinib-related Acute Kidney Injury, Cryoglobuinemia, Crystalglobulin-Induced Nephropathy, Crystal-Induced Acute Kidney injury, Cystic Kidney Disease, Acquired, Cystinuria, Dasatinib-Induced Nephrotic-Range Proteinuria, Dense Deposit Disease (MPGN Type 2), Dent Disease (X-linked Recessive Nephrolithiasis), Dialysis Disequilibrium Syndrome, Diabetes and Diabetic Kidney Disease, Diabetes Insipidus, Dietary Supplements and Renal Failure, Drugs of Abuse and Kidney Disease, Duplicated Ureter, EAST syndrome, Ebola and the Kidney, Ectopic Kidney, Ectopic Ureter, Edema, Swelling, Erdheim-Chester Disease, Fabry's Disease, Familial Hypocalciuric Hypercalcemia, Fanconi Syndrome, Fraser syndrome, Fibronectin Glomerulopathy, Fibrillary Glomerulonephritis and Immunotactoid Glomerulopathy, Fraley syndrome, Focal Segmental Glomerulosclerosis, Focal Sclerosis, Focal Glomerulosclerosis, Galloway Mowat syndrome, Giant Cell (Temporal) Arteritis with Kidney Involvement, Gestational Hypertension, Gitelman Syndrome, Glomerular Diseases, Glomerular Tubular Reflux, Glycosuria, Goodpasture Syndrome, Hair Dye Ingestion and Acute Kidney Injury, Hantavirus Infection Podocytopathy, Hematuria (Blood in Urine), Hemolytic Uremic Syndrome (HUS), Atypical Hemolytic Uremic Syndrome (aHUS), Hemophagocytic Syndrome, Hemorrhagic Cystitis, Hemorrhagic Fever with Renal Syndrome (HFRS, Hantavirus Renal Disease, Korean Hemorrhagic Fever, Epidemic Hemorrhagic Fever, Nephropathis Epidemica), Hemosiderosis related to Paroxysmal Nocturnal Hemoglobinuria and Hemolytic Anemia, Hepatic Glomerulopathy, Hepatic Veno-Occlusive Disease, Sinusoidal Obstruction Syndrome, Hepatitis C-Associated Renal Disease, Hepatorenal Syndrome, Herbal Supplements and Kidney Disease, High Blood Pressure and Kidney Disease, HIV-Associated Nephropathy (HIVAN), Horseshoe Kidney (Renal Fusion), Hunner's Ulcer, Hyperaldosteronism, Hypercalcemia, Hyperkalemia, Hypermagnesemia, Hypernatremia, Hyperoxaluria, Hyperphosphatemia, Hypocalcemia, Hypokalemia, Hypokalemia-induced renal dysfunction, Hypokalemic Periodic Paralysis, Hypomagnesemia, Hyponatremia, Hypophosphatemia, IgA Nephropathy, IgG4 Nephropathy, Interstitial Cystitis, Painful Bladder Syndrome (Questionnaire), Interstitial Nephritis, Ivemark's syndrome, Ketamine-Associated Bladder Dysfunction, Kidney Stones, Nephrolithiasis, Kombucha Tea Toxicity, Lead Nephropathy and Lead-Related Nephrotoxicity, Leptospirosis Renal Disease, Light Chain Deposition Disease, Monoclonal Immunoglobulin Deposition Disease, Liddle Syndrome, Lightwood-Albright Syndrome, Lipoprotein Glomerulopathy, Lithium Nephrotoxicity, LMX1B Mutations Cause Hereditary FSGS, Loin Pain Hematuria, Lupus, Systemic Lupus Erythematosis, Lupus Kidney Disease, Lupus Nephritis, Lupus Nephritis with Antineutrophil Cytoplasmic Antibody Seropositivity, Lyme Disease-Associated Glomerulonephritis, Malarial Nephropathy, Malignancy-Associated Renal Disease, Malignant Hypertension, Malakoplakia, Meatal Stenosis, Medullary Cystic Kidney Disease, Medullary Sponge Kidney, Megaureter, Melamine Toxicity and the Kidney, Membranoproliferative Glomerulonephritis, Membranous Nephropathy, MesoAmerican Nephropathy, Metabolic Acidosis, Metabolic Alkalosis, Methotrexate-related Renal Failure, Microscopic Polyangiitis, Milk-alkalai syndrome, Minimal Change Disease, MDMA (Molly; Ecstacy; 3,4-Methylenedioxymethamphetamine) and Kidney Failure, Multicystic dysplastic kidney, Multiple Myeloma, Myeloproliferative Neoplasms and Glomerulopathy, Nail-patella Syndrome, Nephrocalcinosis, Nephrogenic Systemic Fibrosis, Nephroptosis (Floating Kidney, Renal Ptosis), Nephrotic Syndrome, Neurogenic Bladder, Nodular Glomerulosclerosis, Non-Gonococcal Urethritis, Nutcracker syndrome, Orofaciodigital Syndrome, Orotic Aciduria, Orthostatic Hypotension, Orthostatic Proteinuria, Osmotic Diuresis, Ovarian Hyperstimulation Syndrome, Page Kidney, Papillary Necrosis, Papillorenal Syndrome (Renal-Coloboma Syndrome, Isolated Renal Hypoplasia), Parvovirus B19 and the Kidney, The Peritoneal-Renal Syndrome, Posterior Urethral Valve, Post-infectious Glomerulonephritis, Post-streptococcal Glomerulonephritis, Polyarteritis Nodosa, Polycystic Kidney Disease, Posterior Urethral Valves, Preeclampsia, Propofol infusion syndrome, Proliferative Glomerulonephritis with Monoclonal IgG Deposits (Nasr Disease), Propolis (Honeybee Resin) Related Renal Failure, Proteinuria (Protein in Urine), Pseudohyperaldosteronism, Pseudohypobicarbonatemia, Pseudohypoparathyroidism, Pulmonary-Renal Syndrome, Pyelonephritis (Kidney Infection), Pyonephrosis, Radiation Nephropathy, Ranolazine and the Kidney, Refeeding syndrome, Reflux Nephropathy, Rapidly Progressive Glomerulonephritis, Renal Abscess, Peripnephric Abscess, Renal Agenesis, Renal Arcuate Vein Microthrombi-Associated Acute Kidney Injury, Renal Artery Aneurysm, Renal Artery Stenosis, Renal Cell Cancer, Renal Cyst, Renal Hypouricemia with Exercise-induced Acute Renal Failure, Renal Infarction, Renal Osteodystrophy, Renal Tubular Acidosis, Renin Secreting Tumors (Juxtaglomerular Cell Tumor), Reset Osmostat, Retrocaval Ureter, Retroperitoneal Fibrosis, Rhabdomyolysis, Rhabdomyolysis related to Bariatric surgery, Rheumatoid Arthritis-Associated Renal Disease, Sarcoidosis Renal Disease, Salt Wasting, Renal and Cerebral, Schistosomiasis and Glomerular Disease, Schimke immuno-osseous dysplasia, Scleroderma Renal Crisis, Serpentine Fibula-Polycystic Kidney Syndrome, Exner Syndrome, Sickle Cell Nephropathy, Silica Exposure and Chronic Kidney Disease, Sri Lankan Farmers' Kidney Disease, Sjogren's Syndrome and Renal Disease, Synthetic Cannabinoid Use and Acute Kidney Injury, Kidney Disease Following Hematopoietic Cell Transplantation, Kidney Disease Related to Stem Cell Transplantation, Thin Basement Membrane Disease, Benign Familial Hematuria, Trigonitis, Tuberculosis, Genitourinary, Tuberous Sclerosis, Tubular Dysgenesis, Immune Complex Tubulointerstitial Nephritis Due to Autoantibodies to the Proximal Tubule Brush Border, Tumor Lysis Syndrome, Uremia, Uremic Optic Neuropathy, Ureteritis Cystica, Ureterocele, Urethral Caruncle, Urethral Stricture, Urinary Incontinence, Urinary Tract Infection, Urinary Tract Obstruction, Vesicointestinal Fistula, Vesicoureteral Reflux, Volatile Anesthetics and Acute Kidney Injury, Von Hippel-Lindau Disease, Waldenstrom's Macroglobulinemic Glomerulonephritis, Warfarin-Related Nephropathy, Wasp Stings and Acute Kidney Injury, Wegener's Granulomatosis, Granulomatosis with Polyangiitis, West Nile Virus and Chronic Kidney Disease, and Wunderlich syndrome.

Various cardiovascular diseases may be treated with pharmaceutical compositions, biocircuits, biocircuit components, effector modules including their SREs or payloads of the present disclosure. As a non-limiting example, the cardiovascular disease may be Ischemic heart disease also known as coronary artery disease, Cerebrovascular disease (Stroke), Peripheral vascular disease, Heart failure, Rheumatic heart disease, and Congenital heart disease.

Various antibody deficiencies may be treated with pharmaceutical compositions, biocircuits, biocircuit components, effector modules including their SREs or payloads of the present disclosure. As a non-limiting example, the antibody deficiencies may be X-Linked Agammaglobulinemia (XLA), Autosomal Recessive Agammaglobulinemia (ARA), Common Variable Immune Deficiency (CVID), IgG (IgG1, IgG2, IgG3 and IgG4) Subclass Deficiency, Selective IgA Deficiency, Specific Antibody Deficiency (SAD), Transient Hypogammaglobulinemia of Infancy, Antibody Deficiency with Normal or Elevated Immunoglobulins, Selective IgM Deficiency, Immunodeficiency with Thymoma (Good's Syndrome), Transcobalamin II Deficiency, Warts, Hypogammaglobulinemia, Infection, Myelokathexis (WHIM) Syndrome, Drug-Induced Antibody Deficiency, Kappa Chain Deficiency, Heavy Chain Deficiencies, Post-Meiotic Segregation (PMS2) Disorder, and Unspecified Hypogammaglobulinemia.

Various ocular diseases may be treated with pharmaceutical compositions, biocircuits, biocircuit components, effector modules including their SREs or payloads of the present disclosure. As a non-limiting example, the ocular disease may be thyroid eye disease (TED), Graves' disease (GD) and orbitopathy, Retina Degeneration, Cataract, optic atrophy, macular degeneration, Leber congenital amaurosis, retinal degeneration, cone-rod dystrophy, Usher syndrome, leopard syndrome, photophobia, and photoaversion.

Various neurological diseases may be treated with pharmaceutical compositions, biocircuits, biocircuit components, effector modules including their SREs or payloads of the present disclosure. As a non-limiting example, the neurological disease may be Absence of the Septum Pellucidum, Acid Lipase Disease, Acid Maltase Deficiency, Acquired Epileptiform Aphasia, Acute Disseminated Encephalomyelitis, Attention Deficit-Hyperactivity Disorder (ADHD), Adie's Pupil, Adie's Syndrome, Adrenoleukodystrophy, Agenesis of the Corpus Callosum, Agnosia, Aicardi Syndrome, Aicardi-Goutieres Syndrome Disorder, AIDS—Neurological Complications, Alexander Disease, Alpers' Disease, Alternating Hemiplegia, Alzheimer's Disease, Amyotrophic Lateral Sclerosis (ALS), Anencephaly, Aneurysm, Angelman Syndrome, Angiomatosis, Anoxia, Antiphospholipid Syndrome, Aphasia, Apraxia, Arachnoid Cysts, Arachnoiditis, Arnold-Chiari Malformation, Arteriovenous Malformation, Asperger Syndrome, Ataxia, Ataxia Telangiectasia, Ataxias and Cerebellar or Spinocerebellar Degeneration, Atrial Fibrillation and Stroke, Attention Deficit-Hyperactivity Disorder, Autism Spectrum Disorder, Autonomic Dysfunction, Back Pain, Barth Syndrome, Batten Disease, Becker's Myotonia, Behcet's Disease, Bell's Palsy, Benign Essential Blepharospasm, Benign Focal Amyotrophy, Benign Intracranial Hypertension, Bernhardt-Roth Syndrome, Binswanger's Disease, Blepharospasm, Bloch-Sulzberger Syndrome, Brachial Plexus Birth Injuries, Brachial Plexus Injuries, Bradbury-Eggleston Syndrome, Brain and Spinal Tumors, Brain Aneurysm, Brain Injury, Brown-Sequard Syndrome, Bulbospinal Muscular Atrophy, Cerebral Autosomal Dominant Arteriopathy with Sub-cortical Infarcts and Leukoencephalopathy (CADASIL), Canavan Disease, Carpal Tunnel Syndrome, Causalgia, Cavernomas, Cavernous Angioma, Cavernous Malformation, Central Cervical Cord Syndrome, Central Cord Syndrome, Central Pain Syndrome, Central Pontine Myelinolysis, Cephalic Disorders, Ceramidase Deficiency, Cerebellar Degeneration, Cerebellar Hypoplasia, Cerebral Aneurysms, Cerebral Arteriosclerosis, Cerebral Atrophy, Cerebral Beriberi, Cerebral Cavernous Malformation, Cerebral Gigantism, Cerebral Hypoxia, Cerebral Palsy, Cerebro-Oculo-Facio-Skeletal Syndrome (COFS), Charcot-Marie-Tooth Disease, Chiari Malformation, Cholesterol Ester Storage Disease, Chorea, Choreoacanthocytosis, Chronic Inflammatory Demyelinating Polyneuropathy (CIDP), Chronic Orthostatic Intolerance, Chronic Pain, Cockayne Syndrome Type II, Coffin Lowry Syndrome, Colpocephaly, Coma, Complex Regional Pain Syndrome, Congenital Facial Diplegia, Congenital Myasthenia, Congenital Myopathy, Congenital Vascular Cavernous Malformations, Corticobasal Degeneration, Cranial Arteritis, Craniosynostosis, Cree encephalitis, Creutzfeldt-Jakob Disease, Cumulative Trauma Disorders, Cushing's Syndrome, Cytomegalic Inclusion Body Disease, Cytomegalovirus Infection, Dancing Eyes-Dancing Feet Syndrome, Dandy-Walker Syndrome, Dawson Disease, De Morsier's Syndrome, Dejerine-Klumpke Palsy, Dementia, Dementia—Multi-Infarct, Dementia—Semantic, Dementia—Subcortical, Dementia With Lewy Bodies, Dentate Cerebellar Ataxia, Dentatorubral Atrophy, Dermatomyositis, Developmental Dyspraxia, Devic's Syndrome, Diabetic Neuropathy, Diffuse Sclerosis, Dravet Syndrome, Dysautonomia, Dysgraphia, Dyslexia, Dysphagia, Dyspraxia, Dyssynergia Cerebellaris Myoclonica, Dyssynergia Cerebellaris Progressiva, Dystonias, Early Infantile Epileptic Encephalopathy, Empty Sella Syndrome, Encephalitis, Encephalitis Lethargica, Encephaloceles, Encephalopathy, Encephalopathy (familial infantile), Encephalotrigeminal Angiomatosis, Epilepsy, Epileptic Hemiplegia, Erb's Palsy, Erb-Duchenne and Dejerine-Klumpke Palsies, Essential Tremor, Extrapontine Myelinolysis, Fabry Disease, Fahr's Syndrome, Fainting, Familial Dysautonomia, Familial Hemangioma, Familial Idiopathic Basal Ganglia Calcification, Familial Periodic Paralyses, Familial Spastic Paralysis, Farber's Disease, Febrile Seizures, Fibromuscular Dysplasia, Fisher Syndrome, Floppy Infant Syndrome, Foot Drop, Friedreich's Ataxia, Frontotemporal Dementia, Gaucher Disease, Generalized Gangliosidoses, Gerstmann's Syndrome, Gerstmann-Straussler-Scheinker Disease, Giant Axonal Neuropathy, Giant Cell Arteritis, Giant Cell Inclusion Disease, Globoid Cell Leukodystrophy, Glossopharyngeal Neuralgia, Glycogen Storage Disease, Guillain-Barré Syndrome, Hallervorden-Spatz Disease, Head Injury, Headache, Hemicrania Continua, Hemifacial Spasm, Hemiplegia Alterans, Hereditary Neuropathies, Hereditary Spastic Paraplegia, Heredopathia Atactica Polyneuritiformis, Herpes Zoster, Herpes Zoster Oticus, Hirayama Syndrome, Holmes-Adie syndrome, Holoprosencephaly, HTLV-1 Associated Myelopathy, Hughes Syndrome, Huntington's Disease, Hydranencephaly, Hydrocephalus, Hydrocephalus—Normal Pressure, Hydromyelia, Hypercortisolism, Hypersomnia, Hypertonia, Hypotonia, Hypoxia, Immune-Mediated Encephalomyelitis, Inclusion Body Myositis, Incontinentia Pigmenti, Infantile Hypotonia, Infantile Neuroaxonal Dystrophy, Infantile Phytanic Acid Storage Disease, Infantile Refsum Disease, Infantile Spasms, Inflammatory Myopathies, Iniencephaly, Intestinal Lipodystrophy, Intracranial Cysts, Intracranial Hypertension, Isaacs' Syndrome, Joubert Syndrome, Kearns-Sayre Syndrome, Kennedy's Disease, Kinsbourne syndrome, Kleine-Levin Syndrome, Klippel-Feil Syndrome, Klippel-Trenaunay Syndrome (KTS), Klüver-Bucy Syndrome, Korsakoffs Amnesic Syndrome, Krabbe Disease, Kugelberg-Welander Disease, Kuru, Lambert-Eaton Myasthenic Syndrome, Landau-Kleffner Syndrome, Lateral Femoral Cutaneous Nerve Entrapment, Lateral Medullary Syndrome, Learning Disabilities, Leigh's Disease, Lennox-Gastaut Syndrome, Lesch-Nyhan Syndrome, Leukodystrophy, Levine-Critchley Syndrome, Lewy Body Dementia, Lipid Storage Diseases, Lipoid Proteinosis, Lissencephaly, Locked-In Syndrome, Lou Gehrig's Disease, Lupus—Neurological Sequelae, Lyme Disease—Neurological Complications, Machado-Joseph Disease, Macrencephaly, Megalencephaly, Melkersson-Rosenthal Syndrome, Meningitis, Meningitis and Encephalitis, Menkes Disease, Meralgia Paresthetica, Metachromatic Leukodystrophy, Microcephaly, Migraine, Miller Fisher Syndrome, Mini Stroke, Mitochondrial Myopathy, Moebius Syndrome, Monomelic Amyotrophy, Motor Neuron Diseases, Moyamoya Disease, Mucolipidoses, Mucopolysaccharidosis, Multi-Infarct Dementia, Multifocal Motor Neuropathy, Multiple Sclerosis, Multiple System Atrophy, Multiple System Atrophy with Orthostatic Hypotension, Muscular Dystrophy, Myasthenia—Congenital, Myasthenia Gravis, Myelinoclastic Diffuse Sclerosis, Myoclonic Encephalopathy of Infants, Myoclonus, Myopathy, Myopathy-Congenital, Myopathy—Thyrotoxic, Myotonia, Myotonia Congenita, Narcolepsy, Neuroacanthocytosis, Neurodegeneration with Brain Iron Accumulation, Neurofibromatosis, Neuroleptic Malignant Syndrome, Neurological Complications of AIDS, Neurological Complications of Lyme Disease, Neurological Consequences of Cytomegalovirus Infection, Neurological Manifestations of Pompe Disease, Neurological Sequelae Of Lupus, Neuromyelitis Optica, Neuromyotonia, Neuronal Ceroid Lipofuscinosis, Neuronal Migration Disorders, Neuropathy—Hereditary, Neurosarcoidosis, Neurosyphilis, Neurotoxicity, Nevus Cavernosus, Niemann-Pick Disease, O'Sullivan-McLeod Syndrome, Occipital Neuralgia, Ohtahara Syndrome, Olivopontocerebellar Atrophy, Opsoclonus Myoclonus, Orthostatic Hypotension, Overuse Syndrome, Pain—Chronic, Pantothenate Kinase-Associated Neurodegeneration, Paraneoplastic Syndromes, Paresthesia, Parkinson's Disease, Paroxysmal Choreoathetosis, Paroxysmal Hemicrania, Parry-Romberg, Pelizaeus-Merzbacher Disease, Pena Shokeir II Syndrome, Perineural Cysts, Periodic Paralyses, Peripheral Neuropathy, Periventricular Leukomalacia, Persistent Vegetative State, Pervasive Developmental Disorders, Phytanic Acid Storage Disease, Pick's Disease, Pinched Nerve, Piriformis Syndrome, Pituitary Tumors, Polymyositis, Pompe Disease, Porencephaly, Post-Polio Syndrome, Postherpetic Neuralgia, Post infectious Encephalomyelitis, Postural Hypotension, Postural Orthostatic Tachycardia Syndrome, Postural Tachycardia Syndrome, Primary Dentatum Atrophy, Primary Lateral Sclerosis, Primary Progressive Aphasia, Prion Diseases, Progressive Hemifacial Atrophy, Progressive Locomotor Ataxia, Progressive Multifocal Leukoencephalopathy, Progressive Sclerosing Poliodystrophy, Progressive Supranuclear Palsy, Prosopagnosia, Pseudo-Torch syndrome, Pseudotoxoplasmosis syndrome, Pseudotumor Cerebri, Psychogenic Movement, Ramsay Hunt Syndrome I, Ramsay Hunt Syndrome II, Rasmussen's Encephalitis, Reflex Sympathetic Dystrophy Syndrome, Refsum Disease, Refsum Disease—Infantile, Repetitive Motion Disorders, Repetitive Stress Injuries, Restless Legs Syndrome, Retrovirus-Associated Myelopathy, Rett Syndrome, Reye's Syndrome, Rheumatic Encephalitis, Riley-Day Syndrome, Sacral Nerve Root Cysts, Saint Vitus Dance, Salivary Gland Disease, Sandhoff Disease, Schilder's Disease, Schizencephaly, Seitelberger Disease, Seizure Disorder, Semantic Dementia, Septo-Optic Dysplasia, Severe Myoclonic Epilepsy of Infancy (SMEI), Shaken Baby Syndrome, Shingles, Shy-Drager Syndrome, Sjogren's Syndrome, Sleep Apnea, Sleeping Sickness, Sotos Syndrome, Spasticity, Spina Bifida, Spinal Cord Infarction, Spinal Cord Injury, Spinal Cord Tumors, Spinal Muscular Atrophy, Spinocerebellar Atrophy, Spinocerebellar Degeneration, Steele-Richardson-Olszewski Syndrome, Stiff-Person Syndrome, Striatonigral Degeneration, Stroke, Sturge-Weber Syndrome, Subacute Sclerosing Panencephalitis, Subcortical Arteriosclerotic Encephalopathy, Short-lasting, Unilateral, Neuralgiform (SUNCT) Headache, Swallowing Disorders, Sydenham Chorea, Syncope, Syphilitic Spinal Sclerosis, Syringohydromyelia, Syringomyelia, Systemic Lupus Erythematosus, Tabes Dorsalis, Tardive Dyskinesia, Tarlov Cysts, Tay-Sachs Disease, Temporal Arteritis, Tethered Spinal Cord Syndrome, Thomsen's Myotonia, Thoracic Outlet Syndrome, Thyrotoxic Myopathy, Tic Douloureux, Todd's Paralysis, Tourette Syndrome, Transient Ischemic Attack, Transmissible Spongiform Encephalopathies, Transverse Myelitis, Traumatic Brain Injury, Tremor, Trigeminal Neuralgia, Tropical Spastic Paraparesis, Troyer Syndrome, Tuberous Sclerosis, Vascular Erectile Tumor, Vasculitis Syndromes of the Central and Peripheral Nervous Systems, Von Economo's Disease, Von Hippel-Lindau Disease (VHL), Von Recklinghausen's Disease, Wallenberg's Syndrome, Werdnig-Hoffman Disease, Wernicke-Korsakoff Syndrome, West Syndrome, Whiplash, Whipple's Disease, Williams Syndrome, Wilson Disease, Wolman's Disease, X-Linked Spinal and Bulbar Muscular Atrophy.

Various psychological disorders may be treated with pharmaceutical compositions, biocircuits, biocircuit components, effector modules including their SREs or payloads of the present disclosure. As a non-limiting example, the psychological disorders may be Aboulia, Absence epilepsy, Acute stress Disorder, Adjustment Disorders, Adverse effects of medication NOS, Age related cognitive decline, Agoraphobia, Alcohol Addiction, Alzheimer's Disease, Amnesia (also known as Amnestic Disorder), Amphetamine Addiction, Anorexia Nervosa, Anterograde amnesia, Antisocial personality disorder (also known as Sociopathy), Anxiety Disorder (Also known as Generalized Anxiety Disorder), Anxiolytic related disorders, Asperger's Syndrome (now part of Autism Spectrum Disorder), Attention Deficit Disorder (Also known as ADD), Attention Deficit Hyperactivity Disorder (Also known as ADHD), Autism Spectrum Disorder (also known as Autism), Autophagia, Avoidant Personality Disorder, Barbiturate related disorders, Benzodiazepine related disorders, Bereavement, Bibliomania, Binge Eating Disorder, Bipolar disorder (also known as Manic Depression, includes Bipolar I and Bipolar II), Body Dysmorphic Disorder, Borderline intellectual functioning, Borderline Personality Disorder, Breathing-Related Sleep Disorder, Brief Psychotic Disorder, Bruxism, Bulimia Nervosa, Caffeine Addiction, Cannabis Addiction, Catatonic disorder, Catatonic schizophrenia, Childhood amnesia, Childhood Disintegrative Disorder (now part of Autism Spectrum Disorder), Childhood Onset Fluency Disorder (formerly known as Stuttering), Circadian Rhythm Disorders, Claustrophobia, Cocaine related disorders, Communication disorder, Conduct Disorder, Conversion Disorder, Cotard delusion, Cyclothymia (also known as Cyclothymic Disorder), Delerium, Delusional Disorder, dementia, Dependent Personality Disorder (also known as Asthenic Personality Disorder), Depersonalization disorder (now known as Depersonalization/Derealization Disorder), Depression (also known as Major Depressive Disorder), Depressive personality disorder, Derealization disorder (now known as Depersonalization/Derealization Disorder), Dermotillomania, Desynchronosis, Developmental coordination disorder, Diogenes Syndrome, Disorder of written expression, Dispareunia, Dissocial Personality Disorder, Dissociative Amnesia, Dissociative Fugue, Dissociative Identity Disorder (formerly known as Multiple Personality Disorder), Down syndrome, Dyslexia, Dyspareunia, Dysthymia (now known as Persistent Depressive Disorder), Eating disorder NOS, Ekbom's Syndrome (Delusional Parasitosis), Emotionally unstable personality disorder, Encopresis, Enuresis (bedwetting), Erotomania, Exhibitionistic Disorder, Expressive language disorder, Factitious Disorder, Female Sexual Disorders, Fetishistic Disorder, Folie à deux, Fregoli delusion, Frotteuristic Disorder, Fugue State, Ganser syndrome, Gambling Addiction, Gender Dysphoria (formerly known as Gender Identity Disorder), Generalized Anxiety Disorder, General adaptation syndrome, Grandiose delusions, Hallucinogen Addiction, Haltlose personality disorder, Histrionic Personality Disorder, Primary hypersomnia, Huntington's Disease, Hypoactive sexual desire disorder, Hypochondriasis, Hypomania, Hyperkinetic syndrome, Hypersomnia, Hysteria, Impulse control disorder, Impulse control disorder NOS, Inhalant Addiction, Insomnia, Intellectual Development Disorder, Intermittent Explosive Disorder, Joubert syndrome, Kleptomania, Korsakoff's syndrome, Lacunar amnesia, Language Disorder, Learning Disorders, Major Depression (also known as Major Depressive Disorder), major depressive disorder, Male Sexual Disorders, Malingering, Mathematics disorder, Medication-related disorder, Melancholia, Mental Retardation (now known as Intellectual Development Disorder), Misophonia, Morbid jealousy, Multiple Personality Disorder (now known as Dissociative Identity Disorder), Munchausen Syndrome, Munchausen by Proxy, Narcissistic Personality Disorder, Narcolepsy, Neglect of child, Neurocognitive Disorder (formerly known as Dementia), Neuroleptic-related disorder, Nightmare Disorder, Non Rapid Eye Movement, Obsessive-Compulsive Disorder, Obsessive-Compulsive Personality Disorder (also known as Anankastic Personality Disorder), Oneirophrenia, Onychophagia, Opioid Addiction, Oppositional Defiant Disorder, Orthorexia (ON), Pain disorder, Panic attacks, Panic Disorder, Paranoid Personality Disorder, Parkinson's Disease, Partner relational problem, Passive-aggressive personality disorder, Pathological gambling, Pedophilic Disorder, Perfectionism, Persecutory delusion, Persistent Depressive Disorder (also known as Dysthymia), Personality change due to a general medical condition, Personality disorder, Pervasive developmental disorder (PDD), Phencyclidine related disorder, Phobic disorder, Phonological disorder, Physical abuse, Pica, Polysubstance related disorder, Postpartum Depression, Post-traumatic embitterment disorder (PTED), Post-Traumatic Stress Disorder, Premature ejaculation, Premenstrual Dysphoric Disorder, Psychogenic amnesia, Psychological factor affecting medical condition, Psychoneurotic personality disorder, Psychotic disorder, not otherwise specified, Pyromania, Reactive Attachment Disorder, Reading disorder, Recurrent brief depression, Relational disorder, REM Sleep Behavior Disorder, Restless Leg Syndrome, Retrograde amnesia, Retts Disorder (now part of Autism Spectrum Disorder), Rumination syndrome, Sadistic personality disorder, Schizoaffective Disorder, Schizoid Personality Disorder, Schizophrenia, Schizophreniform disorder, Schizotypal Personality Disorder, Seasonal Affective Disorder, Sedative, Hypnotic, or Anxiolytic Addiction, Selective Mutism, Self-defeating personality disorder, Separation Anxiety Disorder, Sexual Disorders Female, Sexual Disorders Male, Sexual Addiction, Sexual Masochism Disorder, Sexual Sadism Disorder, Shared Psychotic Disorder, Sleep Arousal Disorders, Sleep Paralysis, Sleep Terror Disorder (now part of Nightmare Disorder, Social Anxiety Disorder, Somatization Disorder, Specific Phobias, Stendhal syndrome, Stereotypic movement disorder, Stimulant Addiction, Stuttering (now known as Childhood Onset Fluency Disorder), Substance related disorder, Tardive dyskinesia, Tobacco Addiction, Tourettes Syndrome, Transient tic disorder, Transient global amnesia, Transvestic Disorder, Trichotillomania, Undifferentiated Somatoform Disorder, Vaginismus, and Voyeuristic Disorder.

Various lung diseases may be treated with pharmaceutical compositions, biocircuits, biocircuit components, effector modules including their SREs or payloads of the present disclosure. As a non-limiting example, the lung diseases may be Asbestosis, Asthma, Bronchiectasis, Bronchitis, Chronic Cough, Chronic Obstructive Pulmonary Disease (COPD), Croup, Cystic Fibrosis, Hantavirus, Idiopathic Pulmonary Fibrosis, Pertussis, Pleurisy, Pneumonia, Pulmonary Embolism, Pulmonary Hypertension, Sarcoidosis, Sleep Apnea, Spirometry, Sudden Infant Death Syndrome (SIDS), Tuberculosis, Alagille Syndrome, Autoimmune Hepatitis, Biliary Atresia, Cirrhosis, ERCP (Endoscopic Retrograde Cholangiopancreatography), and Hemochromatosis. Nonalcoholic Steatohepatitis, Porphyria, Primary Biliary Cirrhosis, Primary Sclerosing Cholangitis.

Various bone diseases may be treated with pharmaceutical compositions, biocircuits, biocircuit components, effector modules including their SREs or payloads of the present disclosure. As a non-limiting example, the bone diseases may be osteoporosis, neurofibromatosis, osteogenesis imperfecta (OI), rickets, osteosarcoma, achondroplasia, fracture, osteomyelitis, Ewing tumor of bone, osteomalacia, hip dysplasia, Paget disease of bone, marble bone disease, osteochondroma, bone cancer, bone disease, osteochondrosis, osteoma, fibrous dysplasia, cleidocranial dysostosis, osteoclastoma, bone cyst, metabolic bone disease, melorheostosis, callus, Caffey syndrome, and mandibulofacial dysostosis.

Various blood diseases may be treated with pharmaceutical compositions, biocircuits, biocircuit components, effector modules including their SREs or payloads of the present disclosure. As a non-limiting example, the blood diseases may be Anemia and CKD (for health care professionals), Aplastic Anemia and Myelodysplastic Syndromes, Deep Vein Thrombosis, Hemochromatosis, Hemophilia, Henoch-Schonlein Purpura, Idiopathic Thrombocytopenic Purpura, Iron-Deficiency Anemia, Pernicious Anemia, Pulmonary Embolism, Sickle Cell Anemia, Sickle Cell Trait and Other Hemoglobinopathies, Thalassemia, Thrombotic Thrombocytopenic Purpura, and Von Willebrand Disease.

### 8. Gene Editing

The CRISPR-Cas9 system is a novel genome editing system which has been rapidly developed and implemented in a multitude of model organisms and cell types, and supplants other genome editing technologies, such as TALENs and ZFNs. CRISPRs are sequence motifs are present in bacterial and archaeal genomes and are composed of short (about 24-48 nucleotide) direct repeats separated by similarly sized, unique spacers (Grissa et al. BMC Bioinformatics 8, 172 (2007). They are generally flanked by a set of CRISPR-associated (Cas) protein-coding genes that are required for CRISPR maintenance and function (Barrangou et al., Science 315, 1709 (2007), Brouns et al., Science 321, 960 (2008), Haft et al. PLoS Comput Biol 1, e60 (2005). CRISPR-Cas systems provide adaptive immunity against invasive genetic elements (e.g., viruses, phages and plasmids) (Horvath and Barrangou, Science, 2010, 327: 167-170; Bhaya et al., Annu. Rev. Genet., 2011, 45: 273-297; and Brrangou R, RNA, 2013, 4: 267-278). Three different types of CRISPR-Cas systems have been classified in bacteria and the type II CRISPR-Cas system is most studied. In the bacterial Type II CRISPR-Cas system, small CRISPR RNAs (crRNAs) processed from the pre-repeat-spacer transcript (pre-crRNA) in the presence of a trans-activating RNA (tracrRNA)/Cas9 can form a duplex with the tracrRNA/Cas9 complex. The mature complex is recruited to a target double strand DNA sequence that is complementary to the spacer sequence in the tracrRNA: crRNA duplex to cleave the target DNA by Cas9 endonuclease (Garneau et al., Nature, 2010, 468: 67-71; Jinek et al., Science, 2012, 337: 816-821; Gasiunas et al., Proc. Natl Acad. Sci. USA., 109: E2579-2586; and Haurwitz et al., Science, 2010, 329: 1355-1358). Target recognition and cleavage by the crRNA: tracrRNA/Cas9 complex in the type II CRISPR-CAS system not only requires a sequence in the tracrRNA: crRNA duplex that is a complementary to the target sequence (also called “protospacer” sequence) but also requires a protospacer adjacent motif (PAM) sequence located 3′ end of the protospacer sequence of a target polynucleotide. The PAM motif can vary between different CRISPR-Cas systems.

CRISPR-Cas9 systems have been developed and modified for use in genetic editing and prove to be a high effective and specific technology for editing a nucleic acid sequence even in eukaryotic cells. Many researchers disclosed various modifications to the bacterial CRISPR-Cas systems and demonstrated that CRISPR-Cas systems can be used to manipulate a nucleic acid in a cell, such as in a mammalian cell and in a plant cell. Representative references include U.S. Pat. Nos. 8,993,233; 8,999,641; 8,945,839; 8,932,814; 8,906,616; 8,895,308; 8,889,418; 8,889,356; 8,871,445; 8,865,406; 8,771,945; and 8,697,359; U.S. Patent Publication Nos. 20150031134; 20150203872; 20150218253; 20150176013; 20150191744; 20150071889; 20150067922; and 20150167000; each of which is incorporated herein by reference in their entirety.

However, controlling the effects and activity of the CRISPR-Cas system (e.g., guide RNA and nuclease) has been challenging and often can be problematic.

The biocircuits of the present disclosure and/or any of their components may be utilized in regulating or tuning the CRISPR/Cas9 system in order to optimize its utility.

In some embodiments, the payloads of the effector modules of the disclosure may include alternative isoforms or orthologs of the Cas9 enzyme.

The most commonly used Cas9 is derived from Streptococcus pyogenes and the RuvC domain can be inactivated by a D10A mutation and the HNH domain can be inactivated by an H840A mutation.

In addition to Cas9 derived from S. pyogenes, other RNA guided endonucleases (RGEN) may also be used for programmable genome editing. Cas9 sequences have been identified in more than 600 bacterial strains. Though Cas9 family shows high diversity of amino acid sequences and protein sizes, All Cas9 proteins share a common architecture with a central HNH nuclease domain and a split RuvC/RHase H domain. Examples of Cas9 orthologs from other bacterial strains including but not limited to, Cas proteins identified in Acaryochloris marina MBIC11017; Acetohalobium arabaticum DSM 5501; Acidithiobacillus caldus; Acidithiobacillus ferrooxidans ATCC 23270; Alicyclobacillus acidocaldarius LAA1; Alicyclobacillus acidocaldarius subsp. acidocaldarius DSM 446; Allochromatium vinosum DSM 180; Ammonifex degensii KC4; Anabaena variabilis ATCC 29413; Arthrospira maxima CS-328; Arthrospira platensis str. Paraca; Arthrospira sp. PCC 8005; Bacillus pseudomycoides DSM 12442; Bacillus selenitireducens MLS10; Burkholderiales bacterium 1_1_47; Caldicelulosiruptor becscii DSM 6725; Candidatus desulforudis audaxviator MP104C; Caldicellulosiruptor hydrothermalis 108; Clostridium phage c-st; Clostridium botulinum A3 str. Loch Maree; Clostridium botulinum Ba4 str. 657; Clostridium difficile QCD-63q42; Crocosphaera watsonii WH 8501; Cyanothece sp. ATCC 51142; Cyanothece sp. CCY0110; Cyanothece sp. PCC 7424; Cyanothece sp. PCC 7822; Exiguobacterium sibiricum 255-15; Finegoldia magna ATCC 29328; Ktedonobacter racemifer DSM 44963; Lactobacillus delbrueckii subsp. bulgaricus PB2003/044-T3-4; Lactobacillus salivarius ATCC 11741; Listeria innocua; Lyngbya sp. PCC 8106; Marinobacter sp. ELB17; Methanohalobium evestigatum Z-7303; Microcystis phage Ma-LMM01; Microcystis aeruginosa NIES-843; Microscilla marina ATCC 23134; Microcoleus chthonoplastes PCC 7420; Neisseria meningitidis; Nitrosococcus halophilus Nc4; Nocardiopsis dassonvillei subsp. dassonvillei DSM 43111; Nodularia spumigena CCY9414; Nostoc sp. PCC 7120; Oscillatoria sp. PCC 6506; Pelotomaculum thermopropionicum SI; Petrotoga mobilis SJ95; Polaromonas naphthalenivorans CJ2; Polaromonas sp. J5666; Pseudoalteromonas haloplanktis TAC125; Streptomyces pristinaespiralis ATCC 25486; Streptomyces pristinaespiralis ATCC 25486; Streptococcus thermophilus; Streptomyces viridochromogenes DSM 40736; Streptosporangium roseum DSM 43021; Synechococcus sp. PCC 7335; and Thermosipho africanus TCF52B (Chylinski et al., RNA Biol., 2013; 10(5): 726-737).

In addition to Cas9 orthologs, other Cas9 variants such as fusion proteins of inactive dCas9 and effector domains with different functions may be served as a platform for genetic modulation. Any of the foregoing enzymes may be useful in the present disclosure.

### 9. Stem Cell Applications

The biocircuits of the present disclosure and/or any of their components may be utilized in the regulated reprogramming of cells, stem cell engraftment or other application where controlled or tunable expression of such reprogramming factors are useful.

The biocircuits of the present disclosure may be used in reprogramming cells including stem cells or induced stem cells. Induction of induced pluripotent stem cells (iPSC) was first achieved by Takahashi and Yamanaka (Cell, 2006. 126(4):663-76; herein incorporated by reference in its entirety) using viral vectors to express KLF4, c-MYC, OCT4 and SOX2 otherwise collectively known as KMOS.

Excisable lentiviral and transposon vectors, repeated application of transient plasmid, episomal and adenovirus vectors have also been used to try to derive iPSC (Chang, C.-W., et al., Stem Cells, 2009. 27(5):1042-1049; Kaji, K., et al., Nature, 2009. 458(7239):771-5; Okita, K., et al., Science, 2008. 322(5903):949-53; Stadtfeld, M., et al., Science, 2008. 322(5903):945-9; Woltjen, K., et al., Nature, 2009; Yu, J., et al., Science, 2009:1172482; Fusaki, N., et al., Proc Jpn Acad Ser B Phys Biol Sci, 2009. 85(8):348-62; each of which is herein incorporated by reference in its entirety).

DNA-free methods to generate human iPSC has also been derived using serial protein transduction with recombinant proteins incorporating cell-penetrating peptide moieties (Kim, D., et al., Cell Stem Cell, 2009. 4(6): 472-476; Zhou, H., et al., Cell Stem Cell, 2009. 4(5):381-4; each of which is herein incorporated by reference in its entirety), and infectious transgene delivery using the Sendai virus (Fusaki, N., et al., Proc Jpn Acad Ser B Phys Biol Sci, 2009. 85(8): p. 348-62; herein incorporated by reference in its entirety).

The effector modules of the present disclosure may include a payload comprising any of the genes including, but not limited to, OCT such as OCT4, SOX such as SOX1, SOX2, SOX3, SOX15 and SOX18, NANOG, KLF such as KLF1, KLF2, KLF4 and KLF5, MYC such as c-MYC and n-MYC, REM2, TERT and LIN28 and variants thereof in support of reprogramming cells. Sequences of such reprogramming factors are taught in for example International Application PCT/US2013/074560, the contents of which are incorporated herein by reference in their entirety.

### IV. Dosing and Administrations

The present disclosure provides methods comprising administering any one or more compositions for immunotherapy to a subject in need thereof. These may be administered to a subject using any amount and any route of administration effective for preventing or treating a clinical condition such as cancer, infection diseases and other immunodeficient diseases.

Compositions in accordance with the disclosure are typically formulated in dosage unit form for ease of administration and uniformity of dosage. It will be understood, however, that the total daily usage of the compositions of the present disclosure may be decided by the attending physician within the scope of sound medical judgment. The specific therapeutically effective, or prophylactically effective dose level for any particular patient will depend upon a variety of factors including the disorder being treated and the severity of the disorder; the activity of the specific compound employed; the specific composition employed; the age, body weight, general health, sex and diet of the patient; the time of administration, route of administration, previous or concurrent therapeutic interventions and rate of excretion of the specific compound employed; the duration of the treatment; drugs used in combination or coincidental with the specific compound employed; and like factors well known in the medical arts.

The destabilizing domains (DDs), effector modules and biocircuit systems of the disclosure and compositions comprising the same, may be administered by any route to achieve a therapeutically effective outcome.

These include, but are not limited to enteral (into the intestine), gastroenteral, epidural (into the dura matter), oral (by way of the mouth), transdermal, peridural, intracerebral (into the cerebrum), intracerebroventricular (into the cerebral ventricles), epicutaneous (application onto the skin), intradermal, (into the skin itself), subcutaneous (under the skin), nasal administration (through the nose), intravenous (into a vein), intravenous bolus, intravenous drip, intraarterial (into an artery), intramuscular (into a muscle), intracardiac (into the heart), intraosseous infusion (into the bone marrow), intrathecal (into the spinal canal), intraperitoneal, (infusion or injection into the peritoneum), intravesical infusion, intravitreal, (through the eye), intracavernous injection (into a pathologic cavity) intracavitary (into the base of the penis), intravaginal administration, intrauterine, extra-amniotic administration, transdermal (diffusion through the intact skin for systemic distribution), transmucosal (diffusion through a mucous membrane), transvaginal, insufflation (snorting), sublingual, sublabial, enema, eye drops (onto the conjunctiva), in ear drops, auricular (in or by way of the ear), buccal (directed toward the cheek), conjunctival, cutaneous, dental (to a tooth or teeth), electro-osmosis, endocervical, endosinusial, endotracheal, extracorporeal, hemodialysis, infiltration, interstitial, intra-abdominal, intra-amniotic, intra-articular, intrabiliary, intrabronchial, intrabursal, intracartilaginous (within a cartilage), intracaudal (within the cauda equine), intracisternal (within the cisterna magna cerebellomedularis), intracorneal (within the cornea), dental intracornal, intracoronary (within the coronary arteries), intracorporus cavernosum (within the dilatable spaces of the corporus cavernosa of the penis), intradiscal (within a disc), intraductal (within a duct of a gland), intraduodenal (within the duodenum), intradural (within or beneath the dura), intraepidermal (to the epidermis), intraesophageal (to the esophagus), intragastric (within the stomach), intragingival (within the gingivae), intraileal (within the distal portion of the small intestine), intralesional (within or introduced directly to a localized lesion), intraluminal (within a lumen of a tube), intralymphatic (within the lymph), intramedullary (within the marrow cavity of a bone), intrameningeal (within the meninges), intramyocardial (within the myocardium), intraocular (within the eye), intraovarian (within the ovary), intrapericardial (within the pericardium), intrapleural (within the pleura), intraprostatic (within the prostate gland), intrapulmonary (within the lungs or its bronchi), intrasinal (within the nasal or periorbital sinuses), intraspinal (within the vertebral column), intrasynovial (within the synovial cavity of a joint), intratendinous (within a tendon), intratesticular (within the testicle), intrathecal (within the cerebrospinal fluid at any level of the cerebrospinal axis), intrathoracic (within the thorax), intratubular (within the tubules of an organ), intratumor (within a tumor), intratympanic (within the aurus media), intravascular (within a vessel or vessels), intraventricular (within a ventricle), iontophoresis (by means of electric current where ions of soluble salts migrate into the tissues of the body), irrigation (to bathe or flush open wounds or body cavities), laryngeal (directly upon the larynx), nasogastric (through the nose and into the stomach), occlusive dressing technique (topical route administration which is then covered by a dressing which occludes the area), ophthalmic (to the external eye), oropharyngeal (directly to the mouth and pharynx), parenteral, percutaneous, periarticular, peridural, perineural, periodontal, rectal, respiratory (within the respiratory tract by inhaling orally or nasally for local or systemic effect), retrobulbar (behind the pons or behind the eyeball), intramyocardial (entering the myocardium), soft tissue, subarachnoid, subconjunctival, submucosal, topical, transplacental (through or across the placenta), transtracheal (through the wall of the trachea), transtympanic (across or through the tympanic cavity), ureteral (to the ureter), urethral (to the urethra), vaginal, caudal block, diagnostic, nerve block, biliary perfusion, cardiac perfusion, photopheresis or spinal.

In some embodiments, compositions of the present disclosure, may be administered by any of the methods of administration taught in the co-pending commonly owned U.S. Provisional Patent Application No. 62/320,864 filed on Apr. 11, 2016, or in U.S. Provisional Application No. 62/466,596 filed Mar. 3, 2017, and the International Publication WO2017/180587, the contents of each of which are incorporated herein by reference in their entirety.

### Kits and Applications

The also provides a kit comprising any of the polynucleotides or expression vectors described herein.

The present disclosure includes a variety of kits for conveniently and/or effectively carrying out methods of the present disclosure. Typically, kits will comprise sufficient amounts and/or numbers of components to allow a user to perform one or multiple treatments of a subject(s) and/or to perform one or multiple experiments.

In one embodiment, the present disclosure provides kits for inhibiting genes in vitro or in vivo, comprising a biocircuit of the present disclosure or a combination of biocircuits of the present disclosure, optionally in combination with any other suitable active agents.

The kit may further comprise packaging and instructions and/or a delivery agent to form a formulation composition. The delivery agent may comprise, for example, saline, a buffered solution.

In additional embodiments, assay screening kits are provided. The kit includes a container for the screening assay. An instruction for the use of the assay and the information about the screening method are to be included in the kit.

In some embodiments, the DDs, effector modules and biocircuit system and compositions of the disclosure may be used as research tools to investigate protein activity in a biological system such a cell and a subject. In other embodiments, the DDs, effector modules and biocircuit system and compositions of the disclosure may be used for treating a disease such as a cancer and a genetic disorder.

### V. Delivery Modalities and/or Vector

### Vectors

The present disclosure also provides vectors that package polynucleotides of the disclosure encoding biocircuits, effector modules, SREs (DDs) and payload constructs, and combinations thereof. Vectors of the present disclosure may also be used to deliver the packaged polynucleotides to a cell, a local tissue site or a subject. These vectors may be of any kind, including DNA vectors, RNA vectors, plasmids, viral vectors and particles. Viral vector technology is well known and described in Sambrook et al. (2001, Molecular Cloning: A Laboratory Manual, Cold Spring Harbor Laboratory, New York). Viruses, which are useful as vectors include, but are not limited to lentiviral vectors, adenoviral vectors, adeno-associated viral (AAV) vectors, herpes simplex viral vectors, retroviral vectors, oncolytic viruses, and the like.

In general, vectors contain an origin of replication functional in at least one organism, a promoter sequence and convenient restriction endonuclease site, and one or more selectable markers e.g. a drug resistance gene.

As used herein a promoter is defined as a DNA sequence recognized by transcription machinery of the cell, required to initiate specific transcription of the polynucleotide sequence of the present disclosure. Vectors can comprise native or non-native promoters operably linked to the polynucleotides of the disclosure. The promoters selected may be strong, weak, constitutive, inducible, tissue specific, development stage-specific, and/or organism specific. One example of a suitable promoter is the immediate early cytomegalovirus (CMV) promoter sequence. This promoter sequence is a strong constitutive promoter sequence capable of driving high levels of expression of polynucleotide sequence that is operatively linked to it. Another example of a preferred promoter is Elongation Growth Factor-1. Alpha (EF-1. alpha). Other constitutive promoters may also be used, including, but not limited to simian virus 40 (SV40), mouse mammary tumor virus (MMTV), human immunodeficiency virus (HIV), long terminal repeat (LTR), promoter, an avian leukemia virus promoter, an Epstein-Barr virus immediate early promoter, a Rous sarcoma virus promoter as well as human gene promoters including, but not limited to the phosphoglycerate kinase (PGK) promoter, actin promoter, the myosin promoter, the hemoglobin promoter, the Ubiquitin C (Ubc) promoter, the human U6 small nuclear protein promoter and the creatine kinase promoter. In some instances, inducible promoters such as but not limited to metallothionine promoter, glucocorticoid promoter, a progesterone promoter, and a tetracycline promoter may be used. In some embodiments, the promoter may be selected from the following a CMV promoter, comprising a nucleotide sequence of SEQ ID NO. 363, a PGK promoter, comprising a nucleotide sequence of SEQ ID NO. 364, and an EF1a promoter, comprising a nucleotide sequence of SEQ ID NO. 365, or SEQ ID NO. 366.

In some embodiments, the promoter of the disclosure may be a Tet-ON promoter. Combination of the transcription regulation Tet system with the DDs permits simultaneous control of gene expression and protein stability. Any of the dual-Tet ON-DD systems described by Pedone et al. (2018) doi: https://doi.org/10.1101/404699 may be useful in the present disclosure (the contents of which are herein incorporated by reference in their entirety).

In some embodiments, the optimal promoter may be selected based on its ability to achieve minimal expression of the SREs and payloads of the disclosure in the absence of the ligand and detectable expression in the presence of the ligand.

Additional promoter elements e.g. enhancers may be used to regulate the frequency of transcriptional initiation. Such regions may be located 10-100 base pairs upstream or downstream of the start site. In some instances, two or more promoter elements may be used to cooperatively or independently activate transcription.

In some embodiments, the recombinant expression vector may comprise regulatory sequences, such as transcription and translation initiation and termination codons, which are specific to the type of host cell into which the vector is to be introduced.

### 1. Lentiviral Vectors

In some embodiments, lentiviral vectors/particles may be used as vehicles and delivery modalities. Lentiviruses are subgroup of the Retroviridae family of viruses, named because reverse transcription of viral RNA genomes to DNA is required before integration into the host genome. As such, the most important features of lentiviral vehicles/particles are the integration of their genetic material into the genome of a target/host cell. Some examples of lentivirus include the Human Immunodeficiency Viruses: HIV-1 and HIV-2, the Simian Immunodeficiency Virus (SIV), feline immunodeficiency virus (FIV), bovine immunodeficiency virus (BIV), Jembrana Disease Virus (JDV), equine infectious anemia virus (EIAV), equine infectious anemia virus, visna-maedi and caprine arthritis encephalitis virus (CAEV).

Typically, lentiviral particles making up the gene delivery vehicle are replication defective on their own (also referred to as “self-inactivating”). Lentiviruses are able to infect both dividing and non-dividing cells by virtue of the entry mechanism through the intact host nuclear envelope (Naldini L et al., Curr. Opin. Biotechnol, 1998, 9: 457-463). Recombinant lentiviral vehicles/particles have been generated by multiply attenuating the HIV virulence genes, for example, the genes Env, Vif, Vpr, Vpu, Nef and Tat are deleted making the vector biologically safe. Correspondingly, lentiviral vehicles, for example, derived from HIV-1/HIV-2 can mediate the efficient delivery, integration and long-term expression of transgenes into non-dividing cells. As used herein, the term “recombinant” refers to a vector or other nucleic acid containing both lentiviral sequences and non-lentiviral retroviral sequences.

Lentiviral particles may be generated by co-expressing the virus packaging elements and the vector genome itself in a producer cell such as human HEK293T cells. These elements are usually provided in three (in second generation lentiviral systems) or four separate plasmids (in third generation lentiviral systems). The producer cells are co-transfected with plasmids that encode lentiviral components including the core (i.e. structural proteins) and enzymatic components of the virus, and the envelope protein(s) (referred to as the packaging systems), and a plasmid that encodes the genome including a foreign transgene, to be transferred to the target cell, the vehicle itself (also referred to as the transfer vector). In general, the plasmids or vectors are included in a producer cell line. The plasmids/vectors are introduced via transfection, transduction or infection into the producer cell line. Methods for transfection, transduction or infection are well known by those of skill in the art. As non-limiting example, the packaging and transfer constructs can be introduced into producer cell lines by calcium phosphate transfection, lipofection or electroporation, generally together with a dominant selectable marker, such as neo, DHFR, Gln synthetase or ADA, followed by selection in the presence of the appropriate drug and isolation of clones.

The producer cell produces recombinant viral particles that contain the foreign gene, for example, the effector module of the present disclosure. The recombinant viral particles are recovered from the culture media and titrated by standard methods used by those of skill in the art. The recombinant lentiviral vehicles can be used to infect target cells.

Cells that can be used to produce high-titer lentiviral particles may include, but are not limited to, HEK293T cells, 293G cells, STAR cells (Relander et al., Mol. Ther 2005, 11: 452-459), FreeStyle™ 293 Expression System (ThermoFisher, Waltham, Mass.), and other HEK293T-based producer cell lines (e.g., Stewart et al., Hum Gene Ther. 2011, 22 (3):357-369; Lee et al., Biotechnol Bioeng, 2012, 10996): 1551-1560; Throm et al., Blood. 2009, 113(21): 5104-5110; the contents of each of which are incorporated herein by reference in their entirety).

In some aspects, the envelope proteins may be heterologous envelop proteins from other viruses, such as the G protein of vesicular stomatitis virus (VSV G) or baculoviral gp64 envelop proteins. The VSV-G glycoprotein may especially be chosen among species classified in the vesiculovirus genus: Carajas virus (CJSV), Chandipura virus (CHPV), Cocal virus (COCV), Isfahan virus (ISFV), Maraba virus (MARAV), Piry virus (PIRYV), Vesicular stomatitis Alagoas virus (VSAV), Vesicular stomatitis Indiana virus (V SIV) and Vesicular stomatitis New Jersey virus (VSNJV) and/or stains provisionally classified in the vesiculovirus genus as Grass carp rhabdovirus, BeAn 157575 virus (BeAn 157575), Boteke virus (BTKV), Calchaqui virus (CQIV), Eel virus American (EVA), Gray Lodge virus (GLOV), Jurona virus (JURY), Klamath virus (KLAV), Kwatta virus (KWAV), La Joya virus (LJV), Malpais Spring virus (MSPV), Mount Elgon bat virus (MEBV), Perinet virus (PERV), Pike fry rhabdovirus (PFRV), Porton virus (PORV), Radi virus (RADIV), Spring viremia of carp virus (SVCV), Tupaia virus (TUPV), Ulcerative disease rhabdovirus (UDRV) and Yug Bogdanovac virus (YBV). The gp64 or other baculoviral env protein can be derived from Autographa californica nucleopolyhedrovirus (AcMNPV), Anagrapha falcifera nuclear polyhedrosis virus, Bombyx mori nuclear polyhedrosis virus, Choristoneura fumiferana nucleopolyhedrovirus, Orgyia pseudotsugata single capsid nuclear polyhedrosis virus, Epiphyas postvittana nucleopolyhedrovirus, Hyphantria cunea nucleopolyhedrovirus, Galleria mellonella nuclear polyhedrosis virus, Dhori virus, Thogoto virus, Antheraea pemyi nucleopolyhedrovirus or Batken virus.

Additional elements provided in lentiviral particles may comprise retroviral LTR (long-terminal repeat) at either 5′ or 3′ terminus, a retroviral export element, optionally a lentiviral reverse response element (RRE), a promoter or active portion thereof, and a locus control region (LCR) or active portion thereof. Other elements include central polypurine tract (cPPT) sequence to improve transduction efficiency in non-dividing cells, Woodchuck Hepatitis Virus (WHP) Posttranscriptional Regulatory Element (WPRE) which enhances the expression of the transgene and increases titer. The effector module is linked to the vector.

Methods for generating recombinant lentiviral particles are discussed in the art, for example, U.S. Pat. Nos. 8,846,385; 7,745,179; 7,629,153; 7,575,924; 7,179,903; and 6,808,905; the contents of each of which are incorporated herein by reference in their entirety.

Lentivirus vectors used may be selected from, but are not limited to pLVX, pLenti, pLenti6, pLJM1, FUGW, pWPXL, pWPI, pLenti CMV puro DEST, pLJM1-EGFP, pULTRA, pInducer20, pHIV-EGFP, pCW57.1, pTRPE, pELPS, pRRL, and pLionII.

Lentiviral vehicles known in the art may also be used (See, U.S. Pat. Nos. 9,260,725; 9,068,199; 9,023,646; 8,900,858; 8,748,169; 8,709,799; 8,420,104; 8,329,462; 8,076,106; 6,013,516; and 5,994,136; International Patent Publication No. WO2012079000; the contents of each of which are incorporated herein by reference in their entirety).

### 2. Retroviral Vectors (γ-Retroviral Vectors)

In some embodiments, retroviral vectors may be used to package and deliver the biocircuits, biocircuit components, effector modules, SREs or payload constructs of the present disclosure. Retroviral vectors (RVs) allow the permanent integration of a transgene in target cells. In addition to lentiviral vectors based on complex HIV-1/2, retroviral vectors based on simple gamma-retroviruses have been widely used to deliver therapeutic genes and demonstrated clinically as one of the most efficient and powerful gene delivery systems capable of transducing a broad range of cell types. Example species of Gamma retroviruses include the murine leukemia viruses (MLVs) and the feline leukemia viruses (FeLV).

In some embodiments, gamma-retroviral vectors derived from a mammalian gamma-retrovirus such as murine leukemia viruses (MLVs), are recombinant. The MLV families of gamma retroviruses include the ecotropic, amphotropic, xenotropic and polytropic subfamilies. Ecotropic viruses are able to infect only murine cells using mCAT-1 receptor. Examples of ecotropic viruses are Moloney MLV and AKV. Amphotropic viruses infect murine, human and other species through the Pit-2 receptor. One example of an amphotropic virus is the 4070A virus. Xenotropic and polytropic viruses utilize the same (Xpr1) receptor but differ in their species tropism. Xenotropic viruses such as NZB-9-1 infect human and other species but not murine species, whereas polytropic viruses such as focus-forming viruses (MCF) infect murine, human and other species.

Gamma-retroviral vectors may be produced in packaging cells by co-transfecting the cells with several plasmids including one encoding the retroviral structural and enzymatic (gag-pol) polyprotein, one encoding the envelope (env) protein, and one encoding the vector mRNA comprising polynucleotide encoding the compositions of the present disclosure that is to be packaged in newly formed viral particles.

In some aspects, the recombinant gamma-retroviral vectors are pseudotyped with envelope proteins from other viruses. Envelope glycoproteins are incorporated in the outer lipid layer of the viral particles which can increase/alter the cell tropism. Exemplary envelop proteins include the gibbon ape leukemia virus envelope protein (GALV) or vesicular stomatitis virus G protein (VSV-G), or Simian endogenous retrovirus envelop protein, or Measles Virus H and F proteins, or Human immunodeficiency virus gp120 envelope protein, or cocal vesiculovirus envelop protein (See, e.g., U.S. application publication NO. 2012/164118; the contents of which are incorporated herein by reference in its entirety). In other aspects, envelope glycoproteins may be genetically modified to incorporate targeting/binding ligands into gamma-retroviral vectors, binding ligands including, but not limited to, peptide ligands, single chain antibodies and growth factors (Waehler et al., Nat. Rev. Genet. 2007, 8(8):573-587; the contents of which are incorporated herein by reference in its entirety). These engineered glycoproteins can retarget vectors to cells expressing their corresponding target moieties. In other aspects, a “molecular bridge” may be introduced to direct vectors to specific cells. The molecular bridge has dual specificities: one end can recognize viral glycoproteins, and the other end can bind to the molecular determinant on the target cell. Such molecular bridges, for example ligand-receptor, avidin-biotin, and chemical conjugations, monoclonal antibodies and engineered fusogenic proteins, can direct the attachment of viral vectors to target cells for transduction (Yang et al., Biotechnol. Bioeng., 2008, 101(2): 357-368; and Maetzig et al., Viruses, 2011, 3, 677-713; the contents of each of which are incorporated herein by reference in their entirety).

In some embodiments, the recombinant gamma-retroviral vectors are self-inactivating (SIN) gammaretroviral vectors. The vectors are replication incompetent. SIN vectors may harbor a deletion within the 3′ U3 region initially comprising enhancer/promoter activity. Furthermore, the 5′ U3 region may be replaced with strong promoters (needed in the packaging cell line) derived from Cytomegalovirus or RSV, or an internal promoter of choice, and/or an enhancer element. The choice of the internal promoters may be made according to specific requirements of gene expression needed for a particular purpose of the disclosure.

In some embodiments, polynucleotides encoding the biocircuit, biocircuit components, effector module, SRE are inserted within the recombinant viral genome. The other components of the viral mRNA of a recombinant gamma-retroviral vector may be modified by insertion or removal of naturally occurring sequences (e.g., insertion of an IRES, insertion of a heterologous polynucleotide encoding a polypeptide or inhibitory nucleic acid of interest, shuffling of a more effective promoter from a different retrovirus or virus in place of the wild-type promoter and the like). In some examples, the recombinant gamma-retroviral vectors may comprise modified packaging signal, and/or primer binding site (PBS), and/or 5′-enhancer/promoter elements in the U3-region of the 5′-long terminal repeat (LTR), and/or 3′-SIN elements modified in the U3-region of the 3′-LTR. These modifications may increase the titers and the ability of infection.

Gamma retroviral vectors suitable for delivering biocircuit components, effector modules, SREs or payload constructs of the present disclosure may be selected from those disclosed in U.S. Pat. Nos. 8,828,718; 7,585,676; 7,351,585; U.S. application publication NO. 2007/048285; PCT application publication NOs. WO2010/113037; WO2014/121005; WO2015/056014; and EP Pat. NOs. EP1757702; EP1757703 (the contents of each of which are incorporated herein by reference in their entirety).

### Lentiviral Vectors and Cell Engineering

Lentiviral vectors are used for introducing transgenes into T cells (e.g., primary human T cells or Jurkat cells) for preclinical research and clinical applications, including recently approved products such as Tisagenlecleucel (KYMRIAH®) for relapsed/refractory B-cell lymphoma. VSV-G pseudotyped 3rd generation lentiviral vectors offer high titers, high transduction efficiency and safety, and have become the vectors of choice for T cell engineering. While not wishing to be bound by theory, T cell engineering usually involves T cell activation by CD3/CD28 antibodies, followed by lentivirus transduction, and then cell expansion which can last from 5 to 30 days (e.g., 9 to 14 days or 9 to 15 days). In general, lentivirus transgene integration may take over 7 days to fully stabilize in T cells (e.g., primary human T cells or Jurkat cells). While longer cultures can increase the cell numbers, the longer cultures can also change the T cell phenotype to a more differentiated state. Therefore, the duration of ex vivo culture can impact the persistence and efficacy of CAR T cells. For example, cells cultured for shorter duration may display a less differentiated phenotype and can be highly efficacious in preclinical models.

While not wishing to be bound by theory, the state of T cell differentiation may influence the engraftment and persistence of T cells following adoptive transfer. Ghassemi et al. (Reducing Ex Vivo Culture Improves the Antileukemic Activity of Chimeric Antigen Receptor (CAR) T Cells. Cancer Immunol Res; 6(9) September 2018; the contents of which are herein incorporated by reference in their entirety) describe primary human T cell differentiation over time and saw that early harvested CAR T cells exhibited enhanced effector function and proliferation, as well as enhanced potency and persistence in vivo.

Lentivirus dynamics such as transduction, integration and/or expression kinetics of lentivirally introduced transgenes in T cells (e.g., primary human T cells or Jurkat cells) ex vivo may impact the efficacy and durability of in vivo anti-tumor responses. Some type of T cells may produce different results. For example, the Jurkat cell line may not provide the dynamic range of expression as primary human T cells. Methods to evaluate these lentivirus dynamics are known in the art and are described herein.

In some embodiment, to determine the transgene expression kinetics CD3/CD28 activated primary human T cells can be transduced with lentivirus carrying a transgene (e.g., a regulated transgene or constitutive transgene such as CD19 CAR, IL12, fluorescent protein or any transgene (e.g., payload) described herein). The cells may be analyzed by methods described herein and/or known in the art for viability, viral genomic integration (e.g., by using quantitative PCR), transcript levels (e.g., by using quantitative RT-PCR), and cell surface expression of the transgene if applicable (e.g., if the transgene is or includes CD19 CAR then the surface expression of the CD19 CAR can be evaluated). The cells may be analyzed prior to transduction and/or after transduction such as 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 or more than 30 days after transduction. As a non-limiting example, the cells may be analyzed at various time points between 3 to 14 days after transduction (e.g., 3 days, 4 days, 5 days, 6 days, 7 days, 8 days, 9 days, 10 days, 11 days, 12 days, 13 days, and/or 14 days). As a non-limiting example, the cells may be analyzed 3 to 15 days after transduction. As a non-limiting example, the cells may be analyzed 9 to 15 days after transduction.

In some embodiments, the CD3/CD28 activated primary human T cells can be reactivated with CD3/CD28 beads after transduction. The cells may be reactivated 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 or more than 30 days after transduction. The cells may be analyzed by methods described herein and/or known in the art for viability, viral genomic integration (e.g., by using quantitative PCR), transcript levels (e.g., by using quantitative RT-PCR), cell surface expression of the transgene if applicable (e.g., if the transgene is or includes CD19 CAR then the surface expression of the CD19 CAR can be evaluated), copy number, and/or mRNA levels.

In some embodiments, the cell viability of activated primary human T cells transduced with lentivirus carrying a transgene is greater than 65%, 70%, 75%, 80%, 85%, 90%, 95%, 97%, or 99%. As a non-limiting example, the cell viability is greater than 90%. As a non-limiting example, the cell viability is greater than 85%.

In some embodiments, the cell viability of Jurkat cells transduced with lentivirus carrying a transgene is greater than 65%, 70%, 75%, 80%, 85%, 90%, 95%, 97%, or 99%. As a non-limiting example, the cell viability is greater than 90%. As a non-limiting example, the cell viability is greater than 85%.

In some embodiments, the integration of the transgene into the genome of the cell may be at or above the saturation point. As a non-limiting example, the saturation point may be 3 copies per cell.

In some embodiments, the integration of the transgene into the genome may be high in the initial timepoints evaluated and then decline to a lower integration value before becoming stable for the remainder of the culture. As a non-limiting example, the integration may be up to 20 copies per cell of the transgene into the genome during the early timepoints before declining to 2 copies per cell and being stable throughout the remainder of the culture.

In some embodiments, the transduction of ability of T cells may be evaluated. T cells from at least one donor may be transduced with a lentivirus containing a transgene at a dose that is predicted to reach the saturating levels (e.g., enough virus that each cell should contain a copy if a Poisson distribution is expected) and a higher lentivirus dose that exceeds saturation 5 times. Copies per cell, percentage and MFI of cells (or concentration in media of transgene) may be detected in order to determine if all cells are expressing transgene. As a non-limiting example, T cells from two distinct donors may be transduced with lentivirus which includes a transgene. The transduction may be at two doses, saturation and 5× saturation, and show that 5-10 days after transduction that all groups may reach or exceed a predicted saturating level of integrated transgene and similar expression intensity across groups but not all cells are expressing the transgene. Not all T cells may have equal transduction susceptibility, even when sourced from the same donor. The fraction of total cells that express GFP (above the detection threshold) may vary between donors, lots and/or viral dose. The percent of total cells that express GFP from a single donor may be between 70% and 95%, such as, but not limited to, 70%, 70.1%, 70.2%, 70.3%, 70.4%, 70.5%, 70.6%, 70.7%, 70.8%, 70.9%, 71%, 71.1%, 71.2%, 71.3%, 71.4%, 71.5%, 71.6%, 71.7%, 71.8%, 71.9%, 72%, 72.1%, 72.2%, 72.3%, 72.4%, 72.5%, 72.6%, 72.7%, 72.8%, 72.9%, 73%, 73.1%, 73.2%, 73.3%, 73.4%, 73.5%, 73.6%, 73.7%, 73.8%, 73.9%, 74%, 74.1%, 74.2%, 74.3%, 74.4%, 74.5%, 74.6%, 74.7%, 74.8%, 74.9%, 75%, 75.1%, 75.2%, 75.3%, 75.4%, 75.5%, 75.6%, 75.7%, 75.8%, 75.9%, 76%, 76.1%, 76.2%, 76.3%, 76.4%, 76.5%, 76.6%, 76.7%, 76.8%, 76.9%, 77%, 77.1%, 77.2%, 77.3%, 77.4%, 77.5%, 77.6%, 77.7%, 77.8%, 77.9%, 78%, 78.1%, 78.2%, 78.3%, 78.4%, 78.5%, 78.6%, 78.7%, 78.8%, 78.9%, 79%, 79.1%, 79.2%, 79.3%, 79.4%, 79.5%, 79.6%, 79.7%, 79.8%, 79.9%, 80%, 80.1%, 80.2%, 80.3%, 80.4%, 80.5%, 80.6%, 80.7%, 80.8%, 80.9%, 81%, 81.1%, 81.2%, 81.3%, 81.4%, 81.5%, 81.6%, 81.7%, 81.8%, 81.9%, 82%, 82.1%, 82.2%, 82.3%, 82.4%, 82.5%, 82.6%, 82.7%, 82.8%, 82.9%, 83%, 83.1%, 83.2%, 83.3%, 83.4%, 83.5%, 83.6%, 83.7%, 83.8%, 83.9%, 84%, 84.1%, 84.2%, 84.3%, 84.4%, 84.5%, 84.6%, 84.7%, 84.8%, 84.9%, 85%, 85.1%, 85.2%, 85.3%, 85.4%, 85.5%, 85.6%, 85.7%, 85.8%, 85.9%, 86%, 86.1%, 86.2%, 86.3%, 86.4%, 86.5%, 86.6%, 86.7%, 86.8%, 86.9%, 87%, 87.1%, 87.2%, 87.3%, 87.4%, 87.5%, 87.6%, 87.7%, 87.8%, 87.9%, 88%, 88.1%, 88.2%, 88.3%, 88.4%, 88.5%, 88.6%, 88.7%, 88.8%, 88.9%, 89%, 89.1%, 89.2%, 89.3%, 89.4%, 89.5%, 89.6%, 89.7%, 89.8%, 89.9%, 90%, 90.1%, 90.2%, 90.3%, 90.4%, 90.5%, 90.6%, 90.7%, 90.8%, 90.9%, 91%, 91.1%, 91.2%, 91.3%, 91.4%, 91.5%, 91.6%, 91.7%, 91.8%, 91.9%, 92%, 92.1%, 92.2%, 92.3%, 92.4%, 92.5%, 92.6%, 92.7%, 92.8%, 92.9%, 93%, 93.1%, 93.2%, 93.3%, 93.4%, 93.5%, 93.6%, 93.7%, 93.8%, 93.9%, 94%, 94.1%, 94.2%, 94.3%, 94.4%, 94.5%, 94.6%, 94.7%, 94.8%, 94.9%, or 95%. As a non-limiting example, the percent of total cells expressing GFP in cells from one donor may be 83.8% for a dose of 1 μL and 83.7% or 78.8% for a dose of 5 μL. As another non-limiting example, the percent of total cells expressing GFP in cells from one donor may be 80.6%, 89.1%, or 91.2% for a dose of 1 and 75.1%, 89.6% or 91.7% for a dose of 5 μL.

In some embodiments, a percentage of the cultured T cells (e.g., primary human T cells and/or Jurkat cells) may express the transgene. The percentage of culture T cells expressing the transgene may be, but is not limited to, 5%, 10%, 15%, 20%, 25%, 30%, 35%, 40%, 45%, 50%, 55%, 60%, 65%, 70%, 75%, 80%, 85%, 90%, 95%, 97%, 99% or greater than 99%. As a non-limiting example, the percentage may be greater than 70%. As a non-limiting example, the percentage may be greater than 75%. As a non-limiting example, the percentage may be greater than 80%. As a non-limiting example, the percentage may be greater than 85%. As a non-limiting example, the percentage may be greater than 90%. As a non-limiting example, the percentage may be greater than 95%.

In some embodiments, the mRNA levels from the culture may decline over the duration of the study. The decline may not be limited to a specific transgene and the trend may be seen across multiple classes of expressed proteins. In order to increase the mRNA levels, the cells may be reactivated after the mRNA levels decrease from the initial levels. The cells may be reactivated 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 or more than 30 days after transduction. As a non-limiting example, in order to increase mRNA levels in the culture, the cells may be reactivated with CD3/CD28 beads 13 days after transduction. As a non-limiting example, in order to increase mRNA levels in the culture, the cells may be reactivated with CD3/CD28 beads 14 days after transduction. As a non-limiting example, in order to increase mRNA levels in the culture, the cells may be reactivated with CD3/CD28 beads 15 days after transduction

In some embodiments, the surface expression from the culture may decline over the duration of the study. For example, the surface expression may decline between days 3 to 13 days, 3 to 14 days, or 3 to 15 days after transduction. In order to increase the surface expression, the cells may be reactivated after the surface expression decrease from the initial levels. The cells may be reactivated 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 or more than 30 days after transduction. As a non-limiting example, in order to increase surface expression in the culture, the cells may be reactivated with CD3/CD28 beads 13 days after transduction. As a non-limiting example, in order to increase surface expression in the culture, the cells may be reactivated with CD3/CD28 beads 14 days after transduction. As a non-limiting example, in order to increase surface expression in the culture, the cells may be reactivated with CD3/CD28 beads 15 days after transduction.

In some embodiments, the transgene is a CAR such as, but not limited to, CD19 CAR. As a non-limiting example, the CAR is CD19 CAR. The cell viability may be greater than 90% in cells transduced with a CD19 CAR. The cell viability may be greater than 85% in cells transduced with a CD19 CAR. If the cells are primary T cells transduced with a CD19 CAR, then number of viable cells may increase over the initial timepoints before decreasing. If the cells are Jurkat cells transduced with a CD19 CAR, then the number of viable cells may increase for at least 10 days. The number of copies per cell for CD19 CAR transduced cells may be higher for the initial timepoints before decreasing by 50% or more for the later timepoints. The cell surface expression of CD19 CAR may decrease during the course of the study from about 20000 CAR MFI to less than 5000 CAR MFI over a period of 10 days (e.g., day 3 to day 13). After restimulation on day 15 the MFI may increase to above 5000 CAR MFI. The percentage of primary human T cells expressing CAR may be between 40% and 60% for 3-13 days after transduction. The percentage of Jurkat cells expressing CAR may be between 30% and 70% for 3-13 days after transduction. An initial decline of about 20% may be seen between days 3 and 6 after transduction. Restimulation of the T cells may increase the percent of CAR positive cells back to initial percentage levels (e.g., around 60%).

In some embodiments, the transgene is an interleukin such as, but not limited to, IL12 (e.g., membrane bound IL12 or secreted cytokine IL12), IL15 (e.g., membrane bound IL15), and IL15Ra. As a non-limiting example, the interleukin is IL12. The cell viability may be greater than 90% in cells transduced with IL12. The cell viability may be greater than 85% in cells transduced with IL12. If the cells are primary T cells transduced with IL12, the number of viable cells may increase over the initial timepoints before decreasing. If the cells are Jurkat cells transduced with IL12, the number of viable cells may increase for at least 10 days. The number of copies per cell for IL12 transduced cells may be higher for the initial timepoints before decreasing by 50% or more for the later timepoints. For IL12 transduced primary human T cells, the level of soluble IL12 in the media may drop steadily over the time course of the study with a slight increase visible in the restimulated group. For IL12 transduced Jurkat cells, the level of soluble IL12 in the media may have a drop in IL12 secretion in the first half of the culture with the levels remaining low through the second half of the culture time.

In some embodiments, the transgene encodes a fluorescent protein such as, but not limited to cytosolic green fluorescence protein (GFP), luciferase, and mCherry. As a non-limiting example, the fluorescent protein is GFP. The cell viability may be greater than 90% in cells transduced with GFP. The cell viability may be greater than 85% in cells transduced with GFP. If the cells are primary T cells transduced with GFP, then the number of viable cells may increase over the initial timepoints before decreasing. If the cells are Jurkat cells transduced with GFP, then the number of viable cells may increase for at least 10 days. The number of copies per cell for GFP transduced cells may be higher for the initial timepoints before decreasing by 50% or more for the later timepoints. The surface expression of the cells may have a steady and rapid decline bottoming out at day 10 with a slight increase if restimulated. The highest level of cell surface expression of GFP in Jurkat cells may be at day 10 (about 35000 GFP MFI) before decreasing for the rest of the study. The percentage of primary human T cells expressing GFP may be around 80% for 3-13 days after transduction. The percentage of Jurkat cells expressing GFP may be around 90% for 3-13 days after transduction.

In some embodiments, lentivirally engineered cells described herein have genomic DNA integration that stabilizes after an initial decline of copy number, decreasing RNA and surface expression levels over time, and an increase in RNA and surface expression after restimulation.

In some embodiments, lentivirally engineering cells may be evaluated using the following 14-day method where samples are collected 5 times throughout the culture. On day −1 the T cells (e.g., primary human T cells or Jurkat cells) may be thawed and the CD3/CD28 beads are added. On day 0, the lentivirus for each of the conditions is added (e.g., 4 mL of cells at 0.5e6/mL) and there is a control of non-transduced cells. Double media to 8 mL on day 1 and then double the media to 16 mL on day 2. On day 3, harvest 4 mL and then double media to 24 mL on day 4. Harvest 4 mL on day 6 before doubling media to 40 mL. The cells can be split (e.g., 14 mL 0.5e6 cells/mL) on day 8 and then on day 6 harvest 4 mL before doubling media to 40 mL. 4 mL may be harvested on day 10 before the media is doubled to 20 mL. On day 13, 4 mL are harvested before doubling the media to 32 mL. The culture is split in half and half of the culture is activated (CD3/CD28 activation beads 1:1) and stimulated overnight. On day 14, 4 mL of each stimulated and non-stimulated cells are harvested and the culture is ended. Transgene copy number per cell are assayed by harvesting cells and extracting genomic DNA then quantifying with standard curve qPCR against the endogenous genome and against the transgene sequence, then converting the detected quantities to a ratio. Mean Fluorescence Intensity (MFI) is assayed by FLO on an Attune with appropriate staining for each group. Percent expressing may also be assayed by FLO on an attune quantifying the percent of cells fluorescing above threshold. Soluble payloads can be quantified by harvesting culture supernatant at each marked timepoint and running MesoScale Discovery plate assay (MSD) then normalizing for cell density.

### 3. Adeno-Associated Viral Vectors (AAV)

In some embodiments, polynucleotides of present disclosure may be packaged into recombinant adeno-associated viral (rAAV) vectors. Such vectors or viral particles may be designed to utilize any of the known serotype capsids or combinations of serotype capsids. The serotype capsids may include capsids from any identified AAV serotypes and variants thereof, for example, AAV1, AAV2, AAV2G9, AAV3, AAV4, AAV4-4, AAV5, AAV6, AAV7, AAV8, AAV9, AAV10, AAV11, AAV12 and AAVrh10.

In one embodiment, the AAV serotype may be or have a sequence as described in United States Publication No. US20030138772, herein incorporated by reference in its entirety, such as, but not limited to, AAV1 (SEQ ID NO. 6 and 64 of US20030138772), AAV2 (SEQ ID NO. 7 and 70 of US20030138772), AAV3 (SEQ ID NO. 8 and 71 of US20030138772), AAV4 (SEQ ID NO. 63 of US20030138772), AAV5 (SEQ ID NO. 114 of US20030138772), AAV6 (SEQ ID NO. 65 of US20030138772), AAV7 (SEQ ID NO. 1-3 of US20030138772), AAV8 (SEQ ID NO. 4 and 95 of US20030138772), AAV9 (SEQ ID NO. 5 and 100 of US20030138772), AAV10 (SEQ ID NO. 117 of US20030138772), AAV11 (SEQ ID NO. 118 of US20030138772), AAV12 (SEQ ID NO. 119 of US20030138772), AAVrh10 (amino acids 1 to 738 of SEQ ID NO. 81 of US20030138772) or variants thereof. Non-limiting examples of variants include SEQ ID NOs. 9, 27-45, 47-62, 66-69, 73-81, 84-94, 96, 97, 99, 101-113 of US20030138772, the contents of which are herein incorporated by reference in their entirety.

In one embodiment, the AAV serotype may have a sequence as described in Pulicherla et al. (Molecular Therapy, 2011, 19(6):1070-1078), U.S. Pat. Nos. 6,156,303; 7,198,951; U.S. Patent Publication Nos. US2015/0159173 and US2014/0359799; and International Patent Publication Nos. WO1998/011244, WO2005/033321, and WO2014/14422; the contents of each of which are incorporated herein by reference in their entirety.

AAV vectors include not only single stranded vectors but self-complementary AAV vectors (scAAVs). scAAV vectors contain DNA which anneals together to form double stranded vector genome. By skipping second strand synthesis, scAAVs allow for rapid expression in the cell.

The rAAV vectors may be manufactured by standard methods in the art such as by triple transfection, in sf9 insect cells or in suspension cell cultures of human cells such as HEK293 cells.

The biocircuits, biocircuit components, effector modules, SREs or payload constructs may be encoded in one or more viral genomes to be packaged in the AAV capsids taught herein.

Such vectors or viral genomes may also include, in addition to at least one or two ITRs (inverted terminal repeats), certain regulatory elements necessary for expression from the vector or viral genome. Such regulatory elements are well known in the art and include for example promoters, introns, spacers, stuffer sequences, and the like.

In some embodiments, more than one effector module or SRE (e.g. DD) may be encoded in a viral genome.

### 4. Oncolytic Viruses

In some embodiments, polynucleotides of present disclosure may be packaged into oncolytic viruses, such as vaccine viruses. Oncolytic vaccine viruses may include viral particles of a thymidine kinase (TK)-deficient, granulocyte macrophage (GM)-colony stimulating factor (CSF)-expressing, replication-competent vaccinia virus vector sufficient to induce oncolysis of cells in the tumor (e.g., U.S. Pat. No. 9,226,977).

In some embodiments, the viral vector of the disclosure may comprise two or more immunotherapeutic agents taught herein, wherein the two or more immunotherapeutic agents may be included in one effector module under the regulation of the same DD. In this case, the two or more immunotherapeutic agents are tuned by the same stimulus simultaneously. In other embodiments, the viral vector of the disclosure may comprise two or more effector modules, wherein each effector module comprises a different immunotherapeutic agent. In this case, the two or more effector modules and immunotherapeutic agents are tuned by different stimuli, providing separately independent regulation of the two or more components.

5. Messenger RNA (mRNA)

In some embodiments, the effector modules of the disclosure may be designed as a messenger RNA (mRNA). As used herein, the term “messenger RNA” (mRNA) refers to any polynucleotide which encodes a polypeptide of interest and which is capable of being translated to produce the encoded polypeptide of interest in vitro, in vivo, in situ or ex vivo. Such mRNA molecules may have the structural components or features of any of those taught in International Publication No. WO2018151666, the contents of which are incorporated herein by reference in its entirety.

Polynucleotides of the disclosure may also be designed as taught in, for example, Ribostem Limited in United Kingdom patent application serial number 0316089.2 filed on Jul. 9, 2003, now abandoned, PCT application number PCT/GB2004/002981 filed on Jul. 9, 2004, published as WO2005005622, U.S. patent application national phase entry Ser. No. 10/563,897 filed on Jun. 8, 2006, published as US20060247195, now abandoned, and European patent application national phase entry serial number EP2004743322 filed on Jul. 9, 2004, published as EP1646714, now withdrawn; Novozymes, Inc. in PCT application number PCT/US2007/88060 filed on Dec. 19, 2007, published as WO2008140615, United States patent application national phase entry Ser. No. 12/520,072 filed on Jul. 2, 2009, published as US20100028943, and European patent application national phase entry serial number EP2007874376 filed on Jul. 7, 2009, published as EP2104739; University of Rochester in PCT application number PCT/US2006/46120 filed on Dec. 4, 2006, published as WO2007064952, and U.S. patent application Ser. No. 11/606,995 filed on Dec. 1, 2006, published as US20070141030; BioNTech AG in European patent application serial number EP2007024312 filed Dec. 14, 2007, now abandoned, PCT application number PCT/EP2008/01059 filed on Dec. 12, 2008, published as WO2009077134, European patent application national phase entry serial number EP2008861423 filed on Jun. 2, 2010, published as EP2240572, United States patent application national phase entry Ser. No. 12/735,060 filed Nov. 24, 2010, published as US20110065103, German patent application serial number DE 10 2005 046 490 filed Sep. 28, 2005, PCT application PCT/EP2006/0448 filed Sep. 28, 2006, published as WO2007036366, national phase European patent EP1934345 published Mar. 21, 2012, and national phase U.S. patent application Ser. No. 11/992,638 filed Aug. 14, 2009, published as 20100129877; Immune Disease Institute Inc. in U.S. patent application Ser. No. 13/088,009 filed Apr. 15, 2011, published as US20120046346, and PCT application PCT/US2011/32679 filed Apr. 15, 2011, published as WO20110130624; Shire Human Genetic Therapeutics in U.S. patent application Ser. No. 12/957,340 filed on Nov. 20, 2010, published as US20110244026; Sequitur Inc. in PCT application PCT/US1998/019492 filed on Sep. 18, 1998, published as WO1999014346; The Scripps Research Institute in PCT application number PCT/US2010/00567 filed on Feb. 24, 2010, published as WO2010098861, and United States patent application national phase entry Ser. No. 13/203,229 filed Nov. 3, 2011, published as US20120053333; Ludwig-Maximillians University in PCT application number PCT/EP2010/004681 filed on Jul. 30, 2010, published as WO2011012316; Cellscript Inc. in U.S. Pat. No. 8,039,214 filed Jun. 30, 2008, and granted Oct. 18, 2011, U.S. patent application Ser. No. 12/962,498 filed on Dec. 7, 2010, published as US20110143436, Ser. No. 12/962,468 filed on Dec. 7, 2010, published as US20110143397, Ser. No. 13/237,451 filed on Sep. 20, 2011, published as US20120009649, and PCT applications PCT/US2010/59305 filed Dec. 7, 2010, published as WO2011071931 and PCT/US2010/59317 filed on Dec. 7, 2010, published as WO2011071936; The Trustees of the University of Pennsylvania in PCT application number PCT/US2006/32372 filed on Aug. 21, 2006, published as WO2007024708, and U.S. patent application national phase entry Ser. No. 11/990,646 filed on Mar. 27, 2009, published as US20090286852; Curevac GMBH in German patent application serial numbers DE10 2001 027 283.9 filed Jun. 5, 2001, DE10 2001 062 480.8 filed Dec. 19, 2001, and DE 20 2006 051 516 filed Oct. 31, 2006, all abandoned, European patent numbers EP1392341 granted Mar. 30, 2005, and EP1458410 granted Jan. 2, 2008, PCT application numbers PCT/EP2002/06180 filed Jun. 5, 2002, published as WO2002098443, PCT/EP2002/14577 filed on Dec. 19, 2002, published as WO2003051401, PCT/EP2007/09469 filed on Dec. 31, 2007, published as WO2008052770, PCT/EP2008/03033 filed on Apr. 16, 2008, published as WO2009127230, PCT/EP2006/004784 filed on May 19, 2005, published as WO2006122828, PCT/EP2008/00081 filed on Jan. 9, 2007, published as WO2008083949, and U.S. patent application Ser. No. 10/729,830 filed on Dec. 5, 2003, published as US20050032730, Ser. No. 10/870,110 filed on Jun. 18, 2004, published as US20050059624, Ser. No. 11/914,945 filed on Jul. 7, 2008, published as US20080267873, Ser. No. 12/446,912 filed on Oct. 27, 2009, published as US2010047261, now abandoned, Ser. No. 12/522,214 filed on Jan. 4, 2010, published as US20100189729, Ser. No. 12/787,566 filed on May 26, 2010, published as US20110077287, Ser. No. 12/787,755 filed on May 26, 2010, published as US20100239608, Ser. No. 13/185,119 filed on Jul. 18, 2011, published as US20110269950, and Ser. No. 13/106,548 filed on May 12, 2011, published as US20110311472, all of which are herein incorporated by reference in their entirety.

In some embodiments, the effector modules may be designed as self-amplifying RNA. “Self-amplifying RNA” as used herein refers to RNA molecules that can replicate in the host resulting in the increase in the amount of the RNA and the protein encoded by the RNA. Such self-amplifying RNA may have structural features or components of any of those taught in International Patent Application Publication No. WO2011005799 (the contents of which are incorporated herein by reference in their entirety).

### VI. Definitions

At various places in the present specification, features or functions of the compositions of the present disclosure are disclosed in groups or in ranges. It is specifically intended that the present disclosure include each and every individual sub-combination of the members of such groups and ranges. The following is a non-limiting list of term definitions.

Activity: As used herein, the term “activity” refers to the condition in which things are happening or being done. Compositions of the disclosure may have activity and this activity may involve one or more biological events. In some embodiments, biological events may include cell signaling events. In some embodiments, biological events may include cell signaling events associated protein interactions with one or more corresponding proteins, receptors, small molecules or any of the biocircuit components described herein.

Approximately: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Alkyl: The terms “alkyl”, “alkoxy”, “hydroxyalkyl”, “alkoxy alkyl”, and “alkoxy carbonyl”, as used herein, include both straight and branched chains containing one to twelve carbon atoms, and/or which may or may not be substituted.

Alkenyl: The terms “alkenyl” and “alkynyl” as used herein alone or as part of a larger moiety shall include both straight and branched chains containing two to twelve carbon atoms.

Aryl: The term “aryl” as used herein alone or as part of a larger moiety as in “aralkyl”, “aralkoxy”, or “aryloxyalkyl”, refers to monocyclic, bicyclic and tricyclic carbocyclic ring systems having a total of five to fourteen ring members, wherein at least one ring is aromatic and wherein each ring in the system contains 3 to 8 ring members. The term “aryl” may be used interchangeably with the term “aryl ring.”

Aromatic: The term “aromatic” as used herein, refers to an unsaturated hydrocarbon ring structure with delocalized pi electrons. As used herein “aromatic” may refer to a monocyclic, bicyclic or polycyclic aromatic compound.

Aliphatic: The term “aliphatic” or “aliphatic group” as used herein, refers to a straight or branched C1-C8 hydrocarbon chain or a monocyclic C3-C8hydrocarbon or bicyclic C8-C12 hydrocarbon which are fully saturated or that contains one or more units of unsaturation, that is completely saturated or that contains one or more units of unsaturation, but which is not aromatic (also referred to herein as “carbocycle” or “cycloalkyl”), and that has a single point of attachment to the rest of the molecule wherein any individual ring in said bicyclic ring system has 3-7 members.

Associated with: As used herein, the terms “associated with,” “conjugated,” “linked,” “attached,” and “tethered,” when used with respect to two or more moieties, mean that the moieties are physically associated or connected with one another, either directly or via one or more additional moieties that serve as linking agents, to form a structure that is sufficiently stable so that the moieties remain physically associated under the conditions in which the structure is used, e.g., physiological conditions. An “association” need not be strictly through direct covalent chemical bonding. It may also suggest ionic or hydrogen bonding or a hybridization-based connectivity sufficiently stable such that the “associated” entities remain physically associated.

Biocircuit system: As used herein, a “biocircuit” or “biocircuit system” is defined as a circuit within or useful in biologic systems comprising a stimulus and at least one effector module responsive to a stimulus, where the response to the stimulus produces at least one signal or outcome within, between, as an indicator of, or on a biologic system. Biologic systems are generally understood to be any cell, tissue, organ, organ system or organism, whether animal, plant, fungi, bacterial, or viral. It is also understood that biocircuits may be artificial circuits which employ the stimuli or effector modules taught by the present disclosure and effect signals or outcomes in acellular environments such as with diagnostic, reporter systems, devices, assays or kits. The artificial circuits may be associated with one or more electronic, magnetic, or radioactive components or parts. In the context of the present disclosure, a biocircuit includes a destabilizing domain (DD) biocircuit system.

Conservative amino acid substitution: As used herein a” conservative amino acid substitution is one in which an amino acid residue is substituted by another amino acid residue having a side chain (R group) with similar properties (e.g. charge or hydrophobicity).

Destabilized: As used herein, the term “destable,” “destabilize,” destabilizing region” or “destabilizing domain” means a region or molecule that is less stable than a starting, reference, wild-type or native form of the same region or molecule.

Expression: As used herein, “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end processing); (3) translation of an RNA into a polypeptide or protein; (4) folding of a polypeptide or protein; and (5) post-translational modification of a polypeptide or protein.

Feature: As used herein, a “feature” refers to a characteristic, a property, or a distinctive element.

Formulation: As used herein, a “formulation” includes at least a compound and/or composition of the present disclosure and a delivery agent.

Fragment: A “fragment,” as used herein, refers to a portion. For example, fragments of proteins may comprise polypeptides obtained by digesting full-length protein. In some embodiments, a fragment of a protein includes at least 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, 250 or more amino acids. In some embodiments, fragments of an antibody include portions of an antibody.

Functional: As used herein, a “functional” biological molecule is a biological entity with a structure and in a form in which it exhibits a property and/or activity by which it is characterized.

Heterocycle: The term “heterocycle”, “heterocyclyl”, or “heterocyclic” as used herein refers to monocyclic, bicyclic or tricyclic ring systems having three to fourteen ring members in which one or more ring members is a heteroatom, wherein each ring in the system contains 3 to 7 ring members and is non-aromatic.

Hotspot: As used herein, a “hotspot” or a “mutational hotspot” refers to an amino acid position in a protein coding gene that is mutated (by substitutions) more frequently relative to elsewhere within the same gene.

IC₅₀: As used herein, the term “IC₅₀” refers to the concentration of the ligand where the response or binding is reduced to half.

Immunotherapeutic agent: the term “immunotherapeutic agent” as used herein, refers to the treatment of disease by the induction or restoration of the reactivity of the immune system towards the disease with a biological, pharmaceutical, or chemical compound.

In vitro: As used herein, the term “in vitro” refers to events that occur in an artificial environment, e.g., in a test tube or reaction vessel, in cell culture, in a Petri dish, etc., rather than within an organism (e.g., animal, plant, or microbe).

In vivo: As used herein, the term “in vivo” refers to events that occur within an organism (e.g., animal, plant, or microbe or cell or tissue thereof).

Linker: As used herein, a linker refers to a moiety that connects two or more domains, moieties or entities. In one embodiment, a linker may comprise 10 or more atoms. In a further embodiment, a linker may comprise a group of atoms, e.g., 10-1,000 atoms, and can be comprised of the atoms or groups such as, but not limited to, carbon, amino, alkylamino, oxygen, sulfur, sulfoxide, sulfonyl, carbonyl, and imine. In some embodiments, a linker may comprise one or more nucleic acids comprising one or more nucleotides. In some embodiments, the linker may comprise an amino acid, peptide, polypeptide or protein. In some embodiments, a moiety bound by a linker may include, but is not limited to an atom, a chemical group, a nucleoside, a nucleotide, a nucleobase, a sugar, a nucleic acid, an amino acid, a peptide, a polypeptide, a protein, a protein complex, a payload (e.g., a therapeutic agent), or a marker (including, but not limited to a chemical, fluorescent, radioactive or bioluminescent marker). The linker can be used for any useful purpose, such as to form multimers or conjugates, as well as to administer a payload, as described herein. Examples of chemical groups that can be incorporated into the linker include, but are not limited to, alkyl, alkenyl, alkynyl, amido, amino, ether, thioether, ester, alkylene, heteroalkylene, aryl, or heterocyclyl, each of which can be optionally substituted, as described herein. Examples of linkers include, but are not limited to, unsaturated alkanes, polyethylene glycols (e.g., ethylene or propylene glycol monomeric units, e.g., diethylene glycol, dipropylene glycol, triethylene glycol, tripropylene glycol, tetraethylene glycol, or tetraethylene glycol), and dextran polymers, Other examples include, but are not limited to, cleavable moieties within the linker, such as, for example, a disulfide bond (—S—S—) or an azo bond (—N═N—), which can be cleaved using a reducing agent or photolysis. Non-limiting examples of a selectively cleavable bonds include an amido bond which may be cleaved for example by the use of tris(2-carboxyethyl) phosphine (TCEP), or other reducing agents, and/or photolysis, as well as an ester bond which may be cleaved for example by acidic or basic hydrolysis.

MOI: As used herein, the term “MOI” refers to the multiplicity of infection which is defined as the average number of virus particles infecting a target cell.

Modified: As used herein, the term “modified” refers to a changed state or structure of a molecule or entity as compared with a parent or reference molecule or entity. Molecules may be modified in many ways including chemically, structurally, and functionally. In some embodiments, compounds and/or compositions of the present disclosure are modified by the introduction of non-natural amino acids.

Mutation: As used herein, the term “mutation” refers to a change and/or alteration. In some embodiments, mutations may be changes and/or alterations to proteins (including peptides and polypeptides) and/or nucleic acids (including polynucleic acids). In some embodiments, mutations comprise changes and/or alterations to a protein and/or nucleic acid sequence. Such changes and/or alterations may comprise the addition, substitution and or deletion of one or more amino acids (in the case of proteins and/or peptides) and/or nucleotides (in the case of nucleic acids and or polynucleic acids, e.g., polynucleotides). In some embodiments, wherein mutations comprise the addition and/or substitution of amino acids and/or nucleotides, such additions and/or substitutions may comprise 1 or more amino acid and/or nucleotide residues and may include modified amino acids and/or nucleotides. The resulting construct, molecule or sequence of a mutation, change or alteration may be referred to herein as a mutant.

Off-target: As used herein, “off target” refers to any unintended effect on any one or more target, gene, cellular transcript, cell, and/or tissue.

Operably linked: As used herein, the phrase “operably linked” refers to a functional connection between two or more molecules, constructs, transcripts, entities, moieties or the like.

Protein of interest: As used herein, the terms “proteins of interest” or “desired proteins” include those provided herein and fragments, mutants, variants, and alterations thereof.

Purine: As used herein, “purine” refers to an aromatic heterocyclic structure, wherein one of the heterocycles is an imidazole ring and one of the heterocycles is a pyrimidine ring.

Pyrimidine: As used herein, “pyrimidine” refers to an aromatic heterocyclic structure similar to benzene, but wherein two of the carbon atoms are replaced by nitrogen atoms.

Pyridopyrimidine: As used herein, “Pyridopyrimidine” refers to an aromatic heterocyclic structure, wherein one of the heterocycles is a purine ring and one of the heterocycles is a pyrimidine ring.

Quinazoline: As used herein, the term, “Quinazoline” refers to an aromatic heterocyclic structure, wherein one of the heterocycles is a benzene ring and one of the heterocycles is a pyrimidine ring.

Stable: As used herein “stable” refers to a compound or entity that is sufficiently robust to survive isolation to a useful degree of purity from a reaction mixture, and preferably capable of formulation into an efficacious therapeutic agent.

Stabilized: As used herein, the term “stabilize”, “stabilized,” “stabilized region” means to make or become stable. In some embodiments, stability is measured relative to an absolute value. In some embodiments, stability is measured relative to a secondary status or state or to a reference compound or entity.

Stimulus response element (SRE): the term “stimulus response element (SRE), as used herein, is a component of an effector module which is joined, attached, linked to or associated with one or more payloads of the effector module and in some instances, is responsible for the responsive nature of the effector module to one or more stimuli. As used herein, the “responsive” nature of an SRE to a stimulus may be characterized by a covalent or non-covalent interaction, a direct or indirect association or a structural or chemical reaction to the stimulus. Further, the response of any SRE to a stimulus may be a matter of degree or kind. The response may be a partial response. The response may be a reversible response. The response may ultimately lead to a regulated signal or output. Such output signal may be of a relative nature to the stimulus, e.g., producing a modulatory effect of between 1 and 100 or a factored increase or decrease such as 2-fold, 3-fold, 4-fold, 5-fold, 10-fold or more. One non-limiting example of an SRE is a destabilizing domain (DD).

Subject: As used herein, the term “subject” or “patient” refers to any organism to which a composition in accordance with the disclosure may be administered, e.g., for experimental, diagnostic, prophylactic, and/or therapeutic purposes. Typical subjects include animals (e.g., mammals such as mice, rats, rabbits, non-human primates, and humans) and/or plants.

Therapeutic Agent: The term “therapeutic agent” refers to any agent that, when administered to a subject, has a therapeutic, diagnostic, and/or prophylactic effect and/or elicits a desired biological and/or pharmacological effect. Therapeutic agents of the present disclosure include any of the biocircuit components taught herein either alone or in combination with other therapeutic agents.

Therapeutically effective amount: As used herein, the term “therapeutically effective amount” means an amount of an agent to be delivered (e.g., nucleic acid, drug, therapeutic agent, diagnostic agent, prophylactic agent, etc.) that is sufficient, when administered to a subject suffering from or susceptible to an infection, disease, disorder, and/or condition, to treat, improve symptoms of, diagnose, prevent, and/or delay the onset of the infection, disease, disorder, and/or condition. In some embodiments, a therapeutically effective amount is provided in a single dose. In some embodiments, a therapeutically effective amount is administered in a dosage regimen comprising a plurality of doses. Those skilled in the art will appreciate that in some embodiments, a unit dosage form may be considered to comprise a therapeutically effective amount of a particular agent or entity if it comprises an amount that is effective when administered as part of such a dosage regimen.

Triazine: As used herein, “triazine” is a class of nitrogen containing heterocycles with a structure similar to benzene, but wherein three carbon atoms are replaced by nitrogen atoms.

Treatment or treating: As used herein, the terms “treatment” or “treating” denote an approach for obtaining a beneficial or desired result including and preferably a beneficial or desired clinical result. Such beneficial or desired clinical results include, but are not limited to, one or more of the following: reducing the proliferation of (or destroying) cancerous cells or other diseased, reducing metastasis of cancerous cells found in cancers, shrinking the size of the tumor, decreasing symptoms resulting from the disease, increasing the quality of life of those suffering from the disease, decreasing the dose of other medications required to treat the disease, delaying the progression of the disease, and/or prolonging survival of individuals.

Tune: As used herein, the term “tune” means to adjust, balance or adapt one thing in response to a stimulus or toward a particular outcome. In one non-limiting example, the SREs and/or DDs of the present disclosure adjust, balance or adapt the function or structure of compositions to which they are appended, attached or associated with in response to particular stimuli and/or environments.

Variant: As used herein, the term “variant” refers to a first composition (e.g., a first DD or payload), that is related to a second composition (e.g., a second DD or payload, also termed a “parent” molecule). The variant molecule can be derived from, isolated from, based on or homologous to the parent molecule. The term variant can be used to describe either polynucleotides or polypeptides.

### EQUIVALENTS AND SCOPE

Those skilled in the art will recognize or be able to ascertain using no more than routine experimentation, many equivalents to the specific embodiments in accordance with the disclosure described herein. The scope of the present disclosure is not intended to be limited to the above Description, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or the entire group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the disclosure, to the tenth of the unit of the lower limit of the range, unless the context clearly dictates otherwise.

In addition, it is to be understood that any particular embodiment of the present disclosure that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the disclosure (e.g., any antibiotic, therapeutic or active ingredient; any method of production; any method of use; etc.) can be excluded from any one or more claims, for any reason, whether or not related to the existence of prior art.

It is to be understood that the words which have been used are words of description rather than limitation, and that changes may be made within the purview of the appended claims without departing from the true scope and spirit of the disclosure in its broader aspects.

### EXAMPLES

In the examples below, constructs and DDs are referred to by their identifiers (e.g., OT-001737). Additional information regarding the constructs and DDs may be found throughout the specification.

### Example 1. Generation of Novel Ligand Responsive DDs by Mutagenesis Screening

### Study Design

To engineer constructs that display ligand dependent stability, a candidate ligand binding domain (LBD) is selected and a cell-based screen using yellow fluorescent protein (YFP) as a reporter for protein stability is designed to identify mutants of the candidate LBD possessing the desired characteristics of a destabilizing domain: low protein levels in the absence of a ligand of the LBD, (i.e., low basal stability), large dynamic range, robust and predictable dose-response behavior, and rapid kinetics of degradation (Banaszynski, et al., (2006) Cell; 126(5): 995-1004). The candidate LBD binds to a desired ligand but not endogenous signaling molecules.

The candidate LBD sequence (as a template) is first mutated using a combination of nucleotide analog mutagenesis and error-prone PCR, to generate libraries of mutants based on the template candidate domain sequence. The libraries generated are cloned in-frame at either the 5′- or 3′-ends of the YFP gene, and a retroviral expression system is used to stably transduce the libraries of YFP fusions into NIH3T3 fibroblasts.

The transduced NIH3T3 cells are subjected to three to four rounds of sorting using fluorescence-activated cell sorting (FACS) to screen the libraries of candidate DDs. Transduced NIH3T3 cells are cultured in the absence of the high affinity ligand of the ligand binding domain (LBD), and cells that exhibit low levels of YFP expression are selected through FACS.

### Screening Strategy I

The selected cell population is cultured in the presence of the high affinity ligand of the ligand binding domain for a period of time (e.g., 24 hours), at which point cells are sorted again by FACS. Cells that exhibit high levels of YFP expression are selected through FACS and the selected cell population is split into two groups and treated again with the high affinity ligand of the ligand binding domain at different concentrations; one group is treated with the lower concentration of the ligand and the other is treated with a high concentration of the ligand, for a period of time (e.g., 24 hours), at which point cells are sorted again by FACS. Cells expressing mutants that are responsive to lower concentrations of the ligand are isolated.

The isolated cells responsive to the lower concentration of the ligand are treated with the ligand again and cells exhibiting low fluorescence levels are collected 4 hours following removal of the ligand from the media. This fourth sorting is designed to enrich cells that exhibit fast kinetics of degradation (Iwamoto et al., Chem Biol. 2010 Sep. 24; 17(9): 981-988).

### Screening Strategy II

The selected cell population is subject to additional one or more sorts by FACS in the absence of high affinity ligand of LBD and cells that exhibit low levels of YFP expression are selected for further analysis. Cells are treated with high affinity ligand of the ligand binding domain, for a period of time (e.g. 24 hours) and sorted again by FACS. Cells expressing high levels of YFP are selected for through FACS. Cells with high expression of YFP are treated with ligand again and cells exhibiting low fluorescence levels are collected 4 hours following removal of the ligand from the media to enrich cells that exhibit fast kinetics of degradation. Any of the sorting steps may be repeated to identify DDs with ligand dependent stability.

The cells are recovered after sorting. The identified candidate cells are harvested, and the genomic DNA is extracted. The candidate DDs are amplified by PCR and isolated. The candidate DDs are sequenced and compared to the LBD template to identify the mutations in candidate DDs.

### Example 2. Regulation of IL12 by ER DDs

HEK 293T cells were transiently transfected with constructs containing ER-DDs fused to flexi IL12 with or without intervening furin site to generate constructs: OT-001569, OT-001737. Cells were then treated with 1 μM Bazedoxifene (BZD) or vehicle control for 24 hours and IL12 levels were measured by MSD assay Table 11.

TABLE 11
IL12 expression

| | | 1 μM | Stabilization |
|---|---|---|---|
| Construct | Vehicle | BZD | Ratio |
| OT-001737 | 10186.4 | 79706.8 | 7.8 |
| OT-001569 | 11529.1 | 109878 | 9.5 |

Both constructs showed ligand dependent stabilization of IL12 with comparable stabilization ratios indicating that the furin site did not affect IL12 regulation. Transfected HEK293T cell supernatants of both constructs were incubated for 24 hours with HEK-blue IL12 reporter cells, which express human IL12 receptor and STAT-4 reporter (InvivoGen, San Diego, Calif.). Experiments performed in the presence of BZD indicated increased STAT-4 reporter activity when the activity was compared to vehicle control for both constructs. These data suggest that the expressed IL12 is functional.

### Example 3. ER DD Hotspot Mutation Libraries

Libraries were generated by mutagenizing the estrogen receptor DD mutations N413D and Q502H to every other possible amino acid. The libraries were introduced into HEK293 cells and assayed for ligand-dependent stabilizing activity when fused to flexi IL12 as the payload. The constructs were evaluated in HEK293 cells for basal and ligand-induced expression. Cells were transfected with the constructs and incubated 24 hours post transfection with 1 μM Bazedoxifene (BZD). 24 hours following the incubation with BZD, supernatants were collected and analyzed for IL12p70 using Meso Scale Discovery (MSD) assay. The results are shown in Table 12 as pg/mL.

TABLE 12
IL12 levels with ER DD constructs

| | Mutation | | | |
|---|---|---|---|---|
| | at | | | |
| | positions | | | |
| | Q502 or | | 1 μM | Stabilization |
| Construct | N413 | Vehicle | Bazedoxifene | Ratio |
| OT-001711 | Q502D | 25091.76 | 594674.71 | 23.7 |
| OT-001723 | Q502H | 22806.89 | 310173.70 | 13.6 |
| OT-001691 | N413T | 18307.28 | 236163.91 | 12.9 |
| OT-001708 | Q502R | 14433.4 | 152994.04 | 10.6 |
| OT-001699 | WT | 14165.85 | 131742.41 | 9.3 |
| OT-001707 | N413D | 38747.88 | 333231.77 | 8.6 |
| OT-001703 | N413H | 15560.38 | 129151.15 | 8.3 |
| OT-001690 | N413A | 38146.81 | 278471.71 | 7.3 |
| OT-001702 | N413Q | 32295.36 | 219608.45 | 6.8 |
| OT-001710 | Q502E | 17924.43 | 109339.02 | 6.1 |
| OT-001718 | Q502V | 6651.394 | 37247.81 | 5.6 |
| OT-001697 | N413V | 27365.5 | 150510.25 | 5.5 |
| OT-001712 | Q502A | 17725.2 | 88626.00 | 5 |
| OT-001713 | Q502T | 18899.8 | 92609.02 | 4.9 |
| OT-001720 | Q502N | 38821.67 | 186344.02 | 4.8 |
| OT-001719 | Q502K | 48696.7 | 228874.49 | 4.7 |
| OT-001695 | N413C | 49042.06 | 225593.48 | 4.6 |
| OT-001717 | Q502S | 32837.05 | 137915.61 | 4.2 |
| OT-001698 | N413K | 42083.8 | 155710.06 | 3.7 |
| OT-001700 | N413M | 47368.35 | 161052.39 | 3.4 |
| OT-001692 | N413R | 52035.92 | 171718.54 | 3.3 |
| OT-001725 | Q502L | 26397.36 | 81831.82 | 3.1 |
| OT-001724 | Q502Y | 22833.51 | 68500.53 | 3 |
| OT-001696 | N413S | 41349.58 | 119913.78 | 2.9 |
| OT-001715 | Q502W | 20337.27 | 56944.36 | 2.8 |
| OT-001694 | N413W | 20032.29 | 54087.18 | 2.7 |
| OT-001726 | Q502F | 14250.2 | 35625.50 | 2.5 |
| OT-001722 | Q502I | 28946.44 | 66576.81 | 2.3 |
| OT-001701 | N413I | 53160.05 | 101004.10 | 1.9 |
| OT-001689 | N413E | 13701.46 | 24662.63 | 1.8 |
| OT-001709 | Q502G | 33881.58 | 60986.84 | 1.8 |
| OT-001705 | N413L | 17298.96 | 29408.23 | 1.7 |
| OT-001693 | N413P | 46614.33 | 74582.93 | 1.6 |
| OT-001706 | N413F | 53239.15 | 74534.81 | 1.4 |
| OT-001704 | N413Y | 46751.82 | 65452.55 | 1.4 |
| OT-001688 | N413G | 15876.24 | 20639.11 | 1.3 |
| OT-001714 | Q502P | 23944.71 | 28733.65 | 1.2 |
| OT-001721 | Q502M | 1145726 | 1145726.00 | 1 |
| OT-001716 | Q502C | 49101.45 | 44191.31 | 0.9 |

A diverse set of stabilizing activities were discovered with this approach. Some of the mutations reduced basal expression i.e. in the absence of ligand e.g. OT-001718. Other mutations enhanced the dynamic range of ligand regulation e.g. OT-001711, OT-001723, OT-001691, and OT-001708. Selection of the DD mutation utilized in a construct may depend on the type of regulation desired i.e. low basal expression and/or enhanced dynamic range of ligand regulation.

### Example 4. ER DD Regulated Tandem IL12-CD19 Tandem CAR Constructs in Transiently Transfected HEK293T Cells

HEK293T cells were transiently transfected with 1 μg DNA from each construct listed in the first column of Table 13 and incubated in DMEM with 10% FBS at 37° C., 5% CO2 for 24 hrs. Following the incubation, 1 μM of Bazedoxifene (BZD) or vehicle was added and cells were further incubated for 24 hrs. Following the incubation, CD19 CAR expression on the cell surface was quantified using FACS with CD19-Fc reagent, as a measure of transfection efficiency. IL12 levels in the media were quantified using MSD assay for p40. The IL12 and CD19 levels are shown in Table 13, where SD indicates standard deviation and SR indicates stabilization ratio. The stabilization ratio was calculated using the average IL12 values.

TABLE 13
Payload expression in IL12-CD19 CAR tandem constructs

| | | IL12p40 | | | | | |
|---|---|---|---|---|---|---|---|
| | | 1 μM BZD | | DMSO | | | Surface |
| | | Average | | Average | | | CAR |
| Construct ID | Mutation | (pg/ml) | SD | (pg/ml) | SD | SR | (%) |
| OT-001356 | — | | | 203531.67 | 4796.45 | | 40 |
| OT-001569 | T371A, L384M, | 75542.57 | 494.16 | 6443.64 | 92.65 | 11.72 | 0.1 |
| | M421G, N519S, | | | | | | |
| | G521R, Y537S | | | | | | |
| OT-001640 | R335G, L384M, | 2899.54 | 23.91 | 278.95 | 27.78 | 10.39 | 21 |
| | M421G, G521R, | | | | | | |
| | E523G, Y537S, | | | | | | |
| | A546T | | | | | | |
| OT-001642 | L384M, N413D, | 2570.95 | 20.07 | 103.53 | 16.73 | 24.83 | 11 |
| | M421G, G521R, | | | | | | |
| | Y537S | | | | | | |
| OT-001643 | L384M, M421G, | 2216.92 | 33.82 | 310.82 | 18.54 | 7.13 | 15 |
| | N519S, G521R, | | | | | | |
| | Y537S | | | | | | |
| OT-001644 | L384M, M421G, | 2388.95 | 49.46 | 253.89 | 10.69 | 9.41 | 15 |
| | Q502R, G521R, | | | | | | |
| | Y537S | | | | | | |
| OT-001646 | S305N, L384M, | 3673.33 | 150.27 | 238.69 | 12.87 | 15.39 | 18 |
| | M421G, G442V, | | | | | | |
| | G521R, Y537S | | | | | | |
| OT-001647 | R335G, L384M, | 3458.19 | 88.93 | 875.05 | 88.43 | 3.95 | 17 |
| | M421G, N519S, | | | | | | |
| | G521R, Y537S | | | | | | |
| OT-001648 | L384M, M421G, | 2341.77 | 137.35 | 410.12 | 12.15 | 5.71 | 16 |
| | G521R, Y537S | | | | | | |
| HEK293T | — | 0.00 | 0.00 | | | | |

All constructs tested in Table 13 showed ligand dependent IL12 expression, with stabilization ratios greater than 3. In a similar experimental set up, OT-001641 was tested using 1 μM Bazedoxifene and showed ligand dependent regulation of IL12 levels that was comparable to OT-001640 and OT-001644 with a stabilization ratio of 6. OT-001645 showed 68% CAR surface expression and a stabilization ratio of 5 in the presence of BZD.

OT-001736 construct was also tested using the same experimental set up. In the absence of ligand, the average IL12 p40 levels were 13364 pg/ml, which increased to 99015 pg/ml in the presence of ligand, resulting in a stabilization ratio of 7.4.

### Example 5. ER DD Regulates IL12 Expressed in Tandem with CD19-CAR in Lentivirus Transduced Primary Human T Cells

Primary human T cells were expanded with CD3/CD28 Dynabeads, transduced with lentivirus constructs and expanded for 10 days. Beads were removed and cells were frozen. T cells were later thawed and 100,000 cells were treated overnight with different doses of Bazedoxifene (BZD). Transduction efficiency was assessed by flow cytometry using CD19-Fc staining. The response of IL12 in CD19 CAR-IL12 tandem constructs to increasing concentrations of ligand was measured using OT-001736 construct with increasing doses of BZD starting at 0.01 nM to up to 3 μM for 23 hours. IL12 levels were measured using MSD assay. Controls tested in the experiment included OT-001357, which constitutively expresses IL12 and OT-001407, which is not expected to show any IL12 expression. The results are shown in Table 14.

TABLE 14
IL12 (p70) levels pg/ml

| Bazedoxifene | | | |
|---|---|---|---|
| Concentration | OT- | OT- | OT- |
| (nM) | 001407 | 001357 | 001736 |
| 3000.00 | 0.23 | 1080.34 | 158.42 |
| 1000.00 | 0.21 | 1289.31 | 136.87 |
| 333.33 | 0.26 | 1305.01 | 104.63 |
| 111.11 | 0.17 | 1345.32 | 77.50 |
| 37.04 | 0.22 | 1308.23 | 48.20 |
| 12.35 | 0.16 | 1178.63 | 27.79 |
| 4.12 | 0.21 | 1408.39 | 22.85 |
| 1.37 | 0.17 | 1353.44 | 20.80 |
| 0.46 | 0.20 | 1295.35 | 19.04 |
| 0.15 | 0.18 | 1213.58 | 19.26 |
| 0.05 | 0.34 | 1453.81 | 20.75 |
| 0.01 | — | — | 19.48 |

As little as 0.05 μM BZD induced IL12 expression in the cells expressing OT-001736. A dose dependent increase in IL12 pg/ml levels was observed up until the highest dose tested, i.e., 3 μM. The EC50 was 0.2 μM. As expected, OT-001357, showed high expression of IL12, whereas the expression of IL12 was undetectable in OT-001407 expressing cells.

Additional ER mutants were tested in primary human T cells as described above, except T cells were treated on day 9 of expansion, and not frozen and thawed prior to the experiment (Table 15). A dose dependent response was observed with all constructs tested.

TABLE 15
IL12 (p70) levels pg/ml

| Bazedoxifene | | | | | |
|---|---|---|---|---|---|
| Concentration | OT- | OT- | OT- | OT- | OT- |
| (nM) | 001357 | 001736 | 001649 | 001652 | 001653 |
| 6000.00 | 8056.87 | 410.80 | 544.20 | 656.15 | 456.38 |
| 3000.00 | 10578.75 | 404.48 | 581.10 | 606.65 | 494.18 |
| 1500.00 | 11035.45 | 387.55 | 503.97 | 553.13 | 462.85 |
| 750.00 | 10692.65 | 345.08 | 448.60 | 437.53 | 390.48 |
| 375.00 | 13485.30 | 298.53 | 407.18 | 393.78 | 332.37 |
| 187.50 | 11135.30 | 188.80 | 248.83 | 218.00 | 203.20 |
| 93.75 | 11783.80 | 131.53 | 177.77 | 140.40 | 166.17 |
| 46.88 | 10945.95 | 76.57 | 120.90 | 100.67 | 103.73 |
| 23.44 | 11258.30 | 54.60 | 109.47 | 91.33 | 92.40 |
| 11.72 | 10850.85 | 45.70 | 91.87 | 76.40 | 82.60 |
| 5.86 | 11802.80 | 37.93 | 90.93 | 80.97 | 82.83 |
| 0.10 | 9723.30 | 39.80 | 96.03 | 65.77 | 78.77 |

### Example 6. ER DD Regulated IL12 Expression In Vivo

Primary human T cells were activated with CD3/CD28 Dynabeads, transduced with lentivirus construct OT-001736 and expanded for 10 days. Beads were removed from the cell cultures and the T cells were frozen. Transduction efficiency of the T cells was assessed by flow cytometry for CD19-Fc. To evaluate regulation of IL12 protein production in vivo, tests were performed in NSG mice. All murine studies were done in accordance with the Institutional Animal Care and Use approved protocol. Female NSG mice were intravenously injected with Nalm6-luciferase tagged tumor cells, which is a CD19 positive human leukemia cell line. Seven days after tumor transplant, human T cells engineered to express CD19-targeting CAR with or without co-expression of regulated human IL12, were transferred into the mice by tail vein injection (1×10⁶ CD19-CAR positive cells per mouse). Animals were orally dosed with 10, 30 and 100 mg/kg BZD or vehicle 6, 8, and 13 days after T cell transfer, and peripheral blood samples taken at 0, 6, and 24 hours after each Bazedoxifene (BZD) or vehicle (10% DMSO; 90% (20% 2-Hydroxypropyl-β-cyclodextrin) dosing. IL12p70 levels were measured in the plasma by MSD (Table 16). Flow cytometry was also performed on blood samples to quantitate presence of CAR positive T cell numbers in the blood seven, nine and fourteen days after T cell transfer. CAR-Ts could be detected in the blood at all those timepoints.

TABLE 16
Plasma IL12 (p70) levels pg/ml

| Time | OT-001407 | | OT-001357 | | OT-001736 | | | |
|---|---|---|---|---|---|---|---|---|
| After | | 100 | | 100 | | 10 | 30 | 100 |
| First | | mg/kg | | mg/kg | | mg/kg | mg/kg | mg/kg |
| Dose (h) | Vehicle | BZD | Vehicle | BZD | Vehicle | BZD | BZD | BZD |
| 0 | 0.19 | 0.00 | 13229.50 | 12390.85 | 14.53 | 7.24 | 4.95 | 9.34 |
| 6 | 0.18 | 0.00 | 16221.33 | 14603.10 | 17.79 | 7.08 | 9.32 | 39.92 |
| 24 | 0.61 | 0.08 | 28496.88 | 25488.08 | 17.62 | 11.80 | 6.45 | 12.17 |
| 48 | 0.68 | 0.00 | 42766.25 | 36649.69 | 21.45 | 13.80 | 7.91 | 12.30 |
| 54 | 0.80 | 0.00 | 43510.68 | 35768.21 | 25.99 | 13.39 | 17.22 | 62.85 |
| 72 | 0.87 | 0.00 | 54674.38 | 43454.54 | 30.24 | 17.39 | 9.66 | 15.40 |
| 120 | 4.21 | 0.41 | 356425.33 | 335338.75 | 19.88 | — | — | 18.38 |
| 126 | 4.42 | 0.08 | 452725.33 | 398420.00 | 20.50 | — | — | 154.14 |
| 144 | 6.22 | 0.30 | 498157.67 | 491465.25 | 34.00 | — | — | 30.86 |

Ligand dependent regulation of IL12 was observed at 100 mg/kg. The levels of IL12 demonstrated a pulsatile expression pattern which correlated with the dosing schedule. A 5-9 fold increase in IL12 levels in the plasma was observed 6 hours post dose but declined for 24 hours post dose. Ligand dependent regulation was not observed at the lower doses tested. As expected, OT-001357 showed high IL12 levels both in the presence and absence of ligand while OT-001407 did not show any ligand dependent regulation.

### Example 7. Regulation of CD19 CAR by ER DDs

Primary T cells were activated with CD3/CD28 Dynabeads at 3:1 Bead: Cell ratio overnight, then transduced with OT-001792 lentivirus. On day 7 of the T cell expansion, CAR expression was measured by flow cytometry with CD19-Fc on cells treated overnight in the presence (34% positive) or absence (0.6% positive) of 1 μM Bazedoxifene. After the T cells had expanded for 10 days, they were mixed with CD19-expressing Nalm6 target cells at ratios of 10:1 and 2.5:1 total T cells:Nalm6 cells and cultured for 3 days. CD19-CAR mediated target cell killing, as measured by a decrease in stably transduced red-fluorescent protein expression from Nalm6 cells detected using the Incucyte® instrument, was observed specifically in the presence of 1 μM Bazedoxifene at both effector:target cell ratios tested.

Primary T cells were activated with CD3/CD28 Dynabeads at 3:1 Bead:Cell ratio overnight, then transduced with lentiviruses derived from the indicated constructs. On day 7 or day 9 (as indicated) of the T cell expansion, CAR expression was measured by flow cytometry with CD19-Fc on cells incubated for 24 hours with 1 μM Bazedoxifene or 1 μM Raloxifene or DMSO. Both on day 7, the percentage of CAR positive cells obtained with constructs OT-001789, OT-001788, OT-001787, and OT-001786 was approximately 20-27.5%, which increased to about 40-50% in the presence of both ligands. At day 9 the basal percentage of CAR positive cells for the same constructs was even lower at approximately 20% i.e. in the absence of ligand and about 32-45% in the presence of either ligand. The percentage of CAR positive cells was observed for OT-001791 was undetectable in the absence of ligand and about 5% in the presence of ligand, suggesting overall very low expression. This was true at both day 7 and day 9. Constructs OT-001792, OT-001784 and OT-001785 did not show any ligand dependent regulation of CAR expression.

### Example 8. ER DD Regulated IL15-IL15Ra

T cells were activated with CD3/CD28 beads for 24 hours, transduced with lentivirus corresponding to OT-001727, using either 2 μl or 10 μl of virus. Cells were then expanded for 6 days. T cells were then treated with 1 μM BZD or DMSO for 24 hours.

Expression of membrane bound IL15-IL15Ra were analyzed by FACS using anti IL15 and anti IL15Ra antibodies and the percentage of cells that were positive for IL15 and IL15Ra cells was calculated (Table 17).

TABLE 17
% IL15-IL15Ra positive cells with ER DD constructs

| | Virus | | 1 μM | Stabilization |
|---|---|---|---|---|
| Construct | Dilution (μl) | DMSO | BZD | Ratio |
| Untransduced | — | 0.02 | | |
| OT-001727 | 10 | 27 | 79 | 3 |
| | 2 | 16 | 65 | 4 |

The percentage of IL15-IL15Ra double positive T cells increased in the presence of BZD treatment for OT-001727 which showed strong ligand dependent expression of IL15-IL15Ra. Cells treated with both virus dilutions showed similar ligand dependent regulation.

### Example 9. Effect of Payload Positioning within Effector Modules

The effect of the position of the payload within the construct on its expression was tested. The effect of placement of furin cleavage site between the DD and the payload was also evaluated.

Human donor T cells were thawed and activated overnight in the presence of CD3/CD28 Dynabeads at 3:1 bead:cell ratio, then transduced with lentivirus related to OT-002158, or OT-001786. Cells were expanded over the course of 10 days, with fresh media added to maintain cells at around 0.5×10\hat{ }6 cells/mL. On day 9, an aliquot of cells was removed, and treated with 1 μM Bazedoxifene overnight or DMSO vehicle control, then stained with 10 μg/mL CD19-Fc. Histograms showing CAR expression from a Live Singlet cell gate were plotted. Data expressed as % CAR+ cells are provided in Table 18.

TABLE 18
% CAR positive cells

| | Vehicle | Bazedoxifene |
|---|---|---|
| OT-002158 | 0.12 | 46.4 |
| OT-001786 | 19.6 | 34.1 |

ER DDs regulated CD19-CAR with extremely low basal expression when placed N-terminal with a furin cleavage site.

Similar experiments were performed using constructs shown in Table 19 and Table 20. Human donor T cells were thawed and activated overnight in the presence of CD3/CD28 Dynabeads at 3:1 bead:cell ratio, then transduced with lentivirus from the constructs. Cells were expanded over the course of 10 days, and fresh media was added to maintain cells around 0.5×10\hat{ }6 cells/ml. On day 9, an aliquot of cells were removed, treated with 1 μM Bazedoxifene overnight, then stained with 10 μg/mL CD19-Fc. Histograms showing CAR expression from a Live Singlet cell gate were plotted. The data are shown in Table 19 and Table 20 where data are presented as % CAR positive cells, where SR indicates stabilization ration.

TABLE 19
% CAR positive cells

| | | | Stabilization |
|---|---|---|---|
| Construct | DMSO | Bazedoxifene | Ratio |
| OT-001789 | 40.2 | 45 | 1.12 |
| OT-001788 | 23 | 35.4 | 1.5 |
| OT-001787 | 17 | 39.4 | 2.3 |
| OT-001786 | 19.6 | 34.1 | 1.7 |
| OT-001785 | 14.1 | 38.7 | 2.7 |
| OT-001784 | 45.6 | 44.8 | 1.0 |
| OT-001790 | 0.3 | 0.6 | 2.0 |

TABLE 20
% CAR positive cells

| | | | Stabilization |
|---|---|---|---|
| Construct | DMSO | Bazedoxifene | Ratio |
| OT-002160 | 0.32 | 27.3 | 85.3 |
| OT-002161 | 0.23 | 27.7 | 120.4 |
| OT-002157 | 0.06 | 41.9 | 698.3 |
| OT-002158 | 0.12 | 46.4 | 386.7 |
| OT-002162 | 0.32 | 22.6 | 70.6 |
| OT-002163 | 0.99 | 20 | 20.2 |
| OT-002164 | 0.3 | 21.8 | 72.7 |
| OT-002159 | 0.53 | 29.3 | 55.3 |

Positioning of the CAR payload at either C or the N terminal resulted in ligand dependent regulation. However, in the absence of ligand (Bazedoxifene), the constructs with payload positioned at the C terminus that were preceded by a furin site (and the DD at the N terminus) showed low expression compared to the constructs with the payload at the N terminal. Accordingly the stabilization ratios obtained with the constructs wherein the payload was positioned at the C terminus were higher (see Table 20) than the constructs where the payload was positioned at N terminus (see Table 19).

### Example 10. In Vitro Efficacy of ER Regulated CARs

T cells were transduced with OT-00179, OT-002158, OT-001407 or empty vector and expanded as described herein, frozen on day 10. To confirm the expression and function of this large scale expansion for in vivo use, cells were thawed and incubated overnight in the presence of CD3/28 beads (1:1 bead:cell ratio) and with or without 1 μM Bazedoxifene. FACS staining with 1 μg/mL CD19-Fc was performed to measure the surface expression of CD19-CAR. The data are provided in Table 21 as the percentage CAR positive cells.

TABLE 21
% CAR positive cells

| Construct | Vehicle | Bazedoxifene |
|---|---|---|
| Empty vector | 0.62 | |
| OT-001407 | 53.8 | 56.2 |
| OT-001792 | 0.39 | 39.6 |
| OT-002158 | 1.87 | 47.8 |

Both OT-00179, and OT-002158 showed surface expression in the 40-50% range in the presence of ligand and less than 5% expression in the absence of ligand. As expected, the empty vector did not show any CAR expression and the positive control, OT-001407 showed expression both in the presence and absence of Bazedoxifene.

To measure Interferon gamma production, T cells transduced with the constructs OT-00179, OT-002158, OT-001407 or empty vector were thawed and co-cultured with Nalm6 target cells (stably expressing NucRed®) at different ratios of transduced T cells (herein referred to as the Effector) and Nalm 6 cells (herein referred to as the Target). Target cell viability was determined by measuring cellular fluorescence over time using the Incucyte instrument. Data presented in Table 22 are expressed as Nalm6 cell area at representative time points (0, 24, and 42 hour (hr)). The lower the numerical value of the Nalm6 area, the higher the cytotoxicity effected by the Effector cells.

TABLE 22
Nalm 6 area

| | Time | Total target area | | | |
|---|---|---|---|---|---|
| Description | (hr) | 10:1 | 3:1 | 1:1 | 0.3:1 |
| Empty Vector | 0 | 52554.46 | 59390.65 | 68615.72 | 67604.36 |
| OT-001407 | 0 | 56558.99 | 60570.95 | 60970.58 | 70869.90 |
| OT-001792 | 0 | 57172.20 | 60708.63 | 62673.31 | 71711.59 |
| OT-002158 | 0 | 57551.74 | 61283.89 | 64550.92 | 69377.03 |
| Empty Vector + | 0 | 50387.36 | 53224.98 | 54420.91 | 60160.90 |
| 1 μM | | | | | |
| Bazedoxifene | | | | | |
| OT-001407 + | 0 | 60283.69 | 57968.49 | 58651.67 | 53764.52 |
| 1 μM | | | | | |
| Bazedoxifene | | | | | |
| OT-001792 + | 0 | 55572.92 | 54788.54 | 67768.82 | 57617.98 |
| 1 μM | | | | | |
| Bazedoxifene | | | | | |
| OT-002158 + | 0 | 55604.92 | 54040.62 | 61210.96 | 53330.66 |
| 1 μM | | | | | |
| Bazedoxifene | | | | | |
| Empty Vector | 24 | 91743.88 | 100280.58 | 117103.90 | 117219.25 |
| OT-001407 | 24 | 37404.09 | 68281.58 | 91200.62 | 117301.10 |
| OT-001792 | 24 | 93991.37 | 105474.34 | 104433.20 | 123175.80 |
| OT-002158 | 24 | 91854.77 | 105491.45 | 112198.15 | 120391.75 |
| Empty Vector + | 24 | 82879.75 | 88853.42 | 91512.43 | 100327.45 |
| 1 μM | | | | | |
| Bazedoxifene | | | | | |
| OT-001407 + | 24 | 47568.34 | 65988.71 | 81409.21 | 83498.15 |
| 1 μM | | | | | |
| Bazedoxifene | | | | | |
| OT-001792 + | 24 | 40606.37 | 67794.87 | 103535.70 | 94217.60 |
| 1 μM | | | | | |
| Bazedoxifene | | | | | |
| OT-002158 + | 24 | 44020.75 | 70051.27 | 90702.75 | 90149.08 |
| 1 μM | | | | | |
| Bazedoxifene | | | | | |
| Empty Vector | 42 | 156703.40 | 172065.10 | 199013.95 | 192491.80 |
| OT-001407 | 42 | 23864.92 | 57624.68 | 103493.26 | 168381.30 |
| OT-001792 | 42 | 156073.75 | 180465.55 | 171919.20 | 197082.75 |
| OT-002158 | 42 | 146938.00 | 174785.15 | 182443.65 | 195959.75 |
| Empty Vector + | 42 | 138796.45 | 147287.05 | 153257.00 | 159070.65 |
| 1 μM | | | | | |
| Bazedoxifene | | | | | |
| OT-001407 + | 42 | 30151.15 | 56384.84 | 101357.43 | 132305.55 |
| 1 μM | | | | | |
| Bazedoxifene | | | | | |
| OT-001792 + | 42 | 25302.71 | 58548.97 | 133539.50 | 143903.95 |
| 1 μM | | | | | |
| Bazedoxifene | | | | | |
| OT-002158 + | 42 | 26321.51 | 65046.55 | 119282.15 | 135259.35 |
| 1 μM | | | | | |
| Bazedoxifene | | | | | |

Cytotoxic effects were observed only in the presence of ligand at 3:1 and 10:1 Effector:Target ratios.

Cell culture supernatants were collected from the T cell/Nalm6 cytotoxicity assay co-cultures at 42 hours. Interferon gamma was measured by MSD and is described in Table 23.

TABLE 23
IFN gamma levels (pg/ml)

| | | 1 μM |
|---|---|---|
| Construct | Vehicle | Bazedoxifene |
| Empty vector | 16.36 | 10.42 |
| OT-001407 | 17586.07 | 15316.10 |
| OT-001792 | 91.09 | 7079.60 |
| OT-002158 | 197.70 | 21905.91 |

IFNg was observed only in the presence of ligand with ER regulated constructs, which correlated with the results obtained with the cytotoxicity assay.

### Example 11. Pharmacokinetics of Bazedoxifene

CD-1 mice were injected with a single dose of Bazedoxifene at 10, 100, 200 mg/kg by individual body weights and the plasma Bazedoxifene concentrations were measured over several hours by LC-MS-MS. The vehicle used was (10% DMSO; 90% (20% 2-Hydroxypropyl-β-cyclodextrin)). The plasma concentrations of free and bound Bazedoxifene combined are shown in Table 24.

TABLE 24
Plasma Bazedoxifene Concentrations

| Hours | | | | | | | | | |
|---|---|---|---|---|---|---|---|---|---|
| post | Bazedoxifene | | | Bazedoxifene | | | Bazedoxifene | | |
| dose | 10 mg/kg | | | 100 mg/kg | | | 200 mg/kg | | |
| 0.25 | 0.062 | 0.127 | 0.287 | 1.268 | 0.949 | 2.590 | 1.064 | 1.495 | 1.013 |
| 0.5 | 0.071 | 0.050 | 0.091 | 2.113 | 2.208 | 4.544 | 1.507 | 2.314 | 1.707 |
| 1 | 0.097 | 0.089 | 0.105 | 3.588 | 2.590 | 5.138 | 2.399 | 3.397 | 4.013 |
| 2 | 0.077 | 0.096 | 0.107 | 2.824 | 2.272 | 4.161 | 3.270 | 2.590 | 3.567 |
| 4 | 0.049 | 0.053 | 0.055 | 1.384 | 1.208 | 1.902 | 1.577 | 1.327 | 2.357 |
| 7 | 0.034 | 0.036 | 0.035 | 0.894 | 0.463 | 0.970 | 0.866 | 0.726 | 1.189 |
| 10 | 0.033 | 0.028 | 0.016 | 0.754 | 0.497 | 0.713 | 0.699 | 0.732 | 0.715 |
| 24 | 0.005 | 0.003 | 0.004 | 0.023 | 0.179 | 0.025 | 0.297 | 0.314 | 0.180 |

Murine Cmax value for Bazedoxifene (˜3 μM) is achieved at a dose of 200 mg/kg per os (PO) and human Cmax for Bazedoxifene (˜0.1 μM) value is known to be 3 mg intravenous (IV).

### Example 12. In Vivo Efficacy of ER Regulated CARs

Nalm6 cells were transfected with Redifect Red-FlucPuro (Perkin Elmer) under selection using 2 μg/ml puromycin to generate a line that stably expressed the luciferase reporter; thereafter named Nalm6-Luc. Ten days before tumor implantation, Nalm6-Luc cells were thawed and cultured in 1 μg/ml puromycin-containing media. On day 0, cells were counted, resuspended in PBS and 1 million cells were injected into 6-8-week-old female NSG mice via tail vein. On day 6, mice were imaged for bioluminescent intensity (BLI) and sorted into groups based on their tumor burden, determined by BLI signal intensity, to ensure an even distribution across dosing groups. Animals were administered a preloading dose of vehicle or ligand after randomization into their assigned groups. T cells activated with CD3/CD28 Dynabeads, transduced with lentiviral vectors carrying the constructs OT-00179, OT-002158, OT-001407 and expanded for 10 days then frozen. On day 7 post tumor implant, mice were administered another preloading dose of vehicle or ligand, then T cells were thawed and injected via tail vein into the mice at 5 million CAR+ cells per mouse. Animals were orally dosed once a day for 25 days with the one of the following doses of Bazedoxifene: 50, 100 or 200 mg/kg body weight. Body weight and tumor measurements, performed using bioluminescent imaging, were collected at 6, 14, 21, 24, 27, and 31 days post implant. The total flux (photons/second) was measured and is shown in Table 25. In Table 25, BZA indicates Bazedoxifene, QD indicates quaque die (or daily dosing).

TABLE 25
Total Flux (photons/second)

| | Days post tumor implant | | | |
|---|---|---|---|---|
| Construct | 6 | 14 | 21 | 24 |
| Empty Vector | 1.39E+06 | 5.28E+07 | 1.83E+09 | 2.88E+09 |
| Vehicle | 1.07E+06 | 3.13E+07 | 1.14E+09 | 3.25E+09 |
| | 2.00E+06 | 2.94E+07 | 1.71E+09 | 4.65E+09 |
| | 1.39E+06 | 4.52E+07 | 1.95E+09 | 4.38E+09 |
| | 2.04E+06 | 4.01E+07 | 2.03E+09 | 6.08E+09 |
| | 2.53E+06 | 4.98E+08 | — | — |
| | 1.73E+06 | 5.40E+07 | 1.99E+09 | 7.40E+09 |
| | 1.72E+06 | 7.92E+07 | 2.52E+09 | 5.77E+09 |
| OT-001407- | 1.07E+06 | 1.43E+06 | 4.25E+05 | 1.57E+06 |
| Vehicle (BZA) | 2.48E+06 | 1.57E+07 | 1.05E+06 | 2.48E+08 |
| | 1.72E+06 | 1.48E+06 | 1.34E+06 | 2.20E+06 |
| | 1.40E+06 | 2.69E+06 | 3.11E+06 | 2.09E+06 |
| | 1.73E+06 | 3.57E+05 | 1.29E+06 | 3.52E+06 |
| | 1.97E+06 | 2.75E+06 | 9.84E+05 | 1.66E+06 |
| | 1.37E+06 | 2.11E+06 | 1.08E+06 | 2.05E+06 |
| | 2.04E+06 | 2.23E+06 | 1.59E+06 | 3.48E+06 |
| OT-001407- | 2.40E+06 | 7.79E+05 | 8.59E+05 | 9.27E+05 |
| Vehicle | 1.69E+06 | 7.84E+05 | 7.27E+05 | 6.98E+05 |
| | 1.96E+06 | 7.92E+05 | 7.78E+05 | 9.68E+05 |
| | 1.75E+06 | 4.53E+05 | 6.89E+05 | 8.41E+05 |
| | 2.05E+06 | 7.56E+05 | 7.20E+05 | 9.95E+05 |
| | 1.08E+06 | 7.74E+05 | 6.84E+05 | 9.21E+05 |
| | 1.42E+06 | 6.28E+05 | 8.74E+05 | 7.86E+05 |
| | 1.35E+06 | 7.19E+05 | 7.97E+05 | 1.03E+06 |
| OT-001407- | 1.76E+06 | — | — | — |
| BZA 200 | 1.34E+06 | 1.18E+06 | 8.31E+05 | 5.98E+05 |
| mg/kg QD | 1.69E+06 | 8.51E+05 | 8.46E+05 | 1.42E+06 |
| | 2.40E+06 | 5.16E+05 | 6.93E+05 | 1.35E+06 |
| | 1.93E+06 | 1.21E+06 | 1.28E+06 | 2.24E+06 |
| | 1.42E+06 | 1.07E+06 | 9.35E+05 | 2.03E+06 |
| | 1.11E+06 | 1.07E+06 | 9.87E+05 | 1.52E+06 |
| | 2.07E+06 | 1.17E+06 | 9.04E+05 | 1.26E+06 |
| OT-00179 - | 2.10E+06 | 1.12E+08 | 1.82E+09 | 4.09E+09 |
| Vehicle | 1.32E+06 | 1.28E+08 | 2.64E+07 | 4.49E+07 |
| | 1.17E+06 | 7.67E+07 | 1.45E+08 | 5.61E+09 |
| | 1.78E+06 | 7.18E+07 | 7.93E+06 | 4.90E+09 |
| | 1.50E+06 | 4.32E+07 | 1.31E+09 | 3.75E+09 |
| | 1.66E+06 | 1.20E+08 | 9.32E+08 | 3.92E+09 |
| | 1.88E+06 | — | — | — |
| | 2.34E+06 | 1.60E+06 | 1.66E+09 | 8.84E+08 |
| OT-00179 - | 1.79E+06 | 2.23E+06 | 9.82E+06 | 4.17E+07 |
| BZA 200 | 2.10E+06 | 8.21E+05 | 9.99E+05 | 1.31E+06 |
| mg/kg QD | 2.31E+06 | 4.85E+05 | 2.91E+06 | 1.11E+07 |
| | 1.88E+06 | 5.13E+05 | 7.92E+06 | 3.52E+07 |
| | 1.65E+06 | 1.33E+06 | 1.39E+06 | 5.03E+05 |
| | 1.51E+06 | 1.26E+06 | 4.05E+06 | 6.55E+05 |
| | 1.31E+06 | 1.41E+06 | 3.64E+06 | 1.23E+07 |
| | 1.18E+06 | 1.60E+06 | 2.08E+06 | 8.73E+06 |
| | 1.63E+06 | 1.14E+08 | 1.04E+08 | 6.11E+09 |
| OT-002158- | 2.11E+06 | 1.83E+08 | 2.40E+09 | 4.82E+09 |
| Vehicle (BZA) | 1.53E+06 | 8.65E+07 | 2.13E+09 | 4.26E+09 |
| | 1.30E+06 | 7.39E+07 | 1.50E+09 | 2.72E+09 |
| | 1.18E+06 | 8.01E+07 | 1.47E+09 | 3.54E+09 |
| | 2.25E+06 | 2.13E+08 | 2.39E+09 | 9.56E+09 |
| | 1.79E+06 | 7.48E+07 | 1.36E+09 | 3.95E+09 |
| | 1.87E+06 | 7.44E+07 | 1.77E+09 | 4.34E+09 |
| OT-002158 - | 1.22E+06 | — | — | — |
| BZA 200 | 1.54E+06 | — | — | — |
| mg/kg QD | 2.12E+06 | 1.24E+06 | 5.08E+05 | 4.77E+06 |
| | 1.60E+06 | 2.01E+06 | 1.33E+07 | 4.26E+07 |
| | 1.81E+06 | 1.64E+06 | 6.47E+06 | 4.75E+07 |
| | 1.30E+06 | 1.22E+06 | 5.13E+05 | 1.76E+06 |
| | 2.21E+06 | 1.62E+06 | 3.21E+06 | 1.32E+07 |
| | 1.86E+06 | 1.72E+06 | 9.03E+06 | 2.80E+07 |
| OT-002158 - | 2.20E+06 | 1.21E+07 | 4.85E+06 | 5.13E+07 |
| BZA 100 | 1.22E+06 | 1.58E+06 | 4.83E+05 | 1.59E+06 |
| mg/kg QD | 1.55E+06 | 1.56E+06 | 3.55E+06 | 7.62E+05 |
| | 1.26E+06 | 8.65E+05 | 1.91E+06 | 6.28E+05 |
| | 1.81E+06 | 6.67E+05 | 5.72E+05 | 7.21E+05 |
| | 1.84E+06 | 2.03E+06 | 7.24E+06 | 1.97E+07 |
| | 1.59E+06 | 1.78E+06 | 3.39E+07 | 8.83E+05 |
| | 2.13E+06 | 2.25E+06 | 2.27E+06 | 1.09E+07 |
| OT-002158 - | 2.20E+06 | 2.79E+07 | 9.67E+07 | 5.51E+08 |
| BZA 50 | 1.24E+06 | — | — | — |
| mg/kg QD | 1.56E+06 | 8.12E+06 | 6.20E+06 | 4.40E+07 |
| | 1.83E+06 | 1.27E+07 | 6.62E+05 | 1.42E+06 |
| | 1.82E+06 | 4.78E+06 | 8.05E+05 | 1.96E+08 |
| | 1.58E+06 | 6.94E+06 | — | — |
| | 2.15E+06 | 5.69E+07 | 2.16E+08 | 3.81E+06 |
| | 1.23E+06 | 6.34E+05 | 9.93E+05 | 3.67E+07 |

Mice infused with OT-00179, and OT-002158 expressing T cells demonstrated a reduction in total flux in the cohorts treated with Bazedoxifene compared to vehicle control suggesting a decrease in tumor burden. The extent of reduction in tumor burden correlated with the dose of Bazedoxifene administered to the mice. Mice infused with OT-001407 construct showed similar tumor burden in both the vehicle control and the Bazedoxifene cohorts indicating that the expression and function of the chimeric antigen receptors are not affected by the ligand in the absence of the ER domains.

Nalm6 cells were transfected with Redifect Red-FlucPuro (Perkin Elmer) under selection using 2 μg/ml puromycin to generate a line that stably expressed the luciferase reporter; thereafter named Nalm6-Luc. Ten days before tumor implantation, Nalm6-Luc cells were thawed and cultured in 1 μg/ml puromycin-containing media. On day 0, cells were counted, resuspended in PBS and 1 million cells were injected into 6-8 week-old female NSG mice via tail vein. On day 6, mice were imaged for bioluminescent intensity (BLI) and sorted into groups based on their tumor burden, determined by BLI signal intensity, to ensure an even distribution across dosing groups. Animals were administered a preloading dose of vehicle or ligand after randomization into their assigned groups. T cells activated with CD3/CD28 Dynabeads, transduced with lentiviral vectors carrying the constructs OT-002158, OT-001407 and expanded for 10 days then frozen. On day 7 post tumor implant, mice were administered another preloading dose of vehicle or ligand, then T cells were thawed and injected via tail vein into the mice at 5 million CAR+ cells per mouse. Animals were orally dosed once a day with vehicle or Bazedoxifene 200 mg/kg body weight. Body weight and tumor measurements, performed using bioluminescent imaging, were collected at 6, 13, 19 and 22 days post implant. The total flux (photons/second) was measured and is shown in Table 26. In Table 26, BZA indicates Bazedoxifene, QD indicates quaque die (or daily dosing). Blood was collected from animals at 14 and 21 days post tumor implant and analyzed by flow cytometry for total T cells (Table 27) and frequency of CAR expression (Table 28).

TABLE 26
Total Flux (photons/second)

| | Days post implant | | | |
|---|---|---|---|---|
| Construct | 6 | 13 | 19 | 22 |
| Empty Vector | 2.45E+06 | 1.54E+08 | 2.01E+09 | 4.14E+09 |
| | 3.25E+06 | 1.71E+08 | 1.81E+09 | 3.77E+09 |
| | 4.86E+06 | 3.04E+08 | 1.36E+07 | 3.40E+07 |
| | 3.92E+06 | 2.18E+08 | 1.24E+07 | 4.23E+09 |
| OT-001407 - | 2.48E+06 | 9.14E+05 | 1.04E+06 | 9.00E+05 |
| Vehicle | 5.82E+06 | 8.41E+05 | 9.21E+05 | 6.18E+05 |
| | 4.06E+06 | 7.19E+05 | 8.89E+05 | 7.10E+05 |
| | 3.04E+06 | 1.18E+06 | 4.31E+06 | 5.31E+05 |
| OT-002158 - | 6.01E+06 | 5.15E+08 | 4.38E+09 | 7.63E+09 |
| Vehicle | 2.78E+06 | 1.66E+08 | 2.76E+09 | 4.15E+09 |
| | 2.09E+06 | 2.77E+08 | 2.98E+08 | 3.20E+09 |
| | 3.48E+06 | 1.94E+08 | 3.11E+09 | 4.73E+09 |
| OT-002158 - | 6.05E+06 | 6.77E+05 | 5.68E+05 | 1.11E+06 |
| BZA 200 | 2.06E+06 | 9.95E+05 | 9.14E+05 | 7.18E+05 |
| mg/kg QD | 3.42E+06 | 8.10E+05 | 1.13E+06 | 9.91E+05 |
| | 2.82E+06 | 1.03E+06 | 1.21E+06 | 1.14E+06 |

TABLE 27
Total T cells

| | Day 14 | | | | Day 21 | | | |
|---|---|---|---|---|---|---|---|---|
| | Animal | Animal | Animal | Animal | Animal | Animal | Animal | Animal |
| Construct | 1 | 2 | 3 | 4 | 1 | 2 | 3 | 4 |
| Empty vector | 38 | 38 | 89 | 28 | 20 | 16 | 27 | 8 |
| OT-001407 | 355 | | 276 | 224 | 364 | 171 | 81 | 108 |
| OT-002158 - | 130 | 162 | 216 | 109 | 60 | 82 | 59 | 92 |
| Vehicle | | | | | | | | |
| OT-002158 - | 847 | 737 | 492 | 625 | 317 | 1674 | 159 | 323 |
| BZA | | | | | | | | |

TABLE 28
% CAR positive cells

| Construct | Day 14 | | | | Day 21 | | | |
|---|---|---|---|---|---|---|---|---|
| Empty vector | 0 | 1 | 1 | 0 | 18 | 10 | 12 | 7 |
| OT-001407 | 140 | | 131 | 86 | 276 | 113 | 52 | 70 |
| OT-002158 - | 1 | 1 | 1 | 2 | 42 | 76 | 46 | 68 |
| Vehicle | | | | | | | | |
| OT-002158 - | 278 | 240 | 109 | 168 | 71 | 1286 | 137 | 78 |
| BZA | | | | | | | | |

Mice infused with OT-002158 expressing T cells demonstrated an increase in both total T cell and CAR+ cell counts in the cohorts treated with Bazedoxifene compared to vehicle control suggesting a CAR mediated cell expansion that correlates with a decrease in tumor burden. Mice infused with OT-001407 construct, but not vector control, showed similar increases in T cell and CAR+ cell counts, along with decreased tumor burden.

While the present disclosure has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the disclosure.

All publications, patent applications, patents, and other references mentioned herein are incorporated by reference in their entirety. In case of conflict, the present specification, including definitions, will control. In addition, section headings, the materials, methods, and examples are illustrative only and not intended to be limiting.

1. A composition comprising at least one effector module, said at least one effector module comprising
(a) a stimulus response element (SRE) and
(b) at least one payload, said at least one payload comprising a protein of interest which is attached, appended or associated with said SRE,
wherein said SRE comprises a destabilizing domain (DD), said DD comprising, in whole or in part, the human estrogen receptor (ER; SEQ ID NO. 1).

2. The composition of claim 1, wherein the DD comprises a ligand binding domain of ER (SEQ ID NO. 1), said ligand binding domain comprising amino acids 305 to 509 of ER (SEQ ID NO. 2).

3. The composition of claim 2, wherein the DD comprises at least one mutation, said at least one mutation occurring at position 413 (N413) relative to SEQ ID NO. 1.

4. The composition of claim 3, wherein said mutation is selected from the group consisting of N413D, N413T, N413H, N413A, N413Q, N413V, N413C, N413K, N413M, N413R, N413S, N413W, N413I, N413E, N413L, N413P, N413F, N413Y, and N413G.

5. The composition of claim 4, wherein the mutation is N413D.

6. The composition of claim 4, wherein the mutation is N413T.

7. The composition of claim 2, wherein the DD comprises at least one mutation, said at least one mutation occurring at position 502 (Q502), relative to SEQ ID NO. 1.

8. The composition of claim 7, wherein said mutation is selected from the group consisting of Q502H, Q502D, Q502E, Q502V, Q502A, Q502T, Q502N, Q502K, Q5025, Q502L, Q502Y, Q502W, Q502F, Q502I, Q502G, Q502P, Q502M, and Q502C.

9. The composition of claim 8, wherein the mutation is Q502R.

10. The composition of claim 2, wherein the DD comprises at least two mutations, said at least two mutations occurring at position 413 (N413) and at position 502 (Q502), relative to SEQ ID NO. 1.

11. The composition of claim 10, wherein the mutation occurring at position 413 (N413) is selected from the group consisting of N413D, N413T, N413H, N413A, N413Q, N413V, N413C, N413K, N413M, N413R, N413S, N413W, N413I, N413E, N413L, N413P, N413F, N413Y, and N413G, and wherein the mutation occurring at position at position 502 (Q502) is selected from the group consisting of Q502H, Q502D, Q502E, Q502V, Q502A, Q502T, Q502N, Q502K, Q5025, Q502L, Q502Y, Q502W, Q502F, Q502I, Q502G, Q502P, Q502M, and Q502C.

12. The composition of claim 11, wherein the at least two mutations are N413D and Q502H.

13. The composition of claim 11, wherein the at least two mutations are N413T and Q502H.

14. The composition of any of claims 3-13, wherein the DD further comprises one or more mutations independently selected from L384M, M421G, G521R, or Y537S.

15. The composition of claim 14, wherein the DD is selected from the group consisting of ER (aa 305-549 of WT, L384M, N413F, M421G, G521R, Y537S) (SEQ ID NO. 27), ER (aa 305-549 of WT, L384M, N413L, M421G, G521R, Y537S) (SEQ ID NO. 29), ER (aa 305-549 of WT, L384M, N413Y, M421G, G521R, Y537S) (SEQ ID NO. 31), ER (aa 305-549 of WT, L384M, N413H, M421G, G521R, Y537S) (SEQ ID NO. 33), ER (aa 305-549 of WT, L384M, N413Q, M421G, G521R, Y537S) (SEQ ID NO. 35), ER (aa 305-549 of WT, L384M, N413I, M421G, G521R, Y537S) (SEQ ID NO. 37), ER (aa 305-549 of WT, L384M, N413M, M421G, G521R, Y537S) (SEQ ID NO. 39), ER (aa 305-549 of WT, L384M, N413K, M421G, G521R, Y537S) (SEQ ID NO. 41), ER (aa 305-549 of WT, L384M, N413V, M421G, G521R, Y537S) (SEQ ID NO. 43), ER (aa 305-549 of WT, L384M, N413S, M421G, G521R, Y537S) (SEQ ID NO. 45), ER (aa 305-549 of WT, L384M, N413C, M421G, G521R, Y537S) (SEQ ID NO. 47), ER (aa 305-549 of WT, L384M, N413W, M421G, G521R, Y537S) (SEQ ID NO. 49), ER (aa 305-549 of WT, L384M, N413P, M421G, G521R, Y537S) (SEQ ID NO. 51), ER (aa 305-549 of WT, L384M, N413R, M421G, G521R, Y537S) (SEQ ID NO. 53), ER (aa 305-549 of WT, L384M, N413T, M421G, G521R, Y537S) (SEQ ID NO. 55), ER (aa 305-549 of WT, L384M, N413A, M421G, G521R, Y537S) (SEQ ID NO. 57), ER (aa 305-549 of WT, L384M, N413E, M421G, G521R, Y537S) (SEQ ID NO. 59), ER (aa 305-549 of WT, L384M, N413G, M421G, G521R, Y537S) (SEQ ID NO. 61), ER (aa 305-549 of WT, L384M, M421G, Q502F, G521R, Y537S) (SEQ ID NO. 63), ER (aa 305-549 of WT, L384M, M421G, Q502L, G521R, Y537S) (SEQ ID NO. 65), ER (aa 305-549 of WT, L384M, M421G, Q502Y, G521R, Y537S) (SEQ ID NO. 67), ER (aa 305-549 of WT, L384M, M421G, Q502H, G521R, Y537S) (SEQ ID NO. 69), ER (aa 305-549 of WT, L384M, M421G, Q502I, G521R, Y537S) (SEQ ID NO. 71), ER (aa 305-549 of WT, L384M, M421G, Q502M, G521R, Y537S) (SEQ ID NO. 73), ER (aa 305-549 of WT, L384M, M421G, Q502N, G521R, Y537S) (SEQ ID NO. 75), ER (aa 305-549 of WT, L384M, M421G, Q502K, G521R, Y537S) (SEQ ID NO. 77), ER (aa 305-549 of WT, L384M, M421G, Q502V, G521R, Y537S) (SEQ ID NO. 79), ER (aa 305-549 of WT, L384M, M421G, Q5025, G521R, Y537S) (SEQ ID NO. 81), ER (aa 305-549 of WT, L384M, M421G, Q502C, G521R, Y537S) (SEQ ID NO. 83), ER (aa 305-549 of WT, L384M, M421G, Q502W, G521R, Y537S) (SEQ ID NO. 85), ER (aa 305-549 of WT, L384M, M421G, Q502P, G521R, Y537S) (SEQ ID NO. 87), ER (aa 305-549 of WT, L384M, M421G, Q502T, G521R, Y537S) (SEQ ID NO. 89), ER (aa 305-549 of WT, L384M, M421G, Q502A, G521R, Y537S) (SEQ ID NO. 91), ER (aa 305-549 of WT, L384M, M421G, Q502D, G521R, Y537S) (SEQ ID NO. 93), ER (aa 305-549 of WT, L384M, M421G, Q502E, G521R, Y537S) (SEQ ID NO. 95), and ER (aa 305-549 of WT, L384M, M421G, Q502G, G521R, Y537S) (SEQ ID NO. 97).

16. The composition of claim 1, wherein the at least one payload is a natural protein or a variant thereof or a fusion polypeptide, or an antibody or a fragment thereof or a therapeutic agent or a gene therapy.

17. The composition of claim 16, wherein the payload is therapeutic agent, said therapeutic agent is selected from a cytokine, a cytokine-cytokine receptor fusion protein, a chimeric antigen receptor (CAR) or a combination thereof.

18. The composition of claim 17, wherein the payload is a cytokine, said cytokine comprising IL15.

19. The composition of claim 18, wherein the IL15 comprises amino acid 49-162 of wild-type (SEQ ID NO. 246).

20. The composition of claim 17, wherein the payload is a cytokine-cytokine receptor fusion, said cytokine-cytokine receptor fusion comprises IL15 appended to an IL15 Receptor alpha subunit (IL15Ra), and where the IL15 comprises amino acid 49-162 of wild-type (SEQ ID NO. 246) and the IL15Ra comprises amino acid 31-267 of wild-type (SEQ ID NO. 248).

21. The composition of claim 17, wherein the payload is a cytokine, said cytokine comprising IL12.

22. The composition of claim 21, wherein the IL12 comprises a p40 subunit of SEQ ID NO. 111, appended to a p35 subunit of SEQ ID NO. 113.

23. The composition of claim 22, wherein the effector module comprises the amino acid sequence selected from the group consisting of SEQ ID NO. 232, 208, 158-217, 219-231, and SEQ ID NO. 233-242.

24. The composition of claim 23, wherein the effector module comprises the amino acid sequence of SEQ ID NO. 232.

25. The composition of claim 23, wherein the effector module comprises the amino acid sequence of SEQ ID NO. 208.

26. The composition of claim 16, wherein the payload is a chimeric antigen receptor (CAR).

27. The composition of claim 26, wherein the CAR comprises a CD19 scFv.

28. The composition of claim 27, wherein the CD19 scFv amino acid sequence is selected from the group consisting of SEQ ID NO. 103 and SEQ ID NO. 368.

29. The composition of claim 27, further comprising at least one domain selected from the group consisting of CD8 hinge and transmembrane domain, 4-1BB intracellular signaling domain and CD8 zeta signaling domain.

30. The composition of claim 29, wherein the domain is CD8 hinge and transmembrane domain, and wherein the CD8 hinge and transmembrane domain comprises the amino acid sequence of SEQ ID NO. 105.

31. The composition of claim 29, wherein the domain is 4-1BB intracellular signaling domain, and wherein the 4-1BB intracellular signaling domain comprises the amino acid sequence of SEQ ID NO. 107.

32. The composition of claim 29, wherein the domain is CD8 zeta signaling domain, and wherein the CD8 zeta signaling domain comprises the amino acid sequence of SEQ ID NO. 109.

33. The composition of claim 17, wherein the payload is a cytokine and a CAR.

34. The composition of claim 33, wherein the CAR comprises a CD19 scFv and wherein the cytokine is IL12.

35. The composition of claim 1, wherein the SRE is responsive to one or more stimuli.

36. The composition of claim 35, wherein the stimulus is a small molecule, wherein the small molecule is selected from bazedoxifene, raloxifene 4-hydroxytamoxifen (4-OHT), fulvestrant, oremifene, lasofoxifene, clomifene, femarelle and ormeloxifene.

37. The composition of claim 36, wherein the small molecule is bazedoxifene.

38. The composition of claim 36, wherein the small molecule is raloxifene.

39. A composition comprising at least one effector module, said at least one effector module comprising
(a) a stimulus response element (SRE) and
(b) at least one payload, said at least one payload, said payload comprising a chimeric antigen receptor, a cytokine or a cytokine-cytokine receptor fusion protein, wherein said SRE comprises a destabilizing domain (DD), said DD comprising,
(i) a region of human estrogen receptor (ER) of SEQ ID NO. 1; and
(ii) one or more mutations selected from T371A, N5195, K303R, N3045, 5305N, R335G, T431I, E523G, A546T, and G442V relative to SEQ ID NO. 1.

40. The composition of claim 39, wherein the DD further comprises one or more mutations independently selected from the group consisting of L384M, M421G, G521R, or Y537S.

41. The composition of any of claim 39 or 40, wherein the DD is ER (aa 305-549 of WT, L384M, N413D, M421G, G521R, Y537S) (SEQ ID NO. 19), ER (aa 305-549 of WT, L384M, N413T, M421G, G521R, Y537S) (SEQ ID NO. 55), ER (aa 305-549 of WT, L384M, M421G, Q502H, G521R, Y537S) (SEQ ID NO. 69), ER (aa 305-549 of WT, T371A, L384M, M421G, N5195, G521R, Y537S) (SEQ ID NO. 8), ER (aa 303-549 of WT, K303R, N3045, T371A, L384M, M421G, N5195, G521R, Y537S) (SEQ ID NO. 12), ER (aa 305-549 of WT, R335G, L384M, M421G, N5195, G521R, Y537S) (SEQ ID NO. 13), ER (aa 305-549 of WT, R335G, L384M, M421G, G521R, E523G, Y537S, A546T) (SEQ ID NO. 15), ER (aa 305-549 of WT, L384M, M421G, T431I, G521R, Y537S) (SEQ ID NO. 17), ER (aa 305-549 of WT, L384M, M421G, N5195, G521R, Y537S) (SEQ ID NO. 21), ER (aa 305-549 of WT, L384M, M421G, Q502R, G521R, Y537S) (SEQ ID NO. 23), ER (aa 305-549 of WT, 5305N, L384M, M421G, G442V, G521R, Y537S) (SEQ ID NO. 25), ER (aa 305-549 of WT, L384M, M421G, G521R, Y537S) (SEQ ID NO. 4).

42. The composition of claim 41, wherein the DD is ER (aa 305-549 of WT, L384M, N413D, M421G, G521R, Y537S) (SEQ ID NO. 19).

43. The composition of claim 41, wherein the DD is ER (aa 305-549 of WT, L384M, N413T, M421G, G521R, Y537S) (SEQ ID NO. 55).

44. The composition of claim 41, wherein the DD is ER (aa 305-549 of WT, L384M, M421G, Q502H, G521R, Y537S) (SEQ ID NO. 69).

45. The composition of claim 41, wherein the DD is ER (aa 305-549 of WT, T371A, L384M, M421G, N519S, G521R, Y537S) (SEQ ID NO. 8).

46. The composition of claim 39, wherein the payload is a chimeric antigen receptor, and said chimeric antigen receptor comprises
(a) an extracellular target moiety;
(b) a hinge and transmembrane domain;
(c) an intracellular signaling domain; and
(d) optionally, one or more co-stimulatory domains.

47. The composition of claim 46, wherein the extracellular target of the CAR moiety has an affinity or binds to a target molecule on the surface of a cancer cell.

48. The composition of claim 47, wherein the extracellular target moiety of the CAR is an scFv.

49. The composition of claim 47, wherein the target molecule is CD19.

50. The composition of claim 48 or claim 49, wherein the extracellular target moiety of the CAR is a CD19 scFv (SEQ ID NO. 103; or SEQ ID NO. 368).

51. The composition of claim 46, wherein the hinge and transmembrane domain of the CAR are paired and wherein the paired hinge and transmembrane domain is derived from any of the members of the group consisting of:
(a) a molecule selected from CD8a, CD5, CD4, CD9, CD16, CD22, CD33, CD28, CD37, CD45, CD64, CD80, CD86, CD148, DAP 10, EpoRI, GITR, LAG3, ICOS, Her2, OX40 (CD134), 4-1BB (CD137), CD152, CD154, PD-1, or CTLA-4;
(b) the CD3 epsilon chain of a T-cell receptor;
(c) a transmembrane region of an alpha, beta or zeta chain of a T-cell receptor; and
(d) an immunoglobulin selected from IgG1, IgD, IgG4, and an IgG4 Fc region.

52. The composition of claim 51, wherein the paired hinge and transmembrane domain of the CAR is derived from CD8a and said paired hinge and transmembrane domain comprises the amino acid sequence of SEQ ID NO. 105.

53. The composition of claim 46, wherein
(a) the intracellular signaling domain of the CAR is the signaling domain derived from CD3zeta or a cell surface molecule selected from the group consisting of FcR gamma, FcR beta, CD3 gamma, CD3 delta, CD3 epsilon, CD5, CD22, CD79a, CD79b, and CD66d; and
(b) the co-stimulatory domain is present and is derived from the group consisting of 4-1BB (CD137) 2B4, HVEM, ICOS, LAG3, DAP10, DAP12, CD27, CD28, OX40 (CD134), CD30, CD40, ICOS (CD278), glucocorticoid-induced tumor necrosis factor receptor (GITR), lymphocyte function-associated antigen-1 (LFA-1), CD2, CD7, LIGHT, NKG2C, and B7-H3.

54. The composition of claim 46, wherein the intracellular signaling domain of the CAR is derived from CD3 zeta and said intracellular signaling domain comprises the amino acid sequence of SEQ ID NO. 109.

55. The composition of claim 46, wherein the co-stimulatory domain of the CAR is present, said co-stimulatory domain being derived from 4-1BB, wherein the costimulatory domain comprises the amino acid sequence of SEQ ID NO. 107.

56. The composition of claim 46, wherein the chimeric antigen receptor further comprises a signal sequence.

57. The composition of claim 56, wherein the signal sequence is derived from CD8a and wherein the signal sequence comprises the amino acid sequence of SEQ ID NO. 99.

58. The composition of any of claims 46-57, wherein the effector module comprises the amino acid sequence selected from the group consisting of SEQ ID NO. 412, 410, 418, 414, 416, 420, 422, 394, 398, 388, 390, 392, 396, 400, 402, 404, and 406.

59. The composition of claim 58, wherein the effector module comprises the amino acid sequence of SEQ ID NO. 412.

60. The composition of claim 58, wherein the effector module comprises the amino acid sequence of SEQ ID NO. 410.

61. The composition of claim 58, wherein the effector module comprises the amino acid sequence of SEQ ID NO. 414.

62. The composition of claim 58, wherein the effector module comprises the amino acid sequence of SEQ ID NO. 418.

63. The composition of claim 46, wherein the payload is a cytokine.

64. The composition of claim 63, wherein the cytokine is IL12.

65. The composition of claim 64, wherein the IL12 comprises a p40 subunit of SEQ ID NO. 111, appended to a p35 subunit of SEQ ID NO. 113.

66. The composition of claim 65, wherein the effector module comprises an amino acid sequence selected from the group consisting of SEQ ID NO. 240, 160, 242, 130, 133, 136, 139, 142, 145, 148, 151, 154, and 382.

67. The composition of claim 66, wherein the effector module comprises the amino acid sequence of SEQ ID NO. 240.

68. The composition of claim 66, wherein the effector module comprises the amino acid sequence of SEQ ID NO. 160.

69. The composition of claim 66, wherein the effector module comprises the amino acid sequence of SEQ ID NO. 242.

70. The composition of any of claims 63-69, further comprising a chimeric antigen receptor.

71. The composition of claim 70, wherein the CAR is a CD19 CAR.

72. The composition of any of claim 71, where the composition comprises an amino acid sequence selected from SEQ ID NO. 156, 373, 426, 375, 377, and 384.

73. The composition of claim 72 comprising SEQ ID NO. 156.

74. The composition of claim 72, comprising SEQ ID NO. 373.

75. The composition of claim 72, comprising SEQ ID NO. 426.

76. The composition of claim 39, wherein the payload is a cytokine-cytokine receptor fusion protein.

77. The composition of claim 76, wherein the cytokine-cytokine receptor fusion protein comprises the whole or a portion of IL15, fused to the whole or a portion of IL15Ra to produce a IL15-IL15Ra fusion protein.

78. The composition of claim 76, wherein the cytokine-cytokine receptor fusion protein comprises the amino acid sequence of SEQ ID NO. 246 and the amino acid sequence of SEQ ID NO. 248.

79. The composition of any one of claims 76-78, wherein the effector module comprises the amino acid sequence selected from the group consisting of SEQ ID NO. 252, 256, 268, 254, 258, 260, 262, 264, and 266.

80. The composition of claim 79, comprising SEQ ID NO. 252.

81. The composition of claim 79, comprising SEQ ID NO. 256.

82. The composition of claim 79, comprising SEQ ID NO. 268.

83. The composition of any of claims 39-82, wherein the SRE is responsive to one or more stimuli.

84. The composition of claim 83, wherein the stimulus is a small molecule, wherein the small molecule is selected from bazedoxifene, raloxifene 4-hydroxytamoxifen (4-OHT), fulvestrant, oremifene, lasofoxifene, clomifene, femarelle and ormeloxifene.

85. The composition of claim 84, wherein the small molecule is bazedoxifene.

86. The composition of claim 84, wherein the small molecule is raloxifene.

87. A polynucleotide encoding any of the compositions of claims 1-86.

88. A vector comprising a polynucleotide of claim 87.

89. A pharmaceutical composition comprising the composition of any one of claims 1-86 and a pharmaceutically acceptable excipient.

90. An immune cell for adoptive cell transfer (ACT) which expresses the compositions of any of claims 1-86, the pharmaceutical composition of claim 89, the polynucleotide of claim 87 and/or is transduced or transfected with the vector of claim 88.
